(19) United States
(12) Patent Application Publication
(10) Pub. No.: US 2022/0017634 A1
(43) Pub. Date: Jan. 20, 2022

(54) ENGINEERED BISPECIFIC PROTEINS

(71) Applicant: Denali Therapeutics Inc., South San Francisco, CA (US)

(72) Inventors: Gunasekaran Kannan, South San Francisco, CA (US); Do Jin Kim, South San Francisco, CA (US); Pascal Sanchez, South San Francisco, CA (US); Adam P. Silverman, South San Francisco, CA (US); Hai L. Tran, South San Francisco, CA (US)

(73) Assignee: Denali Therapeutics Inc., South San Francisco, CA (US)

(21) Appl. No.: 17/174,231

(22) Filed: Feb. 11, 2021

Related U.S. Application Data
(63) Continuation of application No. PCT/US2019/046705, filed on Aug. 15, 2019.
(60) Provisional application No. 62/765,095, filed on Aug. 16, 2018.

(51) Int. Cl. C07K 16/28; C07K 16/40; C07K 16/18

(52) U.S. Cl. CPC C07K 16/2881; C07K 16/40; C07K 16/18; C07K 2317/622; C07K 2317/55; C07K 2317/515; C07K 2317/526; C07K 2317/76; C07K 2317/94; C07K 2317/51

(57) ABSTRACT

In one aspect, bispecific proteins having the ability to specifically bind to two antigens, and having an Fc polypeptide that comprises a modified CH3 domain and specifically binds to a transferrin receptor, are provided.

### CROSS-REFERENCE TO RELATED APPLICATIONS

The present application is a continuation of International Application No. PCT/US2019/046705, filed Aug. 15, 2019, which claims priority to U.S. Provisional Patent Application No. 62/765,095, filed Aug. 16, 2018, the disclosures of which are incorporated herein by reference in their entirety for all purposes.

### SEQUENCE LISTING

The present application contains a Sequence Listing which has been submitted electronically in ASCII format and is hereby incorporated by reference in its entirety for all purposes. Said ASCII copy, created on Feb. 9, 2021, is named 102342-002210US-1235823_SL.txt and is 829,066 bytes in size.

### BACKGROUND

Transferrin receptor (TfR) is a carrier protein for transferrin that, among other functions, is needed for the import of iron into the cell and is regulated in response to intracellular iron concentration. Transferrin receptors are expressed on endothelia, including the endothelium of the blood-brain barrier, and are expressed at increased levels on various cancer cells and inflammatory cells. It is one of the receptors that mediates transcytosis of cognate ligands across the blood-brain barrier. Transferrin receptors can thus be desirable targets for introducing an agent into a cell for either endocytosis in the cell or transcytosis across the cell.

### BRIEF SUMMARY

In one aspect, bispecific proteins that contain a modified Fc polypeptide are provided. In some embodiments, a protein comprises:
(a) a first Fc polypeptide that is fused at the N-terminus to an Fd portion of a Fab that specifically binds to a first antigen;
(b) a second Fc polypeptide that is fused at the N-terminus to a single-chain variable fragment (scFv) that specifically binds to a second antigen, wherein the first and second Fc polypeptides form an Fc dimer; and
(c) a light chain polypeptide that pairs with the Fd portion to form the Fab that specifically binds to the first antigen;
wherein the first Fc polypeptide and/or the second Fc polypeptide comprises a modified CH2 domain or modified CH3 domain (e.g., any described herein) and specifically binds to a transferrin receptor.

In some embodiments, the first antigen and the second antigen are the same antigen. In some embodiments, the first antigen and the second antigen are different antigens.

In some embodiments, the second Fc polypeptide is fused to the scFv via a first linker. In some embodiments, the first linker has a length from 1 to 20 amino acids. In some embodiments, the first linker comprises a GGGGS (G₄S) linker, a GGGGSGGGGS ((G₄S)₂) linker, a GGGGSGGGGSGGGGS ((G₄S)₃) linker, or a GGGGSGGGGSGGGG ((G₄S)₂-G₄) linker.

In some embodiments, the scFv comprises a VL region and a VH region that are connected via a second linker, wherein the orientation of the scFv is VL region-second linker-VH region. In some embodiments, the scFv comprises a VL region and a VH region that are connected via a second linker, wherein the orientation of the scFv is VH region-second linker-VL region. In some embodiments, the second linker has a length from 10 to 25 amino acids. In some embodiments, the second linker comprises a (G₄S)₃ linker, a RTVAGGGGSGGGGS (RTVA(G₄S)₂) linker, a RTVAGGGGSGGGGSGGGGS (RTVA(G₄S)₃) linker, a ASTKGGGGSGGGGS (ASTK(G₄S)₂) linker, or a ASTKGGGGSGGGGSGGGGS (ASTK(G₄S)₃) linker. In some embodiments, the scFv comprises an interchain disulfide bridge. In some embodiments, the scFv comprises a cysteine at each of positions VH44 and VL100, according to Kabat variable domain numbering. In some embodiments, the scFv comprises a disulfide bond between the cysteines at positions VH44 and VL100.

In some embodiments, a protein comprises:
(a) a first polypeptide that is fused at the N-terminus to an Fd portion of a Fab that specifically binds to a first antigen, and is fused at the C-terminus to a heavy chain variable region or a light chain variable region of a Fab that specifically binds to a second antigen;
(b) a second Fc polypeptide that is fused at the N-terminus to an Fd portion of a Fab that specifically binds to a first antigen, and is fused at the C-terminus to the other of the first heavy chain variable region or first light chain variable region recited in (a),
wherein the heavy chain variable region and the light chain variable region together form an Fv fragment that specifically binds to the second antigen, and wherein the first and second Fc polypeptides form an Fc dimer; and
(c) a light chain polypeptide that pairs with each of the Fd portions recited in (a) and (b) to form a Fab that specifically binds to the first antigen;
wherein the first Fc polypeptide and/or the second Fc polypeptide comprises a modified CH2 domain or modified CH3 domain (e.g., any described herein) and specifically binds to a transferrin receptor.

In some embodiments, the first antigen and the second antigen are the same antigen. In some embodiments, the first antigen and the second antigen are different antigens.

In some embodiments, the first Fc polypeptide is fused to the heavy chain variable region of the Fv fragment and the second Fc polypeptide is fused to the light chain variable region of the Fv fragment. In some embodiments, the first Fc polypeptide is fused to the light chain variable region of the Fv fragment and the second Fc polypeptide is fused to the heavy chain variable region of the Fv fragment. In some embodiments, the Fd portions recited in (a) and (b) comprise identical sequences. In some embodiments, the first Fc polypeptide and/or the second Fc polypeptide is fused at the C-terminus to the heavy chain variable region or light chain variable region via a first linker. In some embodiments, the first linker has a length from 1 to 20 amino acids. In some embodiments, the first linker comprises a G₄S linker, a (G₄S)₂ linker, a (G₄S)₃ linker, or a (G₄S)₂-G₄ linker.

In some embodiments, a protein comprises:
(a) a first Fc polypeptide that is fused at the N-terminus to an Fd portion of a Fab that specifically binds to a first antigen;
(b) a second Fc polypeptide that is fused at the N-terminus to an Fd portion of a Fab that specifically binds to a first antigen, and wherein the first and second Fc polypeptides form an Fc dimer; and
(c) a light chain polypeptide that pairs with each of the Fd portions recited in (a) and (b) to form a Fab that specifically binds to the first antigen;
wherein the first Fc polypeptide and/or the second Fc polypeptide is fused at the C-terminus to an scFv that specifically binds to a second antigen; and
wherein the first Fc polypeptide and/or the second Fc polypeptide comprises a modified CH2 domain or modified CH3 domain (e.g., any described herein) and specifically binds to a transferrin receptor.

In some embodiments, the first antigen and the second antigen are the same antigen. In some embodiments, the first antigen and the second antigen are different antigens.

In some embodiments, the first Fc polypeptide is fused at the C-terminus to an scFv that specifically binds to the second antigen. In some embodiments, the second Fc polypeptide is fused at the C-terminus to an scFv that specifically binds to the second antigen. In some embodiments, each of the first Fc polypeptide and the second Fc polypeptide is fused at the C-terminus to an scFv that specifically binds to the second antigen. In some embodiments, the scFvs that are fused to the first Fc polypeptide and the second Fc polypeptide comprise identical sequences. In some embodiments, the first Fc polypeptide and/or the second Fc polypeptide is fused to the scFv via a first linker. In some embodiments, the first linker has a length from 1 to 20 amino acids. In some embodiments, the first linker comprises a G₄S linker, a (G₄S)₂ linker, a (G₄S)₃ linker, or a (G₄S)₂-G₄ linker.

In some embodiments, the scFv comprises a VL region and a VH region that are connected via a second linker, wherein the orientation of the scFv is VL region-second linker-VH region. In some embodiments, the scFv comprises a VL region and a VH region that are connected via a second linker, wherein the orientation of the scFv is VH region-second linker-VL region. In some embodiments, the second linker has a length from 10 to 25 amino acids. In some embodiments, the second linker comprises a (G₄S)₃ linker, an RTVA(G₄S)₂ linker, a RTVA(G₄S)₃ linker, an ASTK(G₄S)₂ linker, or a ASTK(G₄S)₃ linker. In some embodiments, the scFv comprises an interchain disulfide bridge. In some embodiments, the scFv comprises a cysteine at each of positions VH44 and VL100, according to Kabat variable domain numbering. In some embodiments, the scFv comprises a disulfide bond between the cysteines at positions VH44 and VL100. In some embodiments, the Fd portions recited in (a) and (b) comprise identical sequences.

In some embodiments, a protein as described herein comprises a first Fc polypeptide that comprises a modified CH3 domain and specifically binds to a transferrin receptor. In some embodiments, a protein as described herein comprises a second Fc polypeptide that comprises a modified CH3 domain and specifically binds to a transferrin receptor. In some embodiments, both the first Fc polypeptide and the second Fc polypeptide comprise a modified CH3 domain and specifically bind to a transferrin receptor.

In some embodiments, the first Fc polypeptide and/or the second Fc polypeptide comprises a modified CH3 domain that comprises one, two, three, four, five, six, seven, eight, nine, ten, or eleven substitutions in a set of amino acid positions comprising 380, 384, 386, 387, 388, 389, 390, 413, 415, 416, and 421, according to EU numbering. In some embodiments, the modified CH3 domain comprises Glu, Leu, Ser, Val, Trp, Tyr, or Gln at position 380; Leu, Tyr, Phe, Trp, Met, Pro, or Val at position 384; Leu, Thr, His, Pro, Asn, Val, or Phe at position 386; Val, Pro, Ile, or an acidic amino acid at position 387; Trp at position 388; an aliphatic amino acid, Gly, Ser, Thr, or Asn at position 389; Gly, His, Gln, Leu, Lys, Val, Phe, Ser, Ala, Asp, Glu, Asn, Arg, or Thr at position 390; an acidic amino acid, Ala, Ser, Leu, Thr, Pro, Ile, or His at position 413; Glu, Ser, Asp, Gly, Thr, Pro, Gln, or Arg at position 415; Thr, Arg, Asn, or an acidic amino acid at position 416; and/or an aromatic amino acid, His, or Lys at position 421, according to EU numbering.

In some embodiments, the first Fc polypeptide and/or the second Fc polypeptide comprises at least two substitutions at positions selected from the group consisting of 384, 386, 387, 388, 389, 390, 413, 416, and 421, according to EU numbering. In some embodiments, the first Fc polypeptide and/or the second Fc polypeptide includes substitutions for at least three, four, five, six, seven, eight, or nine of the positions. In some embodiments, the first Fc polypeptide and/or the second Fc polypeptide further comprises one, two, three, or four substitutions at positions comprising 380, 391, 392, and 415, according to EU numbering.

In some embodiments, the first Fc polypeptide and/or the second Fc polypeptide further comprises one, two, or three substitutions at positions comprising 414, 424, and 426, according to EU numbering.

In some embodiments, the first Fc polypeptide and/or the second Fc polypeptide comprises Trp at position 388.

In some embodiments, the first Fc polypeptide and/or the second Fc polypeptide comprises an aromatic amino acid at position 421. In some embodiments, the aromatic amino acid at position 421 is Trp or Phe.

In some embodiments, the first Fc polypeptide and/or the second Fc polypeptide comprises at least one position selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe. In some embodiments, the first Fc polypeptide and/or the second Fc polypeptide comprises 2, 3, 4, 5, 6, 7, 8, 9, 10, or 11 positions selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe. In some embodiments, the first Fc polypeptide and/or the second Fc polypeptide comprises 11 positions as follows: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

In some embodiments, the first Fc polypeptide and/or the second Fc polypeptide has a CH3 domain with at least 85% identity, at least 90% identity, or at least 95% identity to amino acids 111-217 of any one of SEQ ID NOs: 4-29, 101-164, and 239-252. In some embodiments, the first Fc polypeptide and/or the second Fc polypeptide comprises the amino acid sequence of any one of SEQ ID NOs:4-29, 101-164, and 239-252. In some embodiments, the residues for at least 5, 6, 7, 8, 9, 10, 11, 12, 13, 14, 15, or 16 of the positions corresponding to EU index positions 380, 384, 386, 387, 388, 389, 390, 391, 392, 413, 414, 415, 416, 421, 424 and 426 of any one of SEQ ID NOs:4-29, 101-164, and 239-252 are not deleted or substituted.

In some embodiments, the first Fc polypeptide and/or the second Fc polypeptide binds to the apical domain of the transferrin receptor. In some embodiments, the binding of the protein to the transferrin receptor does not substantially inhibit binding of transferrin to the transferrin receptor.

In some embodiments, the first Fc polypeptide and the second Fc polypeptide each contain one or more modifications that promote heterodimerization. In some embodiments, one of the Fc polypeptides has a T366W substitution and other Fc polypeptide has T366S, L368A, and Y407V substitutions, according to EU numbering. In some embodiments, the first Fc polypeptide contains the T366S, L368A, and Y407V substitutions and the second Fc polypeptide contains the T366W substitution. In some embodiments, the first Fc polypeptide contains the T366W substitution and the second Fc polypeptide contains the T366S, L368A, and Y407V substitutions.

In some embodiments, the first Fc polypeptide and/or the second Fc polypeptide comprises a native FcRn binding site. In some embodiments, the first Fc polypeptide and/or the second Fc polypeptide comprises a modification that alters FcRn binding. In some embodiments, the first Fc polypeptide and/or the second Fc polypeptide comprises one or more modifications that reduce effector function. In some embodiments, the modifications that reduce effector function are substitutions of Ala at position 234 and Ala at position 235, according to EU numbering. In some embodiments, both the first Fc polypeptide and the second Fc polypeptide comprise L234A and L235A substitutions.

In some embodiments, the first Fc polypeptide and/or the second Fc polypeptide comprises modifications relative to the native Fc sequence that extend serum half-life. In some embodiments, the modifications comprise substitutions of Tyr at position 252, Thr at position 254, and Glu at position 256, according to EU numbering. In some embodiments, the modifications comprise substitutions of Leu at position 428 and Ser at position 434, according to EU numbering. In some embodiments, the modifications comprise a substitution of Ser or Ala at position 434, according to EU numbering. In some embodiments, the first Fc polypeptide and/or the second Fc polypeptide comprises M428L and N434 S substitutions.

In some embodiments, the first Fc polypeptide and the second Fc polypeptide do not have effector function.

In some embodiments, the first Fc polypeptide and/or the second Fc polypeptide has an amino acid sequence identity of at least 75%, or at least 80%, 90%, 92%, or 95%, as compared to a corresponding wild-type Fc polypeptide. In some embodiments, the corresponding wild-type Fc polypeptide is a human IgG1, IgG2, IgG3, or IgG4 Fc polypeptide. In some embodiments, the first Fc polypeptide and/or and the second Fc polypeptide comprises an amino acid sequence of any one of SEQ ID NOs:165-238, 253-370, and 377-388.

In another aspect, pharmaceutical compositions are provided. In some embodiments, a pharmaceutical composition comprises a protein as disclosed herein and a pharmaceutically acceptable carrier.

In still another aspect, isolated polynucleotides are provided. In some embodiments, a polynucleotide comprises a nucleotide sequence encoding a protein as disclosed herein.

In yet another aspect, vectors and host cells are provided. In some embodiments, a vector comprises a polynucleotide comprises a nucleotide sequence encoding a protein as disclosed herein. In some embodiments, a host cell comprises a polynucleotide comprises a nucleotide sequence encoding a protein as disclosed herein.

In still another aspect, methods of treating a subject are provided. In some embodiments, the method comprises administering to the subject a protein or pharmaceutical composition as disclosed herein.

In another aspect, the present disclosure provides a non-human transgenic animal (e.g., a mammal) comprising (a) a nucleic acid that encodes a chimeric TfR polypeptide comprising: (i) an apical domain having at least 90% identity to SEQ ID NO:392 and (ii) the transferrin binding site of the native TfR polypeptide of the animal, and (b) a transgene of a mutant microtubule associated protein Tau (MAPT) gene, wherein the chimeric TfR polypeptide and/or the Tau protein is expressed in the brain of the animal.

In some embodiments, the apical domain comprises the amino acid sequence of SEQ ID NO:392. In some embodiments, the apical domain comprises the amino acid sequence of SEQ ID NO:393, SEQ ID NO:394, or SEQ ID NO:395.

In some embodiments, the chimeric TfR polypeptide comprises an amino acid sequence having at least 95% identity to SEQ ID NO:396.

In some embodiments, the animal expresses a level of the chimeric TfR polypeptide in brain, liver, kidney, or lung tissue within 20% (e.g., 18%, 16%, 14%, 12%, 10%, 8%, 6%, or 4%) of the level of expression of TfR in the same tissue of a corresponding wild-type animal of the same species.

In some embodiments, the animal comprises a red blood cell count, level of hemoglobin, or level of hematocrit within 20% (e.g., 18%, 16%, 14%, 12%, 10%, 8%, 6%, or 4%) of the red blood cell count, level of hemoglobin, or level of hematocrit in a corresponding wild-type animal of the same species.

In some embodiments, the nucleic acid sequence encoding the apical domain comprises a nucleic acid sequence having at least 95% (e.g., 97%. 98%, or 99%) identity to SEQ ID NO:397.

In some embodiments, the animal is homozygous or heterozygous for the nucleic acid encoding the chimeric TfR polypeptide.

In some embodiments, the nucleic acid that encodes the chimeric TfR polypeptide replaces the nucleic acid encoding the endogenous TfR polypeptide in the genome of the aminal, e.g., at the endogenous locus.

In some embodiments, the mutant MAPT gene encodes a mutant human Tau protein.

In some embodiments, the mutant human Tau protein comprises the amino acid substitution P272S relative to the sequence of SEQ ID NO:398.

In some embodiments, the animal is a rodent, such as a mouse or a rat.

### BRIEF DESCRIPTION OF THE DRAWINGS

FIG. 1. Exemplary engineered TfR-binding Fc polypeptide asymmetrically fused at the N-termini to a Fab and an scFv via flexible linkers. As shown, the Fab is fused to the knob half, which also comprises the TfR-binding mutations while the scFv is fused to the hole half, though the opposite orientation is possible.

FIG. 2. Exemplary engineered TfR-binding Fc polypeptide fused with N-terminal Fabs and variable domains fused to the C-termini of each Fc half via flexible linkers. As shown, VL is fused to the knob half, which also comprises the TfR-binding mutations while VH is fused to the hole half, though the opposite orientation is possible.

FIGS. 3A and 3B. Exemplary engineered TfR-binding Fc polypeptide fused with with N-terminal Fabs and a C-terminal scFv via a flexible linker. (A) Format where the scFv is fused to the hole half, whereas the knob half comprises the TfR-binding mutations, though the scFv could also be fused to the knob. (B) Format where an scFv is fused to each of the knob and hole C-termini.

FIGS. 4A-4F. Pharmacokinetic properties in mice of BACE1/Tau bispecific proteins comprising TfR-binding Fc polypeptides. BACE1/Tau bispecific proteins comprising TfR-binding Fc polypeptides and an anti-BACE1 control (Ab153) were dosed at 10 mg/kg in wild-type mice and their levels were monitored in plasma over 7 days. (A-B) Fc capture and detection were used to quantify the levels of antibodies in plasma (A) and the clearance value (CL) for each molecule was calculated (B). (D-F) To determine whether the BACE1 scFv and C-terminal Fv's remain intact in vivo, BACE1 (C) and Tau (E) affinity capture with Fc detection was used. Strong correlations between both the BACE1 (D) and Tau (F) antigen capture with the Fc detection indicated that the molecules are largely intact throughout the pharmacokinetic time course.

FIGS. 5A and 5B. Pharmacokinetic properties in mice of additional BACE1/Tau bispecific proteins comprising TfR-binding Fc polypeptides. BACE1/Tau bispecific proteins comprising TfR-binding Fc polypeptides and an anti-RSV negative control antibody (Ab122) were dosed at 10 mg/kg in wild-type mice and their levels were monitored in plasma over 7 days. Fc capture and detection were used to quantify the levels of antibodies in plasma (A) and the clearance value (CL) for each molecule was calculated (B). All BACE1/Tau bispecific proteins comprising TfR-binding Fc polypeptides had acceptable clearance values within 1.5-2 fold of the control antibody and within 1.5 fold of a control anti-Tau antibody comprising a TfR-binding Fc polypeptide.

FIGS. 6A and 6B. Pharmacokinetic properties of additional BACE1/Tau bispecific proteins comprising TfR-binding Fc polypeptides in human TfR knock-in (hTfR^{ms/hu} KI) mice. BACE1/Tau bispecific proteins comprising TfR-binding Fc polypeptides and an anti-RSV negative control antibody (Ab122) were dosed at 10 mg/kg in hTfR^{ms/hu} KI mice and their levels were monitored in plasma over 7 days. Fc capture and detection were used to quantify the levels of antibodies in plasma (A) and the clearance value (CL) for each molecule was calculated (B). All bispecific proteins exhibited faster clearance than control Ab122 or 1C7 due to TfR binding and target-mediated clearance. BACE1/Tau bispecific proteins comprising TfR-binding Fc polypeptides all had acceptable clearance values within 2-fold of a control anti-Tau antibody comprising a TfR-binding Fc polypeptide.

FIGS. 7A-7I. Pharmacokinetic properties of additional BACE1/Tau bispecific proteins comprising TfR-binding Fc polypeptides in PS19/hTfR^{ms/hu} KI mice. (A and B) Plasma and brain huIgG1 concentrations of constructs 28, 46, and 62 in PS19/TfR^{ms/hu} KI mice at various time points following a single 50 mg/kg intravenous injection of the indicated molecules. The missing points for later timepoints for certain molecules are due to values below the lower limit of quantification. (C) Brain-to-plasma ratios of constructs 28, 46, and 62 at 24 hrs post-dose and clearance values from data in (A) and (B). (D) Plasma huIgG1 concentrations of constructs 62 and 75-77. (E) Plasma clearance values of constructs 62 and 75-77 from data in (D). (F) Brain huIgG1 concentrations of constructs 62 and 75-77. (G) Brain huIgG1 concentrations of constructs 62 and 75-77 from data in (F). (H and I) Brain-to-plasma ratios of constructs 62 and 75-77 1 day post-dose in PS19/TfR^{ms/hu} KI mice following a single 50 mg/kg intravenous injection of the indicated molecules. All graphs represent mean±SEM, n=5 mice per group.

FIGS. 8A and 8B. Alternate architectures of BACE1/Tau bispecific proteins comprising TfR-binding Fc polypeptides reduce Aβ in a cell-based assay. (A) Human Aβ 40 was measured from media of CHO cells stably overexpressing human APP treated with the indicated antibodies for 24 hours. Incubation with all versions of 2H8 fused to Clone35.23.4:1C7-1C7 reduced human Aβ in a dose-dependent manner as compared to untreated control. Control IgG (Ab122) had no effect on Aβ reduction. Line graphs represent mean±SEM, n=2 independent experiments. (B) Cellular IC50 and maximum percent Aβ reduction compared to untreated controls from experiment in (A).

FIGS. 9A-9E. Quantification of Aβ40 in PS19/hTfR^{ms/hu} KI mice. (A and B) Brain and CSF Aβ40 of constructs 28, 46, and 62 in PS19/TfR^{ms/hu} KI mice at various time points following a single 50 mg/kg intravenous injection of the indicated molecules. (C) Maximum Aβ40 concentrations in CSF and brain from experiments in (A) and (B). (D) Brain Aβ40 of constructs 62 and 75-77 in PS19/TfR^{ms/hu} KI mice. (E) Percentage of brain Aβ40 reduction of constructs 62 and 75-77. All graphs represent mean±SEM, n=5 mice per group.

### DETAILED DESCRIPTION

### I. Introduction

We have developed several bispecific protein formats that contain a modified Fc polypeptide into which a non-endogenous TfR binding site has been engineered. As described herein, we have discovered that certain amino acids in an Fc region can be modified to generate a novel binding site specific for TfR in the Fc polypeptide. Taking advantage of the fact that TfR is highly-expressed on the blood-brain barrier (BBB) and that TfR naturally moves transferrin from the blood into the brain, modification of an Fc polypeptide to include a TfR binding site can promote the transport of the bispecific proteins across the BBB. This approach can substantially improve brain uptake of the bispecific proteins that specifically bind to two antigens, and is therefore highly useful for treating disorders and diseases where brain delivery is advantageous. In one example, bispecific proteins having the ability to specifically bind to two antigens, and having an Fc polypeptide that comprises a modified CH3 domain and specifically binds to a transferrin receptor, are provided.

As disclosed herein, the bispecific proteins described herein generally can be generated in a single cell without light chain mispairing or steering. These formats also allow for having the protein bind either target monovalently or bivalently. In some embodiments, the bispecific proteins bind to each target antigen monovalently. In some embodiments, the bispecific proteins bind to one target antigen monovalently and the other target antigen bivalently. In some embodiments, the bispecific proteins bind to each target antigen bivalently. In some embodiments, the first antigen and the second antigen are the same antigen. In some embodiments, the first antigen and the second antigen are different antigens.

### II. Definitions

As used herein, the singular forms “a,” “an,” and “the” include plural referents unless the content clearly dictates otherwise. Thus, for example, reference to “an antibody” optionally includes a combination of two or more such molecules, and the like.

As used herein, the terms “about” and “approximately,” when used to modify an amount specified in a numeric value or range indicate that the numeric value as well as reasonable deviations from the value known to the skilled person in the art, for example ±20%, ±10%, or ±5%, are within the intended meaning of the recited value.

As used herein, the term “antibody” refers to a protein with an immunoglobulin fold that specifically binds to an antigen via its variable regions. The term encompasses intact polyclonal antibodies, intact monoclonal antibodies, single chain antibodies, multi specific antibodies such as bispecific antibodies, monospecific antibodies, monovalent antibodies, chimeric antibodies, humanized antibodies, and human antibodies. The term “antibody,” as used herein, also includes antibody fragments that retain antigen-binding specificity, including but not limited to Fab, F(ab′)₂, Fv, scFv, and bivalent scFv. Antibodies can contain light chains that are classified as either kappa or lambda. Antibodies can contain heavy chains that are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains, respectively.

The term “variable region” or “variable domain” refers to a domain in an antibody heavy chain or light chain that is derived from a germline Variable (V) gene, Diversity (D) gene, or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The term “complementarity determining region” or “CDR” refers to the three hypervariable regions in each chain that interrupt the four framework regions established by the light and heavy chain variable regions. The CDRs are primarily responsible for antibody binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 or CDR-H3 is located in the variable region of the heavy chain of the antibody in which it is found, whereas a VL CDR1 or CDR-L1 is the CDR1 from the variable region of the light chain of the antibody in which it is found.

The “framework regions” or “FRs” of different light or heavy chains are relatively conserved within a species. The framework region of an antibody, that is the combined framework regions of the constituent light and heavy chains, serves to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of the CDRs and framework regions can be determined using various well known definitions in the art, e.g., Kabat, Chothia, international ImMunoGeneTics database (IMGT), AbM, and observed antigen contacts (“Contact”). In some embodiments, CDRs are determined according to the Contact definition. See, MacCallum et al., J. Mol. Biol., 262:732-745 (1996). In some embodiments, CDRs are determined by a combination of Kabat, Chothia, and/or Contact CDR definitions.

The term “Fd portion” refers to an N-terminal portion of an immunoglobulin heavy chain. Typically, an Fd portion includes the heavy chain variable (VH) region and a heavy chain constant (CH1) region.

The term “Fab” refers to an antigen-binding fragment consisting of a light chain variable region, a light chain constant region, a heavy chain variable region, and a heavy chain CH1 constant region.

The term “single-chain variable fragment” or “scFv” refers to an antigen-binding fragment consisting of a heavy chain variable region and a light chain variable region linked together via a peptide linker. An scFv lacks constant regions.

The term “Fv fragment” refers to an antigen-binding fragment consisting of a heavy chain variable region and a light chain variable region that together form a binding site for an antigen.

The term “epitope” refers to the area or region of an antigen to which a molecule, e.g., the CDRs of an antibody, specifically binds and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids (e.g., a linear epitope), or amino acids from different parts of the protein that are brought into proximity by protein folding (e.g., a discontinuous or conformational epitope).

As used herein, the phrase “recognizes an epitope,” as used with reference to an antibody, means that the antibody CDRs interact with or specifically bind to the antigen at that epitope or a portion of the antigen containing that epitope.

A “humanized antibody” is a chimeric immunoglobulin derived from a non-human source (e.g., murine) that contains minimal sequences derived from the non-human immunoglobulin outside the CDRs. In general, a humanized antibody will comprise at least one (e.g., two) variable domain(s), in which the CDR regions substantially correspond to those of the non-human immunoglobulin and the framework regions substantially correspond to those of a human immunoglobulin sequence. In some instances, certain framework region residues of a human immunoglobulin can be replaced with the corresponding residues from a non-human species to, e.g., improve specificity, affinity, and/or serum half-life. The humanized antibody can also comprise at least a portion of an immunoglobulin constant region (Fc), typically that of a human immunoglobulin sequence. Methods of antibody humanization are known in the art.

A “human antibody” or a “fully human antibody” is an antibody having human heavy chain and light chain sequences, typically derived from human germline genes. In some embodiments, the antibody is produced by a human cell, by a non-human animal that utilizes human antibody repertoires (e.g., transgenic mice that are genetically engineered to express human antibody sequences), or by phage display platforms.

The term “specifically binds” refers to a molecule (e.g., a Fab, an scFv, or a modified Fc polypeptide (or a target-binding portion thereof) that binds to an epitope or target with greater affinity, greater avidity, and/or greater duration to that epitope or target in a sample than it binds to another epitope or non-target compound (e.g., a structurally different antigen). In some embodiments, a Fab, scFv, or modified Fc polypeptide (or a target-binding portion thereof) that specifically binds to an epitope or target is a Fab, scFv, or modified Fc polypeptide (or a target-binding portion thereof) that binds to the epitope or target with at least 5-fold greater affinity than other epitopes or non-target compounds, e.g., at least 6-fold, 7-fold, 8-fold, 9-fold, 10-fold, 25-fold, 50-fold, 100-fold, 1000-fold, 10,000-fold, or greater affinity. The term “specific binding,” “specifically binds to,” or “is specific for” a particular epitope or target, as used herein, can be exhibited, for example, by a molecule having an equilibrium dissociation constant K_{D} for the epitope or target to which it binds of, e.g., 10⁻⁴ M or smaller, e.g., 10⁻⁵M, 10⁻⁶ M, 10⁻⁷ M, 10⁻⁸ M, 10⁻⁹ M, 10⁻¹⁰M, 10⁻¹¹ M, or 10⁻¹²M. It will be recognized by one of skill that a Fab or scFv that specifically binds to a target from one species may also specifically bind to orthologs of that target.

The term “binding affinity” is used herein to refer to the strength of a non-covalent interaction between two molecules, e.g., between a Fab or scFv and an antigen, or between a modified Fc polypeptide (or a target-binding portion thereof) and a target. Thus, for example, the term may refer to 1:1 interactions between a Fab or scFv and an antigen or between a modified Fc polypeptide (or a target-binding portion thereof) and a target, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet platform). As used herein, “binding affinity” includes not only formal binding affinities, such as those reflecting 1:1 interactions between a Fab or scFv and an antigen or between a modified Fc polypeptide (or a target-binding portion thereof) and a target, but also apparent affinities for which K_{D}'s are calculated that may reflect avid binding.

A “transferrin receptor” or “TfR,” as used herein, refers to transferrin receptor protein 1. The human transferrin receptor 1 polypeptide sequence is set forth in SEQ ID NO:100. Transferrin receptor protein 1 sequences from other species are also known (e.g., chimpanzee, accession number XP 003310238.1; rhesus monkey, NP 001244232.1; dog, NP 001003111.1; cattle, NP 001193506.1; mouse, NP 035768.1; rat, NP 073203.1; and chicken, NP 990587.1). The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region, but does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

As used herein, “FcRn” refers to the neonatal Fc receptor. Binding of Fc polypeptides to FcRn reduces clearance and increases serum half-life of the Fc polypeptide. The human FcRn protein is a heterodimer that is composed of a protein of about 50 kDa in size that is similar to a major histocompatibility (WIC) class I protein and a β2-microglobulin of about 15 kDa in size.

As used herein, an “FcRn binding site” refers to the region of an Fc polypeptide that binds to FcRn. In human IgG, the FcRn binding site, as numbered using the EU index, includes L251, M252, 1253, 5254, R255, T256, M428, H433, N434, H435, and Y436. These positions correspond to positions 21 to 26, 198, and 203 to 206 of SEQ ID NO:1.

As used herein, a “native FcRn binding site” refers to a region of an Fc polypeptide that binds to FcRn and that has the same amino acid sequence as the region of a naturally occurring Fc polypeptide that binds to FcRn.

As used herein, the terms “CH3 domain” and “CH2 domain” refer to immunoglobulin constant region domain polypeptides. For purposes of this application, a CH3 domain polypeptide refers to the segment of amino acids from about position 341 to about position 447 as numbered according to the EU numbering scheme, and a CH2 domain polypeptide refers to the segment of amino acids from about position 231 to about position 340 as numbered according to the EU numbering scheme and does not include hinge region sequences. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering website). CH2 and CH3 domains are part of the Fc region of an immunoglobulin. An Fc region refers to the segment of amino acids from about position 231 to about position 447 as numbered according to the EU numbering scheme, but as used herein, can include at least a part of a hinge region of an antibody. An illustrative hinge region sequence is the human IgG1 hinge sequence EPKSCDKTHTCPPCP (SEQ ID NO:376).

The terms “wild-type,” “native,” and “naturally occurring,” as used with reference to a CH3 or CH2 domain, refer to a domain that has a sequence that occurs in nature.

As used herein, the term “mutant,” as used with reference to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “isolated,” as used with reference to a nucleic acid or protein, denotes that the nucleic acid or protein is essentially free of other cellular components with which it is associated in the natural state. It is preferably in a homogeneous state. Purity and homogeneity are typically determined using analytical chemistry techniques such as electrophoresis (e.g., polyacrylamide gel electrophoresis) or chromatography (e.g., high performance liquid chromatography). In some embodiments, an isolated nucleic acid or protein is at least 85% pure, at least 90% pure, at least 95% pure, or at least 99% pure.

The term “amino acid” refers to naturally occurring and synthetic amino acids, as well as amino acid analogs and amino acid mimetics that function in a manner similar to the naturally occurring amino acids. Naturally occurring are those encoded by the genetic code, as well as those amino acids that are later modified, e.g., hydroxyproline, γ-carboxyglutamate and O-phosphoserine. Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

The term “linker,” as used herein, refers to a moiety that links (e.g., covalently links) two peptides or polypeptides (e.g., between an Fc polypeptide and an scFv) to connect or fuse the peptides or polypeptides. In some embodiments, a linker comprises a chemical linkage. In some embodiments, a linker comprises a peptide having a length of one or more amino acid residues. Suitable linkers for connecting or fusing peptides or polypeptides can be selected based on the properties of the linkers, such as the length, hydrophobicity, flexibility, rigidity, or cleavability of the linker.

The terms “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

The terms “conservative substitution” and “conservative mutation” refer to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region as measured using one a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:1, when the residue aligns with the amino acid in SEQ ID NO:1 when optimally aligned to SEQ ID NO:1. The polypeptide that is aligned to the reference sequence need not be the same length as the reference sequence.

The terms “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human.

The terms “treatment,” “treating,” and the like are used herein to generally mean obtaining a desired pharmacologic and/or physiologic effect. “Treating” or “treatment” may refer to any indicia of success in the treatment or amelioration of a disease, including any objective or subjective parameter such as abatement, remission, improvement in patient survival, increase in survival time or rate, diminishing of symptoms or making the disease more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “pharmaceutically acceptable excipient” refers to a non-active pharmaceutical ingredient that is biologically or pharmacologically compatible for use in humans or animals, such as, but not limited to a buffer, carrier, or preservative.

As used herein, a “therapeutic amount” or “therapeutically effective amount” of an agent is an amount of the agent (e.g., any of the proteins described herein) that treats a disease in a subject.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, a protein as described herein is administered intravenously.

### III. Architectures of Bispecific Proteins

In one aspect, bispecific proteins having the ability to specifically bind to two antigens are provided. In some embodiments, the bispecific protein comprises Fc polypeptides that are fused to an antigen-binding fragment (e.g., a Fab, Fv, or scFv) that specifically binds to a first antigen and to an antigen-binding fragment (e.g., a Fab, Fv, or scFv) that specifically binds to a second antigen. In some embodiments, one or both of the Fc polypeptides of the bispecific protein is a modified Fc polypeptide (e.g., modified to promote TfR binding and/or to enhance heterodimerization of the Fc polypeptides).

In some embodiments, the bispecific protein binds to the first antigen monovalently and binds to the second antigen monovalently. In some embodiments, the bispecific protein binds to the first antigen bivalently and binds to the second antigen monovalently. In some embodiments, the bispecific protein binds to the first antigen monovalently and binds to the second antigen bivalently. In some embodiments, the bispecific protein binds to the first antigen bivalently and binds to the second antigen bivalently.

### Fab-Fc Polypeptide/scFv-Fc Polypeptide

In some embodiments, a bispecific protein comprises Fc polypeptides that are fused to a portion of a Fab that specifically binds to a first antigen and an scFv that specifically binds to a second antigen. In some embodiments, the Fab and the scFv are fused at the N-termini of the Fc polypeptides. In some embodiments, the first antigen and the second antigen are the same antigen. In some embodiments, the first antigen and the second antigen are different antigens.

In some embodiments, the bispecific protein comprises:
(a) a first Fc polypeptide that is fused at the N-terminus to an Fd portion of a Fab that specifically binds to a first antigen;
(b) a second Fc polypeptide that is fused at the N-terminus to a single-chain variable fragment (scFv) that specifically binds to a second antigen, wherein the first and second Fc polypeptides form an Fc dimer; and
(c) a light chain polypeptide that pairs with the Fd portion to form the Fab that specifically binds to the first antigen;
wherein the first Fc polypeptide and/or the second Fc polypeptide comprises a modified CH2 or modified CH3 domain and specifically binds to TfR.

In some embodiments, the first Fc polypeptide comprises a modified CH3 domain and specifically binds to TfR. In some embodiments, the second Fc polypeptide comprises a modified CH3 domain and specifically binds to TfR. In some embodiments, both the first Fc polypeptide and the second Fc polypeptide comprise a modified CH3 domain and specifically bind to TfR. In some embodiments, the first Fc polypeptide and/or the second Fc polypeptide comprises one or more modifications that promote TfR binding and/or enhance heterodimerization. Modified Fc polypeptides are further described in Section IV below. In some embodiments, one of the Fc polypeptides is a native (i.e., wild-type) immunoglobulin heavy chain Fc polypeptide having the sequence of SEQ ID NO:1.

In some embodiments, the protein comprises a Fab that specifically binds to the first antigen. The Fab is formed from the pairing of the Fd portion of the Fab, which is fused to the N-terminus of the first Fc polypeptide, with the light chain.

In some embodiments, the second Fc polypeptide is fused at the N-terminus to the scFv via a first linker. In some embodiments, the first linker has a length from about 1 to about 50 amino acids, e.g., from about 1 to about 40, from about 1 to about 30, from about 1 to about 25, from about 1 to about 20, from about 1 to about 15, from about 1 to about 10, from about 2 to about 40, from about 2 to about 30, from about 2 to about 20, from about 2 to about 10, from about 5 to about 40, from about 5 to about 30, from about 5 to about 25, or from about 5 to about 20 amino acids. In some embodiments, the first linker has a length of about 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50 amino acids.

In some embodiments, the first linker comprises a flexible linker. In some embodiments, the first linker comprises a glycine-serine linker, i.e., a linker that consists primarily of, or entirely of, stretches of glycine and serine residues. In some embodiments, the first linker comprises a (G₄S)ₙ linker (GGGGS)ₙ, in which “n” indicates the number of repeats of the motif. In some embodiments, the first linker comprises a G₄S (GGGGS; SEQ ID NO:371) linker, a (G₄S)₂ (GGGGSGGGGS; SEQ ID NO:372) linker, a (G₄S)₃ (GGGGSGGGGSGGGGS; SEQ ID NO:373) linker, or a (G₄S)₂-G₄ (GGGGSGGGGSGGGG; SEQ ID NO:389) linker. In some embodiments, the first linker comprises a G₄S linker. In some embodiments, the first linker comprises a (G₄S)₂ linker. In some embodiments, the first linker comprises a (G₄S)₃ linker. In some embodiments, the first linker comprises a (G₄S)₂-G₄ linker.

In some embodiments, the scFv that specifically binds to the second antigen comprises a heavy chain variable (VH) region sequence and a light chain variable (VL) region sequence from an antibody or antibody fragment that specifically binds to the second antigen. In some embodiments, the orientation of the VL region and the VH region in the scFv that is fused to the second Fc polypeptide is VL region-VH region (i.e., the VL region is closer to the second Fc polypeptide than the VH region). In some embodiments, the orientation of the VL region and the VH region in the scFv that is fused to the second Fc polypeptide is VH region-VL region (i.e., the VH region is closer to the second Fc polypeptide than the VL region).

In some embodiments, the VL region and the VH region of the scFv are connected via a second linker. In some embodiments, the second linker has a length from about 10 to about 25 amino acids, e.g., from about 10 to about 20, from about 12 to about 25, from about 12 to about 20, from about 14 to about 25, or from about 14 to about 20 amino acids. In some embodiments, the second linker has a length of about 10, 11, 12, 13, 14, 15, 16, 17, 18, 19, 20, 21, 22, 23, 24, or 25 amino acids. In some embodiments, the second linker comprises a flexible linker. In some embodiments, the second linker comprises a glycine-serine linker, e.g., a (G₄S)ₙ linker. In some embodiments, the second linker comprises a (G₄S)₂ linker. In some embodiments, the second linker comprises a (G₄S)₃ linker. In some embodiments, the second linker comprises a (G₄S)₂-G₄ linker. In some embodiments, the second linker comprises a RTVA(G₄S)₂ (RTVAGGGGSGGGGS; SEQ ID NO:374) linker, a (RTVA(G₄S)₃) linker (RTVAGGGGSGGGGSGGGGS; SEQ ID NO:390), a ASTK(G₄S)₂ (ASTKGGGGSGGGGS; SEQ ID NO:375) linker, or a ASTK(G₄S)₃ (ASTKGGGGSGGGGSGGGGS; SEQ ID NO:391) linker.

In some embodiments, for the scFv that is fused to the second Fc polypeptide, the VL region and the VH region are connected via a second linker, wherein the orientation of the scFv is VL region-second linker-VH region (i.e., the VL region is closer to the second Fc polypeptide than the VH region). In some embodiments, the VL region and the VH region are connected via a second linker, wherein the orientation of the scFv is VH region-second linker-VL region (i.e., the VH region is closer to the second Fc polypeptide than the VL region).

In some embodiments, the scFv comprises one or more disulfide bridges between cysteine residues of the VH region and the VL region. In some embodiments, the scFv comprises a cysteine at each of positions VH44 and VL100, as numbered according to Kabat variable domain numbering. In some embodiments, the scFv comprises a disulfide bond between the cysteines at positions VH44 and VL100.

mAb/Fv

In some embodiments, a bispecific protein comprises Fc polypeptides that are fused at each N-terminus to a Fab that specifically binds to a first antigen and at the C-terminus to a heavy chain variable region or a light chain variable region of a Fab that specifically binds to a second antigen, thereby forming a protein that binds to the first antigen bivalently and to the second antigen monovalently. In some embodiments, the first antigen and the second antigen are the same antigen. In some embodiments, the first antigen and the second antigen are different antigens.

In some embodiments, the bispecific protein comprises:
(a) a first Fc polypeptide that is fused at the N-terminus to an Fd portion of a Fab that specifically binds to a first antigen, and is fused at the C-terminus to a heavy chain variable region or a light chain variable region of a Fab that specifically binds to a second antigen;
(b) a second Fc polypeptide that is fused at the N-terminus to an Fd portion of a Fab that specifically binds to a first antigen, and is fused at the C-terminus to the other of a heavy chain variable region or a light chain variable region recited in (a), wherein the heavy chain variable region and the light chain variable region together form an Fv fragment that specifically binds the second antigen, and wherein the first and second Fc polypeptides form an Fc dimer; and
(c) a light chain polypeptide that pairs with each of the Fd portions recited in (a) and (b) to form a Fab that specifically binds to the first antigen;
wherein the first Fc polypeptide and/or the second Fc polypeptide comprises a modified CH2 or modified CH3 domain and specifically binds to TfR.

In some embodiments, the first Fc polypeptide comprises a modified CH3 domain and specifically binds to TfR. In some embodiments, the second Fc polypeptide comprises a modified CH3 domain and specifically binds to TfR. In some embodiments, both the first Fc polypeptide and the second Fc polypeptide comprise a modified CH3 domain and specifically bind to TfR. In some embodiments, the first Fc polypeptide and/or the second Fc polypeptide comprises one or more modifications that promote TfR binding and/or enhance heterodimerization. Modified Fc polypeptides are further described in Section IV below. In some embodiments, one of the Fc polypeptides is a native (i.e., wild-type) immunoglobulin heavy chain Fc polypeptide having the sequence of SEQ ID NO:1.

In some embodiments, the Fd portion recited in (a) comprises identical heavy chain CDR sequences as the Fd portion recited in (b). In some embodiments, the Fd portion recited in (a) comprises an identical heavy chain variable region sequence as the Fd portion recited in (b). In some embodiments, the Fd portion recited in (a) has an identical sequence as the Fd portion recited in (b).

In some embodiments, the Fab that specifically binds to the first antigen that is formed from the pairing of the Fd portion recited in (a) with the light chain polypeptide recited in (c) is identical to the Fab that specifically binds to the first antigen that is formed from the pairing of the Fd portion recited in (b) with the light chain polypeptide recited in (c).

In some embodiments, the first Fc polypeptide is fused to the heavy chain variable region of the Fv fragment and the second Fc polypeptide is fused to the light chain variable region of the Fv fragment. In some embodiments, the first Fc polypeptide is fused to the light chain variable region of the Fv fragment and the second Fc polypeptide is fused to the heavy chain variable region of the Fv fragment.

In some embodiments, the first Fc polypeptide and/or the second Fc polypeptide is fused at the C-terminus to the heavy chain variable region or light chain variable region via a first linker. In some embodiments, the first linker has a length from about 1 to about 50 amino acids, e.g., from about 1 to about 40, from about 1 to about 30, from about 1 to about 25, from about 1 to about 20, from about 1 to about 15, from about 1 to about 10, from about 2 to about 40, from about 2 to about 30, from about 2 to about 20, from about 2 to about 10, from about 5 to about 40, from about 5 to about 30, from about 5 to about 25, or from about 5 to about 20 amino acids. In some embodiments, the first linker has a length of about 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50 amino acids. In some embodiments, the first linker on the first Fc polypeptide is the same as the first linker on the second Fc polypeptide.

In some embodiments, the first linker comprises a flexible linker. In some embodiments, the first linker comprises a glycine-serine linker, e.g., a (G₄S)ₙ linker such as a G₄S linker, a (G₄S)₂ linker, a (G₄S)₃ linker, or a (G₄S)₂-G₄ linker. In some embodiments, the first linker comprises a G₄S linker. In some embodiments, the first linker comprises a (G₄S)₂ linker. In some embodiments, the first linker comprises a (G₄S)₃ linker. In some embodiments, the first linker comprises a (G₄S)₂-G₄ linker.

mAb/scFv

In some embodiments, a bispecific protein comprises Fc polypeptides that are fused at each N-terminus to a Fab that specifically binds to a first antigen and are fused at the C-terminus of one or both Fc polypeptides to an scFv that specifically binds to a second antigen. In some embodiments, the first antigen and the second antigen are the same antigen. In some embodiments, the first antigen and the second antigen are different antigens.

In some embodiments, the bispecific protein comprises:
(a) a first Fc polypeptide that is fused at the N-terminus to an Fd portion of a Fab that specifically binds to a first antigen;
(b) a second Fc polypeptide that is fused at the N-terminus to an Fd portion of a Fab that specifically binds to a first antigen, wherein the first and Fc polypeptides form an Fc dimer; and
(c) a light chain polypeptide that pairs with each of the Fd portions recited in (a) and (b) to form a Fab that specifically binds to the first antigen;
wherein the first Fc polypeptide and/or the second Fc polypeptide is fused at the C-terminus to an scFv that specifically binds to a second antigen;
wherein the first Fc polypeptide and/or the second Fc polypeptide comprises a modified CH2 or modified CH3 domain and specifically binds to TfR.

In some embodiments, the first Fc polypeptide comprises a modified CH3 domain and specifically binds to TfR. In some embodiments, the second Fc polypeptide comprises a modified CH3 domain and specifically binds to TfR. In some embodiments, both the first Fc polypeptide and the second Fc polypeptide comprise a modified CH3 domain and specifically bind to TfR. In some embodiments, the first Fc polypeptide and/or the second Fc polypeptide comprises one or more modifications that promote TfR binding and/or enhance heterodimerization. Modified Fc polypeptides are further described in Section IV below. In some embodiments, one of the Fc polypeptides is a native (i.e., wild-type) immunoglobulin heavy chain Fc polypeptide having the sequence of SEQ ID NO:1.

In some embodiments, the Fd portion recited in (a) comprises identical heavy chain CDR sequences as the Fd portion recited in (b). In some embodiments, the Fd portion recited in (a) comprises an identical heavy chain variable region sequence as the Fd portion recited in (b). In some embodiments, the Fd portion recited in (a) has an identical sequence as the Fd portion recited in (b).

In some embodiments, the Fab that specifically binds to the first antigen that is formed from the pairing of the Fd portion recited in (a) with the light chain polypeptide recited in (c) is identical to the Fab that specifically binds to the first antigen that is formed from the pairing of the Fd portion recited in (b) with the light chain polypeptide recited in (c).

In some embodiments, the first Fc polypeptide is fused at the C-terminus to an scFv that specifically binds to the second antigen. In some embodiments, the second Fc polypeptide is fused at the C-terminus to an scFv that specifically binds to the second antigen. In some embodiments, each of the first Fc polypeptide and the second Fc polypeptide is fused at the C-terminus to an scFv that specifically binds to the second antigen. In some embodiments, the scFv that is fused to the first Fc polypeptide comprises an amino acid sequence that has at least 75% sequence identity (e.g., at least 80%, at least 85%, at least 90%, at least 91%, at least 92%, at least 93%, at least 94%, at least 95%, at least 96%, at least 97%, at least 98%, or at least 99% sequence identity) to the amino acid sequence of the scFv that is fused to the second Fc polypeptide. In some embodiments, the scFv that is fused to the first Fc polypeptide comprises identical CDRs (e.g., identical heavy chain CDRs and identical light chain CDRs) as the scFv that is fused to the second Fc polypeptide. In some embodiments, the scFv that is fused to the first Fc polypeptide comprises identical heavy chain variable region and light chain variable region sequences as the scFv that is fused to the second Fc polypeptide. In some embodiments, the scFv that is fused to the first Fc polypeptide has an identical amino acid sequence as the scFv that is fused to the second Fc polypeptide.

In some embodiments, the first Fc polypeptide and/or the second Fc polypeptide is fused to the scFv via a first linker. In some embodiments, the first linker has a length from about 1 to about 50 amino acids, e.g., from about 1 to about 40, from about 1 to about 30, from about 1 to about 25, from about 1 to about 20, from about 1 to about 15, from about 1 to about 10, from about 2 to about 40, from about 2 to about 30, from about 2 to about 20, from about 2 to about 10, from about 5 to about 40, from about 5 to about 30, from about 5 to about 25, or from about 5 to about 20 amino acids. In some embodiments, the first linker has a length of about 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50 amino acids.

In some embodiments, the first linker comprises a flexible linker. In some embodiments, the first linker comprises a glycine-serine linker, e.g., a (G₄S)ₙ linker such as a G₄S linker, a (G₄S)₂ linker, a (G₄S)₃ linker, or a (G₄S)₂-G₄ linker. In some embodiments, the first linker comprises a G₄S linker. In some embodiments, the first linker comprises a (G₄S)₂ linker. In some embodiments, the first linker comprises a (G₄S)₃ linker. In some embodiments, the first linker comprises a (G₄S)₂-G₄ linker.

In some embodiments, the scFv that is fused to the first Fc polypeptide and/or the second Fc polypeptide comprises a heavy chain variable (VH) region sequence and a light chain variable (VL) region sequence from an antibody or antibody fragment that specifically binds to the second antigen. In some embodiments, the orientation of the VL region and the VH region in the scFv that is fused to the first Fc polypeptide and/or the second Fc polypeptide is VL region-VH region (i.e., the VL region is closer to the second Fc polypeptide than the VH region). In some embodiments, the orientation of the VL region and the VH region in the scFv that is fused to the first Fc polypeptide and/or the second Fc polypeptide is VH region-VL region (i.e., the VH region is closer to the second Fc polypeptide than the VL region).

In some embodiments, the VL region and the VH region of the scFv are connected via a second linker. In some embodiments, the second linker has a length from about 10 to about 25 amino acids, e.g., from about 10 to about 20, from about 12 to about 25, from about 12 to about 20, from about 14 to about 25, or from about 14 to about 20 amino acids. In some embodiments, the second linker has a length of about 10, 11, 12, 13, 14, 15, 16, 17, 18, 19, 20, 21, 22, 23, 24, or 25 amino acids. In some embodiments, the second linker comprises a flexible linker. In some embodiments, the second linker comprises a glycine-serine linker, e.g., a (G₄S)ₙ linker. In some embodiments, the second linker comprises a (G₄S)₂ linker. In some embodiments, the second linker comprises a (G₄S)₃ linker. In some embodiments, the second linker comprises a (G₄S)₂-G₄ linker. In some embodiments, the second linker comprises a RTVA(G₄S)₂ linker, a RTVA(G₄S)₃ linker, a ASTK(G₄S)₂ linker, or a ASTK(G₄S)₃ linker.

In some embodiments, for the scFv that is fused to the first Fc polypeptide and/or the second Fc polypeptide, the VL region and the VH region are connected via a second linker, wherein the orientation of the scFv is VL region-second linker-VH region (i.e., the VL region is closer to the second Fc polypeptide than the VH region). In some embodiments, the VL region and the VH region are connected via a second linker, wherein the orientation of the scFv is VH region-second linker-VL region (i.e., the VH region is closer to the second Fc polypeptide than the VL region).

In some embodiments, the scFv that is fused to the first Fc polypeptide and/or the second Fc polypeptide comprises one or more disulfide bridges between cysteine residues of the VH region and the VL region. In some embodiments, the scFv comprises a cysteine at each of positions VH44 and VL100, as numbered according to Kabat variable domain numbering. In some embodiments, the scFv comprises a disulfide bond between the cysteines at positions VH44 and VL100.

For the bispecific proteins disclosed herein, methods for analyzing binding affinity, binding kinetics, and cross-reactivity are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g., KinExA®), flow cytometry, fluorescence-activated cell sorting (FACS), BioLayer interferometry (e.g., Octet® (FortéBio, Inc., Menlo Park, Calif.)), and Western blot analysis. In some embodiments, ELISA is used to determine binding affinity and/or cross-reactivity. Methods for performing ELISA assays are known in the art and are also described in the Example section below. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity

### IV. Modified Fc Polypeptides for Blood-Brain Barrier (Bbb) Receptor Binding

In some aspects, bispecific proteins that specifically bind to a first antigen and a second antigen are capable of being transported across the blood-brain barrier (BBB). Such a protein comprises a modified Fc polypeptide that binds to a BBB receptor. BBB receptors are expressed on BBB endothelia, as well as other cell and tissue types. In some embodiments, the BBB receptor is TfR.

In some embodiments, a bispecific protein comprises a first Fc polypeptide and optionally a second Fc polypeptide, each of which can be independently modified. In some embodiments, the modifications allow the bispecific protein to specifically bind to a transferrin receptor. The modifications can be introduced into specified sets of amino acids that are present at the surface of the CH3 or CH2 domain. In some embodiments, bispecific proteins comprising an Fc polypeptide comprising modified CH3 or CH2 domains specifically bind to an epitope in the apical domain of the transferrin receptor.

Amino acid residues designated in various Fc modifications, including those introduced in a modified Fc polypeptide that binds to a BBB receptor, e.g., TfR, are numbered herein using EU index numbering. Any Fc polypeptide, e.g., an IgG1, IgG2, IgG3, or IgG4 Fc polypeptide, may have modifications, e.g., amino acid substitutions, in one or more positions as described herein. One of skill understands that CH2 and CH3 domains of other immunoglobulin isotypes, e.g., IgM, IgA, IgE, IgD, etc. may be similarly modified by identifying the amino acids in those domains that correspond to the modifications described herein (e.g., the modifications in sets (i)-(vi) below). Modifications may also be made to corresponding domains from immunoglobulins from other species, e.g., non-human primates, monkey, mouse, rat, rabbit, dog, pig, chicken, and the like.

### TfR-Binding Fc Polypeptides Comprising Mutations in the CH3 Domain

In some embodiments, the domain that is modified for BBB receptor-binding activity is a human Ig CH3 domain, such as an IgG1 CH3 domain. The CH3 domain can be of any IgG subtype, i.e., from IgG1, IgG2, IgG3, or IgG4. In the context of IgG1 antibodies, a CH3 domain refers to the segment of amino acids from about position 341 to about position 447 as numbered according to the EU numbering scheme.

In some embodiments, a modified Fc polypeptide that specifically binds to TfR binds to the apical domain of TfR and may bind to TfR without blocking or otherwise inhibiting binding of transferrin to TfR. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2%, or 1%). Illustrative CH3 domain polypeptides that exhibit this binding specificity include polypeptides having amino acid substitutions at positions 384, 386, 387, 388, 389, 390, 413, 416, and 421, according to EU numbering.

CH3 Transferrin Receptor Binding Set (i): 384, 386, 387, 388, 389, 390, 413, 416, and 421

In some embodiments, a modified Fc polypeptide that specifically binds to TfR comprises at least one, at least two, at least three, or at least four, and typically five, six, seven, eight, or nine substitutions in a set of amino acid positions comprising 384, 386, 387, 388, 389, 390, 413, 416, and 421, according to EU numbering (“set i”). Illustrative substitutions that may be introduced at these positions are shown in Table 6.

In some embodiments, a modified Fc polypeptide that specifically binds to TfR comprises at least one position having a substitution as follows: Leu, Tyr, Met, or Val at position 384; Leu, Thr, His, or Pro at position 386; Val, Pro, or an acidic amino acid at position 387; an aromatic amino acid, e.g., Trp or Gly (e.g., Trp) at position 388; Val, Ser, or Ala at position 389; an acidic amino acid, Ala, Ser, Leu, Thr, or Pro at position 413; Thr or an acidic amino acid at position 416; or Trp, Tyr, His, or Phe at position 421. In some embodiments, a modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set. Thus, for example, Ile may be present at position 384, 386, and/or position 413. In some embodiments, the acidic amino acid at position one, two, or each of positions 387, 413, and 416 is Glu. In other embodiments, the acidic amino acid at one, two or each of positions 387, 413, and 416 is Asp. In some embodiments, two, three, four five, six, seven, or all eight of positions 384, 386, 387, 388, 389, 413, 416, and 421 have an amino acid substitution as specified in this paragraph.

In some embodiments, a modified Fc polypeptide having modifications in set (i) comprises a native Asn at position 390. In some embodiments, the modified Fc polypeptide comprises Gly, His, Gln, Leu, Lys, Val, Phe, Ser, Ala, or Asp at position 390. In some embodiments, the modified Fc polypeptide further comprises one, two, three, or four substitutions at positions comprising 380, 391, 392, and 415. In some embodiments, Trp, Tyr, Leu, or Gln may be present at position 380. In some embodiments, Ser, Thr, Gln, or Phe may be present at position 391. In some embodiments, Gln, Phe, or His may be present at position 392. In some embodiments, Glu may be present at position 415.

In certain embodiments, the modified Fc polypeptide comprises two, three, four, five, six, seven, eight nine, or ten positions selected from the following: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421. In some embodiments, the modified Fc polypeptide comprises all eleven positions as follows: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421.

In certain embodiments, the modified Fc polypeptide comprises Leu or Met at position 384; Leu, His, or Pro at position 386; Val at position 387; Trp at position 388; Val or Ala at position 389; Pro at position 413; Thr at position 416; and/or Trp at position 421. In some embodiments, the modified CH3 domain polypeptide further comprises Ser, Thr, Gln, or Phe at position 391. In some embodiments, a modified Fc polypeptide further comprises Trp, Tyr, Leu, or Gln at position 380 and/or Gln, Phe, or His at position 392. In some embodiments, Trp is present at position 380 and/or Gln is present at position 392. In some embodiments, a modified CH3 domain polypeptide does not have a Trp at position 380.

In other embodiments, a modified Fc polypeptide comprises Tyr at position 384; Thr at position 386; Glu or Val and position 387; Trp at position 388; Ser at position 389; Ser or Thr at position 413; Glu at position 416; and/or Phe at position 421. In some embodiments, the modified Fc polypeptide comprises a native Asn at position 390. In certain embodiments, the modified Fc polypeptide further comprises Trp, Tyr, Leu, or Gln at position 380; and/or Glu at position 415. In some embodiments, the modified Fc polypeptide further comprises Trp at position 380 and/or Glu at position 415.

In some embodiments, the modified Fc polypeptide comprises one or more of the following substitutions: Trp at position 380; Thr at position 386; Trp at position 388; Val at position 389; Ser or Thr at position 413; Glu at position 415; and/or Phe at position 421.

In additional embodiments, the modified Fc polypeptide further comprises one, two, or three positions selected from the following: position 414 is Lys, Arg, Gly, or Pro; position 424 is Ser, Thr, Glu, or Lys; and position 426 is Ser, Trp, or Gly.

In some embodiments, a modified Fc polypeptide that specifically binds to TfR has at least 70% identity, at least 75% identity, at least 80% identity, at least 85% identity, at least 90% identity, or at least 95% identity to amino acids 111-217 of any one of SEQ ID NOS:4-29, 101-164, and 239-252. In some embodiments, such a modified CH3 domain polypeptide comprises the amino acids at EU index positions 384-390 and/or 413-421 of any one of SEQ ID NOS:4-29, 101-164, and 239-252. In some embodiments, such a modified Fc polypeptide comprises the amino acids at EU index positions 380-390 and/or 413-421 of any one of SEQ ID NOS:4-29, 101-164, and 239-252. In some embodiments, a modified Fc polypeptide comprises the amino acids at EU index positions 380-392 and/or 413-426 of any one of SEQ ID NOS:4-29, 101-164, and 239-252.

In some embodiments, a modified Fc polypeptide that specifically binds to TfR has at least 70% identity, at least 75% identity, at least 80% identity, at least 85% identity, at least 90% identity, or at least 95% identity to amino acids 111-217 of SEQ ID NO:1, with the proviso that the percent identity does not include the set of positions 384, 386, 387, 388, 389, 390, 413, 416, and 421.

In some embodiments, a modified Fc polypeptide has at least 70% identity, at least 75% identity, at least 80% identity, at least 85% identity, at least 90% identity, or at least 95% identity to any one of SEQ ID NOS:4-29, 101-164, and 239-252, with the proviso that at least five, six, seven, eight, nine, ten, eleven, twelve, thirteen, fourteen, fifteen, or sixteen of the positions that correspond to positions 380, 384, 386, 387, 388, 389, 390, 391, 392, 413, 414, 415, 416, 421, 424, and 426 of any one of SEQ ID NOS:4-29, 101-164, and 239-252 are not deleted or substituted.

In some embodiments, the modified Fc polypeptide has at least 75% identity, at least 80% identity, at least 85% identity, at least 90% identity, or at least 95% identity to any one of SEQ ID NOS:4-29, 101-164, and 239-252 and also comprises at at least five, six, seven, eight, nine, ten, eleven, twelve, thirteen, fourteen, fifteen or sixteen of the positions as follows: Trp, Tyr, Leu, Gln, or Glu at position 380; Leu, Tyr, Met, or Val at position 384; Leu, Thr, His, or Pro at position 386; Val, Pro, or an acidic amino acid (e.g., Glu) at position 387; an aromatic amino acid, e.g., Trp, at position 388; Val, Ser, or Ala at position 389; Ser or Asn at position 390; Ser, Thr, Gln, or Phe at position 391; Gln, Phe, or His at position 392; an acidic amino acid, Ala, Ser, Leu, Thr, or Pro at position 413; Lys, Arg, Gly or Pro at position 414; Glu or Ser at position 415; Thr or an acidic amino acid at position 416; Trp, Tyr, His or Phe at position 421; Ser, Thr, Glu or Lys at position 424; and Ser, Trp, or Gly at position 426. In some embodiments, the modified Fc polypeptide has at least 75% identity, at least 80% identity, at least 85% identity, at least 90% identity, or at least 95% identity to any one of SEQ ID NOS:4-29, 101-164, and 239-252 and comprises a modified CH3 domain comprising Trp, Tyr, Leu, Gln, or Glu at position 380; Leu, Tyr, Met, or Val at position 384; Leu, Thr, His, or Pro at position 386; Val, Pro, or an acidic amino acid at position 387; an aromatic amino acid, e.g., Trp, at position 388; Val, Ser, or Ala at position 389; Ser or Asn at position 390; Ser, Thr, Gln, or Phe at position 391; Gln, Phe, or His at position 392; an acidic amino acid (e.g., Asp), Ala, Ser, Leu, Thr, or Pro at position 413; Lys, Arg, Gly or Pro at position 414; Glu or Ser at position 415; Thr or an acidic amino acid (e.g., Glu) at position 416; Trp, Tyr, His or Phe at position 421; Ser, Thr, Glu or Lys at position 424; and Ser, Trp, or Gly at position 426.

In some embodiments, a bispecific protein as disclosed herein comprises a modified Fc polypeptide that has at least 85% identity, at least 90% identity, or at least 95% identity to any one of SEQ ID NOs:4-29, 101-164, and 239-252, and comprises the following modifications in the CH3 domain: Glu at position 380; Tyr at position 384; Thr at position 386; Glu at position 387; Trp at position 388; Val or Ser at position 389; Asn at position 390; Ser, Thr, Gln, or Phe at position 391; Gln, Phe, or His at position 392; Asp, Ser, or Thr at position 413; Lys at position 414; Glu at position 415; Glu at position 416; Phe at position 421; Ser at position 424; and Ser at position 426.

CH3 Transferrin Receptor Binding Set (ii): 345, 346, 347, 349, 437, 438, 439, and 440

In some embodiments, a modified Fc polypeptide that specifically binds to TfR comprises at least one, at least two, at least three, or at least four, and typically five, six, seven, eight, or nine substitutions in a set of amino acid positions comprising 345, 346, 347, 349, 437, 438, 439, and 440, according to EU numbering (“set ii”). Illustrative substitutions that may be introduced at these positions are shown in Table 5.

In some embodiments, the modified Fc polypeptide comprises Gly at position 437; Phe at position 438; and/or Asp at position 213. In some embodiments, Glu is present at position 440. In certain embodiments, a modified CH3 domain polypeptide comprises at least one substitution at a position as follows: Phe or Ile at position 345; Asp, Glu, Gly, Ala, or Lys at position 346; Tyr, Met, Leu, Ile, or Asp at position 347; Thr or Ala at position 349; Gly at position 437; Phe at position 438; His Tyr, Ser, or Phe at position 439; or Asp at position 440. In some embodiments, two, three, four, five, six, seven, or all eight of positions 345, 346, 347, 349, 437, 438, 439, and 440 have a substitution as specified in this paragraph. In some embodiments, a modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, a modified Fc polypeptide that specifically binds to TfR has at least 70% identity, at least 75% identity, at least 80% identity, at least 85% identity, at least 90% identity, or at least 95% identity to amino acids 111-217 of any one of SEQ ID NOS:30-46. In some embodiments, such a modified Fc polypeptide comprises the amino acids at EU index positions 345-349 and/or 437-440 of any one of SEQ ID NOS:30-46.

In some embodiments, a modified Fc polypeptide that specifically binds to TfR has at least 70% identity, at least 75% identity, at least 80% identity, at least 85% identity, at least 90% identity, or at least 95% identity to amino acids 111-217 of SEQ ID NO:1, with the proviso that the percent identity does not include the set of positions 345, 346, 347, 349, 437, 438, 439, and 440, according to EU numbering. In some embodiments, the modified Fc polypeptide comprises the amino acids at EU index positions 345-349 and/or 437-440 as set forth in any one of SEQ ID NOS:30-46.

In some embodiments, a modified Fc polypeptide that specifically binds to TfR has at least 70% identity, at least 75% identity, at least 80% identity, at least 85% identity, at least 90% identity, or at least 95% identity to any one of SEQ ID NOS:30-46 and comprises a modified CH3 domain comprising Phe or Ile at position 345; Asp, Glu, Gly, Ala, or Lys at position 346; Tyr, Met, Leu, Ile, or Asp at position 347; Thr or Ala at position 349; Gly at position 437; Phe at position 438; His Tyr, Ser, or Phe at position 439; or Asp at position 440.

TfR-Binding Fc Polypeptides Comprising Mutations in the CH2 Domain

In some embodiments, the domain that is modified for BBB receptor-binding activity is a human Ig CH2 domain, such as an IgG CH2 domain. The CH2 domain can be of any IgG subtype, i.e., from IgG1, IgG2, IgG3, or IgG4. In the context of IgG1 antibodies, a CH2 domain refers to the segment of amino acids from about position 231 to about position 340 as numbered according to the EU numbering scheme.

As indicated above, sets of residues of a CH2 domain that can be modified in accordance with the invention are numbered according to EU numbering. Any CH2 domain, e.g., an IgG1, IgG2, IgG3, or IgG4 CH2 domain, may have modifications, e.g., amino acid substitutions, in one or more sets of residues that correspond to residues at the noted positions.

In one embodiment, a modified Fc polypeptide that specifically binds to TfR binds to an epitope in the apical domain of the transferrin receptor. The modified Fc polypeptide may bind to TfR without blocking or otherwise inhibiting binding of transferrin to the receptor. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2%, or 1%).

CH2 Transferrin Receptor Binding Set (iii): 274, 276, 283, 285, 286, 287, 288, 289, and 290

In some embodiments, a modified Fc polypeptide that specifically binds to TfR comprises at least one, at least two, at least three, or at least four, and typically five, six, seven, eight, or nine substitutions in a set of amino acid positions comprising 274, 276, 283, 285, 286, 287, 288, and 290, according to EU numbering (“set iii”). Illustrative substitutions that may be introduced at these positions are shown in Table 1.

In some embodiments, the modified Fc polypeptide comprises Glu at position 287 and/or Trp at position 288. In some embodiments, the modified Fc polypeptide comprises at least one substitution at a position as follows: Glu, Gly, Gln, Ser, Ala, Asn, Tyr, or Trp at position 274; Ile, Val, Asp, Glu, Thr, Ala, or Tyr at position 276; Asp, Pro, Met, Leu, Ala, Asn, or Phe at position 283; Arg, Ser, Ala, or Gly at position 285; Tyr, Trp, Arg, or Val at position 286; Glu at position 287; Trp or Tyr at position 288; Gln, Tyr, His, Ile, Phe, Val, or Asp at position 289; or Leu, Trp, Arg, Asn, Tyr, or Val at position 290. In some embodiments, two, three, four, five, six, seven, eight, or all nine of positions 274, 276, 283, 285, 286, 287, 288, and 290 have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide comprises Glu, Gly, Gln, Ser, Ala, Asn, or Tyr at position 274; Ile, Val, Asp, Glu, Thr, Ala, or Tyr at position 276; Asp, Pro, Met, Leu, Ala, or Asn at position 283; Arg, Ser, or Ala at position 285; Tyr, Trp, Arg, or Val at position 286; Glu at position 287; Trp at position 288; Gln, Tyr, His, Ile, Phe, or Val at position 289; and/or Leu, Trp, Arg, Asn, or Tyr at position 290. In some embodiments, the modified Fc polypeptide comprises Arg at position 285; Tyr or Trp at position 286; Glu at position 287; Trp at position 288; and/or Arg or Trp at position 290.

In some embodiments, a modified Fc polypeptide that specifically binds to TfR has at least 70% identity, at least 75% identity, at least 80% identity, at least 85% identity, at least 90% identity, or at least 95% identity to amino acids 1-110 of any one of SEQ ID NOS:47-62. In some embodiments, such a modified Fc polypeptide comprises the amino acids at EU index positions 374-276 and/or 283-290 of any one of SEQ ID NOS:47-62.

In some embodiments, a modified Fc polypeptide that specifically binds to TfR has at least 70% identity, at least 75% identity, at least 80% identity, at least 85% identity, at least 90% identity, or at least 95% identity to amino acids 1-110 of SEQ ID NO:1, with the proviso that the percent identity does not include the set of positions 274, 276, 283, 285, 286, 287, 288, and 290, according to EU numbering. In some embodiments, the modified CH2 domain polypeptide comprises the amino acids at EU index positions 374-276 and/or 283-290 as set forth in any one of SEQ ID NOS:47-62.

In additional embodiments, a modified Fc polypeptide that specifically binds to TfR has at least 70% identity, at least 75% identity, at least 80% identity, at least 85% identity, at least 90% identity, or at least 95% identity to any one of SEQ ID NOS:47-62 and comprises a modified CH2 domain comprising Glu, Gly, Gln, Ser, Ala, Asn, or Tyr at position 274; Ile, Val, Asp, Glu, Thr, Ala, or Tyr at position 276; Asp, Pro, Met, Leu, Ala, or Asn at position 283; Arg, Ser, or Ala at position 285; Tyr, Trp, Arg, or Val at position 286; Glu at position 287; Trp at position 288; Gln, Tyr, His, Ile, Phe, or Val at position 289; and/or Leu, Trp, Arg, Asn, or Tyr at position 290.

CH2 Transferrin Receptor Binding Set (iv): 274, 276, 283, 285, 286, 287, 288, 289, and 290

In some embodiments, a modified Fc polypeptide that specifically binds to TfR comprises at least one, at least two, at least three, or at least four, and typically five, six, seven, eight, nine, or ten substitutions in a set of amino acid positions comprising 266, 267, 268, 269, 270, 271, 295, 297, 298, and 299, according to EU numbering (“set iv”). Illustrative substitutions that may be introduced at these positions are shown in Table 2.

In some embodiments, a modified Fc polypeptide that specifically binds to TfR comprises Pro at position 270, Glu at position 295, and/or Tyr at position 297. In some embodiments, the modified Fc polypeptide comprises at least one substitution at a position as follows: Pro, Phe, Ala, Met, or Asp at position 266; Gln, Pro, Arg, Lys, Ala, Ile, Leu, Glu, Asp, or Tyr at position 267; Thr, Ser, Gly, Met, Val, Phe, Trp, or Leu at position 268; Pro, Val, Ala, Thr, or Asp at position 269; Pro, Val, or Phe at position 270; Trp, Gln, Thr, or Glu at position 271; Glu, Val, Thr, Leu, or Trp at position 295; Tyr, His, Val, or Asp at position 297; Thr, His, Gln, Arg, Asn, or Val at position 298; or Tyr, Asn, Asp, Ser, or Pro at position 299. In some embodiments, two, three, four, five, six, seven, eight, nine, or all ten of positions 266, 267, 268, 269, 270, 271, 295, 297, 298, and 299 have a substitution as specified in this paragraph. In some embodiments, a modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide comprises Pro, Phe, or Ala at position 266; Gln, Pro, Arg, Lys, Ala, or Ile at position 267; Thr, Ser, Gly, Met, Val, Phe, or Trp at position 268; Pro, Val, or Ala at position 269; Pro at position 270; Trp or Gln at position 271; Glu at position 295; Tyr at position 297; Thr, His, or Gln at position 298; and/or Tyr, Asn, Asp, or Ser at position 299.

In some embodiments, the modified Fc polypeptide comprises Met at position 266; Leu or Glu at position 267; Trp at position 268; Pro at position 269; Val at position 270; Thr at position 271; Val or Thr at position 295; His at position 197; His, Arg, or Asn at position 198; and/or Pro at position 299.

In some embodiments, the modified Fc polypeptide comprises Asp at position 266; Asp at position 267; Leu at position 268; Thr at position 269; Phe at position 270; Gln at position 271; Val or Leu at position 295; Val at position 297; Thr at position 298; and/or Pro at position 299.

In some embodiments, a modified Fc polypeptide that specifically binds to TfR has at least 70% identity, at least 75% identity, at least 80% identity, at least 85% identity, at least 90% identity, or at least 95% identity to amino acids 1-110 of any one of SEQ ID NOS:63-85. In some embodiments, such a modified Fc polypeptide comprises the amino acids at EU index positions 266-271 and/or 295-299 as set forth in any one of SEQ ID NOS:63-85.

In some embodiments, a modified Fc polypeptide that specifically binds to TfR has at least 70% identity, at least 75% identity, at least 80% identity, at least 85% identity, at least 90% identity, or at least 95% identity to amino acids 1-110 of SEQ ID NO:1, with the proviso that the percent identity does not include the set of positions 266, 267, 268, 269, 270, 271, 295, 297, 298, and 299, according to EU numbering. In some embodiments, the modified CH2 domain polypeptide comprises the amino acids at EU index positions 266-271 and/or 295-299 as set forth in any one of SEQ ID NOS:63-85.

In some embodiments, a modified Fc polypeptide that specifically binds to TfR comprises an amino acid sequence having at least 75% identity, at least 80% identity, at least 85% identity, at least 90% identity, or at least 95% identity to amino acids 39-72 of any one of SEQ ID NOS:63-85 and comprises a modified CH2 domain comprising Pro, Phe, or Ala at position 266; Gln, Pro, Arg, Lys, Ala, or Ile at position 267; Thr, Ser, Gly, Met, Val, Phe, or Trp at position 268; Pro, Val, or Ala at position 269; Pro at position 270; Trp or Gln at position 271; Glu at position 295; Tyr at position 297; Thr, His, or Gln at position 298; and/or Tyr, Asn, Asp, or Ser at position 299.

CH2 Transferrin Receptor Binding Set (v): 268, 269, 270, 271, 272, 292, 293, 294, 296, and 300

In some embodiments, a modified Fc polypeptide that specifically binds to TfR comprises at least one, at least two, at least three, or at least four, and typically five, six, seven, eight, nine, or ten substitutions in a set of amino acid positions comprising 268, 269, 270, 271, 272, 292, 293, 294, 296, and 300, according to EU numbering (“set v”). Illustrative substitutions that may be introduced at these positions are shown in Table 3.

In some embodiments, a modified Fc polypeptide that specifically binds to TfR comprises at least one substitution at a position as follows: Val or Asp at position 268; Pro, Met, or Asp at position 269; Pro or Trp at position 270; Arg, Trp, Glu, or Thr at position 271; Met, Tyr, or Trp at position 272; Leu or Trp at position 292; Thr, Val, Ile, or Lys at position 293; Ser, Lys, Ala, or Leu at position 294; His, Leu, or Pro at position 296; or Val or Trp at position 300. In some embodiments, two, three, four, five, six, seven, eight, nine, or all ten of positions 268, 269, 270, 271, 272, 292, 293, 294, and 300 have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide comprises Val at position 268; Pro at position 269; Pro at position 270; Arg or Trp at position 271; Met at position 272; Leu at position 292; Thr at position 293; Ser at position 294; His at position 296; and/or Val at position 300.

In some embodiments, the modified Fc polypeptide comprises Asp at position 268; Met or Asp at position 269; Trp at position 270; Glu or Thr at position 271; Tyr or Trp at position 272; Trp at position 292; Val, Ile, or Lys at position 293; Lys, Ala, or Leu at position 294; Leu or Pro at position 296; and/or Trp at position 300.

In some embodiments, a modified Fc polypeptide that specifically binds to TfR has at least 70% identity, at least 75% identity, at least 80% identity, at least 85% identity, at least 90% identity, or at least 95% identity to amino acids 1-110 of any one of SEQ ID NOS:86-90. In some embodiments, such a modified Fc polypeptide comprises the amino acids at EU index positions 268-272 and/or 292-300 as set forth in any one of SEQ ID NOS:86-90.

In some embodiments, a modified Fc polypeptide that specifically binds to TfR has at least 70% identity, at least 75% identity, at least 80% identity, at least 85% identity, at least 90% identity, or at least 95% identity to amino acids 1-110 of SEQ ID NO:1, with the proviso that the percent identity does not include the set of positions 268, 269, 270, 271, 272, 292, 293, 294, 296, and 300, according to EU numbering. In some embodiments, the modified CH2 domain polypeptide comprises the amino acids at EU index positions 268-272 and/or 292-300 as set forth in any one of SEQ ID NOS:86-90.

In some embodiments, a modified Fc polypeptide that specifically binds to TfR comprises a sequence having at least 75% identity, at least 80% identity, at least 85% identity, at least 90% identity, or at least 95% identity to amino acids 41-73 of any one of SEQ ID NOS:86-90 and comprises a modified CH2 domain comprising Val or Asp at position 268; Pro, Met, or Asp at position 269; Pro or Trp at position 270; Arg, Trp, Glu, or Thr at position 271; Met, Tyr, or Trp at position 272; Leu or Trp at position 292; Thr, Val, Ile, or Lys at position 293; Ser, Lys, Ala, or Leu at position 294; His, Leu, or Pro at position 296; or Val or Trp at position 300.

CH2 Transferrin Receptor Binding Set (vi): 272, 274, 276, 322, 324, 326, 329, 330, and 331

In some embodiments, a modified Fc polypeptide that specifically binds to TfR comprises at least one, at least two, at least three, or at least four, and typically five, six, seven, eight, or nine substitutions in a set of amino acid positions comprising 272, 274, 276, 322, 324, 326, 329, 330, and 331, according to EU numbering (“set vi”). Illustrative substitutions that may be introduced at these positions are shown in Table 4.

In some embodiments, a modified Fc polypeptide that specifically binds to TfR comprises Trp at position 330. In some embodiments, the modified Fc polypeptide comprises at least one substitution at a position as follows: Trp, Val, Ile, or Ala at position 272; Trp or Gly at position 274; Tyr, Arg, or Glu at position 276; Ser, Arg, or Gln at position 322; Val, Ser, or Phe at position 324; Ile, Ser, or Trp at position 326; Trp, Thr, Ser, Arg, or Asp at position 329; Trp at position 330; or Ser, Lys, Arg, or Val at position 331. In some embodiments, two, three, four, five, six, seven, eight, or all nine of positions 272, 274, 276, 322, 324, 326, 329, 330, and 331 have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide comprises two, three, four, five, six, seven, eight, or nine positions selected from the following: position 272 is Trp, Val, Ile, or Ala; position 274 is Trp or Gly; position 276 is Tyr, Arg, or Glu; position 322 is Ser, Arg, or Gln; position 324 is Val, Ser, or Phe; position 326 is Ile, Ser, or Trp; position 329 is Trp, Thr, Ser, Arg, or Asp; position 330 is Trp; and position 331 is Ser, Lys, Arg, or Val. In some embodiments, the modified Fc polypeptide comprises Val or Ile at position 272; Gly at position 274; Arg at position 276; Arg at position 322; Ser at position 324; Ser at position 326; Thr, Ser, or Arg at position 329; Trp at position 330; and/or Lys or Arg at position 331.

In some embodiments, a modified Fc polypeptide that specifically binds to TfR has at least 70% identity, at least 75% identity, at least 80% identity, at least 85% identity, at least 90% identity, or at least 95% identity to amino acids 1-110 of any one of SEQ ID NOS:91-95. In some embodiments, such a modified Fc polypeptide comprises the amino acids at EU index positions 272-276 and/or 322-331 as set forth in any one of SEQ ID NOS:91-95.

In some embodiments, a modified Fc polypeptide that specifically binds to TfR has at least 70% identity, at least 75% identity, at least 80% identity, at least 85% identity, at least 90% identity, or at least 95% identity to amino acids 4-113 of SEQ ID NO:1, with the proviso that the percent identity does not include the set of positions 272, 274, 276, 322, 324, 326, 329, 330, and 331, according to EU numbering. In some embodiments, the modified CH2 domain polypeptide comprises the amino acids at EU index positions 272-276 and/or 322-331 as set forth in any one of SEQ ID NOS:91-95.

In some embodiments, a transferrin receptor-binding polypeptide comprises an amino acid sequence having at least 75% identity, at least 80% identity, at least 85% identity, at least 90% identity, or at least 95% identity to any one of SEQ ID NOS:91-95 and comprises a modified CH2 domain comprising Trp, Val, Ile, or Ala at position 272; Trp or Gly at position 274; Tyr, Arg, or Glu at position 276; Ser, Arg, or Gln at position 322; Val, Ser, or Phe at position 324; Ile, Ser, or Trp at position 326; Trp, Thr, Ser, Arg, or Asp at position 329; Trp at position 330; or Ser, Lys, Arg, or Val at position 331.

### Additional Fc Polypeptide Modifications

In some embodiments, one or both Fc polypeptides contains one or more additional modifications. Non-limiting examples of other mutations that can be introduced into one or both Fc polypeptides include, e.g., mutations to increase serum stability and/or half-life, to modulate effector function, to influence glycosylation, to reduce immunogenicity in humans, and/or to provide for knob and hole heterodimerization of the Fc polypeptides.

In some embodiments, one or both Fc polypeptides has an amino acid sequence identity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a corresponding wild-type Fc polypeptide (e.g., a human IgG1, IgG2, IgG3, or IgG4 Fc polypeptide).

In some embodiments, the Fc polypeptides include knob and hole mutations to promote heterodimer formation and hinder homodimer formation. Generally, the modifications introduce a protuberance (“knob”) at the interface of a first polypeptide and a corresponding cavity (“hole”) in the interface of a second polypeptide, such that the protuberance can be positioned in the cavity so as to promote heterodimer formation and thus hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). In some embodiments, such additional mutations are at a position in the Fc polypeptide that does not have a negative effect on binding of the polypeptide to a BBB receptor, e.g., TfR.

In one illustrative embodiment of a knob and hole approach for dimerization, position 366 (numbered according to the EU numbering scheme) of one of the Fc polypeptides present in the bispecific antibody comprises a tryptophan in place of a native threonine. The other Fc polypeptide of the bispecific protein has a valine at position 407 (numbered according to the EU numbering scheme) in place of the native tyrosine. The other Fc polypeptide may further comprise a substitution in which the native threonine at position 366 (numbered according to the EU numbering scheme) is substituted with a serine and a native leucine at position 368 (numbered according to the EU numbering scheme) is substituted with an alanine. Thus, one of the Fc polypeptides of the bispecific protein has the T366W knob mutation and the other Fc polypeptide has the Y407V mutation, which is typically accompanied by the T366S and L368A hole mutations.

In some embodiments, one or both Fc polypeptides comprises a modification at one or more of positions 251, 252, 254, 255, 256, 307, 308, 309, 311, 312, 314, 385, 386, 387, 389, 428, 433, 434, or 436, according to the EU numbering scheme. In some embodiments, mutations are introduced into one, two, or three of positions 255, 254, and 256 according to EU numbering. In some embodiments, the mutations are M252Y, S254T, and T256E according to EU numbering. Thus, one or both Fc polypeptides may have M252Y, S254T, and T256E substitutions. In some embodiments, a modified Fc polypeptide further comprises mutations M252Y, S254T, and T256E. In some embodiments, mutations are introduced into one or two of positions 428 and 434 according to the EU numbering scheme. In some embodiments, the mutations are M428L and N434S (“LS”) according to EU numbering. In some embodiments, a modified Fc polypeptide further comprises mutation N434S with or without M428L. In some embodiments, a modified Fc polypeptide comprises a substitution at one, two or all three of positions T307, E380, and N434 according to EU numbering. In some embodiments, one or both Fc polypeptides comprises M428L and N434S substitutions. In some embodiments, one or both Fc polypeptides comprises an N434S or N434A substitution. In some embodiments, the mutations are T307Q and N434A. In some embodiments, a modified Fc polypeptide comprises mutations T307A, E380A, and 434A. In some embodiments, a modified Fc polypeptide comprises substitutions at positions T250 and M428 according to EU numbering. In some embodiments, a modified Fc polypeptide comprises mutations T250Q and M428L. In some embodiments, a modified Fc polypeptide comprises substitutions at positions M428 and N434 according to EU numbering. In some embodiments, a modified Fc polypeptide comprises substitutions M428L and N434S. In some embodiments, a modified Fc polypeptide comprises an N434S or N434A substitution. In some embodiments, an Fc polypeptide that comprises one or more modifications that promote binding to TfR does not comprise LS substitutions. In some embodiments, an Fc polypeptide that does not comprise one or more modifications that promote binding to TfR comprises LS substitutions. In some embodiments, an Fc polypeptide that comprises one or more modifications that promote binding to TfR does not comprise LS substitutions, and an Fc polypeptide that does not comprise one or more modifications that promote binding to TfR comprises LS substitutions. In some embodiments, both Fc polypeptides comprise LS substitutions.

In some embodiments, one or both Fc polypeptides may comprise modifications that reduce effector function, i.e., having a reduced ability to induce certain biological functions upon binding to an Fc receptor expressed on an effector cell that mediates the effector function. Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one or both Fc polypeptides may also be engineered to contain other modifications for heterodimerization, e.g., electrostatic engineering of contact residues within a CH3-CH3 interface that are naturally charged or hydrophobic patch modifications.

In some embodiments, one or both Fc polypeptides may include additional modifications that modulate effector function.

In some embodiments, one or both Fc polypeptides may comprise modifications that reduce or eliminate effector function. Illustrative Fc polypeptide mutations that reduce effector function include, but are not limited to, substitutions in a CH2 domain, e.g., at positions 234 and 235, according to the EU numbering scheme. For example, in some embodiments, one or both Fc polypeptides can comprise alanine residues at positions 234 and 235. Thus, one or both Fc polypeptides may have L234A and L235A (“LALA”) substitutions. In some embodiments, an Fc polypeptide that comprises one or more modifications that promote binding to TfR further comprises LALA substitutions. In some embodiments, an Fc polypeptide that does not comprise one or more modifications that promote binding to TfR comprises LALA substitutions. In some embodiments, both Fc polypeptides comprise LALA substitutions.

Additional Fc polypeptide mutations that modulate an effector function include, but are not limited to, one or more substitutions at positions 238, 265, 269, 270, 297, 327 and 329, according to the EU numbering scheme. Illustrative substitutions include the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331S, according to the EU numbering scheme. Multiple substitutions may also be present, e.g., L234A and L235A of a human IgG1 Fc region; L234A, L235A, and P329G of an IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme.

In some embodiments, one or both Fc polypeptides may comprise a modification that removes the C-terminal lysine from the Fc polypeptide. For example, for a polypeptide that comprises an Fc polypeptide that is fused at the C-terminus to an scFv or an Fv, in some embodiments the Fc polypeptide lacks a C-terminal lysine. In some embodiments, removal of a C-terminal lysine from the Fc polypeptide may reduce or prevent proteolytic cleavage of an scFv or Fv that is fused to the Fc polypeptide.

### Illustrative Modified Fc Polypeptides

By way of non-limiting example, one or both Fc polypeptides present in a bispecific protein as disclosed herein may comprise additional mutations including a knob mutation (e.g., T366W as numbered according to the EU numbering scheme), hole mutations (e.g., T366S, L368A, and Y407V as numbered according to the EU numbering scheme), mutations that modulate effector function (e.g., L234A, L235A, and/or P329G (e.g., L234A and L235A) as numbered according to the EU numbering scheme), and/or mutations that increase serum stability (e.g., (i) M252Y, S254T, and T256E as numbered according to the EU numbering scheme, or (ii) N434S with or without M428L as numbered with reference to EU numbering). In some embodiments, a bispecific protein comprise (i) a first Fc polypeptide that comprises one or more modifications that promote TfR binding and further comprises one or more additional modifications (e.g., a knob mutation, hole mutations, mutations that modulate effector function, and/or mutations that increase serum stability), and (ii) a second Fc polypeptide that comprises one or more modifications (e.g., mutations that promote TfR binding, a knob mutation, hole mutations, mutations that modulate effector function, and/or mutations that increase serum stability).

In some embodiments, the modified Fc polypeptide has at least 85% identity, at least 90% identity, or at least 95% identity to the sequence of any one of SEQ ID NOs:1, 4-95, or 101-388 and comprises a knob mutation (e.g., T366W as numbered with reference to EU numbering). In some embodiments, the modified Fc polypeptide comprises the sequence of any one of SEQ ID NOs:167, 179, 191, 203, 215, 227, 253, 265, 277, 289, or 383.

In some embodiments, the modified Fc polypeptide has at least 85% identity, at least 90% identity, or at least 95% identity to the sequence of any one of SEQ ID NOs:1, 4-95, or 101-388 and comprises a knob mutation (e.g., T366W as numbered with reference to EU numbering) and mutations that modulate effector function (e.g., L234A, L235A, and/or P329G (e.g., L234A and L235A) as numbered with reference to EU numbering). In some embodiments, the modified Fc polypeptide comprises the sequence of any one of SEQ ID NOs:168, 169, 180, 181, 192, 193, 204, 205, 216, 217, 228, 229, 254, 255, 266, 267, 278, 279, 290, 291, or 384.

In some embodiments, the modified Fc polypeptide has at least 85% identity, at least 90% identity, or at least 95% identity to the sequence of any one of SEQ ID NOs:1, 4-95, or 101-388 and comprises a knob mutation (e.g., T366W as numbered with reference to EU numbering) and mutations that increase serum half-life (e.g., M252Y, S254T, and T256E, or N434S with or without M428L, as numbered with reference to EU numbering). In some embodiments, the modified Fc polypeptide comprises the sequence of any one of SEQ ID NOs:170, 182, 194, 206, 218, 230, 256, 268, 280, 292, 302, 309, 316, 323, 330, 337, 344, 351, 358, 365, 385, or 387.

In some embodiments, the modified Fc polypeptide has at least 85% identity, at least 90% identity, or at least 95% identity to the sequence of any one of SEQ ID NOs:1, 4-95, or 101-388 and comprises a knob mutation (e.g., T366W as numbered with reference to EU numbering), mutations that modulate effector function (e.g., L234A, L235A, and/or P329G (e.g., L234A and L235A) as numbered with reference to EU numbering), and mutations that increase serum half-life (e.g., M252Y, S254T, and T256E, or N434S with or without M428L, as numbered with reference to EU numbering). In some embodiments, the modified Fc polypeptide comprises the sequence of any one of SEQ ID NOs:171, 172, 183, 184, 195, 196, 207, 208, 219, 220, 231, 232, 257, 258, 269, 270, 281, 282, 293, 294, 303, 304, 310, 311, 317, 318, 324, 325, 331, 332, 338, 339, 345, 346, 352, 353, 359, 360, 366, 367, 386, or 388.

In some embodiments, the modified Fc polypeptide has at least 85% identity, at least 90% identity, or at least 95% identity to the sequence of any one of SEQ ID NOs:1, 4-95, or 101-388 and comprises hole mutations (e.g., T366S, L368A, and Y407V as numbered with reference to EU numbering). In some embodiments, the modified Fc polypeptide comprises the sequence of any one of SEQ ID NOs:173, 185, 197, 209, 221, 233, 259, 271, 283, 295, or 377.

In some embodiments, the modified Fc polypeptide has at least 85% identity, at least 90% identity, or at least 95% identity to the sequence of any one of SEQ ID NOs:1, 4-95, or 101-388 and comprises hole mutations (e.g., T366S, L368A, and Y407V as numbered with reference to EU numbering) and mutations that modulate effector function (e.g., L234A, L235A, and/or P329G (e.g., L234A and L235A) as numbered with reference to EU numbering). In some embodiments, the modified Fc polypeptide comprises the sequence of any one of SEQ ID NOs:174, 175, 186, 187, 198, 199, 210, 211, 222, 223, 234, 235, 260, 261, 272, 273, 284, 285, 296, 297, or 378.

In some embodiments, the modified Fc polypeptide has at least 85% identity, at least 90% identity, or at least 95% identity to the sequence of any one of SEQ ID NOs:1, 4-95, or 101-388 and comprises hole mutations (e.g., T366S, L368A, and Y407V as numbered with reference to EU numbering) and mutations that increase serum half-life (e.g., M252Y, S254T, and T256E, or N434S with or without M428L, as numbered with reference to EU numbering). In some embodiments, the modified Fc polypeptide comprises the sequence of any one of SEQ ID NOs:176, 188, 200, 212, 224, 236, 262, 274, 286, 298, 305, 312, 319, 326, 333, 340, 347, 354, 361, 368, 379, or 381.

In some embodiments, the modified Fc polypeptide has at least 85% identity, at least 90% identity, or at least 95% identity to the sequence of any one of SEQ ID NOs:1, 4-95, or 101-388 and comprises hole mutations (e.g., T366S, L368A, and Y407V as numbered with reference to EU numbering), mutations that modulate effector function (e.g., L234A, L235A, and/or P329G (e.g., L234A and L235A) as numbered with reference to EU numbering), and mutations that increase serum half-life (e.g., M252Y, S254T, and T256E, or as numbered with reference to EU numbering). In some embodiments, the modified Fc polypeptide comprises the sequence of any one of SEQ ID NOs:177, 178, 189, 190, 201, 202, 213, 214, 225, 226, 237, 238, 263, 264, 275, 276, 287, 288, 299, 300, 306, 307, 313, 314, 320, 321, 327, 328, 334, 335, 341, 342, 348, 349, 355, 356, 362, 363, 369, 370, 380, or 382.

In some embodiments, a bispecific protein as disclosed herein (e.g., a bispecific protein having an architecture disclosed in Section III above) comprises (i) a first Fc polypeptide that comprises the TfR binding site of a clone having the sequence of any one of SEQ ID NOs:SEQ ID NOs:4-95, 101-164, and 239-252 and further comprises a knob mutation (e.g., T366W according to EU numbering), L234A and L235A mutations as numbered with reference to EU numbering, and optionally M428L and N434S mutations as numbered with reference to EU numbering; and (ii) a second Fc polypeptide that comprises hole mutations (e.g., T366S, L368A, and Y407V according to EU numbering) and L234A and L235A mutations as numbered with reference to EU numbering, and optionally, M428L and N434S mutations as numbered with reference to EU numbering. In some embodiments, the first Fc polypeptide comprises the TfR binding site of a clone having the sequence of SEQ ID NO:105, SEQ ID NO:145, or SEQ ID NO:146 and further comprises a knob mutation (e.g., T366W), L234A and L235A, and optionally M428L and N434S mutations. In some embodiments, the first Fc polypeptide comprises the amino acid sequence of any one of SEQ ID NOs:192, 204, 228, 316, 324, or 337. In some embodiments, the second Fc polypeptide comprises the amino acid sequence of SEQ ID NO:378 or SEQ ID NO:382.

### V. Preparation of Bispecific Proteins

For preparing a bispecific protein as described herein, many techniques known in the art can be used. In some embodiments, the genes encoding the heavy and light chains of an antibody of interest (e.g., an antibody that binds to a first antigen or an antibody that binds to a second antigen) can be cloned from a cell, e.g, from a hybridoma. Gene libraries encoding heavy and light chains of monoclonal antibodies can also be made from hybridoma or plasma cells. Alternatively, phage or yeast display technology can be used to identify antibodies and Fab fragments that specifically bind to selected antigens.

Bispecific proteins can be produced using any number of expression systems, including prokaryotic and eukaryotic expression systems. In some embodiments, the expression system is a mammalian cell expression system, such as a hybridoma, or a CHO cell expression system. Many such systems are widely available from commercial suppliers. In some embodiments, the polynucleotides encoding the polypeptides that comprise the bispecific protein may be expressed using a single vector, e.g., in a di-cistronic expression unit, or under the control of different promoters. In other embodiments, the polynucleotides encoding the polypeptides that comprise the bispecific protein may be expressed using separate vectors.

In some aspects, the disclosure provides isolated nucleic acids comprising a nucleic acid sequence encoding any of the polypeptides comprising bispecific proteins as described herein; vectors comprising such nucleic acids; and host cells into which the nucleic acids are introduced that are used to replicate the nucleic acids and/or to express the bispecific proteins.

In some embodiments, a polynucleotide (e.g., an isolated polynucleotide) comprises a nucleotide sequence encoding a polynucleotide comprising a polypeptide that comprises the bispecific protein as disclosed herein (e.g., as described in the Section III above). In some embodiments, a polynucleotide as described herein is operably linked to a heterologous nucleic acid, e.g., a heterologous promoter.

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

Suitable host cells for cloning or expressing a polynucleotide or vector as described herein include prokaryotic or eukaryotic cells. In some embodiments, the host cell is prokaryotic. In some embodiments, the host cell is eukaryotic, e.g., Chinese Hamster Ovary (CHO) cells or lymphoid cells. In some embodiments, the host cell is a human cell, e.g., a Human Embryonic Kidney (HEK) cell.

In another aspect, methods of making a bispecific protein as described herein are provided. In some embodiments, the method includes culturing a host cell as described herein (e.g., a host cell expressing a polynucleotide or vector as described herein) under conditions suitable for expression of the bispecific protein. In some embodiments, the bispecific protein is subsequently recovered from the host cell (or host cell culture medium). In some embodiments, the bispecific protein is purified, e.g., by chromatography.

### VI. Therapeutic Methods

In another aspect, therapeutic methods using bispecific proteins having the ability to specifically bind to two antigens as described herein are provided. In some embodiments, methods of treating a disease are provided. In some embodiments, methods of modulating one or more biological activities associated with a disease are provided.

In some embodiments, a bispecific protein comprising a first Fc polypeptide and/or a second Fc polypeptide comprises a modified CH3 domain and specifically binds to a transferrin receptor is used to transfer the bispecific protein across an endothelium, e.g., the blood-brain barrier, to be taken up by the brain.

In some embodiments, a bispecific protein as disclosed herein may be used to treat a neurological disorder such as a disease of the brain or central nervous system (CNS). Illustrative diseases include Alzheimer's Disease, Parkinson's disease, amyotrophic lateral sclerosis, frontotemporal dementia, vascular dementia, Lewy body dementia, Pick's disease, primary age-related tauopathy, or progressive supranuclear palsy. In some embodiments, the disease may be a tauopathy, a prion disease (such as bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, or a nervous system heterodegenerative disorders (such as Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, Friedreich's ataxia, Spinal muscular atrophy, and Unverricht-Lundborg syndrome). In some embodiments, the disease is stroke or multiple sclerosis. In some embodiments, the patient may be asymptomatic, but has a marker that is associated with the disease of the brain or CNS. In some embodiments, the use of a bispecific protein as disclosed herein in the manufacture of a medicament for treating a neurological disorder is provided.

In some embodiments, a bispecific protein as disclosed herein is used for the treatment of cancer. In certain embodiments, the cancer is a primary cancer of the CNS, such as glioma, glioblastoma multiforme, meningioma, astrocytoma, acoustic neuroma, chondroma, oligodendroglioma, medulloblastomas, ganglioglioma, Schwannoma, neurofibroma, neuroblastoma, or extradural, intramedullary or intradural tumors. In some embodiments, the cancer is a solid tumor, or in other embodiments, the cancer is a non-solid tumor. Solid-tumor cancers include tumors of the central nervous system, breast cancer, prostate cancer, skin cancer (including basal cell carcinoma, cell carcinoma, squamous cell carcinoma and melanoma), cervical cancer, uterine cancer, lung cancer, ovarian cancer, testicular cancer, thyroid cancer, astrocytoma, glioma, pancreatic cancer, mesotheliomas, gastric cancer, liver cancer, colon cancer, rectal cancer, renal cancer including nephroblastoma, bladder cancer, oesophageal cancer, cancer of the larynx, cancer of the parotid, cancer of the biliary tract, endometrial cancer, adenocarcinomas, small cell carcinomas, neuroblastomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas, and rhabdomyosarcomas. In some embodiments, the use of a bispecific protein as disclosed herein in the manufacture of a medicament for treating cancer is provided.

In some embodiments, a bispecific protein as disclosed herein may be used in the treatment of an autoimmune or inflammatory disease. Examples of such diseases include, but are not limited to, ankylosing spondylitis, arthritis, osteoarthritis, rheumatoid arthritis, psoriatic arthritis, asthma, scleroderma, stroke, atherosclerosis, Crohn's disease, colitis, ulcerative colitis, dermatitis, diverticulitis, fibrosis, idiopathic pulmonary fibrosis, fibromyalgia, hepatitis, irritable bowel syndrome (IBS), lupus, systemic lupus erythematous (SLE), nephritis, multiple sclerosis, and ulcerative colitis. In some embodiments, the use of a bispecific protein as disclosed herein in the manufacture of a medicament for treating an autoimmune or inflammatory disease is provided.

In some embodiments, a bispecific protein as disclosed herein may be used in the treatment of a cardiovascular disease, such as coronary artery disease, heart attack, abnormal heart rhythms or arrhythmias, heart failure, heart valve disease, congenital heart disease, heart muscle disease, cardiomyopathy, pericardial disease, aorta disease, marfan syndrome, vascular disease, and blood vessel disease. In some embodiments, the use of a bispecific protein as disclosed herein in the manufacture of a medicament for treating a cardiovascular disease is provided.

In some embodiments, the method further comprises administering to the subject one or more additional therapeutic agents. For example, in some embodiments for treating a disease of the brain or central nervous system, the method may comprise administering to the subject a neuroprotective agent, e.g., an anticholinergic agent, a dopaminergic agent, a glutamatergic agent, a histone deacetylase (HDAC) inhibitor, a cannabinoid, a caspase inhibitor, melatonin, an anti-inflammatory agent, a hormone (e.g., estrogen or progesterone), or a vitamin. In some embodiments, the method comprises administering to the subject an agent for use in treating a cognitive or behavioral symptom of a neurological disorder (e.g., an antidepressant, a dopamine agonist, or an anti-psychotic).

A bispecific protein as disclosed herein is administered to a subject at a therapeutically effective amount or dose. Illustrative dosages include a daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In some embodiments, a patient initially is given a low dose, which is then increased to an efficacious dosage tolerable to the patient. Determination of an effective amount is well within the capability of those skilled in the art.

In various embodiments, a bispecific protein as disclosed herein is administered parenterally. In some embodiments, the bispecific protein is administered intravenously. Intravenous administration can be by infusion, e.g., over a period of from about 10 to about 30 minutes, or over a period of at least 1 hour, 2 hours, or 3 hours. In some embodiments, the bispecific protein is administered as an intravenous bolus. Combinations of infusion and bolus administration may also be used.

In some parenteral embodiments, a bispecific protein is administered intraperiotneally, subcutaneously, intradermally, or intramuscularly. In some embodiments, the bispecific protein is administered intradermally or intramuscularly. In some embodiments, the bispecific protein is administered intrathecally, such as by epidural administration, or intracerebroventricularly.

In other embodiments, the bispecific protein may be administered orally, by pulmonary administration, intranasal administration, intraocular administration, or by topical administration. Pulmonary administration can also be employed, e.g., by use of an inhaler or nebulizer, and formulation with an aerosolizing agent.

### VII. Pharmaceutical Compositions and Kits

In another aspect, pharmaceutical compositions and kits comprising a bispecific protein having the ability to specifically bind to two antigens are provided. In some embodiments, the bispecific protein is a bispecific protein as described in Section III above.

### Pharmaceutical Compositions

In some embodiments, a pharmaceutical composition comprises a bispecific protein as described herein (e.g., a bispecific protein having the ability to specifically bind to two antigens) and further comprises one or more pharmaceutically acceptable carriers and/or excipients. Guidance for preparing formulations can be found in any number of handbooks for pharmaceutical preparation and formulation that are known to those of skill in the art.

A pharmaceutically acceptable carrier includes any solvents, dispersion media, or coatings that are physiologically compatible and that preferably does not interfere with or otherwise inhibit the activity of the active agent. Various pharmaceutically acceptable excipients are well-known in the art.

In some embodiments, the carrier is suitable for intravenous, intramuscular, oral, intraperitoneal, intrathecal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known in the art.

Pharmaceutical compositions can be manufactured in a manner that is known to those of skill in the art, e.g., by means of conventional mixing, dissolving, granulating, dragee-making, emulsifying, encapsulating, entrapping or lyophilizing processes. The methods and excipients disclosed herein are merely exemplary and are in no way limiting.

For oral administration, a bispecific protein as disclosed herein can be formulated by combining it with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

A bispecific protein as disclosed herein can be formulated for parenteral administration by injection, e.g., by bolus injection or continuous infusion. For injection, the bispecific protein can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some embodiments, bispecific proteins can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

In some embodiments, a bispecific protein as disclosed herein is prepared for delivery in a sustained-release, controlled release, extended-release, timed-release or delayed-release formulation, for example, in semi-permeable matrices of solid hydrophobic polymers containing the active agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients. Sustained-release delivery systems can, depending on their design, release the compounds over the course of hours or days, for instance, over 4, 6, 8, 10, 12, 16, 20, or 24 hours or more. Usually, sustained release formulations can be prepared using naturally occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions of the disclosure may vary depending on the particular use envisioned. The determination of the appropriate dosage or route of administration is well within the skill of one in the art. Suitable dosages are also described in Section VI above.

### Kits

In some embodiments, a kit comprising a bispecific protein as described herein (e.g., a bispecific protein having the ability to specifically bind to two antigens) for use according to a method disclosed herein are provided. In some embodiments, the kit is for use in treating a neurodegenerative disease, e.g., Alzheimer's disease. preventing or treating a neurological disorder such as a disease of the brain or central nervous system (CNS).

In some embodiments, the kit further comprises one or more additional therapeutic agents. For example, in some embodiments, the kit comprises a bispecific protein as described herein and further comprises one or more additional therapeutic agents for use in the treatment of a neurodegenerative disease. In some embodiments, the kit further comprises instructional materials containing directions (i.e., protocols) for the practice of the methods described herein (e.g., instructions for using the kit for treating a neurodegenerative disease). While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD-ROM), and the like. Such media may include addresses to internet sites that provide such instructional materials.

### VIII. Transgenic Animals

Further, the disclosure also provides non-human transgenic animals (e.g., a rodent such as a mouse or a rat) that comprise (a) a nucleic acid that encodes a chimeric TfR polypeptide comprising: (i) an apical domain having at least 90% identity to SEQ ID NO:392 and (ii) the transferrin binding site of the native TfR polypeptide of the animal, and (b) a transgene of a mutant microtubule associated protein Tau (MAPT) gene, e.g., wherein the chimeric TfR polypeptide and/or the Tau protein is expressed in the brain of the animal. The chimeric forms of the transferrin receptor include a non-human (e.g., mouse) mammalian transferrin binding site and an apical domain that is heterologous to the domain containing the transferrin binding site. These chimeric receptors can be expressed in transgenic animals, particularly where the transferrin binding site is derived from the transgenic animal species and where the apical domain is derived from a primate (e.g., human or monkey). The nucleic acid encoding the chimeric TfR polypeptide can be “knocked-in” to the genome of the aminal (e.g., at the endogenous locus), resulting in the animal expressing the chimeric TfR polypeptide in place of the endogenous TfR polypeptide. The chimeric TfR polypeptide can comprise an amino acid sequence having at least 95% (e.g., 97%, 98%, or 99%) identity to SEQ ID NO:396. Also described herein is a polynucleotide encoding a chimeric transferrin receptor that comprises a non-human mammalian transferrin binding site and an apical domain having an amino acid sequence at least 80%, 90%, 95%, or 98% identical to SEQ ID NO:392. The nucleic acid sequence encoding the apical domain can comprise a nucleic acid sequence having at least 95% (e.g., 97%, 98%, or 99%) identity to SEQ ID NO:397. The transgenic animal can be homozygous or heterozygous for the nucleic acid encoding the chimeric TfR polypeptide. Further, in some embodiments, the mutant MAPT gene encodes a mutant human Tau protein. For example, the mutant human Tau protein comprises the amino acid substitution P272S relative to the sequence of SEQ ID NO:398.

The disclosure also provides a non-human, for example, non-primate, transgenic animal (e.g., a rodent such as a mouse or a rat) expressing such chimeric TfRs and a transgene of a mutant microtubule associated protein Tau (MAPT) gene and the use of the non-human transgenic animal to screen for polypeptides that can cross the BBB by binding to human transferrin receptor (huTfR) in vivo. In some embodiments, the non-human transgenic animal contains a native transferrin receptor (such as a mouse transferrin receptor (mTfR)), in which the apical domain is replaced with an orthologous apical domain having an amino acid sequence at least 80%, 90%, 95%, or 98% identical to SEQ ID NO:392, thereby leaving the native transferrin binding site and the majority, e.g., at least 70%, or at least 75%, of the sequence encoding the transferrin receptor intact. This non-human transgenic animal thus maximally retains the transferrin-binding functionality of the endogenous transferrin receptor of the non-human animal, including the ability to maintain proper iron homeostasis as well as bind and transport transferrin. As a result, the transgenic animal is healthy and suitable for use in discovery and development of therapeutics for treating brain diseases.

### IX. Examples

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results. Efforts have been made to ensure accuracy with respect to numbers used (e.g., amounts, temperatures, etc.), but some experimental error and deviation may be present. The practice of the present invention will employ, unless otherwise indicated, conventional methods of protein chemistry, biochemistry, recombinant DNA techniques and pharmacology, within the skill of the art. Such techniques are explained fully in the literature. Additionally, it should be apparent to one of skill in the art that the methods for engineering as applied to certain libraries can also be applied to other libraries described herein.

### Example 1. Design and Characterization of Engineered Transferrin Receptor Binding Polypeptides

This example describes the design, generation, and characterization of polypeptides of the present invention. For the purposes of this example and comparing the amino acids that are the same in clone sequences, a “conserved” mutation is considered to be one that occurred in all of the identified clones (not a conservative amino acid substitution), while a “semi-conserved” mutation is one that occurs in >50% of clones.

Unless otherwise indicated, the positions of amino acid residues in this section are numbered based on EU index numbering for a human IgG1 wild-type Fc region.

### Design of Polypeptide Fc Region Domain Libraries

New molecular recognition was engineered into polypeptide Fc regions by selecting certain solvent exposed surface patches for modification, constructing surface display libraries in which the amino acid composition of the selected patch was altered by randomization and then screening the surface-displayed sequence variants for desired functionality using standard expression display techniques. As used herein, the term “randomization” includes partial randomization as well as sequence changes with pre-defined nucleotide or amino acid mixing ratios. Typical surface-exposed patches selected for randomization had areas between about 600 to 1500 Å², and comprised about 7 to 15 amino acids.

### Clone Registers

The following registers were designed and generated according to the methods described herein. As used herein, the term “register” refers to a series of surface-exposed amino acid residues that form a contiguous surface that can be altered (e.g., by the introduction of mutations into the peptide coding gene sequences to produce amino acid substitutions, insertions, and/or deletions at the positions listed in the registers).

CH2 Register A2—Set (iii)

The CH2A2 register (Table 1) included amino acid positions 274, 276, 283, 285, 286, 287, 288, 289, and 290, according to EU numbering. The CH2A2 register was designed to form a surface along a beta sheet, an adjacent turn, and a following loop. It is well removed from both the FcγR and FcRn binding sites.

CH2 Register C—Set (iv)

The CH2C register (Table 2) included amino acid positions 266, 267, 268, 269, 270, 271, 295, 2972, 298, and 299, according to EU numbering. The CH2C register utilizes solvent-exposed residues along a series of loops near the hinge and very close to the FcγR binding site of the CH2 region.

CH2 Register D—Set (v)

The CH2D register (Table 3) included amino acid positions 268, 269, 270, 271, 272, 292, 293, 294, 296, and 300, according to EU numbering. 41, 42, 43, 44, 45, 65, 66, 67, 69, and 73. The CH2D register, similar to CH2C, utilizes solvent-exposed residues along a series of loops at the top of the CH2 region, very close to the FcγR binding site. The CH2C and CH2D registers largely share one loop and differ in the second loop utilized for binding.

CH2 Register E—Set (vi)

The CH2E3 register (Table 4) included amino acid positions 272, 274, 276, 322, 324, 326, 329, 330, and 331, according to EU numbering. 45, 47, 49, 95, 97, 99, 102, 103, and 104. The CH2E3 register positions are also close to the FcγR binding site, but utilize solvent-exposed residues on beta sheets that are adjacent to the loops near the FcγR binding site, in addition to some of the loop residues.

CH3 Register B—Set (ii)

The CH3B register (Table 5) included amino acid positions 345, 346, 347, 349, 437, 438, 439, and 440, according to EU numbering. 118, 119, 120, 122, 210, 211, 212, and 213. The CH3B register is largely made up of solvent-exposed residues on two parallel beta sheets along with several less-structured residues near the C-terminus of the CH3 region. It is distant from the FcγR and FcRn binding sites.

CH3 Register C—Set (i)

The CH3C register (Table 6) included amino acid positions 384, 386, 387, 388, 389, 390, 413, 416, and 421, according to EU numbering. The CH3C register positions form a contiguous surface by including surface-exposed residues from two loops, both distant from the FcγR and FcRn binding sites.

TABLE 1
CH2A2 Register Positions and Mutations

| Sequence | Seq. | | | | | | | | | | | | |
|---|---|---|---|---|---|---|---|---|---|---|---|---|---|
| name | group | 274 | 275 | 276 | . . . | 283 | 284 | 285 | 286 | 287 | 288 | 289 | 290 |
| Wild- | n/a | K | F | N | . . . | E | V | H | N | A | K | T | K |
| type | | | | | | | | | | | | | |
| CH2A2.1 | 1 | E | F | I | . . . | D | V | R | Y | E | W | Q | L |
| CH2A2.2 | 1 | G | F | V | . . . | P | V | S | W | E | W | Y | W |
| CH2A2.3 | 1 | Q | F | D | . . . | M | V | R | R | E | W | H | R |
| CH2A2.4 | 1 | S | F | E | . . . | P | V | R | W | E | W | Q | W |
| CH2A2.5 | 1 | A | F | T | . . . | P | V | R | W | E | W | Q | N |
| CH2A2.6 | 1 | N | F | D | . . . | L | V | R | R | E | W | H | R |
| CH2A2.7 | 1 | Q | F | V | . . . | A | V | R | W | E | W | I | R |
| CH2A2.8 | 1 | E | F | I | . . . | E | V | A | W | E | W | F | W |
| CH2A2.9 | 1 | G | F | A | . . . | N | V | R | V | E | W | Q | Y |
| CH2A2.10 | 1 | G | F | V | . . . | E | V | R | R | E | W | V | R |
| CH2A2.11 | 1 | S | F | D | . . . | L | V | R | R | E | W | Q | R |
| CH2A2.12 | 1 | E | F | T | . . . | D | V | R | Y | E | W | Y | Y |
| CH2A2.13 | 1 | Q | F | T | . . . | D | V | R | Y | E | W | V | R |
| CH2A2.14 | 1 | Q | F | Y | . . . | N | V | R | R | E | W | H | R |
| CH2A2.15 | 1 | Y | F | D | . . . | M | V | R | R | E | W | H | R |
| CH2A2.I6 | 2 | W | F | E | . . . | F | V | G | V | A | Y | D | V |

TABLE 2
CH2C Register Positions and Mutations

| Se- | Seq. | | | | | | | | | | | | |
|---|---|---|---|---|---|---|---|---|---|---|---|---|---|
| quence | | | | | | | | | | | | | |
| name | group | 266 | 267 | 268 | 269 | 270 | 271 | . . . | 295 | 296 | 297 | 298 | 299 |
| Wild- | n/a | V | S | H | E | D | P | . . . | Q | Y | N | S | T |
| type | | | | | | | | | | | | | |
| CH2C.1 | 1 | P | Q | T | P | P | W | . . . | E | Y | Y | T | Y |
| CH2C.2 | 1 | P | P | S | P | P | W | . . . | E | Y | Y | S | N |
| CH2C.3 | 1 | P | Q | T | P | P | W | . . . | E | Y | Y | S | N |
| CH2C.4 | 1 | F | R | G | P | P | W | . . . | E | Y | Y | H | D |
| CH2C.5 | 1 | P | Q | T | V | P | W | . . . | E | Y | Y | S | N |
| CH2C.6 | 1 | P | K | M | P | P | W | . . . | E | Y | Y | T | Y |
| CH2C.7 | 1 | P | P | V | P | P | W | . . . | E | Y | Y | S | N |
| CH2C.8 | 1 | P | A | F | P | P | W | . . . | E | Y | Y | Q | N |
| CH2C.9 | 1 | A | I | W | P | P | W | . . . | E | Y | Y | S | N |
| CH2C.10 | 1 | P | P | V | A | P | W | . . . | E | Y | Y | S | S |
| CH2C.11 | 1 | P | Q | M | P | P | Q | . . . | E | Y | Y | S | N |
| CH2C.12 | 1 | P | Q | T | A | P | W | . . . | E | Y | Y | T | Y |
| CH2C.13 | 1 | P | Q | T | P | P | Q | . . . | E | Y | Y | S | N |
| CH2C.14 | 1 | P | Q | T | P | P | W | . . . | E | Y | Y | T | Y |
| CH2C.15 | 1 | P | R | V | P | P | W | . . . | E | Y | Y | Q | N |
| CH2C.16 | 1 | P | S | V | P | P | W | . . . | E | Y | Y | S | N |
| CH2C.17 | 2 | M | L | W | P | V | P | . . . | V | Y | H | R | P |
| CH2C.18 | 2 | M | L | W | P | V | P | . . . | T | Y | H | N | P |
| CH2C.19 | 2 | M | E | W | P | V | T | . . . | T | Y | H | H | P |
| CH2C.20 | 2 | M | L | W | P | V | P | . . . | T | Y | H | H | P |
| CH2C.21 | 3 | D | D | L | T | F | Q | . . . | V | Y | V | T | P |
| CH2C.22 | 3 | D | D | L | T | F | Q | . . . | L | Y | V | T | P |
| CH2C.23 | 4 | A | Y | G | D | P | E | . . . | W | Y | D | V | P |

TABLE 3
CH2D Register Positions and Mutations

| Sequence | Seq. | | | | | | | | | | | | | | | |
|---|---|---|---|---|---|---|---|---|---|---|---|---|---|---|---|---|
| name | group | 268 | 269 | 270 | 271 | 272 | . . . | 292 | 293 | 294 | 295 | 296 | 297 | 298 | 299 | 300 |
| Wild- | n/a | H | E | D | P | E | . . . | R | E | E | Q | Y | N | S | T | Y |
| type | | | | | | | | | | | | | | | | |
| CH2D.1 | 1 | V | P | P | R | M | . . . | L | T | S | Q | H | N | S | T | V |
| CH2D.2 | 1 | V | P | P | W | M | . . . | L | T | S | Q | H | N | S | T | V |
| CH2D.3 | 2 | D | M | W | E | Y | . . . | W | V | K | Q | L | N | S | T | W |
| CH2D.4 | 2 | D | D | W | T | W | . . . | W | I | A | Q | P | N | S | T | W |
| CH2D.5 | 2 | D | D | W | E | W | . . . | W | K | L | Q | L | N | S | T | W |

TABLE 4
CH2E3 Register Positions and Mutations

| Se- | | | | | | | | | | | | | | | | | |
|---|---|---|---|---|---|---|---|---|---|---|---|---|---|---|---|---|---|
| quence | Seq. | | | | | | | | | | | | | | | | |
| name | group | 272 | 273 | 274 | 275 | 276 | . . . | 322 | 323 | 324 | 325 | 326 | 327 | 328 | 329 | 330 | 331 |
| Wild- | n/a | E | V | K | F | N | . . . | K | V | S | N | K | A | L | P | A | P |
| type | | | | | | | | | | | | | | | | | |
| CH2E3.1 | 1 | W | V | W | F | Y | . . . | S | V | V | N | I | A | L | W | W | S |
| CH2E3.2 | 2 | V | V | G | F | R | . . . | R | V | S | N | S | A | L | T | W | K |
| CH2E3.3 | 2 | V | V | G | F | R | . . . | R | V | S | N | S | A | L | S | W | R |
| CH2E3.4 | 2 | I | V | G | F | R | . . . | R | V | S | N | S | A | L | R | W | R |
| CH2E3.5 | 3 | A | V | G | F | E | . . . | Q | V | F | N | W | A | L | D | W | V |

TABLE 5
CH3B Register Positions and Mutations

| Sequence | Seq. | | | | | | | | | | |
|---|---|---|---|---|---|---|---|---|---|---|---|
| name | group | 345 | 346 | 347 | 348 | 349 | . . . | 437 | 438 | 439 | 440 |
| Wild- | n/a | E | P | Q | V | Y | . . . | T | Q | K | S |
| type | | | | | | | | | | | |
| CH3B.1 | 1 | F | D | Y | V | T | . . . | G | F | H | D |
| CH3B.2 | 1 | F | D | M | V | T | . . . | G | F | H | D |
| CH3B.3 | 1 | F | E | Y | V | T | . . . | G | F | H | D |
| CH3B.4 | 1 | F | E | M | V | T | . . . | G | F | H | D |
| CH3B.5 | 1 | F | E | L | V | T | . . . | G | F | H | D |
| CH3B.6 | 1 | F | E | I | V | T | . . . | G | F | H | D |
| CH3B.7 | 1 | F | D | I | V | T | . . . | G | F | H | D |
| CH3B.8 | 1 | F | D | Y | V | T | . . . | G | F | H | D |
| CH3B.9 | 1 | F | G | M | V | T | . . . | G | F | H | D |
| CH3B.10 | 1 | F | A | D | V | T | . . . | G | F | Y | D |
| CH3B.11 | 1 | F | G | L | V | T | . . . | G | F | H | D |
| CH3B.12 | 1 | F | D | Y | V | T | . . . | G | F | S | D |
| CH3B.13 | 1 | 1 | D | Y | V | T | . . . | G | F | S | D |
| CH3B.14 | 1 | F | K | D | V | T | . . . | G | F | F | D |
| CH3B.15 | 1 | F | D | L | V | T | . . . | G | F | Y | D |
| CH3B.16 | 1 | 1 | D | Y | V | T | . . . | G | F | S | D |
| CH3B.17 | 1 | F | E | L | V | A | . . . | G | F | H | D |

TABLE 6
CH3C Register Positions and Mutations

| Se- | | | | | | | | | | | | | | | | | | | |
|---|---|---|---|---|---|---|---|---|---|---|---|---|---|---|---|---|---|---|---|
| quence | Seq. | | | | | | | | | | | | | | | | | | |
| name | group | 384 | 385 | 386 | 387 | 388 | 389 | 390 | 391 | . . . | 413 | 414 | 415 | 416 | 417 | 418 | 419 | 420 | 421 |
| Wild- | n/a | N | G | Q | P | E | N | N | Y | . . . | D | K | S | R | W | Q | Q | G | N |
| type | | | | | | | | | | | | | | | | | | | |
| CH3C.1 | | L | G | L | V | W | V | G | Y | . . . | A | K | S | T | W | Q | Q | G | W |
| CH3C.2 | | Y | G | T | V | W | S | H | Y | . . . | S | K | S | E | W | Q | Q | G | Y |
| CH3C.3 | | Y | G | T | E | W | S | Q | Y | . . . | E | K | S | D | W | Q | Q | G | H |
| CH3C.4 | | V | G | T | P | W | A | L | Y | . . . | L | K | S | E | W | Q | Q | G | W |
| CH3C. | 2 | Y | G | T | V | W | S | K | Y | . . . | S | K | S | E | W | Q | Q | G | F |
| 17 | | | | | | | | | | | | | | | | | | | |
| CH3C. | 1 | L | G | H | V | W | A | V | Y | . . . | P | K | S | T | W | Q | Q | G | W |
| 18 | | | | | | | | | | | | | | | | | | | |
| CH3C. | 1 | L | G | L | V | W | V | G | Y | . . . | P | K | S | T | W | Q | Q | G | W |
| 21 | | | | | | | | | | | | | | | | | | | |
| CH3C. | 1 | M | G | H | V | W | V | G | Y | . . . | D | K | S | T | W | Q | Q | G | W |
| 25 | | | | | | | | | | | | | | | | | | | |
| CH3C. | 1 | L | G | L | V | W | V | F | S | . . . | P | K | S | T | W | Q | Q | G | W |
| 34 | | | | | | | | | | | | | | | | | | | |
| CH3C. | 2 | Y | G | T | E | W | S | S | Y | . . . | T | K | S | E | W | Q | Q | G | F |
| 35 | | | | | | | | | | | | | | | | | | | |
| CH3C. | 2 | Y | G | T | E | W | S | N | Y | . . . | S | K | S | E | W | Q | Q | G | F |
| 44 | | | | | | | | | | | | | | | | | | | |
| CH3C. | 1/2 | L | G | H | V | W | V | G | Y | . . . | S | K | S | E | W | Q | Q | G | W |
| 51 | | | | | | | | | | | | | | | | | | | |
| CH3C. | 1 | L | G | H | V | W | V | A | T | . . . | P | K | S | T | W | Q | Q | G | W |
| 3.1-3 | | | | | | | | | | | | | | | | | | | |
| CH3C. | 1 | L | G | P | V | W | V | H | T | . . . | P | K | S | T | W | Q | Q | G | W |
| 3.1-9 | | | | | | | | | | | | | | | | | | | |
| CH3C. | 1 | L | G | H | V | W | V | D | Q | . . . | P | K | S | T | W | Q | Q | G | W |
| 3.2-5 | | | | | | | | | | | | | | | | | | | |
| CH3C. | 1 | L | G | H | V | W | V | N | Q | . . . | P | K | S | T | W | Q | Q | G | W |
| 3.2- | | | | | | | | | | | | | | | | | | | |
| 19 | | | | | | | | | | | | | | | | | | | |
| CH3C. | 1 | L | G | H | V | W | V | N | F | . . . | P | K | S | T | W | Q | Q | G | W |
| 3.2-1 | | | | | | | | | | | | | | | | | | | |
| CH3C. | | W | G | F | V | W | S | T | Y | . . . | P | K | S | N | W | Q | Q | G | F |
| 3.4-1 | | | | | | | | | | | | | | | | | | | |
| CH3C. | | W | G | H | V | W | S | T | Y | . . . | P | K | S | N | W | Q | Q | G | Y |
| 3.4- | | | | | | | | | | | | | | | | | | | |
| 19 | | | | | | | | | | | | | | | | | | | |
| CH3C. | | L | G | H | V | W | V | E | Q | . . . | P | K | S | T | W | Q | Q | G | W |
| 3.2-3 | | | | | | | | | | | | | | | | | | | |
| CH3C. | | L | G | H | V | W | V | G | V | . . . | P | K | S | T | W | Q | Q | G | W |
| 3.2- | | | | | | | | | | | | | | | | | | | |
| 14 | | | | | | | | | | | | | | | | | | | |
| CH3C. | | L | G | H | V | W | V | H | T | . . . | P | K | S | T | W | Q | Q | G | W |
| 3.2- | | | | | | | | | | | | | | | | | | | |
| 24 | | | | | | | | | | | | | | | | | | | |
| CH3C. | | W | G | T | V | W | G | T | Y | | P | K | S | N | W | Q | Q | G | Y |
| 3.4- | | | | | | | | | | | | | | | | | | | |
| 26 | | | | | | | | | | | | | | | | | | | |
| CH3C. | | L | G | H | V | W | V | G | T | | P | K | S | T | W | Q | Q | G | W |
| 3.2- | | | | | | | | | | | | | | | | | | | |
| 17 | | | | | | | | | | | | | | | | | | | |

### Generation of Phage-Display Libraries

A DNA template coding for the wild-type human Fc sequence was synthesized and incorporated into a phagemid vector. The phagemid vector contained an ompA or pelB leader sequence, the Fc insert fused to c-Myc and 6×His epitope tags, and an amber stop codon followed by M13 coat protein pIII.

Primers containing “NNK” tricodons at the corresponding positions for randomization were generated, where N is any DNA base (i.e., A, C, G, or T) and K is either G or T. Alternatively, primers for “soft” randomization were used, where a mix of bases corresponding to 70% wild-type base and 10% of each of the other three bases was used for each randomization position. Libraries were generated by performing PCR amplification of fragments of the Fc region corresponding to regions of randomization and then assembled using end primers containing SfiI restriction sites, then digested with SfiI and ligated into the phagemid vectors. Alternatively, the primers were used to conduct Kunkel mutagenesis. Methods of performing Kunkel mutagenesis will be known to one of skill in the art. The ligated products or Kunkel products were transformed into electrocompetent E. coli cells of strain TG1 (obtained from Lucigen®). The E. coli cells were infected with M13K07 helper phage after recovery and grown overnight, after which library phage were precipitated with 5% PEG/NaCl, resuspended in 15% glycerol in PBS, and frozen until use. Typical library sizes ranged from about 10⁹ to about 10¹¹ transformants. Fc-dimers were displayed on phage via pairing between pIII-fused Fc and soluble Fc not attached to pIII (the latter being generated due to the amber stop codon before pIII).

### Generation of Yeast-Display Libraries

A DNA template coding for the wild-type human Fc sequence was synthesized and incorporated into a yeast display vector. For CH2 and CH3 libraries, the Fc polypeptides were displayed on the Aga2p cell wall protein. Both vectors contained prepro leader peptides with a Kex2 cleavage sequence, and a c-Myc epitope tag fused to the terminus of the Fc.

Yeast display libraries were assembled using methods similar to those described for the phage libraries, except that amplification of fragments was performed with primers containing homologous ends for the vector. Freshly prepared electrocompetent yeast (i.e., strain EBY100) were electroporated with linearized vector and assembled library inserts. Electroporation methods will be known to one of skill in the art. After recovery in selective SD-CAA media, the yeast were grown to confluence and split twice, then induced for protein expression by transferring to SG-CAA media. Typical library sizes ranged from about 10⁷ to about 10⁹ transformants. Fc-dimers were formed by pairing of adjacently displayed Fc monomers.

### General Methods for Phage Selection

Phage methods were adapted from Phage Display: A Laboratory Manual (Barbas, 2001). Additional protocol details can be obtained from this reference.

Plate Sorting Methods

Human TfR target was coated on MaxiSorpx microtiter plates (typically 200 μL at 1-10 μg/mL in PBS) overnight at 4° C. All binding was done at room temperature unless otherwise specified. The phage libraries were added into each well and incubated overnight for binding. Microtiter wells were washed extensively with PBS containing 0.05% Tween® 20 (PBST) and bound phage were eluted by incubating the wells with acid (typically 50 mM HCl with 500 mM KCl, or 100 mM glycine, pH 2.7) for 30 minutes. Eluted phage were neutralized with 1 M Tris (pH 8) and amplified using TG1 cells and M13/K07 helper phage and grown overnight at 37° C. in 2YT media containing 50 μg/mL carbenacillin and 50 ug/mL Kanamycin. The titers of phage eluted from a target-containing well were compared to titers of phage recovered from a non-target-containing well to assess enrichment. Selection stringency was increased by subsequently decreasing the incubation time during binding and increasing washing time and number of washes.

Bead Sorting Methods

Human TfR target was biotinylated through free amines using NHS-PEG4-Biotin (obtained from Pierce™). For biotinylation reactions, a 3- to 5-fold molar excess of biotin reagent was used in PBS. Reactions were quenched with Tris followed by extensive dialysis in PBS. The biotinylated target was immobilized on streptavidin-coated magnetic beads, (i.e., M280-streptavidin beads obtained Thermo Fisher). The phage display libraries were incubated with the target-coated beads at room temperature for 1 hour. The unbound phage were then removed and beads were washed with PBST. The bound phage were eluted by incubating with 50 mM HCl containing 500 mM KCl (or 0.1 M glycine, pH 2.7) for 30 minutes, and then neutralized and propagated as described above for plate sorting.

After three to five rounds of panning, single clones were screened by either expressing Fc on phage or solubly in the E. coli periplasm. Such expression methods will be known to one of skill in the art. Individual phage supernatants or periplasmic extracts were exposed to blocked ELISA plates coated with target or a negative control and were subsequently detected using HRP-conjugated goat anti-Fc (obtained from Jackson Immunoresearch) for periplasmic extracts or anti-M13 (GE Healthcare) for phage, and then developed with TMB reagent (obtained from Thermo Fisher). Wells with OD₄₅₀ values greater than around 5-fold over background were considered positive clones and sequenced, after which some clones were expressed either as a soluble Fc fragment or fused to Fab fragments.

### General Methods for Yeast Selection

Bead Sorting (Magnetic-Assisted Cell Sorting (MACS)) Methods

MACS and FACS selections were performed similarly to as described in Ackerman, et al. 2009 Biotechnol. Prog. 25(3), 774. Streptavidin magnetic beads (e.g., M-280 streptavidin beads from ThermoFisher) were labeled with biotinylated target and incubated with yeast (typically 5-10× library diversity). Unbound yeast were removed, the beads were washed, and bound yeast were grown in selective media and induced for subsequent rounds of selection.

Bead Sorting (Magnetic-Assisted Cell Sorting (MACS)) Methods

Yeast were labeled with anti-c-Myc antibody to monitor expression and biotinylated target (concentration varied depending on the sorting round). In some experiments, the target was pre-mixed with streptavidin-Alexa Fluor® 647 in order to enhance the avidity of the interaction. In other experiments, the biotinylated target was detected after binding and washing with streptavidin-Alexa Fluor® 647. Singlet yeast with binding were sorted using a FACS Aria III cell sorter. The sorted yeast were grown in selective media then induced for subsequent selection rounds.

After an enriched yeast population was achieved, yeast were plated on SD-CAA agar plates and single colonies were grown and induced for expression, then labeled as described above to determine their propensity to bind to the target. Positive single clones were subsequently sequenced for binding target, after which some clones were expressed either as a soluble Fc fragment or as fused to Fab fragments.

### General Methods for Screening

Screening by ELISA

Clones were selected from panning outputs and grown in individual wells of 96-well deep-well plates. The clones were either induced for periplasmic expression using autoinduction media (obtained from EMD Millipore) or infected with helper phage for phage-display of the individual Fc variants on phage. The cultures were grown overnight and spun to pellet E. coli. For phage ELISA, phage containing supernatant was used directly. For periplasmic expression, pellets were resuspended in 20% sucrose, followed by dilution at 4:1 with water, and shaken at 4° C. for 1 hour. Plates were spun to pellet the solids and supernatant was used in the ELISA.

ELISA plates were coated with target, typically at 0.5 mg/mL overnight, then blocked with 1% BSA before addition of phage or periplasmic extracts. After a 1-hour incubation and washing off unbound protein, HRP-conjugated secondary antibody was added (i.e., anti-Fc or anti-M13 for soluble Fc or phage-displayed Fc, respectively) and incubated for 30 minutes. The plates were washed again, and then developed with TMB reagent and quenched with 2N sulfuric acid. Absorbance at 450 nm was quantified using a plate reader (BioTek®) and binding curves were plotted using Prism software where applicable. Absorbance signal for tested clones was compared to negative control (phage or paraplasmic extract lacking Fc). In some assays, soluble holo-transferrin was added during the binding step, typically at significant molar excess (greater than 10-fold excess).

Screening by Flow Cytometry

Fc variant polypeptides (expressed either on phage, in periplasmic extracts, or solubly as fusions to Fab fragments) were added to cells in 96-well V-bottom plates (about 100,000 cells per well in PBS+1% BSA (PBSA)), and incubated at 4° C. for 1 hour. The plates were subsequently spun and the media was removed, and then the cells were washed once with PBSA. The cells were resuspended in PBSA containing secondary antibody (goat anti-human-IgG-Alexa Fluor® 647 (obtained from Thermo Fisher)). After 30 minutes, the plates were spun and the media was removed, the cells were washed 1-2 times with PBSA, and then the plates were read on a flow cytometer (i.e., a FACSCanto™ II flow cytometer). Median fluorescence values were calculated for each condition using FlowJo software and binding curves were plotted with Prism software.

### CH2A2 Clone Generation and Characterization

Selections with CH2A2 Library Against Transferrin Receptor (TfR)

Phage and yeast libraries against CH2A2 were panned and sorted against TfR as described above. Clones binding human and/or cynomolgous (cyno) TfR were identified in ELISA assays, as described in the section titled “Screening by ELISA” above, after four rounds of phage panning. Sequences of representative clones fell into two groups: group 1 containing 15 unique sequences (i.e., SEQ ID NOS:47-61) and group 2 containing a single unique sequence (i.e., SEQ ID NO:62). Group 1 sequences had a conserved Glu-Trp motif at positions 287-288. No consensus appeared at any other positions, though position 285 favored Arg and position 286 favored Trp or Tyr.

Characterization of CH2A2 Clones

Individual CH2A2 variants were expressed on the surface of phage and assayed for binding to human TfR, cyno TfR, or an irrelevant control by ELISA. Expression of Fc was confirmed by ELISA against anti-Myc antibody 9E10, which bound to the C-terminal c-Myc epitope tag. The data for four representative clones, CH2A2.5, CH2A2.1, CH2A2.4, and CH2A2.16, demonstrated that all were well-expressed and bound to human TfR, while none bound to the irrelevant control. The three clones from group 1 also bound to cyno TfR, whereas the one clone from group 2 (i.e., clone 2A2.16) was specific for human TfR.

In a second assay, the concentration of phage was kept constant (i.e., at the approximate EC₅₀) and a varying concentration of a soluble competitor, either holo-transferrin or human TfR, was added. It was found that binding was not appreciably impacted by addition of holo-transferrin at concentrations up to 5 μM. Conversely, soluble human TfR could compete for binding to surface-adsorbed human TfR, indicating a specific interaction.

The CH2A2 variants are expressed as Fc fusions to anti-BACE1 Fab fragments by cloning into an expression vector containing an anti-BACE1 variable region sequence. After expression in 293 or CHO cells, the resulting CH2A2-Fab fusions were purified by Protein A and size-exclusion chromatography, and then assayed for binding using ELISAs, surface plasmon resonance (SPR; i.e., using a Biacore™ instrument), biolayer inferometry (i.e., using an Octetx RED system), cell binding (e.g., flow cytometry), and other methods described herein. Additionally, the resulting polypeptide-Fab fusions are characterized for stability by thermal melting, freeze-thaw, and heat-accelerated denaturation.

Additional Engineering of CH2A2 Clones

Two secondary libraries were constructed to enhance the binding affinity of the initial hits against human and cyno TfR. The first library was generated based on the group 1 clones. The conserved EW motif at positions 287-288 was held invariant, and the semi-conserved R at position 285 was mutated using soft randomization. The other library positions (i.e., positions 274, 276, 283, 286, 289, and 290) were mutated by saturation mutagenesis. The second library was constructed based on the group 2 clone. This library was generated by soft randomization of the original CH2A2 library positions, but used clone 2A2.16 (SEQ ID NO:62) as the template (rather than wild-type Fc). Both libraries were constructed for phage and yeast display using methods described above.

The libraries were screened using methods described above and several clones that bound human TfR by ELISA were identified (Table 1).

### CH2C Clone Generation and Characterization

Selections with CH2C Library Against Transferrin Receptor (TfR)

Phage and yeast libraries against CH2C were panned and sorted against TfR as described above. Clones binding human and/or cynomolgous (cyno) TfR were identified in ELISA assays, as described in the section titled “Screening by ELISA” above, after four rounds of phage panning (i.e., group 1 and 4 clones), and additional clones were identified after four or five yeast sort rounds (i.e., group 2 and 3 clones), by yeast binding assays as described in the section titled “General Methods for Yeast Selection” above. Sequences of representative clones fell into four groups: group 1 containing 16 unique sequences (i.e, SEQ ID NOS:63-78), group 2 containing 4 unique sequences (i.e., SEQ ID NOS:79-82), group 3 containing 2 unique sequences (i.e., SEQ ID NOS:83-84), and group 4 containing a single sequence (i.e., SEQ ID NO:85) (Table 2). The group 1 sequences had a semi-conserved Pro at position 266, a semi-conserved Pro at position 269, a conserved Pro at position 270, a semi-conserved Trp at position 271, a semi-conserved Glu at position 295, a conserved Tyr at position 297, and little specific preference at other library positions. The group 2 sequences had a conserved Met at position 266, a semi-conserved L at position 267, a conserved Pro at position 269, a conserved Val at position 270, a semi-conserved Pro at position 271, a semi-conserved Thr at position 295, a conserved His at position 297, and a conserved Pro at position 299. The two group 3 sequences only differed at position 295, where either a Val or Leu was present. Group 4 consisted of a single clone (i.e., CH2C.23) with a sequence as indicated in SEQ ID NO:85.

Characterization of CH2C Clones

The CH2C variants were expressed as Fc fusions to Fab fragments by cloning into an expression vector containing an anti-BACE1 benchmark variable region sequence. After expression in 293 or CHO cells, the resulting polypeptide-Fab fusions were purified by Protein A and size-exclusion chromatography, then assayed for binding to human or cyno TfR. The group 4 clone CH2C.23 competed with holo-transferrin. Clones belonging to sequence group 1 were tested in binding titrations against human and cyno TfR. Representative clones from other sequence groups were tested on phage for binding in the presence or absence of holo-Tf, and clone CH2C.7 was tested for binding to human TfR in the presence of holo-transferrin by biolayer interferometry (i.e., using an Octet® RED system). Most clones showed some cross-reactivity to cyno TfR, and except for clone CH2C.23, the clones that were tested did not compete with holo-Tf.

### CH2D Clone Generation and Characterization

Selections with CH2D Library Against Transferrin Receptor (TfR)

Phage libraries against CH2D were panned against TfR as described above. Clones binding human and/or cyno TfR were identified in ELISA assays, as described in the section titled “Screening by ELISA” above. Five unique clones were identified which were grouped into two sequence families of 2 and 3 sequences, respectively (Table 3). Sequence group 1 (i.e., clones CH2D.1 (SEQ ID NO:86) and CH2D.2 (SEQ ID NO:87)) had a conserved VPPXM (SEQ ID NO:111) motif at positions 268-272, an SLTS (SEQ ID NO:112) motif at positions 291-295, and V at position 300. Mutations at position 267 were not included in the design and were likely due to PCR error or recombination. Sequence group 2 (i.e., clones CH2D.3 (SEQ ID NO:88), CH2D.4 (SEQ ID NO:89), and CH2D.5 (SEQ ID NO:90)) had a conserved D at position 268, a semi-conserved D at position 269, a conserved W at position 270, a semi-conserved E at position 271, a conserved aromatic (W or Y) at position 272, a conserved PW motif at positions 291-292, and a conserved W at position 300.

Characterization and Additional Engineering of CH2D Clones

CH2D variants were expressed as fusions to Fab fragments by cloning into an expression vector containing an anti-BACE1 variable region sequence. After expression in 293 or CHO cells, the resulting polypeptide-Fab fusions were purified by Protein A and size-exclusion chromatography, then assayed for binding to cyno and human TfR in the presence or absence of holo-Tf using methods previously described herein.

### CH2E Clone Generation and Characterization

Selections with CH2E3 Library Against Transferrin Receptor (TfR)

Phage libraries against CH2E3 were panned against TfR as described above. Clones binding human and/or cyno TfR were identified in ELISA assays, as described in the section titled “Screening by ELISA” above. Three sequence groups were identified from 5 sequences, though two of the groups only consisted of one unique sequence each (Table 4). Sequence group 2, which had 3 unique sequences (i.e., clones CH2E3.2 (SEQ ID NO:92), CH2E3.3 (SEQ ID NO:93), and CH2E3.4 (SEQ ID NO:94)), had a semi-conserved Val at position 272, a conserved Gly at position 274, a conserved Arg at position 276, a conserved Arg at position 322, a conserved Ser at positions 324 and 326, a conserved Trp at position 330, and an Arg or Lys at position 331.

Characterization and Additional Engineering of CH2E3 Clones

CH2E3 variants were expressed as fusions to Fab fragments by cloning into an expression vector containing an anti-BACE1 benchmark variable region sequence. After expression in 293 or CHO cells, the resulting polypeptide-Fab fusions were purified by Protein A and size-exclusion chromatography, then assayed for binding to cyno and human TfR in the presence or absence of holo-Tf using methods for binding previously described herein.

### CH3B Clone Generation and Characterization

Selections with CH3B Library Against Transferrin Receptor (TfR)

Phage and yeast libraries against CH3B were panned and sorted against TfR as described above. Clones binding human and/or cyno TfR were identified in ELISA assays, as described in the section titled “Screening by ELISA” above, after four rounds of phage panning, and additional clones were identified after four or five yeast sort rounds, by yeast binding assays as described in the section titled “General Methods for Yeast Selection” above. All 17 clones (i.e., SEQ ID NOS:30-46) identified from both phage and yeast had related sequences; the sequences had a semi-conserved Phe at position 345, a semi-conserved negatively charged Asp or Glu at position 346, a semi-conserved Thr at position 349, a conserved G at position 437, a conserved Phe at position 438, a semi-conserved His at position 439, and a conserved Asp at position 440. Several clones had a T350I mutation, which was not a position intentionally mutated in the library design, but presumably was introduced by recombination or PCR error.

Characterization of CH3B Clones

Two representative clones, CH3B.11 (SEQ ID NO:40) and CH3B.12 (SEQ ID NO:41), were expressed on the surface of phage and tested for binding to human and cyno TfR in the presence or absence of holo-Tf. Neither clone was affected by the addition of holo-Tf. Additionally, the CH3B variants were expressed as fusions to Fab fragments by cloning into an expression vector containing an anti-BACE1 variable region sequence. After expression in 293 or CHO cells, the resulting polypeptide-Fab fusions were purified by Protein A and size-exclusion chromatography, then assayed for binding to human or cyno TfR. All showed specific binding to both orthologs.

Additional Engineering of CH3B Clones

Additional engineering methods, similar to those described above for CH2A2 for the design and screening of additional libraries, were used to improve the affinity of CH3B clones. In particular, several series of four to seven residue patches near the paratope were selected for additional diversification. Clone CH3B.12 (SEQ ID NO:41) was used as a starting point; the residues selected for saturation (i.e., NNK) mutagenesis were as follows:

CH3B-patch1: amino acid positions 354, 355, 356, 358, 359, 360, and 361;
CH3B-patch2: amino acid positions 348, 433, 434, and 436;
CH3B-patch3: amino acid positions 352, 441, 444, 445, 446, and 447;
CH3B-patch4: amino acid positions 342, 344, 370, 401, and 403; and
CH3B-patch5: amino acid positions 382, 384, 385, 420, 421, and 422.

The libraries were generated using PCR mutagenesis and put into yeast and phage as described in the sections titled “Generation of Phage-Display Libraries” and “Generation of Yeast-Display Libraries” above. The libraries were screened using methods described above and several clones that bound human TfR by ELISA were identified (Table 5).

### CH3C Clone Generation and Characterization

Selections with CH3C Library Against Transferrin Receptor (TfR)

Yeast libraries against CH3C were panned and sorted against TfR as described above. Population enrichment FACS was performed for the first three sort rounds. After an additional two rounds of sorting, single clones were sequenced and four unique sequences (i.e., clones CH3C.1 (SEQ ID NO:4), CH3C.2 (SEQ ID NO:5), CH3C.3 (SEQ ID NO:6), and CH3C.4 (SEQ ID NO:7)) were identified (Table 6). These sequences had a conserved Trp at position 388, and all had an aromatic residue (i.e., Trp, Tyr, or His) at position 421. There was a great deal of diversity at other positions.

Characterization of First Generation CH3C Clones

The four clones selected from the CH3C library were expressed as Fc fusions to Fab fragments in CHO or 293 cells, and purified by Protein A and size-exclusion chromatography, and then screened for binding to cyno and human TfR in the presence or absence of holo-Tf by ELISA. The clones all bound to human TfR and the binding was not affected by the addition of excess (5 μM) holo-Tf. However, the clones did not bind appreciably to cyno TfR. Clones were also tested for binding to 293F cells, which endogenously express human TfR. While the clones bound to 293F cells, the overall binding was substantially weaker than the high-affinity positive control.

Next it was tested whether clone CH3C.3 could internalize in TfR-expressing cells. Adherent HEK293 cells were grown in 96-well plates to about 80% confluence, media was removed, and samples were added at 1 μM concentrations: CH3C.3 anti-TfR benchmark positive control antibody (Ab204), anti-BACE1 benchmark negative control antibody (Ab107), and human IgG isotype control (obtained from Jackson Immunoresearch). The cells were incubated at 37° C. and 8% CO₂ concentration for 30 minutes, then washed, permeabilized with 0.1% Triton™ X-100, and stained with anti-human-IgG-Alexa Fluor® 488 secondary antibody. After additional washing, the cells were imaged under a high content fluorescence microscope (i.e., an Opera Phenix™ system), and the number of puncta per cell was quantified. At 1 clone CH3C.3 showed a similar propensity for internalization to the positive anti-TfR control, while the negative controls showed no internalization.

Secondary Engineering of CH3C Clones

Additional libraries were generated to improve the affinity of the initial CH3C hits against human TfR, and to attempt to introduce binding to cyno TfR. A soft randomization approach was used, wherein DNA oligos were generated to introduce soft mutagenesis based on each of the original four hits. The first portion of the register (WESXGXXXXXYK) and the second portion of the register (TVXKSXWQQGXV) were built via separate fragments, so the soft randomized registers were shuffled during PCR amplification (e.g., the first portion of the register from clone CH3C.1 was mixed with the second portion of the register from clones CH3C.1, CH3C.2, CH3C.3, and CH3C.4, and so forth). The fragments were all mixed and then introduced into yeast for surface expression and selection.

After one round of MACS and three rounds of FACS, individual clones were sequenced (clones CH3C.17 (SEQ ID NO:8), CH3C.18 (SEQ ID NO:9), CH3C.21 (SEQ ID NO:10), CH3C.25 (SEQ ID NO:11), CH3C.34 (SEQ ID NO:12), CH3C.35 (SEQ ID NO:13), CH3C.44 (SEQ ID NO:14), and CH3C.51 (SEQ ID NO:15)). The selected clones fell into two general sequence groups (Table 6). Group 1 clones (i.e., clones CH3C.18, CH3C.21, CH3C.25, and CH3C.34) had a semi-conserved Leu at position 384, a Leu or His at position 386, a conserved and a semi-conserved Val at positions 387 and 389, respectively, and a semi-conserved P-T-W motif at positions 413, 416, and 421, respectively. Group 2 clones had a conserved Tyr at position 384, the motif TXWSX at positions 386-390, and the conserved motif S/T-E-F at positions 413, 416, and 421, respectively. Clones CH3C.18 and CH3.35 were used in additional studies as representative members of each sequence group. It was noted that clone CH3C.51 had the first portion of its register from group 1 and the second portion of its register from group 2.

Binding Characterization of CH3C Clones from the Soft Mutagenesis Library

Clones from the soft mutagenesis library were reformatted as Fc-Fab fusion polypeptides and expressed and purified as described above. These variants had improved ELISA binding to human TfR as compared to the top clone from the initial library selections (CH3C.3), and also did not compete with holo-Tf. The EC₅₀ values were not appreciably affected beyond the margin of error of the experiment by the presence or absence of holo-Tf.

Notably, clone CH3C.35 bound to human TfR about as well as the high affinity anti-TfR control antibody Ab204. The clones selected from the soft randomization library also had improved cell binding to 293F cells. In a similar cell binding assay, these clones were tested for binding to CHO-K1 cells that stably express high levels of human or cyno TfR on their surface. The clones selected from the soft randomization library bound to cells expressing human TfR as well as cyno TfR and did not bind to the parental CHO-K1 cells. The magnitude and binding EC₅₀ values were substantially lower for cyno TfR as compared to human TfR.

Epitope Mapping

To determine whether the engineered CH3C Fc regions bound to the apical domain of TfR, TfR apical domain (SEQ ID NOS:96 and 97 for human and cyno, respectively) was expressed on the surface of phage. To properly fold and display the apical domain, one of the loops had to be truncated and the sequence needed to be circularly permuted; the sequences expressed on phage are identified as SEQ ID NOS:98 and 99 for human and cyno, respectively. Clones CH3C.18 and CH3C.35 were coated on ELISA plates and the previously described phage ELISA protocol was followed. Briefly, after washing and blocking with 1% PBSA, dilutions of phage displaying were added and incubated at room temperature for 1 hour. The plates were subsequently washed and anti-M13-HRP was added, and after additional washing the plates were developed with TMB substrate and quenched with 2N H2504. Both CH3C.18 and CH3C.35 bound to the apical domain in this assay.

Since binding to cyno TfR was known to be much weaker than binding to human TfR, it was hypothesized that one or more of the amino acid differences between cyno and human apical domains was likely responsible for the binding difference. Therefore, a series of six point mutations was made in the human TfR apical domain where the human residue was replaced with the corresponding cyno residue. These mutants were displayed on phage and the phage concentrations were normalized by OD₂₆₈ and binding to CH3C.18 and CH3C.35 was tested by phage ELISA titration. Capture on anti-Myc antibody 9E10 showed that display levels for all mutants were similar. Binding to the human TfR mutations clearly showed a strong effect of the R435G mutation, which suggested that this residue is a key part of the epitope and is negatively impacted by the cyno residue at this position. The G435R mutation was made on phage-displayed cyno apical domain and it was shown that this mutation dramatically improved binding to cyno apical domain. These results show that the CH3C clones bound to the apical domain of TfR and that position 435 was important for binding, while positions 474, 519, 591, 597, and 599 were significantly less important.

Paratope Mapping

To understand which residues in the Fc domain were most critical for TfR binding, a series of mutant CH3C.18 and CH3C.35 clones was created in which each mutant had a single position in the TfR-binding register mutated back to wild-type. The resulting variants were expressed recombinantly as CH3C Fc-Fab fusions and tested for binding to human or cyno TfR. For CH3C.35, positions 388 and 421 were absolutely critical for binding; reversion of either of these to wild-type completely ablated binding to human TfR. Surprisingly, reverting position 390 to wild-type provided a dramatic boost to cyno TfR binding, while having little effect on human binding. Conversely, the reversion of residue 390 to wild-type had little effect in CH3C.18, but in this variant reversion of positions 416 and 421 completely abolished binding to human TfR. In both variants, other single reversions had modest (detrimental) impact on human TfR binding, while in many cases binding to cyno TfR was abolished.

Additional Engineering to Improve Binding to Cyno TfR

Additional libraries were prepared to further increase the affinity of the CH3C variants for cyno TfR. These libraries were designed to be of less than about 10⁷ clones in terms of theoretical diversity, so that the full diversity space could be explored using yeast surface display. Four library designs were used; all libraries were generated using degenerate oligos with NNK or other degenerate codon positions, and amplified by overlap PCR, as described above.

The first library was based on the consensus of CH3C.35-like sequences. Here, positions 384-388 were held constant as YGTEW, while positions 389, 390, 413, 416, and 421 were mutated using saturation mutagenesis.

The second library was based on the consensus of CH3C.18-like sequences. Here, position 384 was restricted to Leu and Met, position 386 was restricted to Leu and His, position 387 was held constant as Val, position 388 was restricted to Trp and Gly, position 389 was restricted to Val and Ala, position 390 was fully randomized, position 391 was added to the register and fully randomized, position 413 was soft randomized, position 416 was fully randomized, and position 421 was restricted to aromatic amino acids and Leu.

The third library added new randomized positions to the library. Two versions were generated, one each with CH3C.18 and CH3C.35 as the starting register, and then additional positions were randomized by saturation mutagenesis: E153, E155, Y164, 5188, and Q192.

The fourth library held certain positions constant for CH3C.18 but allowed variation at other positions, with less bias than the consensus library. Positions 387, 388, and 413 were fixed, and positions 384, 386, 389, 390, and 416 were randomized by saturating mutagenesis; position 421 was mutated but restricted to aromatic residues and Leu.

The libraries were selected in yeast for four to five rounds against cynoTfR and single clones were sequenced and converted to polypeptide-Fab fusions, as described above. The greatest enrichment in cynoTfR binding was observed from the second library (i.e., derivatives of the CH3C.18 parent), though there was also some loss in huTfR binding.

Binding Characteristics of CH3C Maturation Clones

Binding ELISAs were conducted with purified CH3C Fc-Fab fusion variants with human or cyno TfR coated on the plate, as described above. The variants from the CH3C.18 maturation library, CH3C3.2-1, CH3C.3.2-5, and CH3C.3.2-19, bound human and cyno TfR with approximately equivalent EC₅₀ values, whereas the parent clone CH3C.18, and CH3C.35, had greater than 10-fold better binding to human versus cyno TfR.

Next, it was tested whether the new polypeptides internalized in human and monkey cells. Using the protocol previously described above in the section titled “Characterization of first generation CH3C clones,” internalization in human HEK293 cells and rhesus LLC-MK2 cells was tested. The variants that similarly bound human and cyno TfR, CH3C.3.2-5 and CH3C.3.2-19, had significantly improved internalization in LLC-MK2 cells as compared with CH3C.35.

Additional Engineering of CH3C Clones

Additional engineering to further affinity mature clones CH3C.18 and CH3C.35 involved adding additional mutations to the backbone (i.e., non-register) positions that enhanced binding through direct interactions, second-shell interactions, or structure stabilization. This was achieved via generation and selection from an “NNK walk” or “NNK patch” library. The NNK walk library involved making one-by-one NNK mutations of residues that are near to the paratope. By looking at the structure of Fc bound to FcgRI (PDB ID: 4W4O), 44 residues near the original library register were identified as candidates for interrogation. Specifically, the following residues were targeted for NNK mutagenesis: K248, R255, Q342, R344, E345, Q347, T359, K360, N361, Q362, 5364, K370, E380, E382, S383, G385, Y391, K392, T393, D399, 5400, D401, 5403, K409, L410, T411, V412, K414, S415, Q418, Q419, G420, V422, F423, 5424, 5426, Q438, 5440, 5442, L443, 5444, P4458, G446, and K447. The 44 single point NNK libraries were generated using Kunkel mutagenesis, and the products were pooled and introduced to yeast via electroporation, as described above for other yeast libraries.

The combination of these mini-libraries (each of which had one position mutated, resulting in 20 variants) generated a small library that was selected using yeast surface display for any positions that lead to higher affinity binding. Selections were performed as described above, using TfR apical domain proteins. After three rounds of sorting, clones from the enriched yeast library were sequenced, and several “hot-spot” positions were identified where certain point mutations significantly improved the binding to apical domain proteins. For CH3C.35, these mutations included E380 (mutated to Trp, Tyr, Leu, or Gln) and 5415 (mutated to Glu). The sequences of the CH3C.35 single and combination mutants are set forth in SEQ ID NOS:21-23, 101-106, and 162-164. For CH3C.18, these mutations included E380 (mutated to Trp, Tyr, or Leu) and K392 (mutated to Gln, Phe, or His). The sequences of the CH3C.18 single mutants are set forth in SEQ ID NOS:107-112.

### Additional Maturation Libraries to Improve CH3C.35 Affinity

An additional library to identify combinations of mutations from the NNK walk library, while adding several additional positions on the periphery of these, was generated as described for previous yeast libraries. In this library, the YxTEWSS and TxxExxxxF motifs were kept constant, and six positions were completely randomized: E380, K392, K414, 5415, S424, and S426. Positions E380 and 5415 were included because they were “hot spots” in the NNK walk library. Positions K392, S424, and S426 were included because they make up part of the core that may position the binding region, while K414 was selected due to its adjacency to position 415.

This library was sorted, as previously described, with the cyno TfR apical domain only. The enriched pool was sequenced after five rounds, and the sequences of the CH3 regions of the identified unique clones are set forth in SEQ ID NOS:113-130.

Exploration of Acceptable Diversity within the Original Register and Hot Spots for CH3C.35.21

The next libraries were designed to explore the totality of acceptable diversity in the main binding paratope. The approach taken was similar to the NNK walk libraries. Each of the original register positions (384, 386, 387, 388, 389, 390, 413, 416, and 421) plus the two hot spots (380 and 415) were individually randomized with NNK codons to generate a series of single-position saturation mutagenesis libraries on yeast. In addition, each position was individually reverted to the wild-type residue, and these individual clones were displayed on yeast. It was noted that positions 380, 389, 390, and 415 were the only positions that retained substantial binding to TfR upon reversion to the wild-type residue (some residual but greatly diminished binding was observed for reversion of 413 to wild-type).

The single-position NNK libraries were sorted for three rounds against the human TfR apical domain to collect the top ˜5% of binders, and then at least 16 clones were sequenced from each library. The results indicate what amino acids at each position can be tolerated without significantly reducing binding to human TfR, in the context of the CH3C.35 clone. A summary is below:

### Position 380: Trp, Leu, or Glu;

### Position 384: Tyr or Phe;

Position 386: Thr only;
Position 387: Glu only;
Position 388: Trp only;
Position 389: Ser, Ala, or Val (although the wild type Asn residue seems to retain some binding, it did not appear following library sorting);

### Position 390: Ser or Asn;

### Position 413: Thr or Ser;

### Position 415: Glu or Ser;

Position 416: Glu only; and
Position 421: Phe only.

The above residues, when substituted into clone CH3C.35 as single changes or in combinations, represent paratope diversity that retains binding to TfR apical domain. Clones having mutations at these positions are shown in Table 7, and the sequences of the CH3 domains of these clones are set forth in SEQ ID NOS:102-106, 129, and 131-161.

TABLE 7
Exploration of Acceptable Diversity Within Register and
Hot Spot Positions for CH3C.35.21

| | 378 | 379 | 380 | 381 | 382 | 383 | 384 | 385 | 386 | 387 | 388 | 389 | 390 | 391 | 392 | 411 | 412 | 413 | 414 | 415 | 416 | 417 | 418 | 419 | 420 | 421 | 422 | 423 |
|---|---|---|---|---|---|---|---|---|---|---|---|---|---|---|---|---|---|---|---|---|---|---|---|---|---|---|---|---|
| Wild- | A | V | E | W | E | S | N | G | Q | P | E | N | N | Y | K | T | V | D | K | S | R | W | Q | Q | G | N | V | F |
| type | | | | | | | | | | | | | | | | | | | | | | | | | | | | |
| CH3C.35. | . | . | . | . | . | . | F | . | T | E | W | S | S | . | . | . | . | T | . | E | E | . | . | . | . | F | . | . |
| 20.1 | | | | | | | | | | | | | | | | | | | | | | | | | | | | |
| CH3C.35. | . | . | . | . | . | . | Y | . | T | E | W | A | S | . | . | . | . | T | . | E | E | . | . | . | . | F | . | . |
| 20.2 | | | | | | | | | | | | | | | | | | | | | | | | | | | | |
| CH3C.35. | . | . | . | . | . | . | Y | . | T | E | W | V | S | . | . | . | . | T | . | E | E | . | . | . | . | F | . | . |
| 20.3 | | | | | | | | | | | | | | | | | | | | | | | | | | | | |
| CH3C.35. | . | . | . | . | . | . | Y | . | T | E | W | S | S | . | . | . | . | S | . | E | E | . | . | . | . | F | . | . |
| 20.4 | | | | | | | | | | | | | | | | | | | | | | | | | | | | |
| CH3C.35. | . | . | . | . | . | . | F | . | T | E | W | A | S | . | . | . | . | T | . | E | E | . | . | . | . | F | . | . |
| 20.5 | | | | | | | | | | | | | | | | | | | | | | | | | | | | |
| CH3C.35. | . | . | . | . | . | . | F | . | T | E | W | V | S | . | . | . | . | T | . | E | E | . | . | . | . | F | . | . |
| 20.6 | | | | | | | | | | | | | | | | | | | | | | | | | | | | |
| CH3C.35. | . | . | W | . | . | . | F | . | T | E | W | S | S | . | . | . | . | T | . | E | E | . | . | . | . | F | . | . |
| 21.A.1 | | | | | | | | | | | | | | | | | | | | | | | | | | | | |
| CH3C.35. | . | . | W | . | . | . | Y | . | T | E | W | A | S | . | . | . | . | T | . | E | E | . | . | . | . | F | . | . |
| 21.A.2 | | | | | | | | | | | | | | | | | | | | | | | | | | | | |
| CH3C.35. | . | . | W | . | . | . | Y | . | T | E | W | V | S | . | . | . | . | T | . | E | E | . | . | . | . | F | . | . |
| 21.A.3 | | | | | | | | | | | | | | | | | | | | | | | | | | | | |
| CH3C.35. | . | . | W | . | . | . | Y | . | T | E | W | S | S | . | . | . | . | S | . | E | E | . | . | . | . | F | . | . |
| 21.A.4 | | | | | | | | | | | | | | | | | | | | | | | | | | | | |
| CH3C.35. | . | . | W | . | . | . | F | . | T | E | W | A | S | . | . | . | . | T | . | E | E | . | . | . | . | F | . | . |
| 21.A.5 | | | | | | | | | | | | | | | | | | | | | | | | | | | | |
| CH3C.35. | . | . | W | . | . | . | F | . | T | E | W | V | S | . | . | . | . | T | . | E | E | . | . | . | . | F | . | . |
| 21.A.6 | | | | | | | | | | | | | | | | | | | | | | | | | | | | |
| CH3C.35. | . | . | . | . | . | . | F | . | T | E | W | S | . | . | . | . | . | T | . | E | E | . | . | . | . | F | . | . |
| 23.1 | | | | | | | | | | | | | | | | | | | | | | | | | | | | |
| CH3C.35. | . | . | . | . | . | . | Y | . | T | E | W | A | . | . | . | . | . | T | . | E | E | . | . | . | . | F | . | . |
| 23.2 | | | | | | | | | | | | | | | | | | | | | | | | | | | | |
| CH3C.35. | . | . | . | . | . | . | Y | . | T | E | W | V | . | . | . | . | . | T | . | E | E | . | . | . | . | F | . | . |
| 23.3 | | | | | | | | | | | | | | | | | | | | | | | | | | | | |
| CH3C.35. | . | . | . | . | . | . | Y | . | T | E | W | S | . | . | . | . | . | S | . | E | E | . | . | . | . | F | . | . |
| 23.4 | | | | | | | | | | | | | | | | | | | | | | | | | | | | |
| CH3C.35. | . | . | . | . | . | . | F | . | T | E | W | A | . | . | . | . | . | T | . | E | E | . | . | . | . | F | . | . |
| 23.5 | | | | | | | | | | | | | | | | | | | | | | | | | | | | |
| CH3C.35. | . | . | . | . | . | . | F | . | T | E | W | V | . | . | . | . | . | T | . | E | E | . | . | . | . | F | . | . |
| 23.6 | | | | | | | | | | | | | | | | | | | | | | | | | | | | |
| CH3C.35. | . | . | W | . | . | . | F | . | T | E | W | S | . | . | . | . | . | T | . | E | E | . | . | . | . | F | . | . |
| 24.1 | | | | | | | | | | | | | | | | | | | | | | | | | | | | |
| CH3C.35. | . | . | W | . | . | . | Y | . | T | E | W | A | . | . | . | . | . | T | . | E | E | . | . | . | . | F | . | . |
| 24.2 | | | | | | | | | | | | | | | | | | | | | | | | | | | | |
| CH3C.35. | . | . | W | . | . | . | Y | . | T | E | W | V | . | . | . | . | . | T | . | E | E | . | . | . | . | F | . | . |
| 24.3 | | | | | | | | | | | | | | | | | | | | | | | | | | | | |
| CH3C.35. | . | . | W | . | . | . | Y | . | T | E | W | S | . | . | . | . | . | S | . | E | E | . | . | . | . | F | . | . |
| 24.4 | | | | | | | | | | | | | | | | | | | | | | | | | | | | |
| CH3C.35. | . | . | W | . | . | . | F | . | T | E | W | A | . | . | . | . | . | T | . | E | E | . | . | . | . | F | . | . |
| 24.5 | | | | | | | | | | | | | | | | | | | | | | | | | | | | |
| CH3C.35. | . | . | W | . | . | . | F | . | T | E | W | V | . | . | . | . | . | T | . | E | E | . | . | . | . | F | . | . |
| 24.6 | | | | | | | | | | | | | | | | | | | | | | | | | | | | |
| CH3C.35. | . | . | L | . | . | . | F | . | T | E | W | S | S | . | . | . | . | T | . | E | E | . | . | . | . | F | . | . |
| 21.17.1 | | | | | | | | | | | | | | | | | | | | | | | | | | | | |
| CH3C.35. | . | . | L | . | . | . | Y | . | T | E | W | A | S | . | . | . | . | T | . | E | E | . | . | . | . | F | . | . |
| 21.17.2 | | | | | | | | | | | | | | | | | | | | | | | | | | | | |
| CH3C.35. | . | . | L | . | . | . | Y | . | T | E | W | V | S | . | . | . | . | T | . | E | E | . | . | . | . | F | . | . |
| 21.17.3 | | | | | | | | | | | | | | | | | | | | | | | | | | | | |
| CH3C.35. | . | . | L | . | . | . | Y | . | T | E | W | S | S | . | . | . | . | S | . | E | E | . | . | . | . | F | . | . |
| 21.17.4 | | | | | | | | | | | | | | | | | | | | | | | | | | | | |
| CH3C.35. | . | . | L | . | . | . | F | . | T | E | W | A | S | . | . | . | . | T | . | E | E | . | . | . | . | F | . | . |
| 21.17.5 | | | | | | | | | | | | | | | | | | | | | | | | | | | | |
| CH3C.35. | . | . | L | . | . | . | F | . | T | E | W | V | S | . | . | . | . | T | . | E | E | . | . | . | . | F | . | . |
| 21.17.6 | | | | | | | | | | | | | | | | | | | | | | | | | | | | |
| CH3C.35.20 | . | . | . | . | . | . | Y | . | T | E | W | S | S | . | . | . | . | T | . | E | E | . | . | . | . | F | . | . |
| CH3C.35.21 | . | . | W | . | . | . | Y | . | T | E | W | S | S | . | . | . | . | T | . | E | E | . | . | . | . | F | . | . |
| CH3C.35.22 | . | . | W | . | . | . | Y | . | T | E | W | S | . | . | . | . | . | T | . | | E | . | . | . | . | F | . | . |
| CH3C.35.23 | . | . | . | . | . | . | Y | . | T | E | W | S | . | . | . | . | . | T | . | E | E | . | . | . | . | F | . | . |
| CH3C.35.24 | . | . | W | . | . | . | Y | . | T | E | W | S | . | . | . | . | . | T | . | E | E | . | . | . | . | F | . | . |
| CH3C.35.21. | . | . | L | . | . | . | Y | . | T | E | W | S | S | . | . | . | . | T | . | E | E | . | . | . | . | F | . | . |
| 17 | | | | | | | | | | | | | | | | | | | | | | | | | | | | |
| CH3C.35. | . | . | . | . | . | . | Y | . | T | E | W | S | . | . | . | . | . | T | . | | E | . | . | . | . | F | . | . |
| N390 | | | | | | | | | | | | | | | | | | | | | | | | | | | | |
| CH3C.35. | | | | | | | F | | T | E | W | S | S | | | | | S | | E | E | | | | | F | | |
| 20.1.1 | | | | | | | | | | | | | | | | | | | | | | | | | | | | |
| CH3C.35. | | | | | | | Y | | T | E | W | A | | | | | | S | | | E | | | | | F | | |
| 23.2.1 | | | | | | | | | | | | | | | | | | | | | | | | | | | | |
| CH3C.35. | | | | | | | F | | T | E | w | S | | | | | | S | | E | E | | | | | F | | |
| 23.1.1 | | | | | | | | | | | | | | | | | | | | | | | | | | | | |
| CH3C.35. | | | | | | | Y | | T | E | W | S | S | | | | | S | | | E | | | | | F | | |
| S413 | | | | | | | | | | | | | | | | | | | | | | | | | | | | |
| CH3C.35. | | | | | | | Y | | T | E | W | V | | | | | | S | | E | E | | | | | F | | |
| 23.3.1 | | | | | | | | | | | | | | | | | | | | | | | | | | | | |
| CH3C.35. | | | | | | | Y | | T | E | W | S | | | | | | S | | | E | | | | | F | | |
| N390.1 | | | | | | | | | | | | | | | | | | | | | | | | | | | | |
| CH3C.35. | | | | | | | F | | T | E | W | V | | | | | | S | | E | E | | | | | F | | |
| 23.6.1 | | | | | | | | | | | | | | | | | | | | | | | | | | | | |

### Monovalent Polypeptide-Fab Fusions

Generation of Monovalent TfR-Binding Polypeptide-Fab Fusions

Although Fc domains naturally form homodimers, a series of asymmetric mutations known as “knobs-in-holes” can lead to preferential heterodimerization of two Fc fragments, where one Fc unit has the T366W knob mutation and the other Fc unit has the T366S, L368A, and Y407V hole mutations. In some embodiments, a modified CH3 domain of the invention comprises a Trp at position 366. In some embodiments, a modified CH3 domain of the invention comprises a Ser at position 366, an Ala at position 368, and a Val at position 407. Heterodimeric TfR-binding polypeptides were expressed in 293 or CHO cells by transient co-transfection of two plasmids (i.e., a knob-Fc and a hole-Fc), while polypeptide-Fab fusions were expressed by transient co-transfection of three plasmids (i.e., a knob-Fc-Fab heavy chain, a hole-Fc-Fab heavy chain, and a common light chain). Purification of secreted heterodimeric polypeptides or polypeptide-Fab fusions was performed identically to that for homodimers (i.e., a two-column purification using Protein A followed by size-exclusion, and then concentration and buffer exchange if required). Mass-spectrometry or hydrophobic interaction chromatography was used to determine the amount of heterodimer versus homodimer (e.g., knob-knob or hole-hole paired Fe's) formed. From typical preps, greater than 95% of polypeptides, and often greater than 98%, were heterodimers. For heterodimeric polypeptides and polypeptide-Fab fusions, the mutations that conferred TfR binding included the “knob” mutation, whereas a non-TfR-binding Fc region was used with the “hole” region, unless otherwise indicated. In some cases, additional mutations that alter Fc properties were also included in these constructs, such as L234A/L235A, M252Y/S254T/T256E, N434S, or N434S/M428L for modified FcγR or FcRn binding, respectively.

Binding Characterization of CH3C.Mono Fc Polypeptides

Binding of monovalent CH3C polypeptides was measured in an ELISA using a modification of the procedure described above. Streptavidin was coated on 96-well ELISA plates overnight at 1 μg/mL in PBS. After washing, the plates were blocked with 1% BSA in PBS, then biotinylated human or cyno TfR was added at 1 μg/mL and incubated for 30 minutes. After additional washing, polypeptides were added to the plates at serial dilutions, and incubated for 1 hour. The plates were washed and secondary antibody (i.e., anti-kappa-HRP, 1:5,000) was added for 30 minutes and the plates were washed again. The plates were developed with TMB substrate and quenched with 2N H2504 and then absorbance at 450 nm was read on a BioTek® plate reader. Bivalent TfR-binding and monovalent TfR-binding polypeptides were compared. Ab204 was used as a high affinity anti-TfR control antibody.

Additional testing was performed for binding to 293F cells, which endogenously express human TfR, as well as CHO-K1 cells that were stably transfected with human TfR or cyno TfR.

In general, substantially reduced binding to human TfR for monovalent polypeptides was observed as compared to bivalent polypeptides, and cyno binding was too weak to be detected in these assays for the monovalent polypeptides.

Next it was tested whether monovalent versions of CH3C polypeptides could internalize in human-TfR expressing HEK293 cells. Methods described above for internalization assays were used. The monovalent peptides could also internalize, but the overall signal was weaker than for the respective bivalent versions, presumably due to the loss of binding affinity/avidity.

Kinetics of Binding for CH3C Polypeptides Measured by Biolayer Inferometry

Binding kinetics were determined for several monovalent and bivalent CH3C polypeptide variants, fused to anti-BACE1 Fabs, and compared to their bivalent equivalents using biolayer inferometry (i.e., using an Octet® RED system). TfR was captured on a streptavidin sensor, then CH3C polypeptides were bound and washed off. Sensograms were fitted to a 1:1 binding model; the K_{D} (app) value for bivalent polypeptides represented avid binding to the TfR dimer.

The polypeptides that were converted to monovalent format had significantly weaker K_{D} (app) values, due to loss of avidity. Clones CH3C.3.2-1, CH3C.3.2-5, and CH3C.3.2-19, which were previously shown to have similar human and cyno TfR binding by ELISA, also had very similar K_{D} (app) values between human and cyno TfR. An attempt was made to test the monovalent forms of these polypeptides, but the binding in this assay was too weak to calculate kinetic parameters.

### Example 2. Single Amino Acid Substitution of CH3C.35.21

This example describes the construction of a library of CH3C.35.21 single amino acid mutants.

### Methods

A library of CH3C.35.21 mutants each containing a single amino acid substitution of CH3C.35.21 was constructed using Kunkel mutagenesis (Kunkel, Proc Natl Acad Sci USA. 82(2):488-92, 1985). For CH3C.35.21, each of positions W380, Y384, T386, E387, W388, S389, S390, K392, T413, K414, E415, E416, F421, S424, and S426, as numbered according to the EU numbering scheme, were mutated individually to the codon NNK using degenerate mutagenic oligos. To avoid obtaining the original CH3C.35.21 clone in the library, the single-stranded DNA (ssDNA) Kunkel template encoded a wild-type IgG1 Fc was used. Two mutagenic oligos (one with an NNK and the other encoding the other CH3C.35.21 region) were used in combination so that when both oligos were incorporated it yielded the CH3C.35.21 amino acid sequence, but with an NNK codon at the desired library positon. Because the template is a wild-type Fc, a single oligo insertion or no oligo insertion will not bind TfR, therefore, these constructs were easily eliminated from any analysis. Similarly, stop codons arising from the NNK positon were excluded. Libraries were transfected into EBY100 yeast. Eight colonies were sequenced from each library to ensure the naïve library contains the desired position randomization.

The top approximately 10% of the circularly permuted TfR apical domain bound population measured by yeast display and flow cytometry, were collected at a TfR concentration providing the best range for distinguishing affinities. Sequences were obtained for 12 clones for each positon. For libraries with distinct populations, the same experiment was done with better defined high, medium, low gates. There were 36 clones sequenced for each collected population. Further, in order to compare the binding of a mutant to the binding of the corresponding mutant having the wild-type residue at the corresponding amino acid position, the amino acid at the same position was reverted back to the wild-type IgG1 residue using a mutagenic oligo in similar methods.

Table 8 shows the library of CH3C.35.21 mutants. Each mutant contained a single amino acid substitution of CH3C.35.21. For example, one mutant may contain W380E and the amino acids at the rest of the positions are the same as those in CH3C.35.21. The positions shown in Table 8 are numbered according to the EU numbering scheme.

TABLE 8
CH3C.35.21 single amino acid mutants

| Position | 380 | 384 | 386 | 387 | 388 | 389 | 390 | 413 | 415 | 416 | 421 | 424 | 426 |
|---|---|---|---|---|---|---|---|---|---|---|---|---|---|
| Wild- | E | N | Q | P | E | N | N | D | S | R | N | S | S |
| type Fc | | | | | | | | | | | | | |
| CH3C. | | | | | | | | | | | | | |
| 35.21 | W | Y | T | E | W | S | S | T | E | E | F | S | S |
| Residues | E | Y | T | E | W | S | S | T | S | E | F | S | S |
| found | L | F | N | I | | A | N | H | D | R | H | T | c |
| to have | S | M | V | P | | I | R | S | G | | K | W | P |
| affinity | V | P | | V | | T | T | | T | | Y | | M |
| in the | W | V | | | | V | | | P | | | | W |
| range: | Y | W | | | | | | | 0 | | | | |
| <190 nM | | | | | | | | | R | | | | |
| to | | | | | | | | | | | | | |
| about | | | | | | | | | | | | | |
| ~500 nM | | | | | | | | | | | | | |

### Example 3. Generation of CH3C.18 Variants

This example describes the generation of CH3C.18 variants.

Single clones were isolated, and grown overnight in SG-CAA media supplemented with 0.2% glucose overnight to induce surface expression of CH3C.18 variants. For each clone, two million cells were washed three times in PBS+0.5% BSA at pH 7.4. Cells were stained with biotinylated target, 250 nM human TfR, 250 nM cyno TfR, or 250 nM of an unrelated biotinylated protein for 1 hour at 4° C. with shaking, then washed twice with the same buffer. Cells were stained with nuetravidin-Alexafluor647 (AF647) for 30 minutes at 4° C., then washed twice again. Expression was measured using anti-c-myc antibody with anti-chicken-Alexfluor488 (AF488) secondary antibody. Cells were resuspended, and median fluorescence intensity (MFI) of AF647 and AF488 was measured on a BD FACS CantoII. MFI was calculated for the TfR-binding population for each population and plotted with human TfR, cyno TfR, or control binding.

Table 9 shows the library of CH3C.18 variants. Each row represents a variant that contains the indicated amino acid substitutions at each position and the amino acids at the rest of the positions are the same as those in the CH3C.18 Fc. The positions shown in Table 9 are numbered according to the EU numbering scheme.

TABLE 9
CH3C.18 variants

| Position | 384 | 386 | 387 | 389 | 390 | 391 | 413 | 416 | 421 |
|---|---|---|---|---|---|---|---|---|---|
| Wild-type | N | Q | P | N | N | Y | D | R | N |
| Fc | | | | | | | | | |
| CH3C.4 | V | T | P | A | L | Y | L | E | W |
| (CH3C. | | | | | | | | | |
| 18.1) | | | | | | | | | |
| CH3C.2 | Y | T | V | S | H | Y | S | E | Y |
| (CH3C. | | | | | | | | | |
| 18.2) | | | | | | | | | |
| CH3C.3 | Y | T | E | S | Q | Y | E | D | H |
| (CH3C. | | | | | | | | | |
| 18.3) | | | | | | | | | |
| CH3C.1 | L | L | V | V | G | Y | A | T | W |
| (CH3C. | | | | | | | | | |
| 18.4) | | | | | | | | | |
| CH3C.18 | L | H | V | A | V | Y | P | T | W |
| (CH3C.18. | | | | | | | | | |
| 1.18) | | | | | | | | | |
| CH3C.3. | L | H | V | V | A | T | P | T | W |
| 1-3 | | | | | | | | | |
| (CH3C.18. | | | | | | | | | |
| 3.1-3) | | | | | | | | | |
| CH3C.3. | L | P | V | V | H | T | P | T | W |
| 1-9 | | | | | | | | | |
| (CH3C.18. | | | | | | | | | |
| 3.1-9) | | | | | | | | | |
| CH3C.3. | L | H | V | V | N | F | P | T | W |
| 2-1 | | | | | | | | | |
| (CH3C.18. | | | | | | | | | |
| 3.2-1) | | | | | | | | | |
| CH3C.3. | L | H | V | V | D | Q | P | T | W |
| 2-5 | | | | | | | | | |
| (CH3C.18. | | | | | | | | | |
| 3.2-5) | | | | | | | | | |
| CH3C.3. | L | H | V | V | N | Q | P | T | W |
| 2-19 | | | | | | | | | |
| (CH3C.18. | | | | | | | | | |
| 3.2-19) | | | | | | | | | |
| CH3C.3. | W | F | V | S | T | Y | P | N | F |
| 4-1 | | | | | | | | | |
| (CH3C.18. | | | | | | | | | |
| 3.4-1) | | | | | | | | | |
| CH3C.3. | W | H | V | S | T | Y | P | N | Y |
| 4-19 | | | | | | | | | |
| (CH3C.18. | | | | | | | | | |
| 3.4-19) | | | | | | | | | |
| CH3C.3. | L | H | V | V | E | Q | P | T | W |
| 2-3 | | | | | | | | | |
| (CH3C.18. | | | | | | | | | |
| 3.2-3) | | | | | | | | | |
| CH3C.3. | L | H | V | V | G | V | P | T | W |
| 2-14 | | | | | | | | | |
| (CH3C.18. | | | | | | | | | |
| 3.2-14) | | | | | | | | | |
| CH3C.3. | L | H | V | V | H | T | P | T | W |
| 2-24 | | | | | | | | | |
| (CH3C.18. | | | | | | | | | |
| 3.2-24) | | | | | | | | | |
| CH3C.3. | W | T | V | G | T | Y | P | N | Y |
| 4-26 | | | | | | | | | |
| (CH3C.18. | | | | | | | | | |
| 3.4-26) | | | | | | | | | |
| CH3C.3. | L | H | V | V | G | T | P | T | W |
| 2-17 | | | | | | | | | |
| (CH3C.18. | | | | | | | | | |
| 3.2-17) | | | | | | | | | |

### Example 4. Fab-Fc/scFv-Fc with TfR-Binding Fc Polypeptide

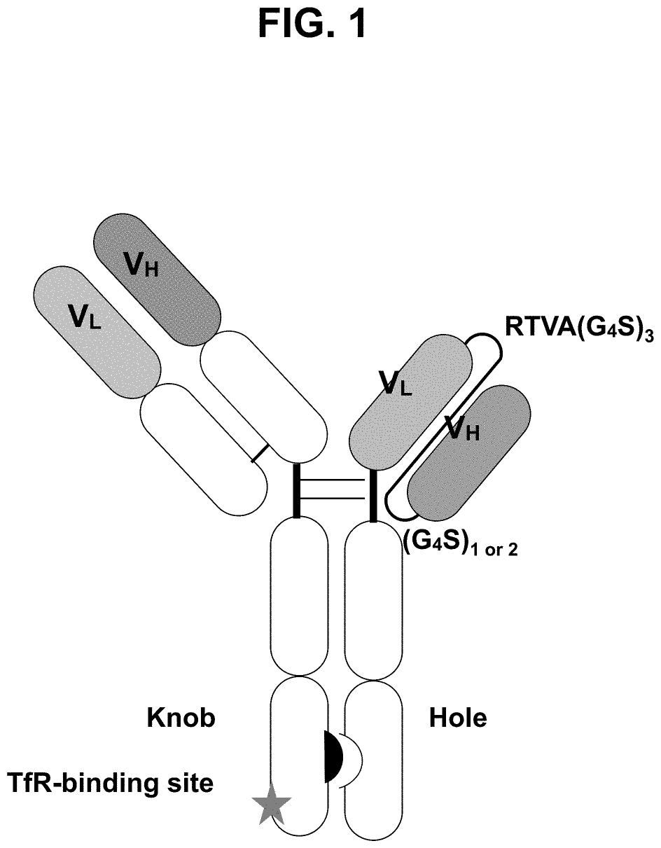

This example describes the generation and characterization of an engineered protein comprising a TfR-binding Fc polypeptide that is fused to two different targeting variable domains (a variable domain that targets a first antigen (BACE1) and a variable domain that targets a second antigen (Tau)). The protein can be generated in a single cell without light chain mispairing or steering by making the asymmetric fusion construct shown in FIG. 1. These constructs comprise three polypeptide chains and were made through simultaneous recombinant expression of each chain. The first chain is a TfR-binding Fc polypeptide comprising a knob mutation for Fc heterodimerization, and a hinge fused at its N-terminus to the Fd portion of a Fab. The second chain is the corresponding light chain, which pairs to form a Fab against antigen target 1. The third chain is an Fc polypeptide comprising a hinge and a N-terminal flexible linker (e.g., G₄S or (G₄S)₂) and an scFv against antigen target 2.

Polypeptides with this architecture were generated using the TfR-binding Fc region 3C.35.23.4 with a knob mutation fused to an anti-BACE1 Fab, paired with a hole Fc fused to an scFv for an anti-Tau antibody. Four versions of the anti-Tau scFv were generated; the linker to the Fc was tested as GGGGS (SEQ ID NO:371) or GGGGSGGGGS (SEQ ID NO:372), and the order of domains was tested as either VL-linker-VH or VH-linker-VL. For the former orientation, the linker RTVAGGGGSGGGGSGGGGS (SEQ ID NO:374) was used, and for the latter orientation the linker ASTKGGGGSGGGGSGGGGS (SEQ ID NO:375) was used. The same anti-BACE1 light chain sequence was used for all constructs. All constructs were made as effector function null Fc's by incorporating L234A/L235A (LALA) mutations.

Genes corresponding to each of the three chains was cloned into expression vectors, and the vectors were co-transfected into ExpiCHO cells for transient expression then purified by Protein A chromotagraphy. The recombinant polypeptides were subsequently tested for their ability to engage the first antigen (BACE1), the second antigen (Tau), and TfR using Biacore. As indicated in Table 10 below, all four variants bound TfR and BACE1, but only variants 2 and 4 bound Tau. This result suggests that the VL-linker-VH orientation is preferred for this scFv.

TABLE 10
Summary of Binding Kinetics of Bispecific
Proteins Having Fab-Fc/scFv-Fc Architecture

| | scFv | | TfR KD | BACE1 KD | Tau KD |
|---|---|---|---|---|---|
| Variant | orientation | Linker | (M) | (M) | (M) |
| V1 | VH-L-VL | (G₄S)₂ | 7.0E−7 | 1.3E−10 | No binding |
| V2 | VL-L-VH | (G₄S)₂ | 3.9E−7 | 6.3E−10 | <E−10 |
| V3 | VH-L-VL | G₄S | 9.9E−7 | 3.9E−10 | No binding |
| V4 | VL-L-VH | G₄S | 3.9E−7 | 9.8E−10 | <E−10 |

### Example 5. C-Terminal Fv Fused to TfR-Binding Fc Polypeptides

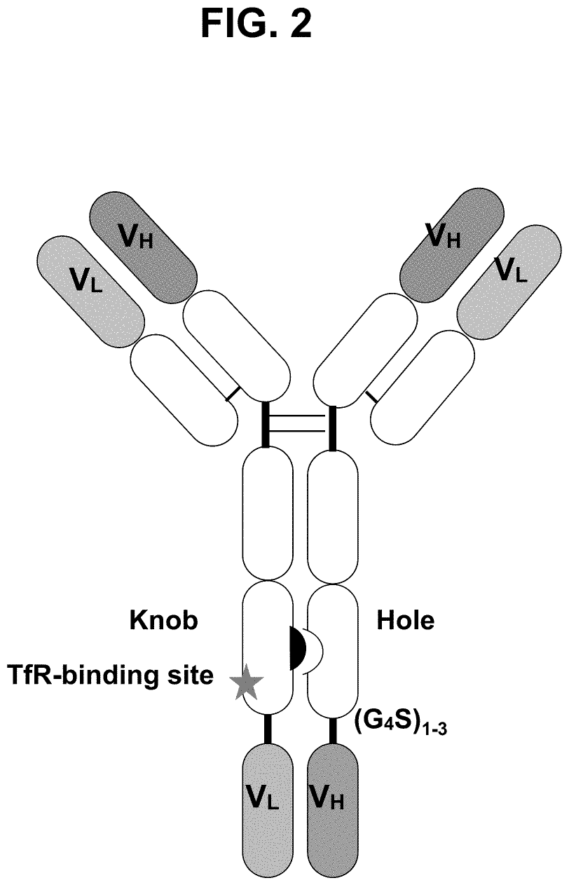

To incorporate target antigen binding, a Fc polypeptide comprising one Fc subunit with TfR-binding mutations and a knob mutation and a second Fc subunit with a hole mutation can be modified by adding a flexible linker to the C-termini of both chains followed by the variable domains from an antibody (VH to one subunit and VL to the other). In an exemplary embodiment of this architecture, the N-termini of the Fc domains are further fused to a Fab that binds to a second antigen, as shown in FIG. 2. The resulting configuration is a four chain polypeptide (two different heavy chains and two copies of the same light chain) that binds to TfR, one target antigen bivalently, and a second antigen monovalently.

Polypeptides of this architecture were generated where Fab arms from an anti-Tau antibody were fused to the N-terminus of TfR-binding polypeptide 3C.35.23.4, thereby allowing bivalent binding to Tau, and VL and VH from an anti-BACE1 antibody were respectively fused to the N-termini of the two Fc chains following a linker. A total of eight molecules were initially generated where three parameters were varied: the fused Fv's were from one of two anti-BACE1 antibody clones, the linker was either GGGGS (SEQ ID NO:371) or GGGGSGGGGS (SEQ ID NO:372), and the orientation of the VH and VL fusions (e.g., either VH was on heavy chain 1 and VL was on heavy chain 2, or vice versa).

These constructs comprise three polypeptide chains and were made through simultaneous recombinant expression of each chain. The first chain is an Fc polypeptide comprising the TfR-binding mutations 3C.35.23.4 and a knob mutation, fused at the N-terminus to the Fd region from an anti-Tau Fab, and fused at the C-terminus to a linker followed by the VH or VL from an anti-BACE1 Fab. The second chain is an Fc polypeptide comprising a hole mutation, fused at the N-terminus to the Fd region from an anti-Tau Fab, and fused at the C-terminus to a linker followed by the alternate variable domain (VH or VL) from the anti-BACE1 Fab. The third chain is the light chain corresponding to the anti-Tau Fab. All heavy chains had the C-terminal lysine from the canonical Fc sequence removed, and were made as effector function null Fc's by incorporating L234A/L235A (LALA) mutations.

Genes corresponding to each of the three chains were cloned into expression vectors, and the vectors were co-transfected into ExpiCHO cells for transient expression, then purified by Protein A chromotagraphy. The recombinant polypeptides were tested for their ability to engage the BACE1, Tau, and TfR using Biacore, as shown in Table 11 below.

Table 11. Summary of Binding Kinetics of Bispecific Proteins Having mAb/Fv Architecture

TABLE 11
Summary of Binding Kinetics of Bispecific
Proteins Having mAb/Fv Architecture

| | Ct-VH | | | | |
|---|---|---|---|---|---|
| | linked to | | TfR KD | BACE1 KD | Tau KD |
| Variant | (knob or hole) | Linker | (M) | (M) | (M) |
| V1 | Hole | G₄S | 5.1E−7 | 1.3E−8 | <1.0E−10 |
| V2 | Knob | G₄S | 4.3E−7 | 1.2E−8 | <1.0E−10 |
| V3 | Hole | (G₄S)₂ | 2.8E−7 | 4.0E−8 | <1.0E−10 |
| V4 | Knob | (G₄S)₂ | 3.7E−7 | 2.6E−8 | <1.0E−10 |
| V5 | Hole | G₄S | 3.2E−7 | 1.1E−8 | <1.0E−10 |
| V6 | Knob | G₄S | Nd | Nd | <1.0E−10 |
| V7 | Hole | (G₄S)₂ | 3.6E−7 | 2.1E−8 | <1.0E−10 |
| V8 | Knob | (G₄S)₂ | 2.9E−7 | 9.0E−9 | <1.0E−10 |

### Example 6. C-Terminal scFv Fusion to TfR-Binding Peptides

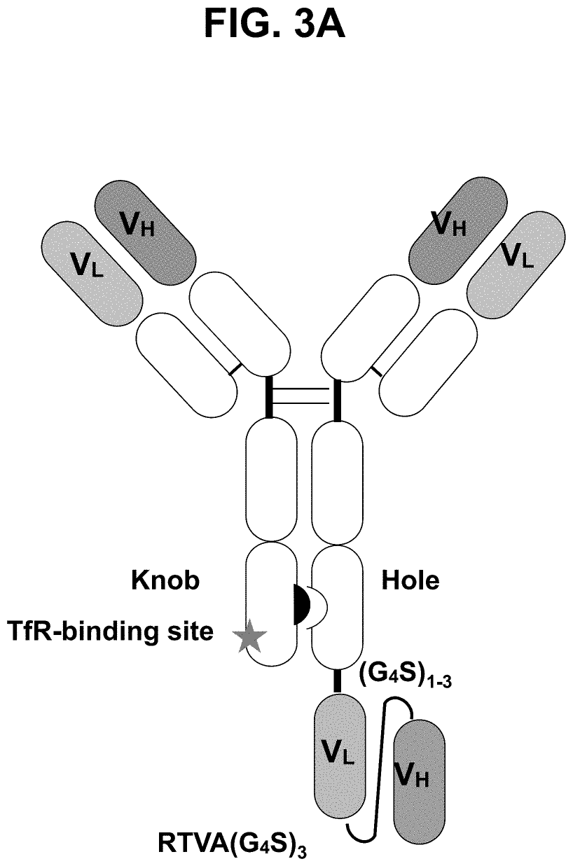

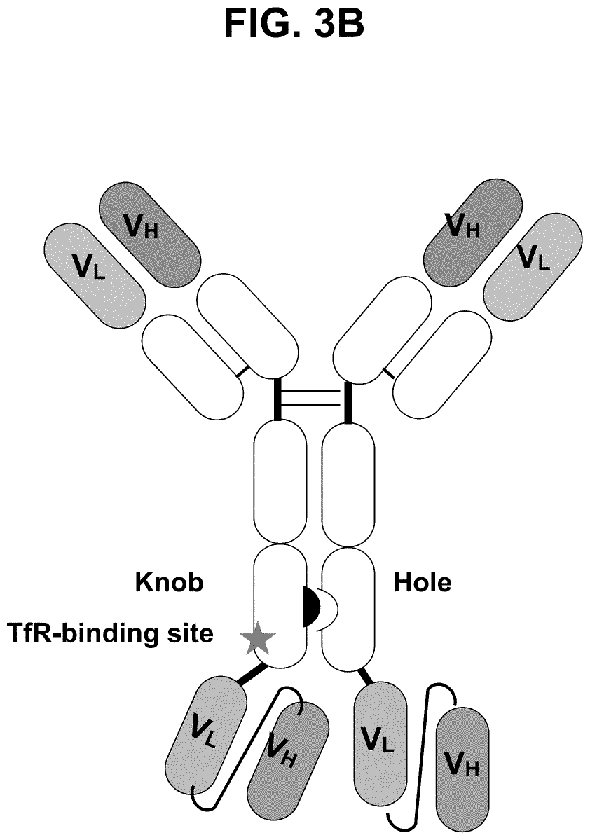

Polypeptides that comprise TfR-binding peptides fused at the N-terminus with Fabs against a first antigen and at the C-terminus with scFv(s) against the second antigen (either on the heavy chain hole only, FIG. 3A, or on both the knob and hole chains, FIG. 3B) were generated similarly to as described in Examples 1 and 2. Three expression plasmids were generated for each construct: a TfR-binding Fc polypeptide comprising a knob mutation along with N-terminal Fd against target 1 with or without a C-terminal scFv against target 2, an Fc polypeptide comprising a hole mutation along with N-terminal Fd against target 1 and a C-terminal scFv against target 2, and a light chain against target 1. The variable domains used were derived from anti-BACE1 antibodies and an anti-Tau antibody; orientations with BACE1 as target 1 and Tau as target 2 or vice versa were generated.

### Example 7. Generation of Bispecific Proteins

Engineered proteins having the architecture of a bispecific protein as shown in FIG. 1, FIG. 2, FIG. 3A, or FIG. 3B were generated to target two different antigens (Tau and BACE1). Architecture and sequence details for the constructs are shown in Tables 12-14 below. All constructs were made as effector function null by incorporating L234A/L235A (LALA) mutations into the Fc polypeptides. For the constructs in Table 13, all of the constructs had the C-terminal lysine immediately preceding the linker removed from both heavy chain 1 and heavy chain 2. For the constructs in Table 14, some constructs were generated in which the C-terminal lysine immediately preceding the linker (“Lys447”) was removed from heavy chain 2 (for proteins having one C-terminal scFv), or from both heavy chain 1 and heavy chain 2 (for proteins having two C-terminal scFvs). For the constructs in Tables 13 and 14, some constructs were generated that incorporated M428L/N434S (LS) mutations into the Fc polypeptides.

TABLE 12
Sequences for Bispecific Proteins Having Fab-Fc/scFv-Fc Architecture

| | | scFv- | | scFv | | | TfR |
|---|---|---|---|---|---|---|---|
| Construct | | CH2 | scFv | Domain | | scFv | Binding |
| No. | Fab | Linker | Antibody | Order | scFv Linker | Disulfide | Clone |
| 1 | Anti-BACE1 | G₄S | Anti-Tau | VL-VH | RTVA(G₄S)₃ | — | 35.23.4 |
| 2 | Anti-BACE1 | G₄S | Anti-Tau | VL-VH | RTVA(G₄S)₃ | H44-L100 | 35.23.4 |
| 3 | Anti-BACE1 | G₄S | Anti-Tau | VL-VH | (G₄S)₃ | — | 35.23.4 |
| 4 | Anti-BACE1 | G₄S | Anti-Tau | VL-VH | (G₄S)₃ | H44-L100 | 35.23.4 |
| 5 | Anti-Tau | G₄S | Anti-BACE1 | VL-VH | RTVA(G₄S)₃ | — | 35.23.4 |
| 6 | Anti-Tau | G₄S | Anti-BACE1 | VL-VH | RTVA(G₄S)₃ | H44-L100 | 35.23.4 |
| 7 | Anti-Tau | G₄S | Anti-BACE1 | VH-VL | ASTK(G₄S)₃ | — | 35.23.4 |
| 8 | Anti-Tau | G₄S | Anti-BACE1 | VH-VL | ASTK(G₄S)₃ | H44-L100 | 35.23.4 |
| 9 | Anti-Tau | G₄S | Anti-BACE1 | VL-VH | RTVA(G₄S)₃ | — | 35.23.4 |
| 10 | Anti-Tau | G₄S | Anti-BACE1 | VL-VH | RTVA(G₄S)₃ | H44-L100 | 35.23.4 |
| 11 | Anti-Tau | G₄S | Anti-BACE1 | VH-VL | ASTK(G₄S)₃ | — | 35.23.4 |
| 12 | Anti-Tau | G₄S | Anti-BACE1 | VH-VL | ASTK(G₄S)₃ | H44-L100 | 35.23.4 |

TABLE 13
Sequences for Bispecific Proteins Having C-Terminal-Fv Architecture

| | | | | | | | Half-Life | TfR |
|---|---|---|---|---|---|---|---|---|
| Construct | | Fv | Linker | Fv | Linker | scFv | Extension | Binding |
| No. | Fab | (knob) | (knob) | (hole) | (hole) | Disulfide | Mutation | Clone |
| 13 | Anti-Tau | Anti- | G₄S | Anti- | G₄S | — | — | 35.23.4 |
| | | BACE1 | | BACE1 | | | | |
| 14 | Anti-Tau | Anti- | G₄S | Anti- | G₄S | — | — | 35.23.4 |
| | | BACE1 | | BACE1 | | | | |
| 15 | Anti-Tau | Anti- | (G₄S)₂ | Anti- | (G₄S)₂ | — | — | 35.23.4 |
| | | BACE1 | | BACE1 | | | | |
| 16 | Anti-Tau | Anti- | (G₄S)₂ | Anti- | (G₄S)₂ | — | — | 35.23.4 |
| | | BACE1 | | BACE1 | | | | |
| 17 | Anti-Tau | Anti- | G₄S | Anti- | G₄S | — | — | 35.23.4 |
| | | BACE1 | | BACE1 | | | | |
| 18 | Anti-Tau | Anti- | G₄S | Anti- | G₄S | — | — | 35.23.4 |
| | | BACE1 | | BACE1 | | | | |
| 19 | Anti-Tau | Anti- | (G₄S)₂ | Anti- | (G₄S)₂ | — | — | 35.23.4 |
| | | BACE1 | | BACE1 | | | | |
| 20 | Anti-Tau | Anti- | (G₄S)₂ | Anti- | (G₄S)₂ | — | — | 35.23.4 |
| | | BACE1 | | BACE1 | | | | |
| 21 | Anti-Tau | Anti- | (G₄S)₂-G₄ | Anti- | (G₄S)₂-G₄ | — | — | 35.23.4 |
| | | BACE1 | | BACE1 | | | | |
| 22 | Anti-Tau | Anti- | (G₄S)₂-G₄ | Anti- | (G₄S)₂-G₄ | H44-L100 | — | 35.23.4 |
| | | BACE1 | | BACE1 | | | | |
| 23 | Anti-Tau | Anti- | (G₄S)₂-G₄ | Anti- | (G₄S)₂-G₄ | — | — | 35.23.4 |
| | | BACE1 | | BACE1 | | | | |
| 24 | Anti-Tau | Anti- | (G₄S)₂-G₄ | Anti- | (G₄S)₂-G₄ | H44-L100 | — | 35.23.4 |
| | | BACE1 | | BACE1 | | | | |
| 25 | Anti-Tau | Anti- | (G₄S)₃-G₄ | Anti- | (G₄S)₃-G₄ | — | — | 35.23.4 |
| | | BACE1 | | BACE1 | | | | |
| 26 | Anti-Tau | Anti- | (G₄S)₃-G₄ | Anti- | (G₄S)₃-G₄ | H44-L100 | — | 35.23.4 |
| | | BACE1 | | BACE1 | | | | |
| 27 | Anti-Tau | Anti- | (G₄S)₃-G₄ | Anti- | (G₄S)₃-G₄ | — | — | 35.23.4 |
| | | BACE1 | | BACE1 | | | | |
| 28 | Anti-Tau | Anti- | (G₄S)₃-G₄ | Anti- | (G₄S)₃-G₄ | H44-L100 | — | 35.23.4 |
| | | BACE1 | | BACE1 | | | | |
| 29 | Anti-Tau | Anti- | (G₄S)₂-G₄ | Anti- | G₄S-G₄ | — | — | 35.23.4 |
| | | BACE1 | | BACE1 | | | | |
| 30 | Anti-Tau | Anti- | (G₄S)₂-G₄ | Anti- | G₄S-G₄ | H44-L100 | — | 35.23.4 |
| | | BACE1 | | BACE1 | | | | |
| 31 | Anti-Tau | Anti- | (G₄S)₂-G₄ | Anti- | G₄S-G₄ | — | — | 35.23.4 |
| | | BACE1 | | BACE1 | | | | |
| 32 | Anti-Tau | Anti- | (G₄S)₂-G₄ | Anti- | G₄S-G₄ | H44-L100 | — | 35.23.4 |
| | | BACE1 | | BACE1 | | | | |
| 33 | Anti-Tau | Anti- | (G₄S)₂-G₄ | Anti- | (G₄S)₂-G₄ | H44-L100 | — | 35.23 |
| | | BACE1 | | BACE1 | | | | |
| 34 | Anti-Tau | Anti- | (G₄S)₂-G₄ | Anti- | (G₄S)₂-G₄ | H44-L100 | — | 35.23.3 |
| | | BACE1 | | BACE1 | | | | |
| 35 | Anti-Tau | Anti- | (G₄S)₂-G₄ | Anti- | (G₄S)₂-G₄ | H44-L100 | LS | 35.23.4 |
| | | BACE1 | | BACE1 | | | | |
| 36 | Anti-Tau | Anti- | (G₄S)₂-G₄ | Anti- | (G₄S)₂-G₄ | H44-L100 | LS | 35.23 |
| | | BACE1 | | BACE1 | | | | |
| 37 | Anti-Tau | Anti- | (G₄S)₂-G₄ | Anti- | (G₄S)₂-G₄ | H44-L100 | LS | 35.23.3 |
| | | BACE1 | | BACE1 | | | | |

TABLE 14
Sequences for Bispecific Proteins Having C-Terminal-scFv Architecture

| | | | | | | | | | |
|---|---|---|---|---|---|---|---|---|---|
| | | CH3- | | scFv | | | Half-Life | Lysine | TfR |
| Construct | | scFv | scFv | Domain | | scFv | Extension | 447 | Binding |
| No. | Fab | Linker | Antibody | Order | scFv Linker | Disulfide | Mutation | Removed? | Clone |

Proteins Having One C-Terminal scFv

| | | | | | | | | | |
|---|---|---|---|---|---|---|---|---|---|
| 38 | Anti- | G₄S | Anti- | VL-VH | RTVA(G₄S)₃ | — | — | No | 35.23.4 |
| | Tau | | BACE1 | | | | | | |
| 39 | Anti- | (G₄S)₂ | Anti- | VL-VH | RTVA(G₄S)₃ | — | — | No | 35.23.4 |
| | Tau | | BACE1 | | | | | | |
| 40 | Anti- | G₄S | Anti- | VL-VH | RTVA(G₄S)₃ | H44- | — | No | 35.23.4 |
| | Tau | | BACE1 | | | L100 | | | |
| 41 | Anti- | (G₄S)₂ | Anti- | VL-VH | RTVA(G₄S)₃ | H44- | — | No | 35.23.4 |
| | Tau | | BACE1 | | | L100 | | | |
| 42 | Anti- | G₄S | Anti- | VH-VL | ASTK(G₄S)₃ | — | — | No | 35.23.4 |
| | Tau | | BACE1 | | | | | | |
| 43 | Anti- | (G₄S)₂ | Anti- | VH-VL | ASTK(G₄S)₃ | — | — | No | 35.23.4 |
| | Tau | | BACE1 | | | | | | |
| 44 | Anti- | G₄S | Anti- | VH-VL | ASTK(G₄S)₃ | H44- | — | No | 35.23.4 |
| | Tau | | BACE1 | | | L100 | | | |
| 45 | Anti- | (G₄S)₂ | Anti- | VH-VL | ASTK(G₄S)₃ | H44- | — | No | 35.23.4 |
| | Tau | | BACE1 | | | L100 | | | |
| 46 | Anti- | (G₄S)₂ | Anti- | VL-VH | RTVA(G₄S)₃ | H44- | — | Yes | 35.23.4 |
| | Tau | | BACE1 | | | L100 | | | |
| 47 | Anti- | (G₄S)₂ | Anti- | VH-VL | ASTK(G₄S)₃ | H44- | — | Yes | 35.23.4 |
| | Tau | | BACE1 | | | L100 | | | |
| 48 | Anti- | (G₄S)₂ | Anti- | VL-VH | RTVA(G₄S)₃ | H44- | — | Yes | 35.23.1.1 |
| | Tau | | BACE1 | | | L100 | | | |
| 49 | Anti- | (G₄S)₂ | Anti- | VL-VH | RTVA(G₄S)₃ | H44- | — | Yes | 35.23.3 |
| | Tau | | BACE1 | | | L100 | | | |
| 50 | Anti- | (G₄S)₂ | Anti- | VL-VH | RTVA(G₄S)₃ | H44- | — | Yes | 35.23 |
| | Tau | | BACE1 | | | L100 | | | |
| 51 | Anti- | (G₄S)₂ | Anti- | VL-VH | RTVA(G₄S)₃ | H44- | — | Yes | 35.23.3 |
| | Tau | | BACE1 | | | L100 | | | |
| 52 | Anti- | (G₄S)₂ | Anti- | VL-VH | RTVA(G₄S)₃ | H44- | LS | Yes | 35.23.4 |
| | Tau | | BACE1 | | | L100 | | | |
| 53 | Anti- | (G₄S)₂ | Anti- | VL-VH | RTVA(G₄S)₃ | H44- | LS | Yes | 35.23 |
| | Tau | | BACE1 | | | L100 | | | |
| 54 | Anti- | (G₄S)₂ | Anti- | VL-VH | RTVA(G₄S)₃ | H44- | LS | Yes | 35.23.3 |
| | Tau | | BACE1 | | | L100 | | | |
| 55 | Anti- | (G₄S)₂ | Anti- | VH-VL | ASTK(G₄S)₃ | H44- | — | Yes | 35.23 |
| | Tau | | BACE1 | | | L100 | | | |

Proteins Having One C-Terminal scFv

| | | | | | | | | | |
|---|---|---|---|---|---|---|---|---|---|
| 56 | Anti- | (G₄S)₂ | Anti- | VH-VL | ASTK(G₄S)₃ | H44- | — | Yes | 35.23.3 |
| | Tau | | BACE1 | | | L100 | | | |
| 57 | Anti- | (G₄S)₂ | Anti- | VH-VL | ASTK(G₄S)₃ | H44- | LS | Yes | 35.23.4 |
| | Tau | | BACE1 | | | L100 | | | |
| 58 | Anti- | (G₄S)₂ | Anti- | VH-VL | ASTK(G₄S)₃ | H44- | LS | Yes | 35.23 |
| | Tau | | BACE1 | | | L100 | | | |
| 59 | Anti- | (G₄S)₂ | Anti- | VH-VL | ASTK(G₄S)₃ | H44- | LS | Yes | 35.23.3 |
| | Tau | | BACE1 | | | L100 | | | |

Proteins Having Two C-Terminal scFvs

| | | | | | | | | | |
|---|---|---|---|---|---|---|---|---|---|
| 60 | Anti- | (G₄S)₂ | Anti-Tau | VL-VH | RTVA(G₄S)₃ | — | — | Yes | 35.23.4 |
| | BACE1 | | | | | | | | |
| 61 | Anti- | (G₄S)₂ | Anti-Tau | VL-VH | RTVA(G₄S)₃ | H44- | — | Yes | 35.23.4 |
| | BACE1 | | | | | L100 | | | |
| 62 | Anti- | (G₄S)₂ | Anti- | VL-VH | RTVA(G₄S)₃ | H44- | — | Yes | 35.23.4 |
| | Tau | | BACE1 | | | L100 | | | |
| 63 | Anti- | (G₄S)₂ | Anti- | VH-VL | ASTK(G₄S)₃ | H44- | — | | 35.23.4 |
| | Tau | | BACE1 | | | L100 | | | |
| 64 | Anti- | (G₄S)₂ | Anti- | VL-VH | RTVA(G₄S)₃ | H44- | — | Yes | 35.23 |
| | Tau | | BACE1 | | | L100 | | | |
| 65 | Anti- | (G₄S)₂ | Anti- | VL-VH | RTVA(G₄S)₃ | H44- | — | Yes | 35.23.3 |
| | Tau | | BACE1 | | | L100 | | | |
| 66 | Anti- | (G₄S)₂ | Anti- | VL-VH | RTVA(G₄S)₃ | H44- | LS | Yes | 35.23.4 |
| | Tau | | BACE1 | | | L100 | | | |
| 67 | Anti- | (G₄S)₂ | Anti- | VL-VH | RTVA(G₄S)₃ | H44- | LS | Yes | 35.23 |
| | Tau | | BACE1 | | | L100 | | | |
| 68 | Anti- | (G₄S)₂ | Anti- | VL-VH | RTVA(G₄S)₃ | H44- | LS | Yes | 35.23.3 |
| | Tau | | BACE1 | | | L100 | | | |
| 69 | Anti- | (G₄S)₂ | Anti- | VH-VL | ASTK(G₄S)₃ | H44- | — | Yes | 35.23.4 |
| | Tau | | BACE1 | | | L100 | | | |
| 70 | Anti- | (G₄S)₂ | Anti- | VH-VL | ASTK(G₄S)₃ | H44- | — | Yes | 35.23 |
| | Tau | | BACE1 | | | L100 | | | |
| 71 | Anti- | (G₄S)₂ | Anti- | VH-VL | ASTK(G₄S)₃ | H44- | — | Yes | 35.23.3 |
| | Tau | | BACE1 | | | L100 | | | |
| 72 | Anti- | (G₄S)₂ | Anti- | VH-VL | ASTK(G₄S)₃ | H44- | LS | Yes | 35.23.4 |
| | Tau | | BACE1 | | | L100 | | | |
| 73 | Anti- | (G₄S)₂ | Anti- | VH-VL | ASTK(G₄S)₃ | H44- | LS | Yes | 35.23 |
| | Tau | | BACE1 | | | L100 | | | |
| 74 | Anti- | (G₄S)₂ | Anti- | VH-VL | ASTK(G₄S)₃ | H44- | LS | Yes | 35.23.3 |
| | Tau | | BACE1 | | | L100 | | | |

### Example 8. Biacore Assessment of Bispecific Proteins

### Biacore Assessment of BACE1/Tau Bispecific Proteins Comprising TfR-Binding Fc Polypeptides

The affinities of BACE1/Tau bispecific proteins comprising TfR-binding Fc polypeptides with its antigens were determined by surface plasmon resonance using a Biacore™ 8K instrument. Bispecific proteins were captured using Human Fab Capture Kit (GE, #28-9583-25) on Biacore Series S CM5 sensor chip (GE, #29149604). Serial 3-fold dilutions of each antigen (BACE1: 300, 100, 33.3, 11.1, 0 nM; Tau: 30, 10, 3.3, 1.1, 0.4 nM) were injected at a flow rate of 30 μL/min. The binding of the antigens to captured Fc polypeptide comprising a TfR binding site was monitored for 300 seconds and then their dissociation was monitored for 600+ seconds in HBS-EP+ running buffer. Binding response was corrected by subtracting the RU from a blank flow cell. A 1:1 Languir model of simultaneous fitting of kₒₙ and k_{off} was used for kinetics analysis. Binding data for bispecific proteins disclosed in Tables 12-14 is shown in Tables 15-17 below. An anti-BACE1/RSV bispecific protein having a TfR binding site (Clone 35.23.4), knobs-into-holes, and L234A/L235A substitutions (“C1”) was used as a control.

### Biacore Assessment of TfR Binding

The affinity of bispecific proteins for recombinant TfR apical domain was determined by surface plasmon resonance using a Biacore™ 8K instrument in 1×HBS-EP+ running buffer (GE Healthcare, BR100669). Biacore™ Series S CM5 sensor chips were immobilized with anti-human Fab (human Fab capture kit from GE Healthcare, 28958325). A fusion protein comprising Fab and an Fc polypeptide comprising a TfR binding site was captured for 30 seconds on each flow cell and serial 3-fold dilutions of human (2, 0.66, 0.22, 0.073, 0.24, and 0 uM) apical domain were injected at a flow rate of 30 μL/min using single cycle kinetics method. Each sample was analyzed with a 80-second association and a 3-minute dissociation. After each cycle, the chip was regenerated using 10 mM glycine-HCl (pH 2.1) for 30 seconds at 50 ul/min. Binding response was corrected by subtracting the RU from a reference flow cell. Steady-state affinities were obtained by fitting the response at equilibrium against the concentration using Biacore™ 8K Evaluation Software. To determine the affinity of the bispecific proteins for recombinant TfR ectodomain (ECD), Biacore™ Series S CM5 sensor chips were immobilized with streptavidin. Biotinylated 0.5 ug/ml human TfR ECD was captured for 45 seconds on each flow cell at 10 ul/min, and serial 3-fold dilutions of bispecific proteins buffer-exchanged in BBS were injected at a flow rate of 30 ul/min. Each sample was analyzed using single cycle kinetics as described above. Binding data for bispecific proteins disclosed in Tables 12-14 is shown in Tables 15-17 below. C1 was used as a control.

TABLE 15
Biacore Binding Data for Bispecific Proteins
Having Fab-Fc/scFv-Fc Architecture

| | Tau KD | hTfR apical |
|---|---|---|
| Construct # | (nM) | SSA (nM) |
| 1 | 1.8 | 370 |
| 2 | 1.6 | 390 |
| 3 | ND | ND |
| 4 | ND | ND |

| | BACE1 KD | hTfR apical | Tau KD |
|---|---|---|---|
| Construct # | (nM) | SSA (nM) | (pM) |
| C1 | 1.4 | 470 | ND |
| 5 | ND | ND | ND |
| 6 | 4.1 | 330 | ND |
| 7 | ND | ND | ND |
| 8 | no binding | 280 | ND |
| 9 | 2.4 | 350 | ND |
| 10 | 2.1 | 360 | 240 |
| 11 | 2.6 | 360 | ND |
| 12 | 2.7 | 360 | ND |

ND = Not Determined

TABLE 16
Biacore Binding Data for Bispecific Proteins
Having C-Terminal-Fc Architecture

| | BACE1 KD | hTfR apical |
|---|---|---|
| Construct # | (nM) | SSA (nM) |
| 13 | 13 | 510 |
| 14 | 12 | 430 |
| 15 | 40 | 280 |
| 16 | 4 | 370 |
| 17 | 11 | 320 |
| 18 | ND | ND |
| 19 | 21 | 360 |
| 20 | 9 | 290 |

| | BACE1 KD | hTfR apical | Tau KD |
|---|---|---|---|
| Construct # | (nM) | SSA (nM) | (pM) |
| C1 | 3.4 | 700 | ND |
| 21 | 8.9 | 470 | ND |
| 22 | 17 | 460 | ND |
| 23 | 11 | ND | ND |
| 24 | 7.8 | 440 | 420 |
| 25 | 8 | 270 | ND |
| 26 | 5 | ND | ND |
| 27 | 18 | 350 | ND |
| 28 | 26 | 210 | ND |
| 29 | 5.3 | 360 | ND |
| 30 | 4.6 | 450 | ND |
| 31 | 15 | 350 | ND |
| 32 | 20 | 340 | ND |

ND = Not Determined

TABLE 17
Biacore Binding Data for Bispecific Proteins
Having C-Terminal-scFv Architecture

| | BACE1 KD | hTfR apical |
|---|---|---|
| Construct # | (nM) | SSA (nM) |
| C1 | 3.4 | 700 |
| 38 | 4.2 | 560 |
| 39 | 3.6 | 610 |
| 40 | 3.3 | 570 |
| 41 | 3.3 | 550 |
| 42 | 3.2 | ND |
| 43 | 10.6 | 600 |
| 44 | 5.9 | 460 |
| 45 | 4.3 | 520 |

| | BACE1 KD | Tau KD | hTfR ECD |
|---|---|---|---|
| | (nM) | (pM) | SSA (nM) |
| 46 | 2.2 | 460 | 780 |
| 47 | ND | ND | ND |

| | BACE1 KD | hTfR apical | hTfR ECD |
|---|---|---|---|
| Construct # | (nM) | SSA (nM) | SSA (nM) |
| C1 | 1.1 | 520 | 380 |
| 48 | 1.5 | 460 | 1480 |
| 49 | 1.6 | 680 | 1540 |

| | BACE1 KD | hTfR apical | hTfR ECD | Tau KD |
|---|---|---|---|---|
| Construct # | (nM) | SSA (nM) | SSA (nM) | (pM) |
| C1 | 1.1 | 520 | 380 | ND |
| 60 | 0.6 | 210 | 870 | ND |
| 61 | ND | ND | ND | ND |
| 62 | 1.4 | 390 | 1490 | 240 |
| 63 | 4.4 | 250 | 1290 | ND |

ND = Not Determined

### Example 9. Quantification of BACE1 Inhibition Using CHO:huAPP Cells

### Culture Conditions

CHO:huAPP KI cells were generated at Genscript and maintained in 50% DMEM/50% F12 media (Gibco, 11320) with 10% FBS (Sigma F8317), 1× penicillin/streptomycin (Gibco 15140122), and 1× Genectin (Gibco 10131027) (referred to herein as “CHO media” (“CM”)).

### Cell Culture Treatments

CHO:huAPP cells (passage #4-18) were treated with various molecules (all chimeric molecules on a human IgG backbone). Molecules were first diluted in CM to a starting concentration of 1 or 2 μM and then diluted either 1:2 or 1:4 to generate a dilution series for measuring the dose response of each molecule. Medium of CHO:huAPP cells was entirely replaced with that containing experimental or control molecules. CHO:huAPP cells were then kept at 37° C. with 5% CO₂ for 24 hours. After 24 hours, media were collected for Aβ measurement by a HTFR assay.

### Aβ Quantification by HTFR

After 24 hours incubation of CHO:huAPP cells with experimental or control molecules, 100 μL of media was harvested. The molecule incubations were performed in duplicate and the Aβ1-40 measurements with technical duplicates. Measurements of human Aβ1-40 were conducted according to the Cisbio Aβ1-40 kit (Cisbio #62B40PEG). In brief: the kit provided two anti-Aβ1-40 antibodies that act as a FRET donor and receptor pair: one antibody was labeled with Eu3+-Cryptate (FRET donor) and the other with XL-665 (FRET receptor). Both antibodies were incubated with 5 μL of media, harvested from CHO:huAPP cultures, in a PerkinElmer OptiPlate 384 for 24 hours at 4° C. The plate was then read and Aβ1-40 concentration was calculated from a 665 nm/620 nm ratio.

Cellular BACE1 inhibition data for bispecific proteins disclosed in Tables 12-14 is shown in Tables 18-20 below. An anti-BACE1 antibody having a TfR binding site (Clone 35.23.4) (“C2”), an anti-BACE1/RSV bispecific protein having a TfR binding site (Clone 35.23.4) and L234A/L235A substitutions (“C3”), and a non-affinity matured anti-BACE1 lacking a TfR binding site or other Fc modifications (“C4”) were used as controls.

TABLE 18
Cellular BACE1 Inhibition for Bispecific Proteins
Having Fab-Fc/scFv-Fc Architecture

| | BACE1 Cellular | BACE1 Max | Concentration for |
|---|---|---|---|
| Construct # | IC50 (nM) | Inhibition (%) | 50% Inhibition (nM) |
| C2 | 35 | 69 | 99 |
| 6 | 6 | 58 | 42 |
| 9 | 18 | 70 | 37 |
| 10 | 8 | 62 | 35 |
| 11 | 3 | 63 | 14 |
| 12 | 11 | 67 | 27 |

TABLE 19
Cellular BACE1 Inhibition for Bispecific Proteins
Having C-Terminal-Fc Architecture

| | BACE1 Cellular | BACE1 Max | Concentration for |
|---|---|---|---|
| Construct # | IC50 (nM) | Inhibition (%) | 50% Inhibition (nM) |
| C3 | 58 | 67 | 157 |
| 14 | 481 | 35 | NA |
| 15 | 1215 | NA | NA |
| 16 | 554 | 65 | NA |
| 17 | 673 | 71 | 1382 |
| 19 | 61 | 57 | 427 |
| 20 | 126 | 60 | 558 |
| C2 | 23 | 73 | 57 |
| 24 | 18.5 | 59 | 124 |
| 30 | 151 | 66 | 378 |
| 31 | 144 | 66 | 487 |
| 32 | | | |
| C2 | 35 | 69 | 99 |
| 25 | 577 | 81 | 627 |
| 26 | 692 | 92 | 661 |
| 27 | 152 | 65 | 547 |
| 28 | 229 | 84 | 238 |

NA = Not Applicable

TABLE 20
Cellular BACE1 Inhibition for Bispecific Proteins
Having C-Terminal-scFv Architecture

| | BACE1 Cellular | BACE1 Max | Concentration for |
|---|---|---|---|
| Construct # | IC50 (nM) | Inhibition (%) | 50% Inhibition (nM) |
| C2 | 23 | 73 | 57 |
| 39 | 6 | 65 | 16 |
| 40 | 24 | 73 | 48 |
| 41 | 5 | 69 | 16 |
| 43 | 8 | 76 | 14 |
| 45 | 8 | 76 | 16 |
| C2 | 26 | 71 | 73 |
| 46 | 12 | 60 | 83 |
| C2 | 31 | 68 | 93 |
| 50 | 7 | 58 | 47 |
| C4 | 10 | 66 | 28 |
| 60 | 4 | 58 | 24 |
| 62 | 5 | 71 | 11 |
| 63 | 4 | 66 | 14 |

### Example 10. Pharmacokinetic Properties of BACE-Tau Bispecific Proteins Having TfR-Binding Fc Polypeptide

This example describes the characterization of pharmacokinetic properties of BACE1-Tau bispecific proteins having TfR-binding Fc polypeptides using mouse models.

### Wild-Type Mouse PK Evaluation

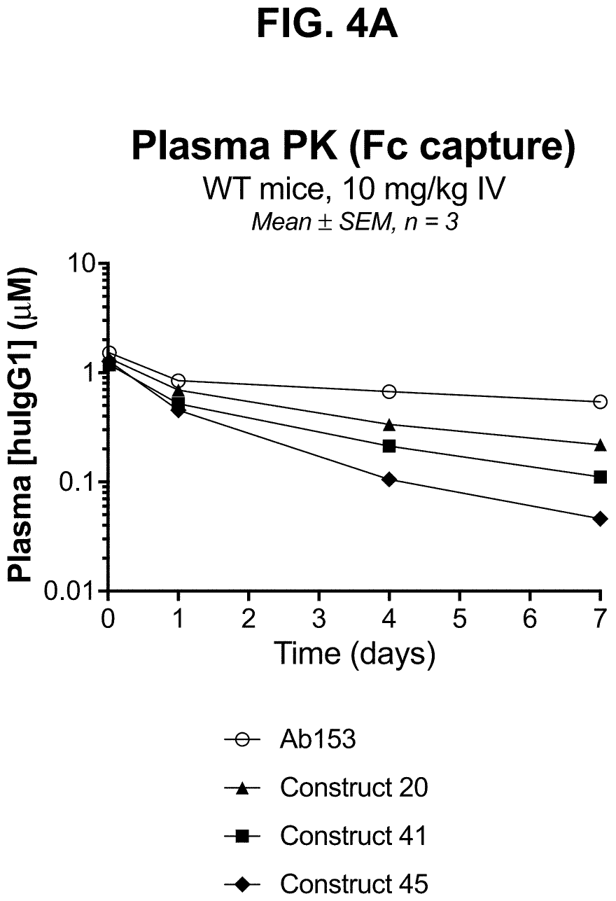

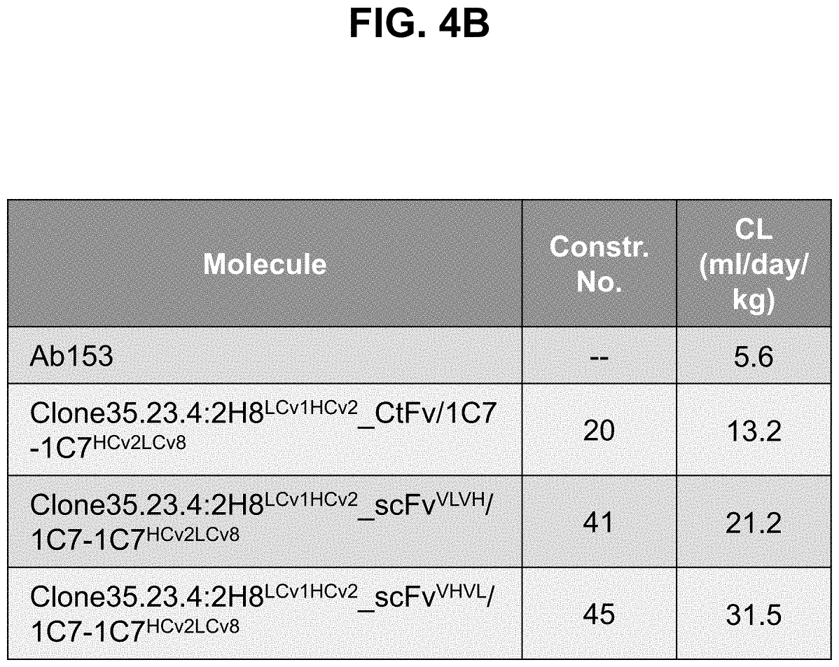

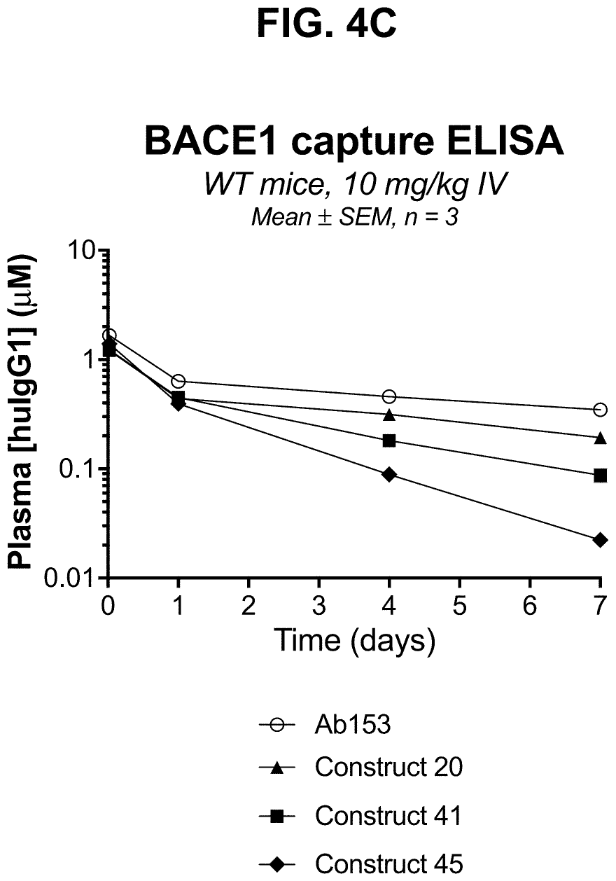

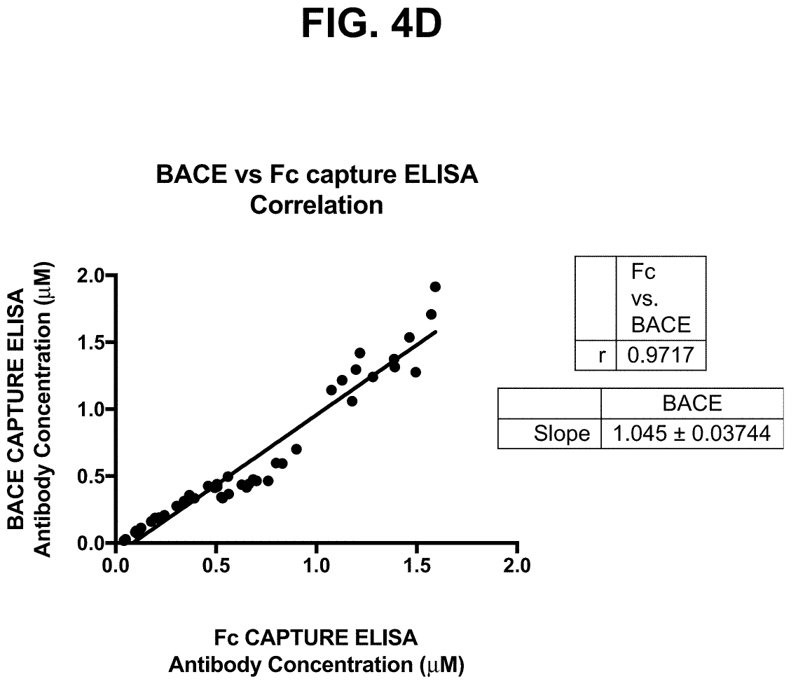

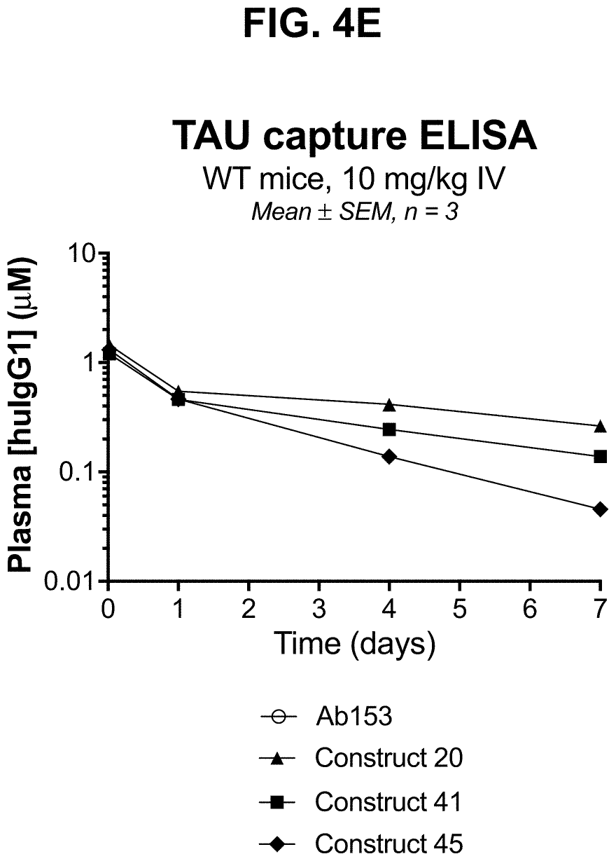

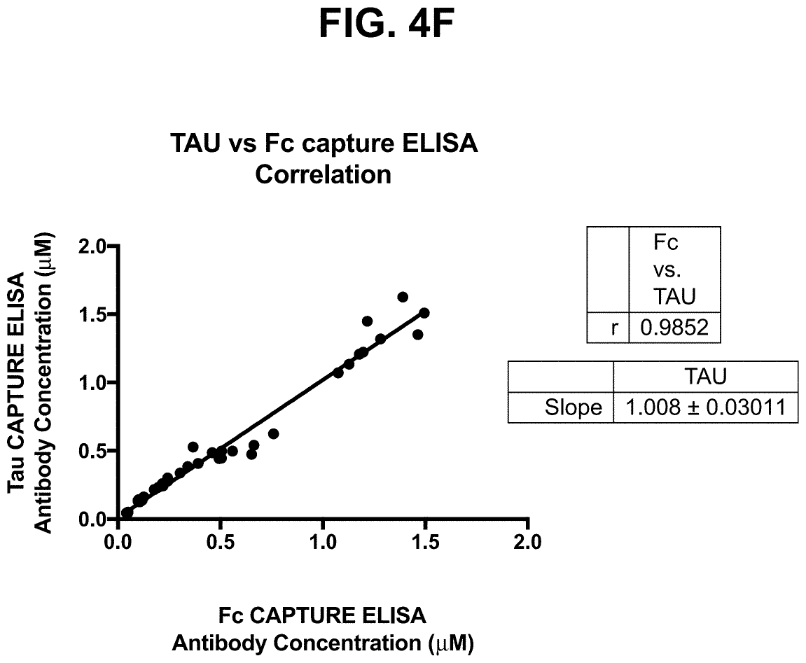

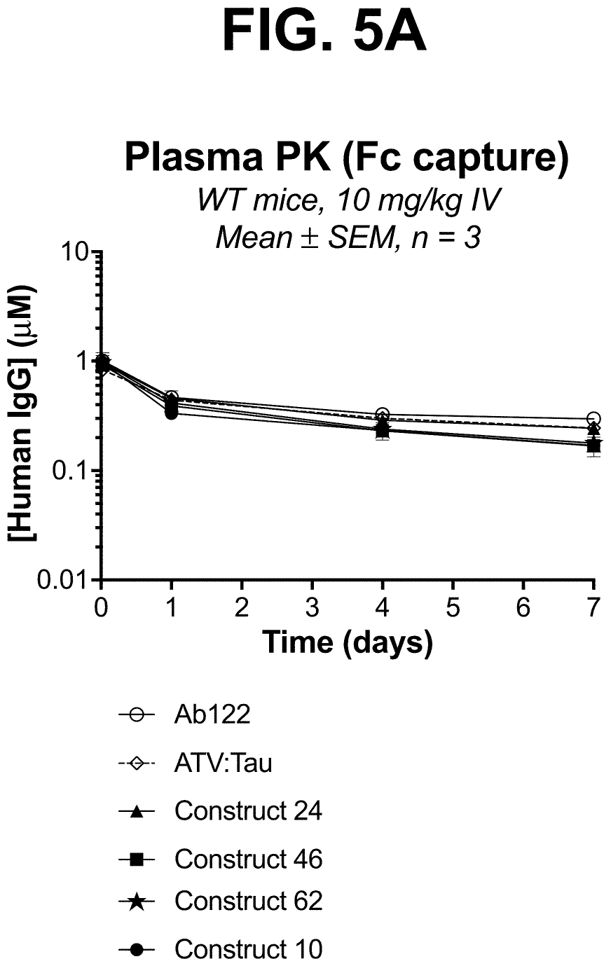

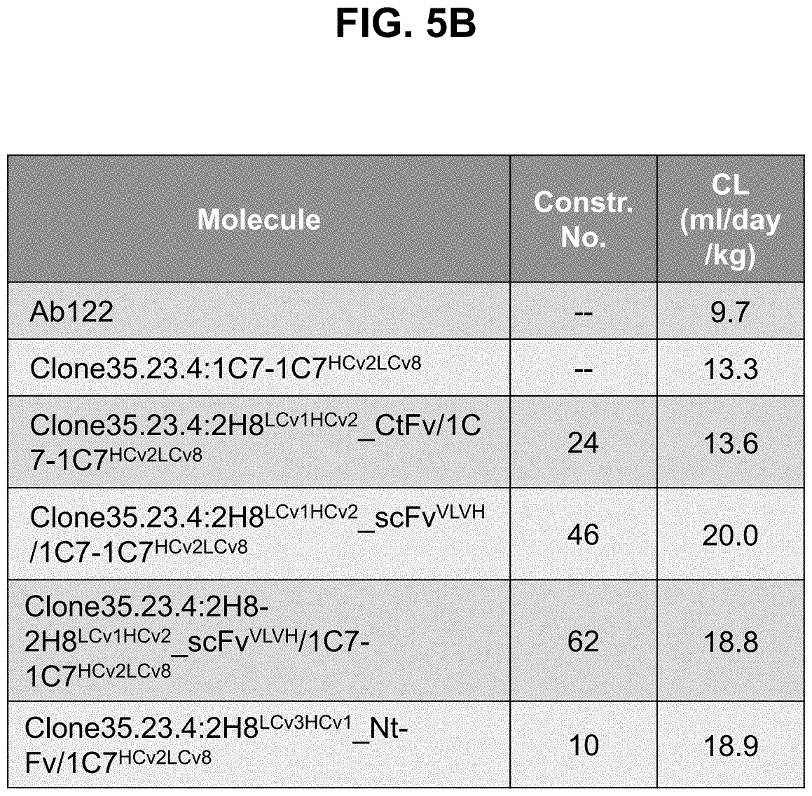

For in vivo pharmacokinetic (PK) evaluation, 6-8 week-old female wild-type C57B16 mice were intravenously dosed at 10 mg/kg with a BACE1-Tau bispecific protein having a Fab-Fc/scFv-Fc architecture (construct 10 as described in Table 12), a BACE1-Tau bispecific protein having a C-terminal Fv architecture (constructs 20 and 24, as described in Table 13), a BACE1-Tau bispecific protein having a C-terminal scFv architecture with an scFv fused to one Fc polypeptide (constructs 41, 45, and 46, as described in Table 14), a BACE1-Tau bispecific protein having a C-terminal architecture with an scFv fused to each Fc polypeptide (construct 62, as described in Table 14), an anti-BACE1 control antibody (Ab153), an anti-RSV negative control antibody (Ab122), or an anti-Tau antibody comprising a TfR-binding Fc polypeptide (ATV:Tau). In-life plasma was taken via submandibular-bleeds at the timepoints indicated in FIG. 4A or FIG. 5A. Blood was collected in EDTA plasma tubes, spun at 14,000 rpm for 5 minutes, and then plasma was isolated for subsequent analysis.

FIGS. 4A and 4B show the data from wild-type mouse PK evaluation of BACE1-Tau C-terminal Fv construct 20, BACE1-Tau C-terminal scFv constructs 41 and 45, and anti-BACE1 control antibody (Ab153). As shown in FIG. 4B, each of the BACE1-Tau bispecific proteins had faster clearance than the control anti-BACE1 antibody.

FIGS. 5A and 5B show the data from wild-type mouse PK evaluation of BACE1-Tau Fab-Fc/scFv-Fc construct 10, BACE1-Tau C-terminal Fv construct 24, BACE1-Tau C-terminal scFv construct 46, BACE1-Tau C-terminal scFv construct 62, anti-RSV negative control antibody (Ab122), and anti-Tau antibody comprising a TfR-binding Fc polypeptide (ATV:Tau). As shown in FIG. 5B, each of the BACE1-Tau bispecific proteins had acceptable clearance values within 1.5-2 fold of an anti-RSV negative control antibody (Ab122) and within 1.5 fold of a control anti-Tau antibody comprising a TfR-binding Fc polypeptide.

hTfR^{ms/hu} KI Mouse PK Evaluation

Human TfR knock-in (TfR^{ms/hu} KI) mice were also used for in vivo pharmacokinetic (PK) evaluation. Such a model can be used, for example, to measure and/or compare maximum brain concentration (Cₘₐₓ) and/or brain exposure, e.g., to determine whether Cₘₐₓ is increased and/or brain exposure is prolonged. TfR^{ms/hu} KI mice were generated using CRISPR/Cas9 technology to express human Tfrc apical domain within the murine Tfrc gene; the resulting chimeric TfR was expressed in vivo under the control of the endogenous promoter. As described in International Patent Application No. PCT/US2018/018302, which is incorporated by reference in its entirety herein, C57B16 mice were used to generate a knock-in of the human apical TfR mouse line via pronuclear microinjection into single cell embryos, followed by embryo transfer to pseudo pregnant females. Specifically, Cas9, single guide RNAs and a donor DNA were introduced into the embryos. The donor DNA comprised a human apical domain coding sequence that had been codon optimized for expression in mouse. The apical domain coding sequence was flanked with a left and a right homology arm. The donor sequence was designed such that the apical domain was inserted after the fourth mouse exon, and was immediately flanked at the 3′ end by the ninth mouse exon. A founder male from the progeny of the female that received the embryos was bred to wild-type females to generate F1 heterozygous mice. Homozygous mice were subsequently generated from breeding of F1 generation heterozygous mice.

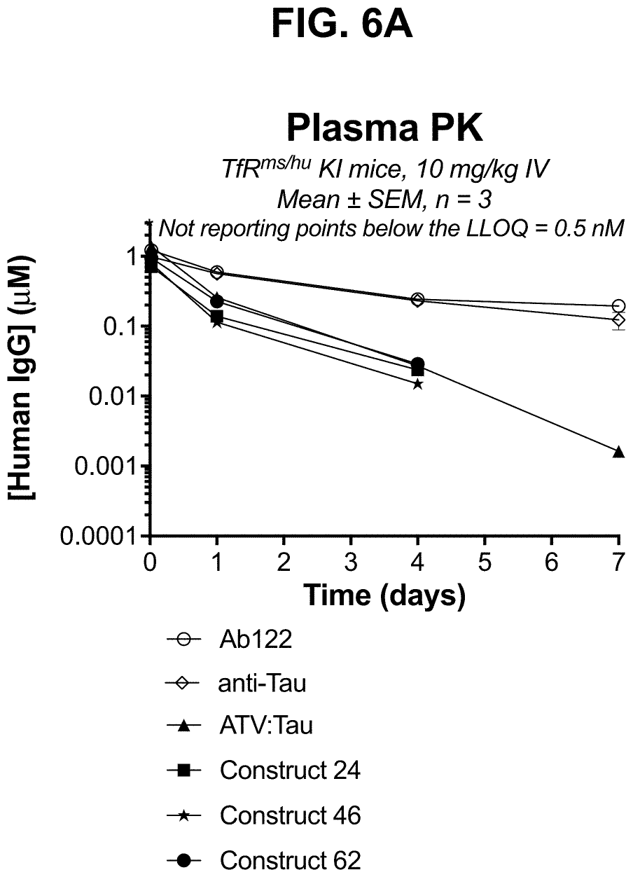

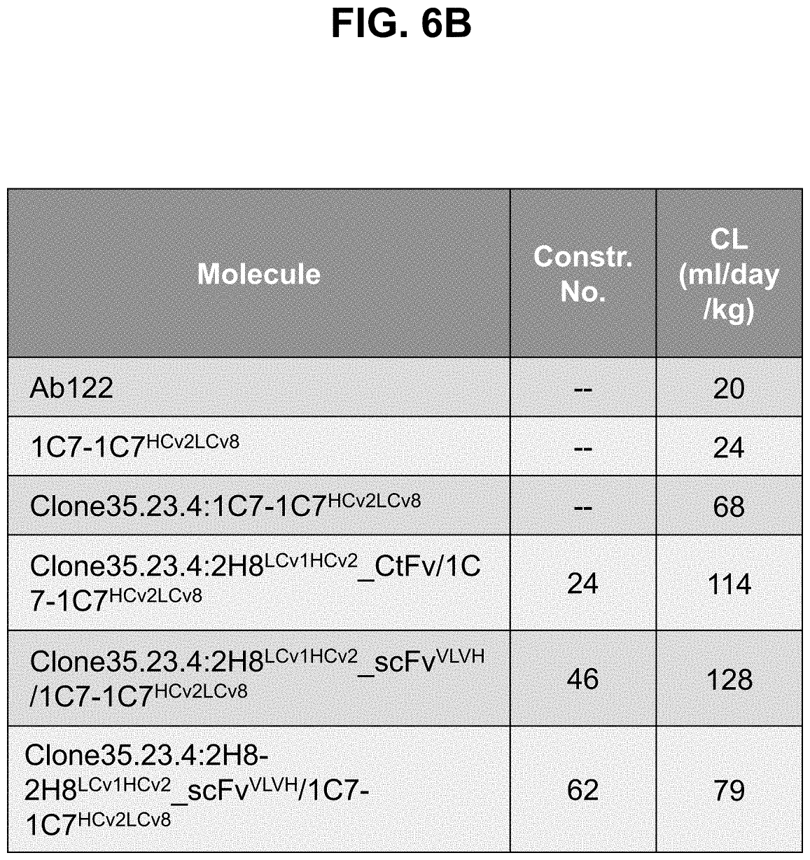

For PK analysis, 6-8 week-old female hTfR^{ms/hu} KI mice were intravenously dosed at 10 mg/kg with a BACE1-Tau bispecific protein having a C-terminal Fv architecture (construct 24, as described in Table 13), a BACE1-Tau bispecific protein having a C-terminal scFv architecture with an scFv fused to one Fc polypeptide (construct 46, as described in Table 14), a BACE1-Tau bispecific protein having a C-terminal scFv architecture with an scFv fused to each Fc polypeptide (construct 62, as described in Table 14), an anti-RSV negative control antibody (Ab122), an anti-Tau 1C7 antibody (anti-Tau), or an anti-Tau antibody comprising a TfR-binding Fc polypeptide (ATV:Tau). In-life plasma was taken via submandibular-bleeds at the timepoints indicated in FIG. 6A. Blood was collected in EDTA plasma tubes, spun at 14,000 rpm for 5 minutes, and then plasma was isolated for subsequent analysis.

As shown in FIGS. 6A and 6B, each of the BACE1-Tau bispecific proteins tested in the hTfR^{ms/hu} KI mouse model exhibited faster clearance than the anti-RSV negative control antibody (Ab122) or anti-Tau 1C7 antibody due to TfR binding and target-mediated clearance, and had acceptable clearance values within 2-fold of a control anti-Tau antibody comprising a TfR-binding Fc polypeptide.

### PS19/hTfR^{ms/hu} KI Mouse PK Evaluation

The pharmacokinetic properties of additional constructs were also evaluated in vivo in PS19/TfR^{ms/hu} KI mice. PS19/TfR^{ms/hu} KI mice were generated to express human Tfrc apical domain within the murine Tfrc gene and a mutant Tau gene that encodes a mutant human Tau protein comprising the amino acid substitution P272S relative to the sequence of SEQ ID NO:398 by crossing the PS19 mice to the TfR^{ms/hu} KI mice to generate a colony of PS19 HEMI (hemizygous) TfR^{ms/hu} HOM (homozygous) mice. Male PS19 HEMI TfR^{ms/hu} KI HOM mice are crossed to female TfR^{ms/hu} HOM mice to maintain the colony.

PS19/TfR^{ms/hu} KI mice were systemically dosed one time via the tail vein at 50 mg/kg. Prior to perfusion with PBS, blood was collected in EDTA plasma tubes via cardiac puncture and spun at 14,000 rpm for 5 minutes. Plasma was then isolated for subsequent PK/PD analysis. Brains were extracted after perfusion and hemi-brains were isolated for homogenization in 10× by tissue weight of 1% NP-40 in PBS (for PK) or 5 M GuHCl (for PD).

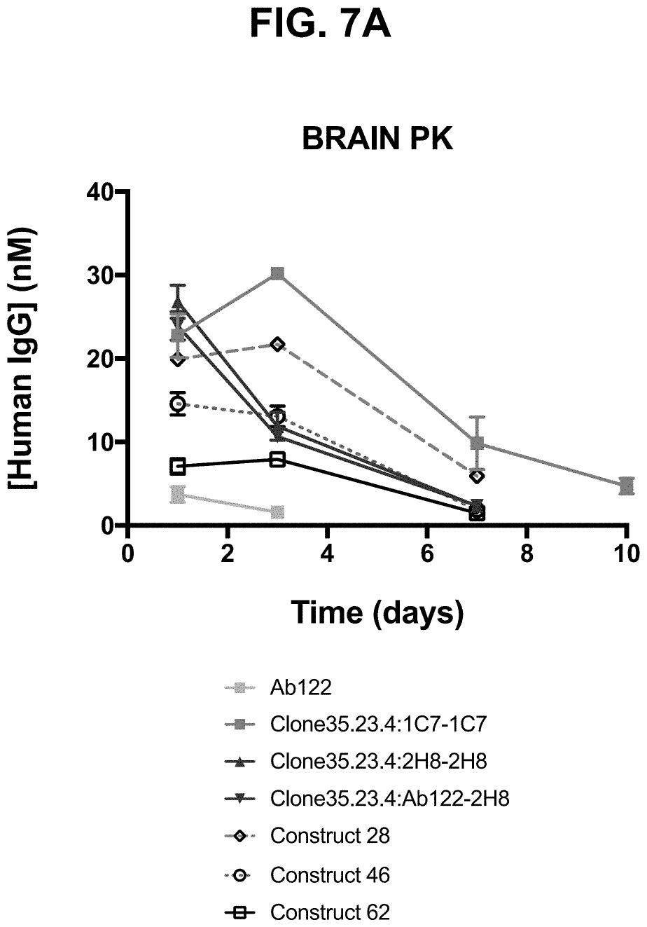

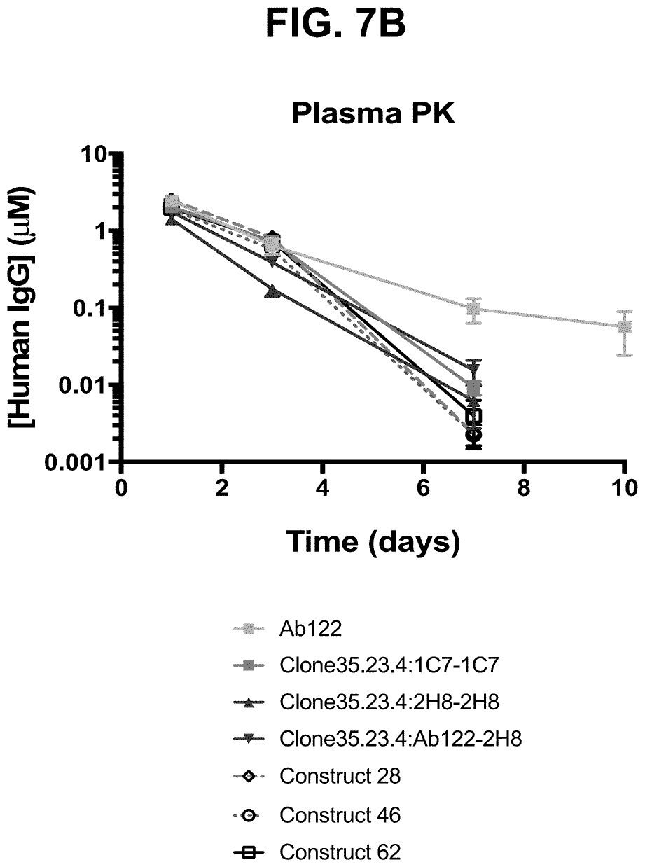

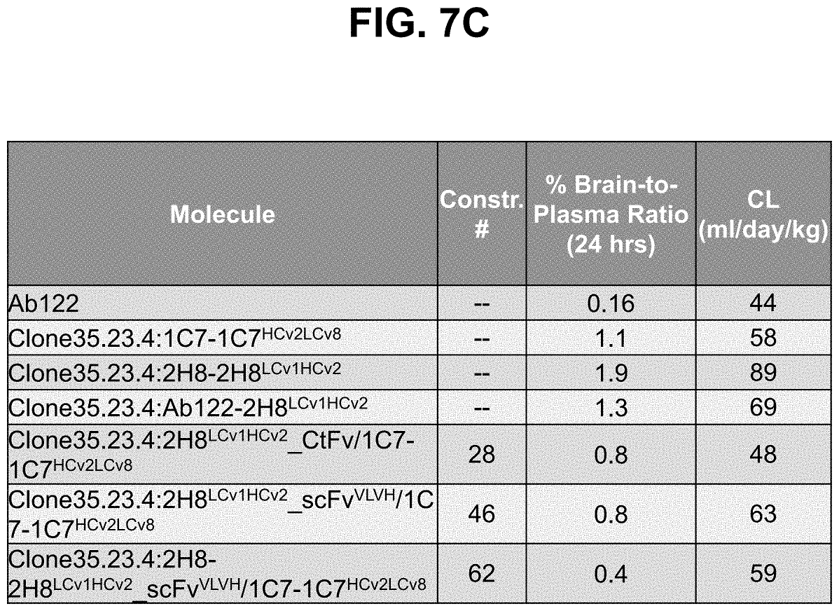

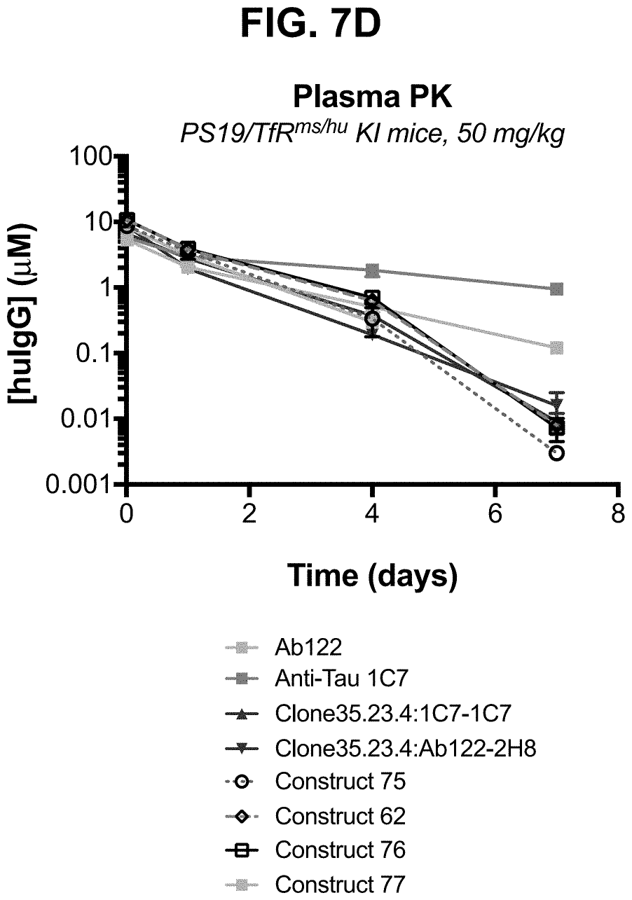

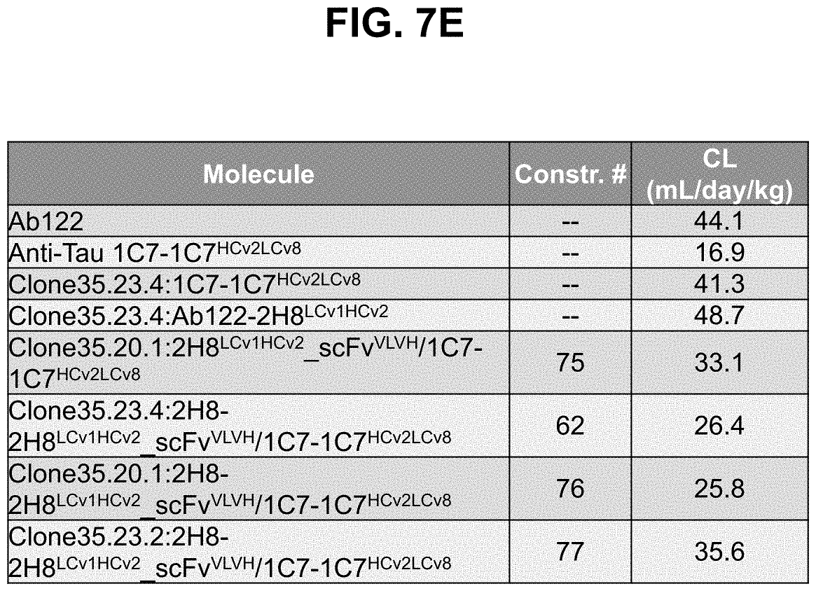

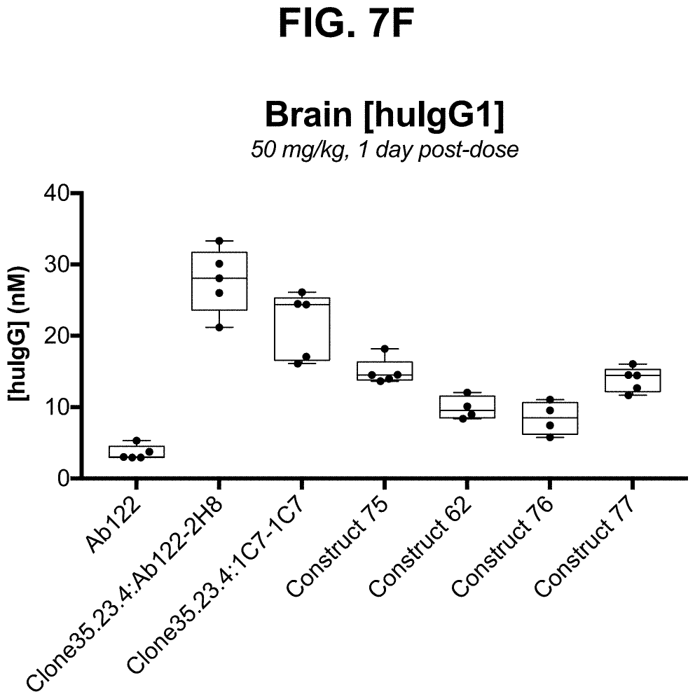

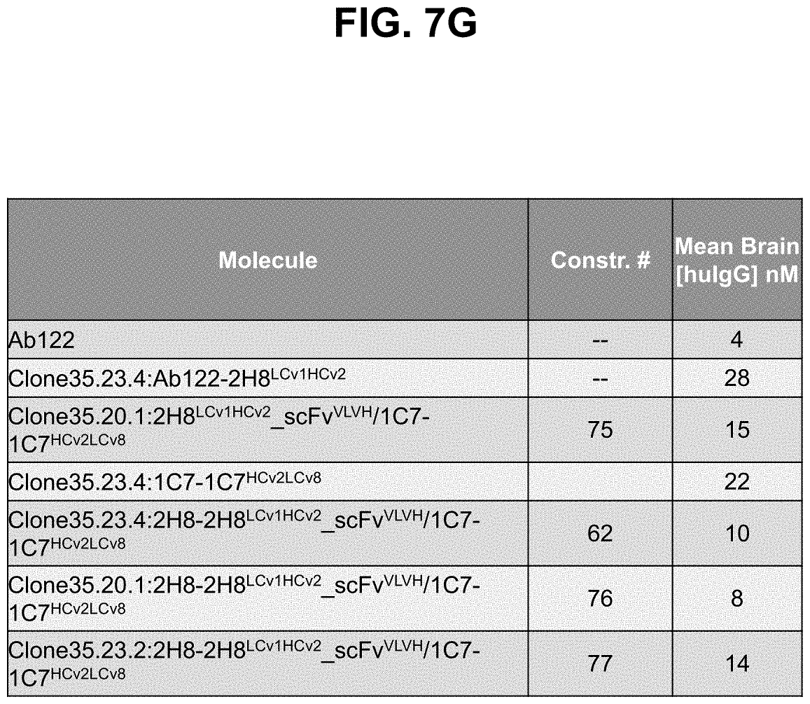

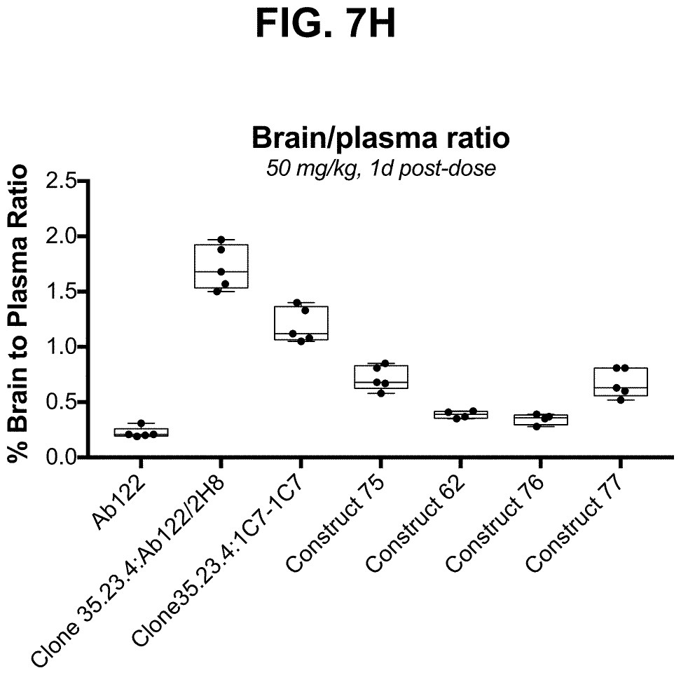

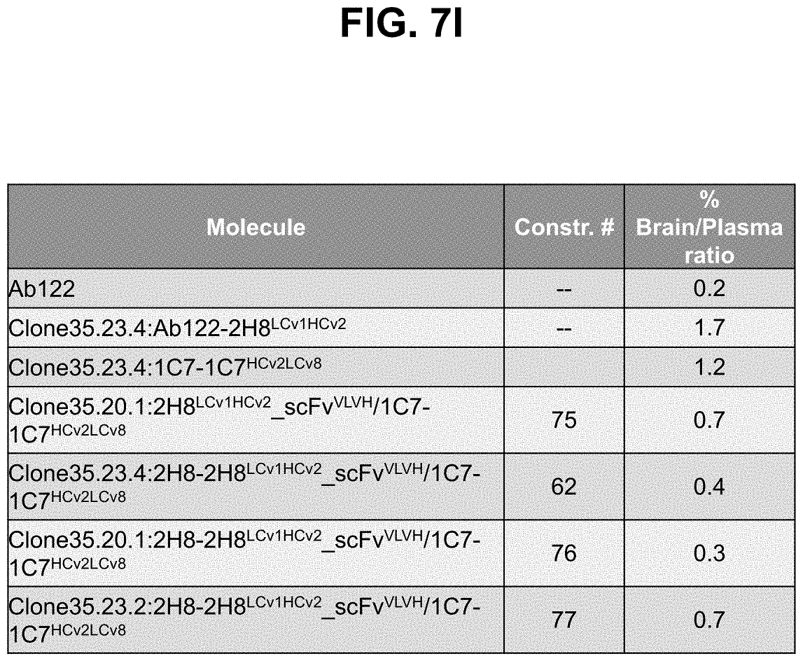

Total antibody concentrations in mouse plasma and brain lysates were quantified using a generic human Ig sandwich ELISA. A 384-well MaxiSorp plate was coated overnight with 1 μg/mL anti-huFc donkey polyclonal (Jackson Immunoresearch). Following incubation with diluted plasma or NP-40 brain lysate, an anti-huFc donkey antibody conjugated to HRP (Jackson Immunoresearch) was added as the detection reagent. The standard curves for each individual molecule, from 2 nM to 2.7 pM using 3-fold dilutions, were fit using a five-parameter logistic regression. The pharmacokinetic properties of constructs 28, 46, 62, and 75-77 are shown in FIGS. 7A-7I.

### Human IgG ELISA, BACE1 Antigen Capture, and Tau Antigen Capture ELISAs

Antibody concentrations in mouse plasma were quantified using three sandwich ELISA formats: anti-huFc, BACE1 antigen capture, and Tau antigen capture. A 384-well MaxiSorp plate was coated overnight with either 1 μg/mL anti-huFc donkey polyclonal (Jackson Immunoresearch), 2 μg/mL huBACE1 (R&D Systems), or 1 μg/mL recombinant huTau. Full length (441 amino acid) recombinant tau (r-tau) was produced in E. coli BL21(DE3) cells by CEPTER Biopartners. r-tau was originally produced with a His6-Smt3 tag which was cleaved and removed during purification.

Following incubation with diluted plasma, all ELISA formats used an anti-huFc donkey antibody conjugated to HRP (Jackson Immunoresearch) as the detection reagent. The standard curves for each individual molecule, from 4 nM to 0.97 pM using a 4-fold dilutions, were fit using a five-parameter logistic regression. The correlation graphs were constructed in GraphPad Prism and the software used to fit the data using linear regression to calculate the slope along with the Pearson correlation coefficient. As shown in FIGS. 4A and 4B, the strong correlations between both the BACE1 (FIGS. 4C and 4D) and Tau (FIGS. 4E and 4F) antigen capture with the Fc detection indicated that the molecules are largely intact throughout the pharmacokinetic time course.

### Example 11. Thermal Stability

Dynamic light scattering (DLS) measurements were collected by DynaPro Plate Reader III (Wyatt Technology). Samples were prepared at 1.0 mg/mL in PBS at pH 7.4 and the temperature was continuously ramped from 40° C. to 80° C. at a rate of 0.25° C./min. Each measurement was collected with 10 DLS acquisitions with 1 second acquisition time. Laser power was set at 20%. Data was analyzed using Dynamics V7.8.2.18 to determine Tₒₙₛₑₜ and T_{agg} values as shown in Table 21 below.

TABLE 21
Thermal Stability

| | T onset | T agg |
|---|---|---|
| Construct | (° C.) | (° C.) |
| Construct 46 | 57.75 | 64.37 |
| Construct 62 | 52.56 | 58.25 |
| Construct 28 | 62.32 | 66.43 |
| Clone35.23A:1C7-1C7^{HCv2LCv8} | 63.11 | 66.10 |

### Example 12. Antibody Treatment of CHO-huAPP Cells and Aβ40 Quantification by ELISA

CHOK1-huAPP cells (15,000/well) were plated on tissue culture-treated 96-well plates (Thermo Sci Nunclon Delta Surface) in 100 μL/well DMEM/F12 media supplemented with 10% FBS. After plating, cells recovered overnight at 37° C. with 5% CO₂. For the treatments, antibodies were first serially-diluted in media at 1000 to 0.06 nM (4-fold dilutions) and 1 respectively. The media was entirely replaced with 100 μL diluted treatment with duplicate wells for each condition. Cells were then kept at 37° C. with 5% CO₂ for 24 h. Following the 24 h treatment, the media was collected for Aβ40 measurement. Measurements of human Aβ1-40 (from the human neuron cultures) were conducted according to the Cisbio Aβ1-40 ELISA kit (Cisbio #62B40PEG). The kit provided two anti-Aβ1-40 antibodies that act as a FRET donor and receptor pair: one antibody was labeled with Eu3⁺-Cryptate (FRET donor) and the other with XL-665 (FRET receptor). Both antibodies were incubated with 5 μL of media, harvested from human neurons cultures, and placed in a PerkinElmer OptiPlate 384 for 24 h at 4° C. The plate was then read, and Aβ1-40 concentration was calculated from a 665 nm/620 nm ratio.

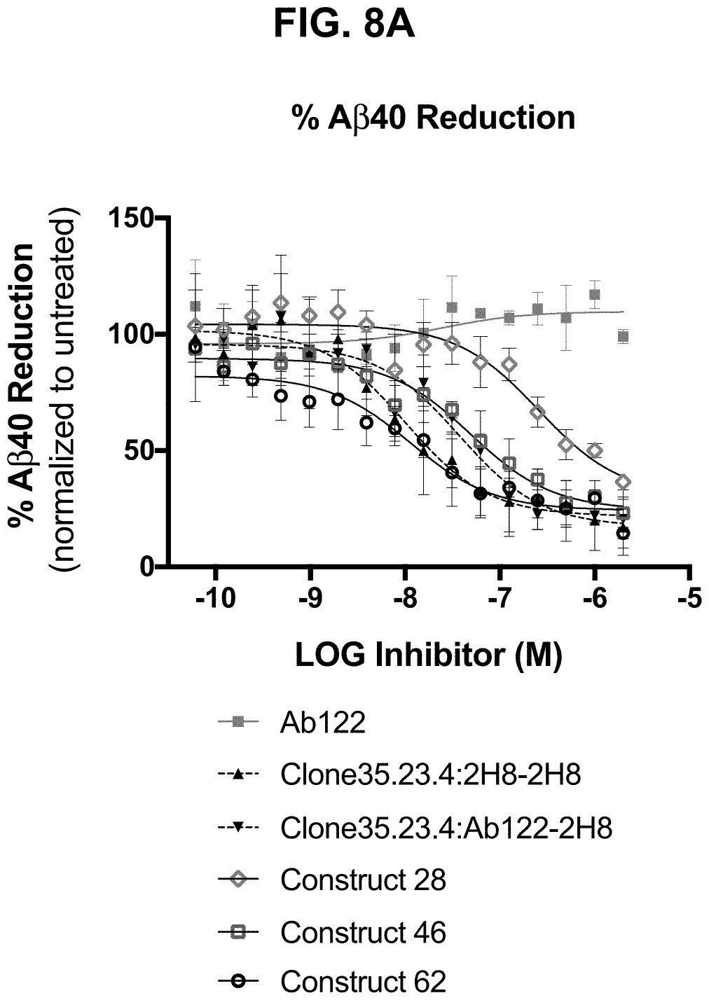

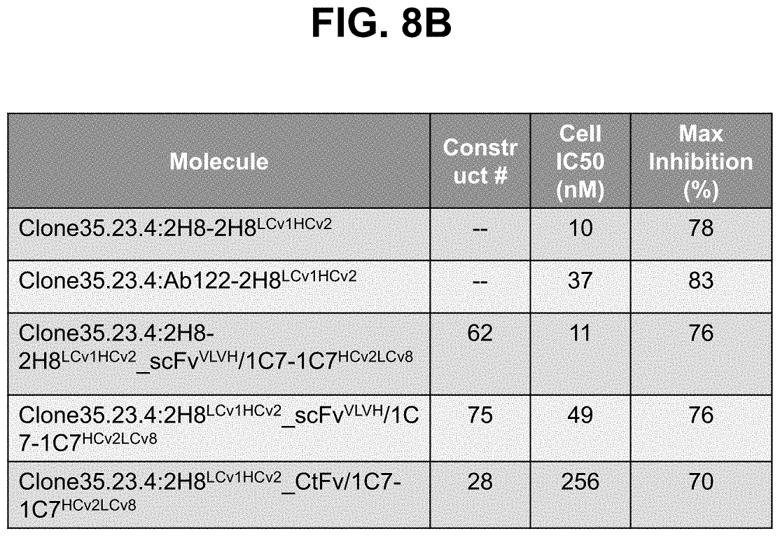

As shown in FIGS. 8A and 8B, all versions of 2H8 fused to Clone35.23.4:1C7-1C7 reduced human Aβ in a dose-dependent manner as compared to untreated control. Control IgG (Ab122) had no effect on Aβ reduction. Line graphs represent mean±SEM, n=2 independent experiments.

### Example 13. Quantification of Aβ40 in PS19/TfR^{ms/hu} KI Mice

PS19/TfR^{ms/hu} KI mice were systemically dosed one time via tail vein at 50 mg/kg. Brains were extracted after perfusion and hemi-brains were isolated for homogenization in 10× by tissue weight of 1% NP-40 in PBS (for PK) or 5 M GuHCl (for PD).

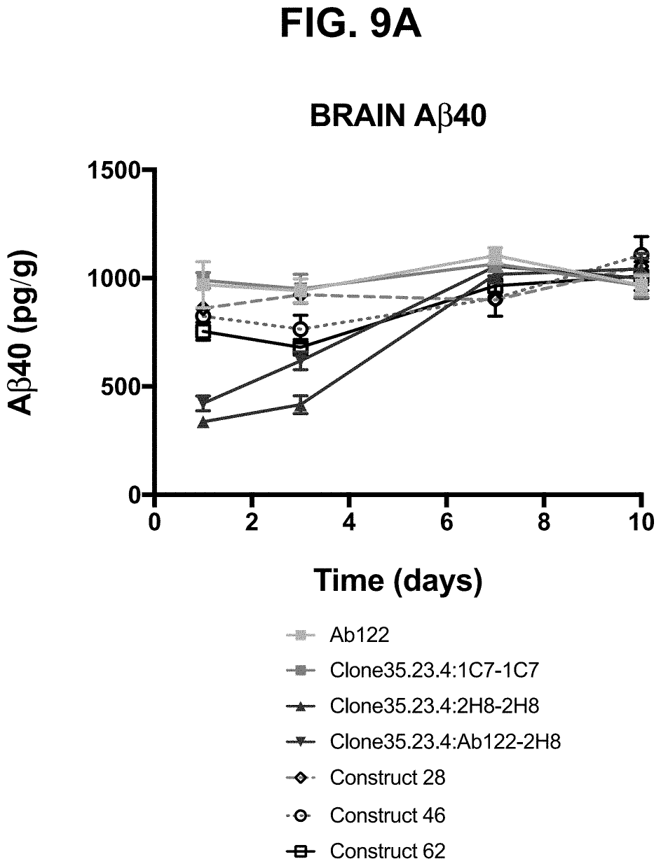

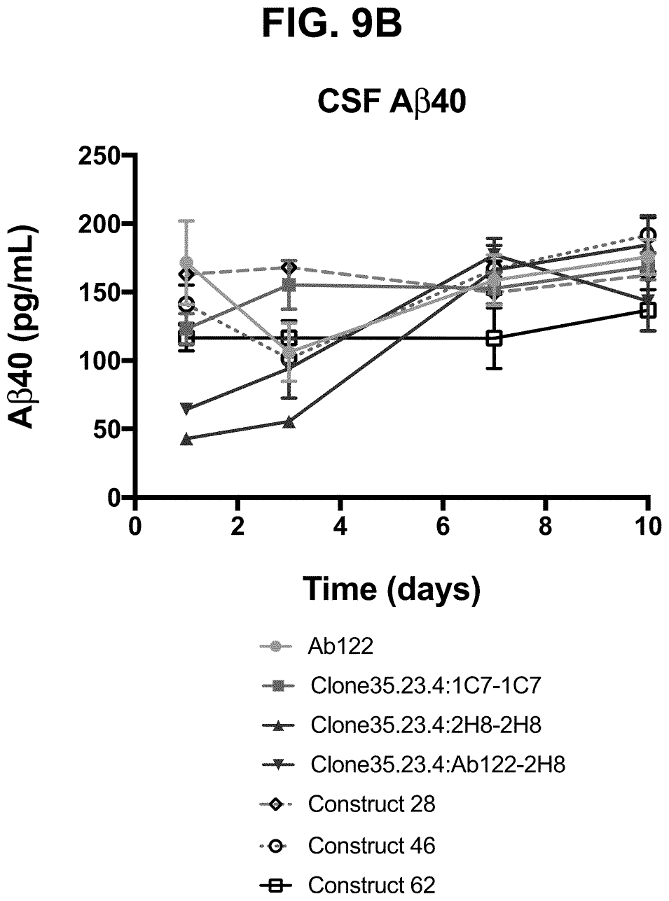

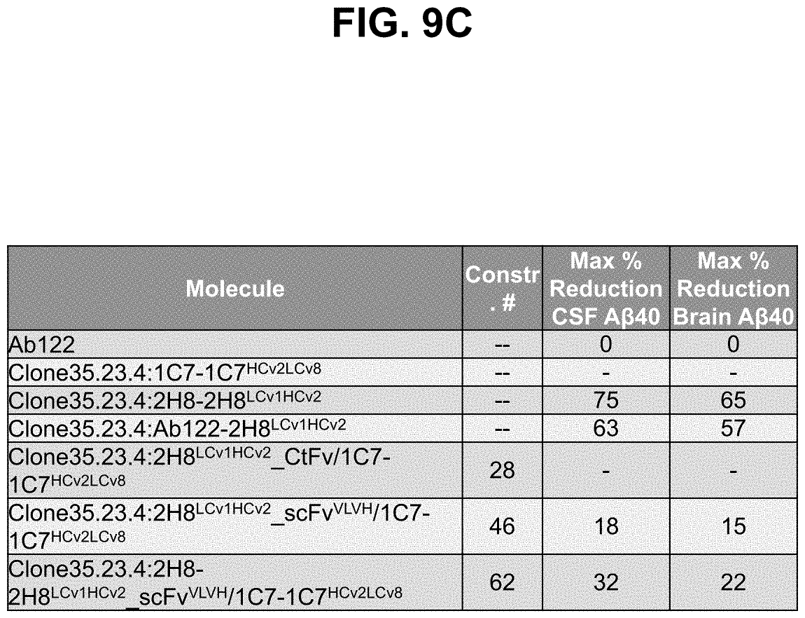

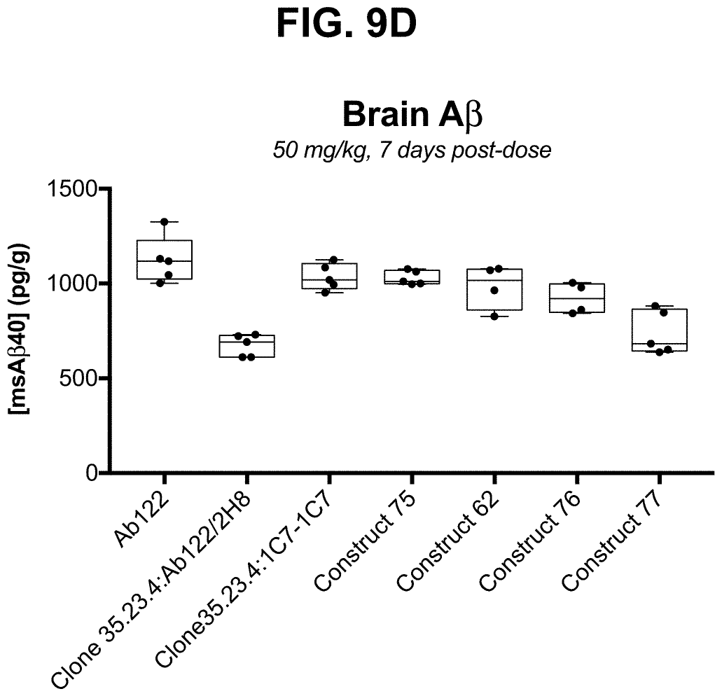

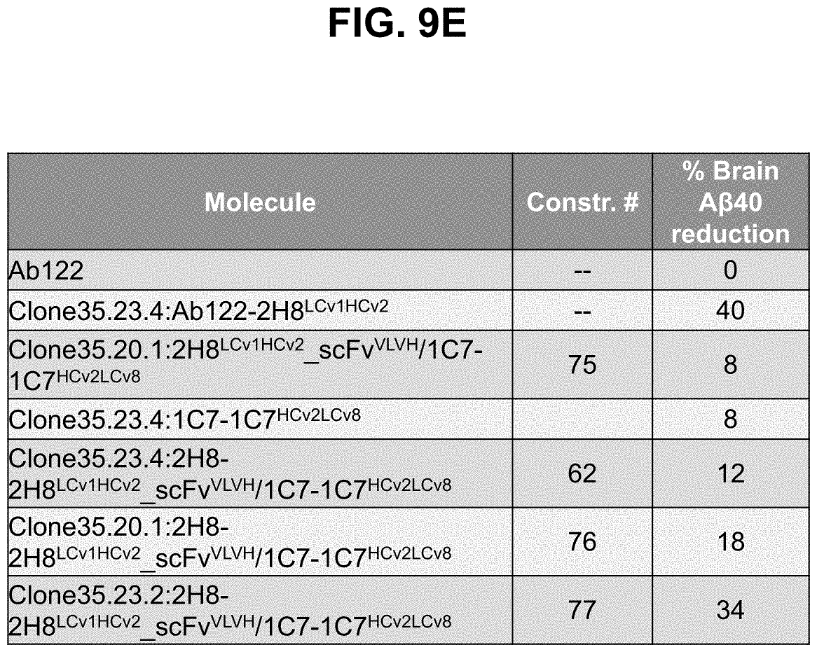

Mouse Aβ40 levels in brain lysate and CSF were measured using a sandwich ELISA. A 384-well MaxiSorp plate was coated overnight with a polyclonal capture antibody specific for the C-terminus of the Aβ40 peptide (Millipore #ABN240). Casein-diluted guanidine brain lysates were further diluted 1:2 on the ELISA plate and added concurrently with the detection antibody, biotinylated M3.2. CSF was analyzed at a 1:20 dilution. Samples were incubated overnight at 4° C. prior to addition of streptavidin-HRP followed by TMB substrate. The standard curve, 0.78-50 pg/mL msAβ40, was fit using a four-parameter logistic regression. FIGS. 9A-9E show the quantifications of brain and CSF Aβ40 in PS19/hTfR^{ms/hu} KI mice following the intravenous injection of construct 28, 46, 62, 75, 76, or 77. The constructs reduced human Aβ as compared to untreated control. Control IgG (Ab122) had no effect on Aβ reduction.

The amino acid substitutions for each clone described in the Tables (e.g., Table 9) dictate the amino acid substitutions at the register positions of that clone over the amino acids found in the sequence set forth in the Sequence Listing, in case of discrepancy.

It is understood that the examples and embodiments described herein are for illustrative purposes only and that various modifications or changes in light thereof will be suggested to persons skilled in the art and are to be included within the spirit and purview of this application and scope of the appended claims. The sequences of the sequence accession numbers cited herein are hereby incorporated by reference.

TABLE 22
Informal Sequence Listing

| SEQ ID | | |
|---|---|---|
| NO | Sequence | Description |
| 1 | APELLGGPSVFLFPPKPKDTLMISRTPEVTCVVVDVSHEDPEVKFNW | Wild-type human Fc |
| | YVDGVEVHNAKTKPREEQYNSTYRVVSVLTVLHQDWLNGKEYKC | sequence |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 2 | APELLGGPSVFLFPPKPKDTLMISRTPEVTCVVVDVSHEDPEVKFNW | CH2 domain sequence, |
| | YVDGVEVHNAKTKPREEQYNSTYRVVSVLTVLHQDWLNGKEYKC | positions 231-340 (EU |
| | KVSNKALPAPIEKTISKAK | index numbering) |
| 3 | GQPREPQVYTLPPSRDELTKNQVSLTCLVKGFYPSDIAVEWESNGQP | CH3 domain sequence, |
| | ENNYKTTPPVLDSDGSFFLYSKLTVDKSRWQQGNVFSCSVMHEALH | positions 341-447 (EU |
| | NHYTQKSLSLSPGK | index numbering) |
| 4 | APELLGGPSVFLFPPKPKDTLMISRTPEVTCVVVDVSHEDPEVKFNW | Clone CH3C.1 |
| | YVDGVEVHNAKTKPREEQYNSTYRVVSVLTVLHQDWLNGKEYKC | (Clone CH3C.18.4) |
| | KVSNKALPAPIEKTISKAKGQPREPQVYTLPPSRDELTKNQVSLTCL | |
| | VKGFYPSDIAVEWESLGLVWVGYKTTPPVLDSDGSFFLYSKLTVAK | |
| | STWQQGWVFSCSVMHEALHNHYTQKSLSLSPGK | |
| 5 | APELLGGPSVFLFPPKPKDTLMISRTPEVTCVVVDVSHEDPEVKFNW | Clone CH3C.2 |
| | YVDGVEVHNAKTKPREEQYNSTYRVVSVLTVLHQDWLNGKEYKC | (Clone CH3C.18.2) |
| | KVSNKALPAPIEKTISKAKGQPREPQVYTLPPSRDELTKNQVSLTCL | |
| | VKGFYPSDIAVEWESYGTVWSHYKTTPPVLDSDGSFFLYSKLTVSKS | |
| | EWQQGYVFSCSVMHEALHNHYTQKSLSLSPGK | |
| 6 | APELLGGPSVFLFPPKPKDTLMISRTPEVTCVVVDVSHEDPEVKFNW | Clone CH3C.3 |
| | YVDGVEVHNAKTKPREEQYNSTYRVVSVLTVLHQDWLNGKEYKC | (Clone CH3C.18.3) |
| | KVSNKALPAPIEKTISKAKGQPREPQVYTLPPSRDELTKNQVSLTCL | |
| | VKGFYPSDIAVEWESYGTEWSQYKTTPPVLDSDGSFFLYSKLTVEKS | |
| | DWQQGHVFSCSVMHEALHNHYTQKSLSLSPGK | |
| 7 | APELLGGPSVFLFPPKPKDTLMISRTPEVTCVVVDVSHEDPEVKFNW | Clone CH3C.4 |
| | YVDGVEVHNAKTKPREEQYNSTYRVVSVLTVLHQDWLNGKEYKC | (Clone CH3C.18.1) |
| | KVSNKALPAPIEKTISKAKGQPREPQVYTLPPSRDELTKNQVSLTCL | |
| | VKGFYPSDIAVEWESVGTPWALYKTTPPVLDSDGSFFLYSKLTVLKS | |
| | EWQQGWVFSCSVMHEALHNHYTQKSLSLSPGK | |
| 8 | APELLGGPSVFLFPPKPKDTLMISRTPEVTCVVVDVSHEDPEVKFNW | Clone CH3C.17 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTVWSKYKTTPPVLDSDGSFFLYSKLTVSKS | |
| | EWQQGFVFSCSVMHEALHNHYTQKSLSLSPGK | |
| 9 | APELLGGPSVFLFPPKPKDTLMISRTPEVTCVVVDVSHEDPEVKFNW | Clone CH3C.18 |
| | YVDGVEVHNAKTKPREEQYNSTYRVVSVLTVLHQDWLNGKEYKC | (Clone CH3C.18.1.18) |
| | KVSNKALPAPIEKTISKAKGQPREPQVYTLPPSRDELTKNQVSLTCL | |
| | VKGFYPSDIAVEWESLGHVWAVYKTTPPVLDSDGSFFLYSKLTVPK | |
| | STWQQGWVFSCSVMHEALHNHYTQKSLSLSPGK | |
| 10 | APELLGGPSVFLFPPKPKDTLMISRTPEVTCVVVDVSHEDPEVKFNW | Clone CH3C.21 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGLVWVGYKTTPPVLDSDGSFFLYSKLTVPK | |
| | STWQQGWVFSCSVMHEALHNHYTQKSLSLSPGK | |
| 11 | APELLGGPSVFLFPPKPKDTLMISRTPEVTCVVVDVSHEDPEVKFNW | Clone CH3C.25 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESMGHVWVGYKTTPPVLDSDGSFFLYSKLTVD | |
| | KSTWQQGWVFSCSVMHEALHNHYTQKSLSLSPGK | |
| 12 | APELLGGPSVFLFPPKPKDTLMISRTPEVTCVVVDVSHEDPEVKFNW | Clone CH3C.34 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGLVWVFSKTTPPVLDSDGSFFLYSKLTVPKS | |
| | TWQQGWVFSCSVMHEALHNHYTQKSLSLSPGK | |
| 13 | APELLGGPSVFLFPPKPKDTLMISRTPEVTCVVVDVSHEDPEVKFNW | Clone CH3C.35 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSSYKTTPPVLDSDGSFFLYSKLTVTKS | |
| | EWQQGFVFSCSVMHEALHNHYTQKSLSLSPGK | |
| 14 | APELLGGPSVFLFPPKPKDTLMISRTPEVTCVVVDVSHEDPEVKFNW | Clone CH3C.44 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSNYKTTPPVLDSDGSFFLYSKLTVSKS | |
| | EWQQGFVFSCSVMHEALHNHYTQKSLSLSPGK | |
| 15 | APELLGGPSVFLFPPKPKDTLMISRTPEVTCVVVDVSHEDPEVKFNW | Clone CH3C.51 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWVGYKTTPPVLDSDGSFFLYSKLTVSK | |
| | SEWQQGWVFSCSVMHEALHNHYTQKSLSLSPGK | |
| 16 | APELLGGPSVFLFPPKPKDTLMISRTPEVTCVVVDVSHEDPEVKFNW | Clone CH3C.3.1-3 |
| | YVDGVEVHNAKTKPREEQYNSTYRVVSVLTVLHQDWLNGKEYKC | (Clone CH3C.18.3.1-3) |
| | KVSNKALPAPIEKTISKAKGQPREPQVYTLPPSRDELTKNQVSLTCL | |
| | VKGFYPSDIAVEWESLGHVWVATKTTPPVLDSDGSFFLYSKLTVPK | |
| | STWQQGWVFSCSVMHEALHNHYTQKSLSLSPGK | |
| 17 | APELLGGPSVFLFPPKPKDTLMISRTPEVTCVVVDVSHEDPEVKFNW | Clone CH3C.3.1-9 |
| | YVDGVEVHNAKTKPREEQYNSTYRVVSVLTVLHQDWLNGKEYKC | (Clone CH3C.18.3.1-9) |
| | KVSNKALPAPIEKTISKAKGQPREPQVYTLPPSRDELTKNQVSLTCL | |
| | VKGFYPSDIAVEWESLGPVWVHTKTTPPVLDSDGSFFLYSKLTVPKS | |
| | TWQQGWVFSCSVMHEALHNHYTQKSLSLSPGK | |
| 18 | APELLGGPSVFLFPPKPKDTLMISRTPEVTCVVVDVSHEDPEVKFNW | Clone CH3C.3.2-5 |
| | YVDGVEVHNAKTKPREEQYNSTYRVVSVLTVLHQDWLNGKEYKC | (Clone CH3C.18.3.2-5) |
| | KVSNKALPAPIEKTISKAKGQPREPQVYTLPPSRDELTKNQVSLTCL | |
| | VKGFYPSDIAVEWESLGHVWVDQKTTPPVLDSDGSFFLYSKLTVPK | |
| | STWQQGWVFSCSVMHEALHNHYTQKSLSLSPGK | |
| 19 | APELLGGPSVFLFPPKPKDTLMISRTPEVTCVVVDVSHEDPEVKFNW | Clone CH3C.3.2-19 |
| | YVDGVEVHNAKTKPREEQYNSTYRVVSVLTVLHQDWLNGKEYKC | (Clone CH3C.18.3.2-19) |
| | KVSNKALPAPIEKTISKAKGQPREPQVYTLPPSRDELTKNQVSLTCL | |
| | VKGFYPSDIAVEWESLGHVWVNQKTTPPVLDSDGSFFLYSKLTVPK | |
| | STWQQGWVFSCSVMHEALHNHYTQKSLSLSPGK | |
| 20 | APELLGGPSVFLFPPKPKDTLMISRTPEVTCVVVDVSHEDPEVKFNW | Clone CH3C.3.2-1 |
| | YVDGVEVHNAKTKPREEQYNSTYRVVSVLTVLHQDWLNGKEYKC | (Clone CH3C.18.3.2-1) |
| | KVSNKALPAPIEKTISKAKGQPREPQVYTLPPSRDELTKNQVSLTCL | |
| | VKGFYPSDIAVEWESLGHVWVNFKTTPPVLDSDGSFFLYSKLTVPKS | |
| | TWQQGWVFSCSVMHEALHNHYTQKSLSLSPGK | |
| 21 | APELLGGPSVFLFPPKPKDTLMISRTPEVTCVVVDVSHEDPEVKFNW | Clone CH3C.18.E153W |
| | YVDGVEVHNAKTKPREEQYNSTYRVVSVLTVLHQDWLNGKEYKC | (CH3C.35.13) |
| | KVSNKALPAPIEKTISKAKGQPREPQVYTLPPSRDELTKNQVSLTCL | |
| | VKGFYPSDIAVWWESLGHVWAVYKTTPPVLDSDGSFFLYSKLTVPK | |
| | STWQQGWVFSCSVMHEALHNHYTQKSLSLSPGK | |
| 22 | APELLGGPSVFLFPPKPKDTLMISRTPEVTCVVVDVSHEDPEVKFNW | Clone CH3C.18.K165Q |
| | YVDGVEVHNAKTKPREEQYNSTYRVVSVLTVLHQDWLNGKEYKC | (CH3C.35.14) |
| | KVSNKALPAPIEKTISKAKGQPREPQVYTLPPSRDELTKNQVSLTCL | |
| | VKGFYPSDIAVEWESLGHVWAVYQTTPPVLDSDGSFFLYSKLTVPK | |
| | STWQQGWVFSCSVMHEALHNHYTQKSLSLSPGK | |
| 23 | APELLGGPSVFLFPPKPKDTLMISRTPEVTCVVVDVSHEDPEVKFNW | Clone CH3C.18.E153W. |
| | YVDGVEVHNAKTKPREEQYNSTYRVVSVLTVLHQDWLNGKEYKC | K165Q (CH3C.35.15) |
| | KVSNKALPAPIEKTISKAKGQPREPQVYTLPPSRDELTKNQVSLTCL | |
| | VKGFYPSDIAVWWESLGHVWAVYQTTPPVLDSDGSFFLYSKLTVPK | |
| | STWQQGWVFSCSVMHEALHNHYTQKSLSLSPGK | |
| 24 | APELLGGPSVFLFPPKPKDTLMISRTPEVTCVVVDVSHEDPEVKFNW | Clone CH3C.35.E153W |
| | YVDGVEVHNAKTKPREEQYNSTYRVVSVLTVLHQDWLNGKEYKC | (CH3C.35.19) |
| | KVSNKALPAPIEKTISKAKGQPREPQVYTLPPSRDELTKNQVSLTCL | |
| | VKGFYPSDIAVWWESYGTEWSSYKTTPPVLDSDGSFFLYSKLTVTK | |
| | SEWQQGFVFSCSVMHEALHNHYTQKSLSLSPGK | |
| 25 | APELLGGPSVFLFPPKPKDTLMISRTPEVTCVVVDVSHEDPEVKFNW | Clone CH3C.35.S188E |
| | YVDGVEVHNAKTKPREEQYNSTYRVVSVLTVLHQDWLNGKEYKC | (CH3C.35.20) |
| | KVSNKALPAPIEKTISKAKGQPREPQVYTLPPSRDELTKNQVSLTCL | |
| | VKGFYPSDIAVEWESYGTEWSSYKTTPPVLDSDGSFFLYSKLTVTKE | |
| | EWQQGFVFSCSVMHEALHNHYTQKSLSLSPGK | |
| 26 | APELLGGPSVFLFPPKPKDTLMISRTPEVTCVVVDVSHEDPEVKFNW | Clone CH3C.35.E153W. |
| | YVDGVEVHNAKTKPREEQYNSTYRVVSVLTVLHQDWLNGKEYKC | S188E (CH3C.35.21) |
| | KVSNKALPAPIEKTISKAKGQPREPQVYTLPPSRDELTKNQVSLTCL | |
| | VKGFYPSDIAVWWESYGTEWSSYKTTPPVLDSDGSFFLYSKLTVTK | |
| | EEWQQGFVFSCSVMHEALHNHYTQKSLSLSPGK | |
| 27 | APELLGGPSVFLFPPKPKDTLMISRTPEVTCVVVDVSHEDPEVKFNW | Clone CH3C.35.N163 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSNYKTTPPVLDSDGSFFLYSKLTVTKS | |
| | EWQQGFVFSCSVMHEALHNHYTQKSLSLSPGK | |
| 28 | APELLGGPSVFLFPPKPKDTLMISRTPEVTCVVVDVSHEDPEVKFNW | Clone CH3C.35.K165Q |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSSYQTTPPVLDSDGSFFLYSKLTVTKS | |
| | EWQQGFVFSCSVMHEALHNHYTQKSLSLSPGK | |
| 29 | APELLGGPSVFLFPPKPKDTLMISRTPEVTCVVVDVSHEDPEVKFNW | Clone CH3C.35.N163. |
| | YVDGVEVHNAKTKPREEQYNSTYRVVSVLTVLHQDWLNGKEYKC | K165Q |
| | KVSNKALPAPIEKTISKAKGQPREPQVYTLPPSRDELTKNQVSLTCL | |
| | VKGFYPSDIAVEWESYGTEWSNYQTTPPVLDSDGSFFLYSKLTVTKS | |
| | EWQQGFVFSCSVMHEALHNHYTQKSLSLSPGK | |
| 30 | APELLGGPSVFLFPPKPKDTLMISRTPEVTCVVVDVSHEDPEVKFNW | Clone CH3B.1 |
| | YVDGVEVHNAKTKPREEQYNSTYRVVSVLTVLHQDWLNGKEYKC | |
| | KVSNKALPAPIEKTISKAKGQPRFDYVTTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYGFHDLSLSPGK | |
| 31 | APELLGGPSVFLFPPKPKDTLMISRTPEVTCVVVDVSHEDPEVKFNW | Clone CH3B.2 |
| | YVDGVEVHNAKTKPREEQYNSTYRVVSVLTVLHQDWLNGKEYKC | |
| | KVSNKALPAPIEKTISKAKGQPRFDMVTTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYGFHDLSLSPGK | |
| 32 | APELLGGPSVFLFPPKPKDTLMISRTPEVTCVVVDVSHEDPEVKFNW | Clone CH3B.3 |
| | YVDGVEVHNAKTKPREEQYNSTYRVVSVLTVLHQDWLNGKEYKC | |
| | KVSNKALPAPIEKTISKAKGQPRFEYVTTLPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYGFHDLSLSPGK | |
| 33 | APELLGGPSVFLFPPKPKDTLMISRTPEVTCVVVDVSHEDPEVKFNW | Clone CH3B.4 |
| | YVDGVEVHNAKTKPREEQYNSTYRVVSVLTVLHQDWLNGKEYKC | |
| | KVSNKALPAPIEKTISKAKGQPRFEMVTTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYGFHDLSLSPGK | |
| 34 | APELLGGPSVFLFPPKPKDTLMISRTPEVTCVVVDVSHEDPEVKFNW | Clone CH3B.5 |
| | YVDGVEVHNAKTKPREEQYNSTYRVVSVLTVLHQDWLNGKEYKC | |
| | KVSNKALPAPIEKTISKAKGQPRFELVTTLPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYGFHDLSLSPGK | |
| 35 | APELLGGPSVFLFPPKPKDTLMISRTPEVTCVVVDVSHEDPEVKFNW | Clone CH3B.6 |
| | YVDGVEVHNAKTKPREEQYNSTYRVVSVLTVLHQDWLNGKEYKC | |
| | KVSNKALPAPIEKTISKAKGQPRFEIVTTLPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYGFHDLSLSPGK | |
| 36 | APELLGGPSVFLFPPKPKDTLMISRTPEVTCVVVDVSHEDPEVKFNW | Clone CH3B.7 |
| | YVDGVEVHNAKTKPREEQYNSTYRVVSVLTVLHQDWLNGKEYKC | |
| | KVSNKALPAPIEKTISKAKGQPRFDIVTTLPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYGFHDLSLSPGK | |
| 37 | APELLGGPSVFLFPPKPKDTLMISRTPEVTCVVVDVSHEDPEVKFNW | Clone CH3B .8 |
| | YVDGVEVHNAKTKPREEQYNSTYRVVSVLTVLHQDWLNGKEYKC | |
| | KVSNKALPAPIEKTISKAKGQPRFDYVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYGFHDLSLSPGK | |
| 38 | APELLGGPSVFLFPPKPKDTLMISRTPEVTCVVVDVSHEDPEVKFNW | Clone CH3B.9 |
| | YVDGVEVHNAKTKPREEQYNSTYRVVSVLTVLHQDWLNGKEYKC | |
| | KVSNKALPAPIEKTISKAKGQPRFGMVTTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYGFHDLSLSPGK | |
| 39 | APELLGGPSVFLFPPKPKDTLMISRTPEVTCVVVDVSHEDPEVKFNW | Clone CH3B.10 |
| | YVDGVEVHNAKTKPREEQYNSTYRVVSVLTVLHQDWLNGKEYKC | |
| | KVSNKALPAPIEKTISKAKGQPRFADVTILPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYGFYDLSLSPGK | |
| 40 | APELLGGPSVFLFPPKPKDTLMISRTPEVTCVVVDVSHEDPEVKFNW | Clone CH3B.11 |
| | YVDGVEVHNAKTKPREEQYNSTYRVVSVLTVLHQDWLNGKEYKC | |
| | KVSNKALPAPIEKTISKAKGQPRFGLVTTLPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYGFHDLSLSPGK | |
| 41 | APELLGGPSVFLFPPKPKDTLMISRTPEVTCVVVDVSHEDPEVKFNW | Clone CH3B.12 |
| | YVDGVEVHNAKTKPREEQYNSTYRVVSVLTVLHQDWLNGKEYKC | |
| | KVSNKALPAPIEKTISKAKGQPRFDYVTTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYGFSDLSLSPGK | |
| 42 | APELLGGPSVFLFPPKPKDTLMISRTPEVTCVVVDVSHEDPEVKFNW | Clone CH3B.13 |
| | YVDGVEVHNAKTKPREEQYNSTYRVVSVLTVLHQDWLNGKEYKC | |
| | KVSNKALPAPIEKTISKAKGQPRIDYVTTLPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYGFSDLSLSPGK | |
| 43 | APELLGGPSVFLFPPKPKDTLMISRTPEVTCVVVDVSHEDPEVKFNW | Clone CH3B.14 |
| | YVDGVEVHNAKTKPREEQYNSTYRVVSVLTVLHQDWLNGKEYKC | |
| | KVSNKALPAPIEKTISKAKGQPRFKDVTILPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYGFFDLSLSPGK | |
| 44 | APELLGGPSVFLFPPKPKDTLMISRTPEVTCVVVDVSHEDPEVKFNW | Clone CH3B.15 |
| | YVDGVEVHNAKTKPREEQYNSTYRVVSVLTVLHQDWLNGKEYKC | |
| | KVSNKALPAPIEKTISKAKGQPRFDLVTILPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYGFYDLSLSPGK | |
| 45 | APELLGGPSVFLFPPKPKDTLMISRTPEVTCVVVDVSHEDPEVKFNW | Clone CH3B.16 |
| | YVDGVEVHNAKTKPREEQYNSTYRVVSVLTVLHQDWLNGKEYKC | |
| | KVSNKALPAPIEKTISKAKGQPRIDYVTTLPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYGFSDLSLSPGK | |
| 46 | APELLGGPSVFLFPPKPKDTLMISRTPEVTCVVVDVSHEDPEVKFNW | Clone CH3B.17 |
| | YVDGVEVHNAKTKPREEQYNSTYRVVSVLTVLHQDWLNGKEYKC | |
| | KVSNKALPAPIEKTISKAKGQPRFELVATLPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYGFHDLSLSPGK | |
| 47 | APELLGGPSVFLFPPKPKDTLMISRTPEVTCVVVDVSHEDPEVEFIWY | Clone CH2A2.1 |
| | VDGVDVRYEWQLPREEQYNSTYRVVSVLTVLHQDWLNGKEYKCK | |
| | VSNKALPAPIEKTISKAKGQPREPQVYTLPPSRDELTKNQVSLTCLV | |
| | KGFYPSDIAVEWESNGQPENNYKTTPPVLDSDGSFFLYSKLTVDKSR | |
| | WQQGNVFSCSVMHEALHNHYTQKSLSLSPGK | |
| 48 | APELLGGPSVFLFPPKPKDTLMISRTPEVTCVVVDVSHEDPEVGFVVV | Clone CH2A2.2 |
| | YVDGVPVSWEWYWPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 49 | APELLGGPSVFLFPPKPKDTLMISRTPEVTCVVVDVSHEDPEVQFDW | Clone CH2A2.3 |
| | YVDGVMVRREWHRPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 50 | APELLGGPSVFLFPPKPKDTLMISRTPEVTCVVVDVSHEDPEVSFEW | Clone CH2A2.4 |
| | YVDGVPVRWEWQWPREEQYNSTYRVVSVLTVLHQDWLNGKEYK | |
| | CKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 51 | APELLGGPSVFLFPPKPKDTLMISRTPEVTCVVVDVSHEDPEVAFTW | Clone CH2A2.5 |
| | YVDGVPVRWEWQNPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 52 | APELLGGPSVFLFPPKPKDTLMISRTPEVTCVVVDVSHEDPEVNFDW | Clone CH2A2.6 |
| | YVDGVLVRREWHRPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 53 | APELLGGPSVFLFPPKPKDTLMISRTPEVTCVVVDVSHEDPEVQFVVV | Clone CH2A2.7 |
| | YVDGVAVRWEWIRPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 54 | APELLGGPSVFLFPPKPKDTLMISRTPEVTCVVVDVSHEDPEVEFIWY | Clone CH2A2.8 |
| | VDGVEVAWEWFWPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 55 | APELLGGPSVFLFPPKPKDTLMISRTPEVTCVVVDVSHEDPEVGFAW | Clone CH2A2.9 |
| | YVDGVNVRVEWQYPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 56 | APELLGGPSVFLFPPKPKDTLMISRTPEVTCVVVDVSHEDPEVGFVVV | Clone CH2A2.10 |
| | YVDGVEVRREWVRPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 57 | APELLGGPSVFLFPPKPKDTLMISRTPEVTCVVVDVSHEDPEVSFDW | Clone CH2A2.11 |
| | YVDGVLVRREWQRPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 58 | APELLGGPSVFLFPPKPKDTLMISRTPEVTCVVVDVSHEDPEVEFTW | Clone CH2A2.12 |
| | YVDGVDVRYEWYYPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 59 | APELLGGPSVFLFPPKPKDTLMISRTPEVTCVVVDVSHEDPEVQFTW | Clone CH2A2.13 |
| | YVDGVDVRYEWVRPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 60 | APELLGGPSVFLFPPKPKDTLMISRTPEVTCVVVDVSHEDPEVQFYW | Clone CH2A2.14 |
| | YVDGVNVRREWHRPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 61 | APELLGGPSVFLFPPKPKDTLMISRTPEVTCVVVDVSHEDPEVYFDW | Clone CH2A2.15 |
| | YVDGVMVRREWHRPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 62 | APELLGGPSVFLFPPKPKDTLMISRTPEVTCVVVDVSHEDPEVVVFEW | Clone CH2A2.16 |
| | YVDGVFVGVAYDVPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 63 | APELLGGPSVFLFPPKPKDTLMISRTPEVTCVVVDPQTPPWEVKFNW | Clone CH2C.1 |
| | YVDGVEVHNAKTKPREEEYYTYYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 64 | APELLGGPSVFLFPPKPKDTLMISRTPEVTCVVVDPPSPPWEVKFNW | Clone CH2C.2 |
| | YVDGVEVHNAKTKPREEEYYSNYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 65 | APELLGGPSVFLFPPKPKDTLMISRTPEVTCVVVDPQTPPWEVKFNW | Clone CH2C.3 |
| | YVDGVEVHNAKTKPREEEYYSNYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 66 | APELLGGPSVFLFPPKPKDTLMISRTPEVTCVVVDFRGPPWEVKFNVV | Clone CH2C.4 |
| | YVDGVEVHNAKTKPREEEYYHDYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 67 | APELLGGPSVFLFPPKPKDTLMISRTPEVTCVVVDPQTVPWEVKFNW | Clone CH2C.5 |
| | YVDGVEVHNAKTKPREEEYYSNYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 68 | APELLGGPSVFLFPPKPKDTLMISRTPEVTCVVVDPKMPPWEVKFN | Clone CH2C.6 |
| | WYVDGVEVHNAKTKPREEEYYTYYRVVSVLTVLHQDWLNGKEYK | |
| | CKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 69 | APELLGGPSVFLFPPKPKDTLMISRTPEVTCVVVDPPVPPWEVKFNW | Clone CH2C.7 |
| | YVDGVEVHNAKTKPREEEYYSNYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 70 | APELLGGPSVFLFPPKPKDTLMISRTPEVTCVVVDPAFPPWEVKFNW | Clone CH2C.8 |
| | YVDGVEVHNAKTKPREEEYYQNYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 71 | APELLGGPSVFLFPPKPKDTLMISRTPEVTCVVVDAIWPPWEVKFNW | Clone CH2C.9 |
| | YVDGVEVHNAKTKPREEEYYSNYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 72 | APELLGGPSVFLFPPKPKDTLMISRTPEVTCVVVDPPVAPWEVKFNW | Clone CH2C.10 |
| | YVDGVEVHNAKTKPREEEYYSSYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 73 | APELLGGPSVFLFPPKPKDTLMISRTPEVTCVVVDPQMPPQEVKFNW | Clone CH2C.11 |
| | YVDGVEVHNAKTKPREEEYYSNYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 74 | APELLGGPSVFLFPPKPKDTLMISRTPEVTCVVVDPQTAPWEVKFNW | Clone CH2C.12 |
| | YVDGVEVHNAKTKPREEEYYTYYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 75 | APELLGGPSVFLFPPKPKDTLMISRTPEVTCVVVDPQTPPQEVKFNW | Clone CH2 C.13 |
| | YVDGVEVHNAKTKPREEEYYSNYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 76 | APELLGGPSVFLFPPKPKDTLMISRTPEVTCVVVDPQTPPWEVKFNW | Clone CH2C.14 |
| | YVDGVEVHNAKTKPREEEYYTYYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 77 | APELLGGPSVFLFPPKPKDTLMISRTPEVTCVVVDPRVPPWEVKFNVV | CloneCH2C.15 |
| | YVDGVEVHNAKTKPREEEYYQNYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 78 | APELLGGPSVFLFPPKPKDTLMISRTPEVTCVVVDPSVPPWEVKFNW | Clone CH2C.16 |
| | YVDGVEVHNAKTKPREEEYYSNYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 79 | APELLGGPSVFLFPPKPKDTLMISRTPEVTCVVVDMLWPVPEVKFN | Clone CH2C.17 |
| | WYVDGVEVHNAKTKPREEVYHRPYRVVSVLTVLHQDWLNGKEYK | |
| | CKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 80 | APELLGGPSVFLFPPKPKDTLMISRTPEVTCVVVDMLWPVPEVKFN | Clone CH2C.18 |
| | WYVDGVEVHNAKTKPREETYHNPYRVVSVLTVLHQDWLNGKEYK | |
| | CKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 81 | APELLGGPSVFLFPPKPKDTLMISRTPEVTCVVVDMEWPVIEVKFN | Clone CH2C.19 |
| | WYVDGVEVHNAKTKPREETYHNPYRVVSVLTVLHQDWLNGKEYK | |
| | CKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 82 | APELLGGPSVFLFPPKPKDTLMISRTPEVTCVVVDMLWPVPEVKFN | Clone CH2C.20 |
| | WYVDGVEVHNAKTKPREETYHHPYRVVSVLTVLHQDWLNGKEYK | |
| | CKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 83 | APELLGGPSVFLFPPKPKDTLMISRTPEVTCVVVDDDLTFQEVKFNW | Clone CH2C.21 |
| | YVDGVEVHNAKTKPREEVYVTPYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 84 | APELLGGPSVFLFPPKPKDTLMISRTPEVTCVVVDDDLTFQEVKFNW | Clone CH2C.22 |
| | YVDGVEVHNAKTKPREELYVTPYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 85 | APELLGGPSVFLFPPKPKDTLMISRTPEVTCVVVDAYGDPEEVKFNW | Clone CH2C.23 |
| | YVDGVEVHNAKTKPREEWYDVPYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 86 | APELLGGPSVFLFPPKPKDTLMISRTPEVTCVVVDVSVPPRMVKFNW | Clone CH2D.1 |
| | YVDGVEVHNAKTKSLTSQHNSTVRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 87 | APELLGGPSVFLFPPKPKDTLMISRTPEVTCVVVDVSVPPWMVKFN | Clone CH2D.2 |
| | WYVDGVEVHNAKTKSLTSQHNSTVRVVSVLTVLHQDWLNGKEYK | |
| | CKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 88 | APELLGGPSVFLFPPKPKDTLMISRTPEVTCVVVDVSDMWEYVKFN | Clone CH2D.3 |
| | WYVDGVEVHNAKTKPWVKQLNSTWRVVSVLTVLHQDWLNGKEY | |
| | KCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTVD | |
| | KSRWQQGNVFSCSVMHEALHNHYTQKSLSLSPGK | |
| 89 | APELLGGPSVFLFPPKPKDTLMISRTPEVTCVVVDVSDDWTWVKFN | Clone CH2D.4 |
| | WYVDGVEVHNAKTKPWIAQPNSTWRVVSVLTVLHQDWLNGKEYK | |
| | CKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 90 | APELLGGPSVFLFPPKPKDTLMISRTPEVTCVVVDVSDDWEWVKFN | Clone CH2D.5 |
| | WYVDGVEVHNAKTKPWKLQLNSTWRVVSVLTVLHQDWLNGKEY | |
| | KCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTVD | |
| | KSRWQQGNVFSCSVMHEALHNHYTQKSLSLSPGK | |
| 91 | APELLGGPSVFLFPPKPKDTLMISRTPEVTCVVVDVSHEDPWVWFY | Clone CH2E3.1 |
| | WYVDGVEVHNAKTKPREEQYNSTYRVVSVLTVLHQDWLNGKEYK | |
| | CSVVNIALWWSIEKTISKAKGQPREPQVYTLPPSRDELTKNQVSLTC | |
| | LVKGFYPSDIAVEWESNGQPENNYKTTPPVLDSDGSFFLYSKLTVDK | |
| | SRWQQGNVFSCSVMHEALHNHYTQKSLSLSPGK | |
| 92 | APELLGGPSVFLFPPKPKDTLMISRTPEVTCVVVDVSHEDPVVGFRW | Clone CH2E3.2 |
| | YVDGVEVHNAKTKPREEQYNSTYRVVSVLTVLHQDWLNGKEYKC | |
| | RVSNSALTWKIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 93 | APELLGGPSVFLFPPKPKDTLMISRTPEVTCVVVDVSHEDPVVGFRW | Clone CH2E3.3 |
| | YVDGVEVHNAKTKPREEQYNSTYRVVSVLTVLHQDWLNGKEYKC | |
| | RVSNSALSWRIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 94 | APELLGGPSVFLFPPKPKDTLMISRTPEVTCVVVDVSHEDPIVGFRW | Clone CH2E3.4 |
| | YVDGVEVHNAKTKPREEQYNSTYRVVSVLTVLHQDWLNGKEYKC | |
| | RVSNSALRWRIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 95 | APELLGGPSVFLFPPKPKDTLMISRTPEVTCVVVDVSHEDPAVGFEW | Clone CH2E3.5 |
| | YVDGVEVHNAKTKPREEQYNSTYRVVSVLTVLHQDWLNGKEYKC | |
| | QVFNWALDWVIEKTISKAKGQPREPQVYTLPPSRDELTKNQVSLTC | |
| | LVKGFYPSDIAVEWESNGQPENNYKTTPPVLDSDGSFFLYSKLTVDK | |
| | SRWQQGNVFSCSVMHEALHNHYTQKSLSLSPGK | |
| 96 | NSVIIVDKNGRLVYLVENPGGYVAYSKAATVTGKLVHANFGTKKD | Human TfR apical domain |
| | FEDLYTPVNGSIVIVRAGKITFAEKVANAESLNAIGVLIYMDQTKFPI | |
| | VNAELSFFGHAHLGTGDPYTPGFPSFNHTQFPPSRSSGLPNIPVQTISR | |
| | AAAEKLFGNMEGDCPSDWKTDSTCRMVTSESKNVKLTVS | |
| 97 | NSVIIVDKNGGLVYLVENPGGYVAYSKAATVTGKLVHANFGTKKD | Cynomolgus TfR apical |
| | FEDLDSPVNGSIVIVRAGKITFAEKVANAESLNAIGVLIYMDQTKFPI | domain |
| | VKADLSFFGHAHLGTGDPYTPGFPSFNHTQFPPSQSSGLPNIPVQTIS | |
| | RAAAEKLFGNMEGDCPSDWKTDSTCKMVTSENKSVKLTVS | |
| 98 | SSGLPNIPVQTISRAAAEKLFGNMEGDCPSDWKTDSTCRMVTSESKN | Loop-truncated human |
| | VKLTVSNDSAQNSVIIVDKNGRLVYLVENPGGYVAYSKAATVTGKL | TfR apical domain |
| | VHANFGTKKDFEDLYTPVNGSIVIVRAGKITFAEKVANAESLNAIGV | displayed on phage |
| | LIYMDQTKFPIVNAELSGP | |
| 99 | SSGLPNIPVQTISRAAAEKLFGNMEGDCPSDWKTDSTCKMVTSENK | Loop-truncated |
| | SVKLTVSNDSAQNSVIIVDKNGGLVYLVENPGGYVAYSKAATVTGK | cynomolgus TfR apical |
| | LVHANFGTKKDFEDLDSPVNGSIVIVRAGKITFAEKVANAESLNAIG | domain displayed on |
| | VLIYMDQTKFPIVKADLSGP | phage |
| 100 | MMDQARSAFSNLFGGEPLSYTRFSLARQVDGDNSHVEMKLAVDEE | Human transferrin receptor |
| | ENADNNTKANVTKPKRCSGSICYGTIAVIVFFLIGFMIGYLGYCKGV | protein 1 (TFR1) |
| | EPKIECERLAGTESPVREEPGEDFPAARRLYWDDLKRKLSEKLDST | |
| | DFTGTIKLLNENSYVPREAGSQKDENLALYVENQFREFKLSKVWRD | |
| | QHFVKIQVKDSAQNSVIIVDKNGRLVYLVENPGGYVAYSKAATVTG | |
| | KLVHANFGTKKDFEDLYTPVNGSIVIVRAGKITFAEKVANAESLNAI | |
| | GVLIYMDQTKFPIVNAELSFFGHAHLGTGDPYTPGFPSFNHTQFPPSR | |
| | SSGLPNIPVQTISRAAAEKLFGNMEGDCPSDWKTDSTCRMVTSESKN | |
| | VKLTVSNVLKEIKILNIFGVIKGFVEPDHYVVVGAQRDAWGPGAAK | |
| | SGVGTALLLKLAQMFSDMVLKDGFQPSRSIIFASWSAGDFGSVGAT | |
| | EWLEGYLSSLHLKAFTYINLDKAVLGTSNFKVSASPLLYTLIEKTMQ | |
| | NVKHPVTGQFLYQDSNWASKVEKLTLDNAAFPFLAYSGIPAVSFCF | |
| | CEDTDYPYLGTTMDTYKELIERIPELNKVARAAAEVAGQFVIKLTH | |
| | DVELNLDYERYNSQLLSFVRDLNQYRADIKEMGLSLQWLYSARGD | |
| | FFRATSRLTTDFGNAEKTDRFVMKKLNDRVMRVEYHFLSPYVSPKE | |
| | SPFRHVFWGSGSHTLPALLENLKLRKQNNGAFNETLFRNQLALATW | |
| | TIQGAANALSGDVWDIDNEF | |
| 101 | APELLGGPSVFLFPPKPKDTLMISRTPEVTCVVVDVSHEDPEVKFNW | Clone CH3C.35.19 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSSYKTTPPVLDSDGSFFLYSKLTVTK | |
| | SEWQQGFVFSCSVMHEALHNHYTQKSLSLSPGK | |
| 102 | APELLGGPSVFLFPPKPKDTLMISRTPEVTCVVVDVSHEDPEVKFNW | Clone CH3C.35.20 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSSYKTTPPVLDSDGSFFLYSKLTVTKE | |
| | EWQQGFVFSCSVMHEALHNHYTQKSLSLSPGK | |
| 103 | APELLGGPSVFLFPPKPKDTLMISRTPEVTCVVVDVSHEDPEVKFNW | Clone CH3C.35.21 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAWWESYGTEWSSYKTTPPVLDSDGSFFLYSKLTVTK | |
| | EEWQQGFVFSCSVMHEALHNHYTQKSLSLSPGK | |
| 104 | APELLGGPSVFLFPPKPKDTLMISRTPEVTCVVVDVSHEDPEVKFNW | Clone CH3C.35.22 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSNYKTTPPVLDSDGSFFLYSKLTVTK | |
| | SEWQQGFVFSCSVMHEALHNHYTQKSLSLSPGK | |
| 105 | APELLGGPSVFLFPPKPKDTLMISRTPEVTCVVVDVSHEDPEVKFNW | Clone CH3C.35.23 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSNYKTTPPVLDSDGSFFLYSKLTVTKE | |
| | EWQQGFVFSCSVMHEALHNHYTQKSLSLSPGK | |
| 106 | APELLGGPSVFLFPPKPKDTLMISRTPEVTCVVVDVSHEDPEVKFNW | Clone CH3C.35.24 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESYGTEWSNYKTTPPVLDSDGSFFLYSKLTVTK | |
| | EEWQQGFVFSCSVMHEALHNHYTQKSLSLSPGK | |
| 107 | APELLGGPSVFLFPPKPKDTLMISRTPEVTCVVVDVSHEDPEVKFNW | CH3C.18 variant |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESLGHVWAVYKTTPPVLDSDGSFFLYSKLTVPK | |
| | STWQQGWVFSCSVMHEALHNHYTQKSLSLSPGK | |
| 108 | APELLGGPSVFLFPPKPKDTLMISRTPEVTCVVVDVSHEDPEVKFNW | CH3C.18 variant |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESLGHVVVAVYKTTPPVLDSDGSFFLYSKLTVPK | |
| | STWQQGWVFSCSVMHEALHNHYTQKSLSLSPGK | |
| 109 | APELLGGPSVFLFPPKPKDTLMISRTPEVTCVVVDVSHEDPEVKFNW | CH3C.18 variant |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVYWESLGHVVVAVYKTTPPVLDSDGSFFLYSKLTVPK | |
| | STWQQGWVFSCSVMHEALHNHYTQKSLSLSPGK | |
| 110 | APELLGGPSVFLFPPKPKDTLMISRTPEVTCVVVDVSHEDPEVKFNW | CH3C.18 variant |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVVVAVYQTTPPVLDSDGSFFLYSKLTVPK | |
| | STWQQGWVFSCSVMHEALHNHYTQKSLSLSPGK | |
| 111 | APELLGGPSVFLFPPKPKDTLMISRTPEVTCVVVDVSHEDPEVKFNW | CH3C.18 variant |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVVVAVYFTTPPVLDSDGSFFLYSKLTVPKS | |
| | TWQQGWVFSCSVMHEALHNHYTQKSLSLSPGK | |
| 112 | APELLGGPSVFLFPPKPKDTLMISRTPEVTCVVVDVSHEDPEVKFNW | CH3C.18 variant |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVVVAVYHTTPPVLDSDGSFFLYSKLTVPK | |
| | STWQQGWVFSCSVMHEALHNHYTQKSLSLSPGK | |
| 113 | APELLGGPSVFLFPPKPKDTLMISRTPEVTCVVVDVSHEDPEVKFNW | Clone CH3C.35.21.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYKTTPPVLDSDGSFFLYSKLTVTKS | |
| | EWQQGFVFSCSVMHEALHNHYTQKSLSLSPGK | |
| 114 | APELLGGPSVFLFPPKPKDTLMISRTPEVTCVVVDVSHEDPEVKFNW | Clone CH3C.35.21.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTKS | |
| | EWQQGFVFSCSVMHEALHNHYTQKSLSLSPGK | |
| 115 | APELLGGPSVFLFPPKPKDTLMISRTPEVTCVVVDVSHEDPEVKFNW | Clone CH3C.35.21.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTRE | |
| | EWQQGFVFSCSVMHEALHNHYTQKSLSLSPGK | |
| 116 | APELLGGPSVFLFPPKPKDTLMISRTPEVTCVVVDVSHEDPEVKFNW | Clone CH3C.35.21.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTGE | |
| | EWQQGFVFSCSVMHEALHNHYTQKSLSLSPGK | |
| 117 | APELLGGPSVFLFPPKPKDTLMISRTPEVTCVVVDVSHEDPEVKFNW | Clone CH3C.35.21.5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTRE | |
| | EWQQGFVFSCWVMHEALHNHYTQKSLSLSPGK | |
| 118 | APELLGGPSVFLFPPKPKDTLMISRTPEVTCVVVDVSHEDPEVKFNW | Clone CH3C.35.21.6 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTKE | |
| | EWQQGFVFSCWVMHEALHNHYTQKSLSLSPGK | |
| 119 | APELLGGPSVFLFPPKPKDTLMISRTPEVTCVVVDVSHEDPEVKFNW | Clone CH3C.35.21.7 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTRE | |
| | EWQQGFVFTCWVMHEALHNHYTQKSLSLSPGK | |
| 120 | APELLGGPSVFLFPPKPKDTLMISRTPEVTCVVVDVSHEDPEVKFNW | Clone CH3C.35.21.8 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTRE | |
| | EWQQGFVFTCGVMHEALHNHYTQKSLSLSPGK | |
| 121 | APELLGGPSVFLFPPKPKDTLMISRTPEVTCVVVDVSHEDPEVKFNW | CloneCH3C.35.21.9 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTRE | |
| | EWQQGFVFECWVMHEALHNHYTQKSLSLSPGK | |
| 122 | APELLGGPSVFLFPPKPKDTLMISRTPEVTCVVVDVSHEDPEVKFNW | Clone CH3C.35.21.10 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTRE | |
| | EWQQGFVFKCWVMHEALHNHYTQKSLSLSPGK | |
| 123 | APELLGGPSVFLFPPKPKDTLMISRTPEVTCVVVDVSHEDPEVKFNW | Clone CH3C.35.21.11 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTPE | |
| | EWQQGFVFKCWVMHEALHNHYTQKSLSLSPGK | |
| 124 | APELLGGPSVFLFPPKPKDTLMISRTPEVTCVVVDVSHEDPEVKFNW | Clone CH3C.35.21.12 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESYGTEWSSYRTTPPVLDSDGSFFLYSKLTVTR | |
| | EEWQQGFVFSCSVMHEALHNHYTQKSLSLSPGK | |
| 125 | APELLGGPSVFLFPPKPKDTLMISRTPEVTCVVVDVSHEDPEVKFNW | Clone CH3C.35.21.13 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESYGTEWSSYRTTPPVLDSDGSFFLYSKLTVTG | |
| | EEWQQGFVFSCSVMHEALHNHYTQKSLSLSPGK | |
| 126 | APELLGGPSVFLFPPKPKDTLMISRTPEVTCVVVDVSHEDPEVKFNW | Clone CH3C.35.21.14 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESYGTEWSSYRTTPPVLDSDGSFFLYSKLTVTR | |
| | EEWQQGFVFTCWVMHEALHNHYTQKSLSLSPGK | |
| 127 | APELLGGPSVFLFPPKPKDTLMISRTPEVTCVVVDVSHEDPEVKFNW | Clone CH3C.35.21.15 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESYGTEWSSYRTTPPVLDSDGSFFLYSKLTVTG | |
| | EEWQQGFVFTCWVMHEALHNHYTQKSLSLSPGK | |
| 128 | APELLGGPSVFLFPPKPKDTLMISRTPEVTCVVVDVSHEDPEVKFNW | Clone CH3C.35.21.16 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESYGTEWSSYRTTPPVLDSDGSFFLYSKLTVTR | |
| | EEWQQGFVFTCGVMHEALHNHYTQKSLSLSPGK | |
| 129 | APELLGGPSVFLFPPKPKDTLMISRTPEVTCVVVDVSHEDPEVKFNW | Clone CH3C.35.21.17 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYKTTPPVLDSDGSFFLYSKLTVTKE | |
| | EWQQGFVFSCSVMHEALHNHYTQKSLSLSPGK | |
| 130 | APELLGGPSVFLFPPKPKDTLMISRTPEVTCVVVDVSHEDPEVKFNW | Clone CH3C.35.21.18 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYRTTPPVLDSDGSFFLYSKLTVTKE | |
| | EWQQGFVFSCSVMHEALHNHYTQKSLSLSPGK | |
| 131 | APELLGGPSVFLFPPKPKDTLMISRTPEVTCVVVDVSHEDPEVKFNW | Clone CH3C.35.20.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWSSYKTTPPVLDSDGSFFLYSKLTVTKE | |
| | EWQQGFVFSCSVMHEALHNHYTQKSLSLSPGK | |
| 132 | APELLGGPSVFLFPPKPKDTLMISRTPEVTCVVVDVSHEDPEVKFNW | Clone CH3C.35.20.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWASYKTTPPVLDSDGSFFLYSKLTVTKE | |
| | EWQQGFVFSCSVMHEALHNHYTQKSLSLSPGK | |
| 133 | APELLGGPSVFLFPPKPKDTLMISRTPEVTCVVVDVSHEDPEVKFNW | Clone CH3C.35.20.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWVSYKTTPPVLDSDGSFFLYSKLTVTKE | |
| | EWQQGFVFSCSVMHEALHNHYTQKSLSLSPGK | |
| 134 | APELLGGPSVFLFPPKPKDTLMISRTPEVTCVVVDVSHEDPEVKFNW | Clone CH3C.35.20.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSSYKTTPPVLDSDGSFFLYSKLTVSKE | |
| | EWQQGFVFSCSVMHEALHNHYTQKSLSLSPGK | |
| 135 | APELLGGPSVFLFPPKPKDTLMISRTPEVTCVVVDVSHEDPEVKFNW | Clone CH3C.35.20.5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWASYKTTPPVLDSDGSFFLYSKLTVTKE | |
| | EWQQGFVFSCSVMHEALHNHYTQKSLSLSPGK | |
| 136 | APELLGGPSVFLFPPKPKDTLMISRTPEVTCVVVDVSHEDPEVKFNW | Clone CH3C.35.20.6 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWVSYKTTPPVLDSDGSFFLYSKLTVTKE | |
| | EWQQGFVFSCSVMHEALHNHYTQKSLSLSPGK | |
| 137 | APELLGGPSVFLFPPKPKDTLMISRTPEVTCVVVDVSHEDPEVKFNW | Clone CH3C35.21.a.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESFGTEWSSYKTTPPVLDSDGSFFLYSKLTVTKE | |
| | EWQQGFVFSCSVMHEALHNHYTQKSLSLSPGK | |
| 138 | APELLGGPSVFLFPPKPKDTLMISRTPEVTCVVVDVSHEDPEVKFNW | Clone CH3C.35.21.a.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESYGTEWASYKTTPPVLDSDGSFFLYSKLTVTK | |
| | EEWQQGFVFSCSVMHEALHNHYTQKSLSLSPGK | |
| 139 | APELLGGPSVFLFPPKPKDTLMISRTPEVTCVVVDVSHEDPEVKFNW | Clone CH3C.35.21.a.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESYGTEWVSYKTTPPVLDSDGSFFLYSKLTVTK | |
| | EEWQQGFVFSCSVMHEALHNHYTQKSLSLSPGK | |
| 140 | APELLGGPSVFLFPPKPKDTLMISRTPEVTCVVVDVSHEDPEVKFNW | Clone CH3C.35.21.a.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESYGTEWSSYKTTPPVLDSDGSFFLYSKLTVSK | |
| | EEWQQGFVFSCSVMHEALHNHYTQKSLSLSPGK | |
| 141 | APELLGGPSVFLFPPKPKDTLMISRTPEVTCVVVDVSHEDPEVKFNW | Clone CH3C.35.21.a.5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESFGTEWASYKTTPPVLDSDGSFFLYSKLTVTK | |
| | EEWQQGFVFSCSVMHEALHNHYTQKSLSLSPGK | |
| 142 | APELLGGPSVFLFPPKPKDTLMISRTPEVTCVVVDVSHEDPEVKFNW | Clone CH3C.35.21.a.6 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESFGTEWVSYKTTPPVLDSDGSFFLYSKLTVTK | |
| | EEWQQGFVFSCSVMHEALHNHYTQKSLSLSPGK | |
| 143 | APELLGGPSVFLFPPKPKDTLMISRTPEVTCVVVDVSHEDPEVKFNW | Clone CH3C.35.23.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWSNYKTTPPVLDSDGSFFLYSKLTVTKE | |
| | EWQQGFVFSCSVMHEALHNHYTQKSLSLSPGK | |
| 144 | APELLGGPSVFLFPPKPKDTLMISRTPEVTCVVVDVSHEDPEVKFNW | Clone CH3C.35.23.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWANYKTTPPVLDSDGSFFLYSKLTVTK | |
| | EEWQQGFVFSCSVMHEALHNHYTQKSLSLSPGK | |
| 145 | APELLGGPSVFLFPPKPKDTLMISRTPEVTCVVVDVSHEDPEVKFNW | Clone CH3C.35.23.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWVNYKTTPPVLDSDGSFFLYSKLTVTK | |
| | EEWQQGFVFSCSVMHEALHNHYTQKSLSLSPGK | |
| 146 | APELLGGPSVFLFPPKPKDTLMISRTPEVTCVVVDVSHEDPEVKFNW | Clone CH3C.35.23.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSNYKTTPPVLDSDGSFFLYSKLTVSKE | |
| | EWQQGFVFSCSVMHEALHNHYTQKSLSLSPGK | |
| 147 | APELLGGPSVFLFPPKPKDTLMISRTPEVTCVVVDVSHEDPEVKFNW | Clone CH3C.35.23.5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWANYKTTPPVLDSDGSFFLYSKLTVTKE | |
| | EWQQGFVFSCSVMHEALHNHYTQKSLSLSPGK | |
| 148 | APELLGGPSVFLFPPKPKDTLMISRTPEVTCVVVDVSHEDPEVKFNW | Clone CH3C.35.23.6 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWVNYKTTPPVLDSDGSFFLYSKLTVTKE | |
| | EWQQGFVFSCSVMHEALHNHYTQKSLSLSPGK | |
| 149 | APELLGGPSVFLFPPKPKDTLMISRTPEVTCVVVDVSHEDPEVKFNW | Clone CH3C.35.24.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESFGTEWSNYKTTPPVLDSDGSFFLYSKLTVTK | |
| | EEWQQGFVFSCSVMHEALHNHYTQKSLSLSPGK | |
| 150 | APELLGGPSVFLFPPKPKDTLMISRTPEVTCVVVDVSHEDPEVKFNW | Clone CH3C.35.24.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESYGTEWANYKTTPPVLDSDGSFFLYSKLTVTK | |
| | EEWQQGFVFSCSVMHEALHNHYTQKSLSLSPGK | |
| 151 | APELLGGPSVFLFPPKPKDTLMISRTPEVTCVVVDVSHEDPEVKFNW | Clone CH3C.35.24.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESYGTEWVNYKTTPPVLDSDGSFFLYSKLTVTK | |
| | EEWQQGFVFSCSVMHEALHNHYTQKSLSLSPGK | |
| 152 | APELLGGPSVFLFPPKPKDTLMISRTPEVTCVVVDVSHEDPEVKFNW | Clone CH3C.35.24.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESYGTEWSNYKTTPPVLDSDGSFFLYSKLTVSK | |
| | EEWQQGFVFSCSVMHEALHNHYTQKSLSLSPGK | |
| 153 | APELLGGPSVFLFPPKPKDTLMISRTPEVTCVVVDVSHEDPEVKFNW | Clone CH3C.35.24.5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESFGTEWANYKTTPPVLDSDGSFFLYSKLTVTK | |
| | EEWQQGFVFSCSVMHEALHNHYTQKSLSLSPGK | |
| 154 | APELLGGPSVFLFPPKPKDTLMISRTPEVTCVVVDVSHEDPEVKFNW | Clone CH3C.35.24.6 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESFGTEWVNYKTTPPVLDSDGSFFLYSKLTVTK | |
| | EEWQQGFVFSCSVMHEALHNHYTQKSLSLSPGK | |
| 155 | APELLGGPSVFLFPPKPKDTLMISRTPEVTCVVVDVSHEDPEVKFNW | Clone CH3C.35.21.17.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESFGTEWSSYKTTPPVLDSDGSFFLYSKLTVTKE | |
| | EWQQGFVFSCSVMHEALHNHYTQKSLSLSPGK | |
| 156 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWASYKTTPPVLDSDGSFFLYSKLTVTKE | |
| | EWQQGFVFSCSVMHEALHNHYTQKSLSLSPGK | |
| 157 | APELLGGPSVFLFPPKPKDTLMISRTPEVTCVVVDVSHEDPEVKFNW | Clone CH3C.35.21.17.3 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWVSYKTTPPVLDSDGSFFLYSKLTVTKE | |
| | EWQQGFVFSCSVMHEALHNHYTQKSLSLSPGK | |
| 158 | APELLGGPSVFLFPPKPKDTLMISRTPEVTCVVVDVSHEDPEVKFNW | Clone CH3C.35.21.17.4 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESYGTEWSSYKTTPPVLDSDGSFFLYSKLTVSKE | |
| | EWQQGFVFSCSVMHEALHNHYTQKSLSLSPGK | |
| 159 | APELLGGPSVFLFPPKPKDTLMISRTPEVTCVVVDVSHEDPEVKFNW | Clone CH3C.35.21.17.5 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESFGTEWASYKTTPPVLDSDGSFFLYSKLTVTKE | |
| | EWQQGFVFSCSVMHEALHNHYTQKSLSLSPGK | |
| 160 | APELLGGPSVFLFPPKPKDTLMISRTPEVTCVVVDVSHEDPEVKFNW | Clone CH3C.35.21.7.6 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVLWESFGTEWVSYKTTPPVLDSDGSFFLYSKLTVTKE | |
| | EWQQGFVFSCSVMHEALHNHYTQKSLSLSPGK | |
| 161 | APELLGGPSVFLFPPKPKDTLMISRTPEVTCVVVDVSHEDPEVKFNW | Clone CH3C.35.N390 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSNYKTTPPVLDSDGSFFLYSKLTVTKS | |
| | EWQQGFVFSCSVMHEALHNHYTQKSLSLSPGK | |
| 162 | APELLGGPSVFLFPPKPKDTLMISRTPEVTCVVVDVSHEDPEVKFNW | Clone CH3C.35.16 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVVVWESLGHVVVVNQKTTPPVLDSDGSFFLYSKLTVPK | |
| | STWQQGWVFSCSVMHEALHNHYTQKSLSLSPGK | |
| 163 | APELLGGPSVFLFPPKPKDTLMISRTPEVTCVVVDVSHEDPEVKFNW | Clone CH3C.35.17 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESLGHVWVNQQTTPPVLDSDGSFFLYSKLTVPK | |
| | STWQQGWVFSCSVMHEALHNHYTQKSLSLSPGK | |
| 164 | APELLGGPSVFLFPPKPKDTLMISRTPEVTCVVVDVSHEDPEVKFNW | Clone CH3C.35.18 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVWWESLGHVWVNQQTTPPVLDSDGSFFLYSKLTVPK | |
| | STWQQGWVFSCSVMHEALHNHYTQKSLSLSPGK | |
| 165 | APEAAGGPSVFLFPPKPKDTLYITREPEVTCVVVDVSHEDPEVKFNW | Clone CH3C.35.8 |
| | YVDGVEVHNAKTKPREEQYNSTYRVVSVLTVLHQDWLNGKEYKC | (Clone CH3C.35.20 with |
| | KVSNKALGAPIEKTISKAKGQPREPQVYTLPPSRDELTKNQVSLTCL | YTE and LALAPG |
| | VKGFYPSDIAVEWESYGTEWSSYKTTPPVLDSDGSFFLYSKLTVTKE | mutations) |
| | EWQQGFVFSCSVMHEALHNHYTQKSLSLSPGK | |
| 166 | APEAAGGPSVFLFPPKPKDTLYITREPEVTCVVVDVSHEDPEVKFNW | Clone CH3C.35.9 |
| | YVDGVEVHNAKTKPREEQYNSTYRVVSVLTVLHQDWLNGKEYKC | (Clone CH3C.35.21 with |
| | KVSNKALGAPIEKTISKAKGQPREPQVYTLPPSRDELTKNQVSLTCL | YTE and LALAPG |
| | VKGFYPSDIAVEWESFGTEWSSYKTTPPVLDSDGSFFLYSKLTVTKE | mutations) |
| | EWQQGFVFSCSVMHEALHNHYTQKSLSLSPGK | |
| 167 | APELLGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKC | knob mutation |
| | KVSNKALPAPIEKTISKAKGQPREPQVYTLPPSRDELTKNQVSLWCL | |
| | VKGFYPSDIAVEWESFGTEWSSYKTTPPVLDSDGSFFLYSKLTVTKE | |
| | EWQQGFVFSCSVMHEALHNHYTQKSLSLSPGK | |
| 168 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKC | knob and LALA mutations |
| | KVSNKALPAPIEKTISKAKGQPREPQVYTLPPSRDELTKNQVSLWCL | |
| | VKGFYPSDIAVEWESFGTEWSSYKTTPPVLDSDGSFFLYSKLTVTKE | |
| | EWQQGFVFSCSVMHEALHNHYTQKSLSLSPGK | |
| 169 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKC | knob and LALAPG |
| | KVSNKALGAPIEKTISKAKGQPREPQVYTLPPSRDELTKNQVSLWCL | mutations |
| | VKGFYPSDIAVEWESFGTEWSSYKTTPPVLDSDGSFFLYSKLTVTKE | |
| | EWQQGFVFSCSVMHEALHNHYTQKSLSLSPGK | |
| 170 | APELLGGPSVFLFPPKPKDTLYITREPEVTCVVVDVSHEDPEVKFNW | Clone CH3C.35.20.1 with |
| | YVDGVEVHNAKTKPREEQYNSTYRVVSVLTVLHQDWLNGKEYKC | knob and YTE mutations |
| | KVSNKALPAPIEKTISKAKGQPREPQVYTLPPSRDELTKNQVSLWCL | |
| | VKGFYPSDIAVEWESFGTEWSSYKTTPPVLDSDGSFFLYSKLTVTKE | |
| | EWQQGFVFSCSVMHEALHNHYTQKSLSLSPGK | |
| 171 | APEAAGGPSVFLFPPKPKDTLYITREPEVTCVVVDVSHEDPEVKFNW | Clone CH3C.35.20.1 with |
| | YVDGVEVHNAKTKPREEQYNSTYRVVSVLTVLHQDWLNGKEYKC | knob, LALA, and YTE |
| | KVSNKALPAPIEKTISKAKGQPREPQVYTLPPSRDELTKNQVSLWCL | mutations |
| | VKGFYPSDIAVEWESFGTEWSSYKTTPPVLDSDGSFFLYSKLTVTKE | |
| | EWQQGFVFSCSVMHEALHNHYTQKSLSLSPGK | |
| 172 | APEAAGGPSVFLFPPKPKDTLYITREPEVTCVVVDVSHEDPEVKFNW | Clone CH3C.35.20.1 with |
| | YVDGVEVHNAKTKPREEQYNSTYRVVSVLTVLHQDWLNGKEYKC | knob, LALAPG, and YTE |
| | KVSNKALGAPIEKTISKAKGQPREPQVYTLPPSRDELTKNQVSLWCL | mutations |
| | VKGFYPSDIAVEWESFGTEWSSYKTTPPVLDSDGSFFLYSKLTVTKE | |
| | EWQQGFVFSCSVMHEALHNHYTQKSLSLSPGK | |
| 173 | APELLGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKC | hole mutations |
| | KVSNKALPAPIEKTISKAKGQPREPQVYTLPPSRDELTKNQVSLSCA | |
| | VKGFYPSDIAVEWESFGTEWSSYKTTPPVLDSDGSFFLVSKLTVTKE | |
| | EWQQGFVFSCSVMHEALHNHYTQKSLSLSPGK | |
| 174 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKC | hole and LALA mutations |
| | KVSNKALPAPIEKTISKAKGQPREPQVYTLPPSRDELTKNQVSLSCA | |
| | VKGFYPSDIAVEWESFGTEWSSYKTTPPVLDSDGSFFLVSKLTVTKE | |
| | EWQQGFVFSCSVMHEALHNHYTQKSLSLSPGK | |
| 175 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKC | hole and LALAPG |
| | KVSNKALGAPIEKTISKAKGQPREPQVYTLPPSRDELTKNQVSLSCA | mutations |
| | VKGFYPSDIAVEWESFGTEWSSYKTTPPVLDSDGSFFLVSKLTVTKE | |
| | EWQQGFVFSCSVMHEALHNHYTQKSLSLSPGK | |
| 176 | APELLGGPSVFLFPPKPKDTLYITREPEVTCVVVDVSHEDPEVKFNW | Clone CH3C.35.20.1 with |
| | YVDGVEVHNAKTKPREEQYNSTYRVVSVLTVLHQDWLNGKEYKC | hole and YTE mutations |
| | KVSNKALPAPIEKTISKAKGQPREPQVYTLPPSRDELTKNQVSLSCA | |
| | VKGFYPSDIAVEWESFGTEWSSYKTTPPVLDSDGSFFLVSKLTVTKE | |
| | EWQQGFVFSCSVMHEALHNHYTQKSLSLSPGK | |
| 177 | APEAAGGPSVFLFPPKPKDTLYITREPEVTCVVVDVSHEDPEVKFNW | Clone CH3C.35.20.1 with |
| | YVDGVEVHNAKTKPREEQYNSTYRVVSVLTVLHQDWLNGKEYKC | hole, LALA, and YTE |
| | KVSNKALPAPIEKTISKAKGQPREPQVYTLPPSRDELTKNQVSLSCA | mutations |
| | VKGFYPSDIAVEWESFGTEWSSYKTTPPVLDSDGSFFLVSKLTVTKE | |
| | EWQQGFVFSCSVMHEALHNHYTQKSLSLSPGK | |
| 178 | APEAAGGPSVFLFPPKPKDTLYITREPEVTCVVVDVSHEDPEVKFNW | Clone CH3C.35.20.1 with |
| | YVDGVEVHNAKTKPREEQYNSTYRVVSVLTVLHQDWLNGKEYKC | hole, LALAPG, and YTE |
| | KVSNKALGAPIEKTISKAKGQPREPQVYTLPPSRDELTKNQVSLSCA | mutations |
| | VKGFYPSDIAVEWESFGTEWSSYKTTPPVLDSDGSFFLVSKLTVTKE | |
| | EWQQGFVFSCSVMHEALHNHYTQKSLSLSPGK | |
| 179 | APELLGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | knob mutation |
| | KVSNKALPAPIEKTISKAKGQPREPQVYTLPPSRDELTKNQVSLWCL | |
| | VKGFYPSDIAVEWESYGTEWANYKTTPPVLDSDGSFFLYSKLTVTK | |
| | EEWQQGFVFSCSVMHEALHNHYTQKSLSLSPGK | |
| 180 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | knob and LALA mutations |
| | KVSNKALPAPIEKTISKAKGQPREPQVYTLPPSRDELTKNQVSLWCL | |
| | VKGFYPSDIAVEWESYGTIEWANYKTTPPVLDSDGSFFLYSKLTVTK | |
| | EEWQQGFVFSCSVMHEALHNHYTQKSLSLSPGK | |
| 181 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | knob and LALAPG |
| | KVSNKALGAPIEKTISKAKGQPREPQVYTLPPSRDELTKNQVSLWCL | mutations |
| | VKGFYPSDIAVEWESYGTEWANYKTTPPVLDSDGSFFLYSKLTVTK | |
| | EEWQQGFVFSCSVMHEALHNHYTQKSLSLSPGK | |
| 182 | APELLGGPSVFLFPPKPKDTLYITREPEVTCVVVDVSHEDPEVKFNW | Clone H3 35.23.2 with |
| | YVDGVEVHNAKTKPREEQYNSTYRVVSVLTVLHQDWLNGKEYKC | knob and YTE mutations |
| | KVSNKALPAPIEKTISKAKGQPREPQVYTLPPSRDELTKNQVSLWCL | |
| | VKGFYPSDIAVEWESYGTEWANYKTTPPVLDSDGSFFLYSKLTVTK | |
| | EEWQQGFVFSCSVMHEALHNHYTQKSLSLSPGK | |
| 183 | APEAAGGPSVFLFPPKPKDTLYITREPEVTCVVVDVSHEDPEVKFNW | Clone CH3C.35.23.2 with |
| | YVDGVEVHNAKTKPREEQYNSTYRVVSVLTVLHQDWLNGKEYKC | knob, LALA, and YTE |
| | KVSNKALPAPIEKTISKAKGQPREPQVYTLPPSRDELTKNQVSLWCL | mutations |
| | VKGFYPSDIAVEWESYGTEWANYKTTPPVLDSDGSFFLYSKLTVTK | |
| | EEWQQGFVFSCSVMHEALHNHYTQKSLSLSPGK | |
| 184 | APEAAGGPSVFLFPPKPKDTLYITREPEVTCVVVDVSHEDPEVKFNW | Clone CH3C.35.23.2 with |
| | YVDGVEVHNAKTKPREEQYNSTYRVVSVLTVLHQDWLNGKEYKC | knob, LALAPG, and YTE |
| | KVSNKALGAPIEKTISKAKGQPREPQVYTLPPSRDELTKNQVSLWCL | mutations |
| | VKGFYPSDIAVEWESYGTEWANYKTTPPVLDSDGSFFLYSKLTVTK | |
| | EEWQQGFVFSCSVMHEALHNHYTQKSLSLSPGK | |
| 185 | APELLGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | hole mutations |
| | KVSNKALPAPIEKTISKAKGQPREPQVYTLPPSRDELTKNQVSLSCA | |
| | VKGFYPSDIAVEWESYGTEWANYKTTPPVLDSDGSFFLVSKLTVTK | |
| | EEWQQGFVFSCSVMHEALHNHYTQKSLSLSPGK | |
| 186 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | hole and LALA mutations |
| | KVSNKALPAPIEKTISKAKGQPREPQVYTLPPSRDELTKNQVSLSCA | |
| | VKGFYPSDIAVEWESYGTEWANYKTTPPVLDSDGSFFLVSKLTVTK | |
| | EEWQQGFVFSCSVMHEALHNHYTQKSLSLSPGK | |
| 187 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | hole and LALAPG |
| | KVSNKALGAPIEKTISKAKGQPREPQVYTLPPSRDELTKNQVSLSCA | mutations |
| | VKGFYPSDIAVEWESYGTEWANYKTTPPVLDSDGSFFLVSKLTVTK | |
| | EEWQQGFVFSCSVMHEALHNHYTQKSLSLSPGK | |
| 188 | APELLGGPSVFLFPPKPKDTLYITREPEVTCVVVDVSHEDPEVKFNW | Clone CH3C.35.23.2 with |
| | YVDGVEVHNAKTKPREEQYNSTYRVVSVLTVLHQDWLNGKEYKC | hole and YTE mutations |
| | KVSNKALPAPIEKTISKAKGQPREPQVYTLPPSRDELTKNQVSLSCA | |
| | VKGFYPSDIAVEWESYGTEWANYKTTPPVLDSDGSFFLVSKLTVTK | |
| | EEWQQGFVFSCSVMHEALHNHYTQKSLSLSPGK | |
| 189 | APEAAGGPSVFLFPPKPKDTLYITREPEVTCVVVDVSHEDPEVKFNW | Clone CH3C.35.23.2 with |
| | YVDGVEVHNAKTKPREEQYNSTYRVVSVLTVLHQDWLNGKEYKC | hole, LALA, and YTE |
| | KVSNKALPAPIEKTISKAKGQPREPQVYTLPPSRDELTKNQVSLSCA | mutations |
| | VKGFYPSDIAVEWESYGTEWANYKTTPPVLDSDGSFFLVSKLTVTK | |
| | EEWQQGFVFSCSVMHEALHNHYTQKSLSLSPGK | |
| 190 | APEAAGGPSVFLFPPKPKDTLYITREPEVTCVVVDVSHEDPEVKFNW | Clone CH3C.35.23.2 with |
| | YVDGVEVHNAKTKPREEQYNSTYRVVSVLTVLHQDWLNGKEYKC | hole, LALAPG, and YTE |
| | KVSNKALGAPIEKTISKAKGQPREPQVYTLPPSRDELTKNQVSLSCA | mutations |
| | VKGFYPSDIAVEWESYGTEWANYKTTPPVLDSDGSFFLVSKLTVTK | |
| | EEWQQGFVFSCSVMHEALHNHYTQKSLSLSPGK | |
| 191 | APELLGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | knob mutation |
| | KVSNKALPAPIEKTISKAKGQPREPQVYTLPPSRDELTKNQVSLWCL | |
| | VKGFYPSDIAVEWESYGTEWVNYKTTPPVLDSDGSFFLYSKLTVTK | |
| | EEWQQGFVFSCSVMHEALHNHYTQKSLSLSPGK | |
| 192 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | knob and LALA mutations |
| | KVSNKALPAPIEKTISKAKGQPREPQVYTLPPSRDELTKNQVSLWCL | |
| | VKGFYPSDIAVEWESYGTEWVNYKTTPPVLDSDGSFFLYSKLTVTK | |
| | EEWQQGFVFSCSVMHEALHNHYTQKSLSLSPGK | |
| 193 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | knob and LALAPG |
| | KVSNKALGAPIEKTISKAKGQPREPQVYTLPPSRDELTKNQVSLWCL | mutations |
| | VKGFYPSDIAVEWESYGTEWVNYKTTPPVLDSDGSFFLYSKLTVTK | |
| | EEWQQGFVFSCSVMHEALHNHYTQKSLSLSPGK | |
| 194 | APELLGGPSVFLFPPKPKDTLYITREPEVTCVVVDVSHEDPEVKFNW | Clone CH3C.35.23.3 with |
| | YVDGVEVHNAKTKPREEQYNSTYRVVSVLTVLHQDWLNGKEYKC | knob and YTE mutations |
| | KVSNKALPAPIEKTISKAKGQPREPQVYTLPPSRDELTKNQVSLWCL | |
| | VKGFYPSDIAVEWESYGTEWVNYKTTPPVLDSDGSFFLYSKLTVTK | |
| | EEWQQGFVFSCSVMHEALHNHYTQKSLSLSPGK | |
| 195 | APEAAGGPSVFLFPPKPKDTLYITREPEVTCVVVDVSHEDPEVKFNW | Clone CH3C.35.23.3 with |
| | YVDGVEVHNAKTKPREEQYNSTYRVVSVLTVLHQDWLNGKEYKC | knob, LALA, and YTE |
| | KVSNKALPAPIEKTISKAKGQPREPQVYTLPPSRDELTKNQVSLWCL | mutations |
| | VKGFYPSDIAVEWESYGTEWVNYKTTPPVLDSDGSFFLYSKLTVTK | |
| | EEWQQGFVFSCSVMHEALHNHYTQKSLSLSPGK | |
| 196 | APEAAGGPSVFLFPPKPKDTLYITREPEVTCVVVDVSHEDPEVKFNW | Clone CH3C.35.23.3 with |
| | YVDGVEVHNAKTKPREEQYNSTYRVVSVLTVLHQDWLNGKEYKC | knob, LALAPG, and YTE |
| | KVSNKALGAPIEKTISKAKGQPREPQVYTLPPSRDELTKNQVSLWCL | mutations |
| | VKGFYPSDIAVEWESYGTEWVNYKTTPPVLDSDGSFFLYSKLTVTK | |
| | EEWQQGFVFSCSVMHEALHNHYTQKSLSLSPGK | |
| 197 | APELLGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | hole mutations |
| | KVSNKALPAPIEKTISKAKGQPREPQVYTLPPSRDELTKNQVSLSCA | |
| | VKGFYPSDIAVEWESYGTEWVNYKTTPPVLDSDGSFFLVSKLTVTK | |
| | EEWQQGFVFSCSVMHEALHNHYTQKSLSLSPGK | |
| 198 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | hole and LALA mutations |
| | KVSNKALPAPIEKTISKAKGQPREPQVYTLPPSRDELTKNQVSLSCA | |
| | VKGFYPSDIAVEWESYGTEWVNYKTTPPVLDSDGSFFLVSKLTVTK | |
| | EEWQQGFVFSCSVMHEALHNHYTQKSLSLSPGK | |
| 199 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | hole and LALAPG |
| | KVSNKALGAPIEKTISKAKGQPREPQVYTLPPSRDELTKNQVSLSCA | mutations |
| | VKGFYPSDIAVEWESYGTEWVNYKTTPPVLDSDGSFFLVSKLTVTK | |
| | EEWQQGFVFSCSVMHEALHNHYTQKSLSLSPGK | |
| 200 | APELLGGPSVFLFPPKPKDTLYITREPEVTCVVVDVSHEDPEVKFNW | Clone CH3C.35.23.3 with |
| | YVDGVEVHNAKTKPREEQYNSTYRVVSVLTVLHQDWLNGKEYKC | hole and YTE mutations |
| | KVSNKALPAPIEKTISKAKGQPREPQVYTLPPSRDELTKNQVSLSCA | |
| | VKGFYPSDIAVEWESYGTEWVNYKTTPPVLDSDGSFFLVSKLTVTK | |
| | EEWQQGFVFSCSVMHEALHNHYTQKSLSLSPGK | |
| 201 | APEAAGGPSVFLFPPKPKDTLYITREPEVTCVVVDVSHEDPEVKFNW | Clone CH3C.35.23.3 with |
| | YVDGVEVHNAKTKPREEQYNSTYRVVSVLTVLHQDWLNGKEYKC | hole, LALA, and YTE |
| | KVSNKALPAPIEKTISKAKGQPREPQVYTLPPSRDELTKNQVSLSCA | mutations |
| | VKGFYPSDIAVEWESYGTEWVNYKTTPPVLDSDGSFFLVSKLTVTK | |
| | EEWQQGFVFSCSVMHEALHNHYTQKSLSLSPGK | |
| 202 | APEAAGGPSVFLFPPKPKDTLYITREPEVTCVVVDVSHEDPEVKFNW | Clone CH3C.35.23.3 with |
| | YVDGVEVHNAKTKPREEQYNSTYRVVSVLTVLHQDWLNGKEYKC | hole, LALAPG, and YTE |
| | KVSNKALGAPIEKTISKAKGQPREPQVYTLPPSRDELTKNQVSLSCA | mutations |
| | VKGFYPSDIAVEWESYGTEWVNYKTTPPVLDSDGSFFLVSKLTVTK | |
| | EEWQQGFVFSCSVMHEALHNHYTQKSLSLSPGK | |
| 203 | APELLGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKC | knob mutation |
| | KVSNKALPAPIEKTISKAKGQPREPQVYTLPPSRDELTKNQVSLWCL | |
| | VKGFYPSDIAVEWESYGTEWSNYKTTPPVLDSDGSFFLYSKLTVSKE | |
| | EWQQGFVFSCSVMHEALHNHYTQKSLSLSPGK | |
| 204 | APEAAGGPSVFLFPPKPKDTLMISRTPEVTCVVVDVSHEDPEVKFNW | Clone H3 35.23.4 with |
| | YVDGVEVHNAKTKPREEQYNSTYRVVSVLTVLHQDWLNGKEYKC | knob and LALA mutations |
| | KVSNKALPAPIEKTISKAKGQPREPQVYTLPPSRDELTKNQVSLWCL | |
| | VKGFYPSDIAVEWESYGTEWSNYKTTPPVLDSDGSFFLYSKLTVSKE | |
| | EWQQGFVFSCSVMHEALHNHYTQKSLSLSPGK | |
| 205 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKC | knob and LALAPG |
| | KVSNKALGAPIEKTISKAKGQPREPQVYTLPPSRDELTKNQVSLWCL | mutations |
| | VKGFYPSDIAVEWESYGTEWSNYKTTPPVLDSDGSFFLYSKLTVSKE | |
| | EWQQGFVFSCSVMHEALHNHYTQKSLSLSPGK | |
| 206 | APELLGGPSVFLFPPKPKDTLYITREPEVTCVVVDVSHEDPEVKFNW | Clone CH3C.35.23.4 with |
| | YVDGVEVHNAKTKPREEQYNSTYRVVSVLTVLHQDWLNGKEYKC | knob and YTE mutations |
| | KVSNKALPAPIEKTISKAKGQPREPQVYTLPPSRDELTKNQVSLWCL | |
| | VKGFYPSDIAVEWESYGTEWSNYKTTPPVLDSDGSFFLYSKLTVSKE | |
| | EWQQGFVFSCSVMHEALHNHYTQKSLSLSPGK | |
| 207 | APEAAGGPSVFLFPPKPKDTLYITREPEVTCVVVDVSHEDPEVKFNW | Clone CH3C.35.23.4 with |
| | YVDGVEVHNAKTKPREEQYNSTYRVVSVLTVLHQDWLNGKEYKC | knob, LALA, and YTE |
| | KVSNKALPAPIEKTISKAKGQPREPQVYTLPPSRDELTKNQVSLWCL | mutations |
| | VKGFYPSDIAVEWESYGTEWSNYKTTPPVLDSDGSFFLYSKLTVSKE | |
| | EWQQGFVFSCSVMHEALHNHYTQKSLSLSPGK | |
| 208 | APEAAGGPSVFLFPPKPKDTLYITREPEVTCVVVDVSHEDPEVKFNW | Clone CH3C.35.23.4 with |
| | YVDGVEVHNAKTKPREEQYNSTYRVVSVLTVLHQDWLNGKEYKC | knob, LALAPG, and YTE |
| | KVSNKALGAPIEKTISKAKGQPREPQVYTLPPSRDELTKNQVSLWCL | mutations |
| | VKGFYPSDIAVEWESYGTEWSNYKTTPPVLDSDGSFFLYSKLTVSKE | |
| | EWQQGFVFSCSVMHEALHNHYTQKSLSLSPGK | |
| 209 | APELLGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKC | hole mutations |
| | KVSNKALPAPIEKTISKAKGQPREPQVYTLPPSRDELTKNQVSLSCA | |
| | VKGFYPSDIAVEWESYGTEWSNYKTTPPVLDSDGSFFLVSKLTVSKE | |
| | EWQQGFVFSCSVMHEALHNHYTQKSLSLSPGK | |
| 210 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKC | hole and LALA mutations |
| | KVSNKALPAPIEKTISKAKGQPREPQVYTLPPSRDELTKNQVSLSCA | |
| | VKGFYPSDIAVEWESYGTEWSNYKTTPPVLDSDGSFFLVSKLTVSKE | |
| | EWQQGFVFSCSVMHEALHNHYTQKSLSLSPGK | |
| 211 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKC | hole and LALAPG |
| | KVSNKALGAPIEKTISKAKGQPREPQVYTLPPSRDELTKNQVSLSCA | mutations |
| | VKGFYPSDIAVEWESYGTEWSNYKTTPPVLDSDGSFFLVSKLTVSKE | |
| | EWQQGFVFSCSVMHEALHNHYTQKSLSLSPGK | |
| 212 | APELLGGPSVFLFPPKPKDTLYITREPEVTCVVVDVSHEDPEVKFNW | Clone CH3C.35.23.4 with |
| | YVDGVEVHNAKTKPREEQYNSTYRVVSVLTVLHQDWLNGKEYKC | hole and YTE mutations |
| | KVSNKALPAPIEKTISKAKGQPREPQVYTLPPSRDELTKNQVSLSCA | |
| | VKGFYPSDIAVEWESYGTEWSNYKTTPPVLDSDGSFFLVSKLTVSKE | |
| | EWQQGFVFSCSVMHEALHNHYTQKSLSLSPGK | |
| 213 | APEAAGGPSVFLFPPKPKDTLYITREPEVTCVVVDVSHEDPEVKFNW | Clone CH3C.35.23.4 with |
| | YVDGVEVHNAKTKPREEQYNSTYRVVSVLTVLHQDWLNGKEYKC | hole, LALA, and YTE |
| | KVSNKALPAPIEKTISKAKGQPREPQVYTLPPSRDELTKNQVSLSCA | mutations |
| | VKGFYPSDIAVEWESYGTEWSNYKTTPPVLDSDGSFFLVSKLTVSKE | |
| | EWQQGFVFSCSVMHEALHNHYTQKSLSLSPGK | |
| 214 | APEAAGGPSVFLFPPKPKDTLYITREPEVTCVVVDVSHEDPEVKFNW | Clone CH3C.35.23.4 with |
| | YVDGVEVHNAKTKPREEQYNSTYRVVSVLTVLHQDWLNGKEYKC | hole, LALAPG, and YTE |
| | KVSNKALGAPIEKTISKAKGQPREPQVYTLPPSRDELTKNQVSLSCA | mutations |
| | VKGFYPSDIAVEWESYGTEWSNYKTTPPVLDSDGSFFLVSKLTVSKE | |
| | EWQQGFVFSCSVMHEALHNHYTQKSLSLSPGK | |
| 215 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with knob mutation |
| | KVSNKALPAPIEKTISKAKGQPREPQVYTLPPSRDELTKNQVSLWCL | |
| | VKGFYPSDIAVLWESYGTEWASYKTTPPVLDSDGSFFLYSKLTVTKE | |
| | EWQQGFVFSCSVMHEALHNHYTQKSLSLSPGK | |
| 216 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with knob and LALA |
| | KVSNKALPAPIEKTISKAKGQPREPQVYTLPPSRDELTKNQVSLWCL | mutations |
| | VKGFYPSDIAVLWESYGTEWASYKTTPPVLDSDGSFFLYSKLTVTKE | |
| | EWQQGFVFSCSVMHEALHNHYTQKSLSLSPGK | |
| 217 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with knob and LALAPG |
| | KVSNKALGAPIEKTISKAKGQPREPQVYTLPPSRDELTKNQVSLWCL | mutations |
| | VKGFYPSDIAVLWESYGTEWASYKTTPPVLDSDGSFFLYSKLTVTKE | |
| | EWQQGFVFSCSVMHEALHNHYTQKSLSLSPGK | |
| 218 | APELLGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKC | with knob and YTE |
| | KVSNKALPAPIEKTISKAKGQPREPQVYTLPPSRDELTKNQVSLWCL | mutations |
| | VKGFYPSDIAVLWESYGTEWASYKTTPPVLDSDGSFFLYSKLTVTKE | |
| | EWQQGFVFSCSVMHEALHNHYTQKSLSLSPGK | |
| 219 | APEAAGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKC | with knob, LALA, and |
| | KVSNKALPAPIEKTISKAKGQPREPQVYTLPPSRDELTKNQVSLWCL | YTE mutations |
| | VKGFYPSDIAVLWESYGTEWASYKTTPPVLDSDGSFFLYSKLTVTKE | |
| | EWQQGFVFSCSVMHEALHNHYTQKSLSLSPGK | |
| 220 | APEAAGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKC | with knob, LALAPG, and |
| | KVSNKALGAPIEKTISKAKGQPREPQVYTLPPSRDELTKNQVSLWCL | YTE mutations |
| | VKGFYPSDIAVLWESYGTEWASYKTTPPVLDSDGSFFLYSKLTVTKE | |
| | EWQQGFVFSCSVMHEALHNHYTQKSLSLSPGK | |
| 221 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with hole mutations |
| | KVSNKALPAPIEKTISKAKGQPREPQVYTLPPSRDELTKNQVSLSCA | |
| | VKGFYPSDIAVLWESYGTEWASYKTTPPVLDSDGSFFLVSKLTVTKE | |
| | EWQQGFVFSCSVMHEALHNHYTQKSLSLSPGK | |
| 222 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with hole and LALA |
| | KVSNKALPAPIEKTISKAKGQPREPQVYTLPPSRDELTKNQVSLSCA | mutations |
| | VKGFYPSDIAVLWESYGTEWASYKTTPPVLDSDGSFFLVSKLTVTKE | |
| | EWQQGFVFSCSVMHEALHNHYTQKSLSLSPGK | |
| 223 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with hole and LALAPG |
| | KVSNKALGAPIEKTISKAKGQPREPQVYTLPPSRDELTKNQVSLSCA | mutations |
| | VKGFYPSDIAVLWESYGTEWASYKTTPPVLDSDGSFFLVSKLTVTKE | |
| | EWQQGFVFSCSVMHEALHNHYTQKSLSLSPGK | |
| 224 | APELLGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVLWESYGTEWASYKTTPPVLDSDGSFFLVSKLTVTKE | |
| | EWQQGFVFSCSVMHEALHNHYTQKSLSLSPGK | |
| 225 | APEAAGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKC | with hole, LALA, and |
| | KVSNKALPAPIEKTISKAKGQPREPQVYTLPPSRDELTKNQVSLSCA | YTE mutations |
| | VKGFYPSDIAVLWESYGTEWASYKTTPPVLDSDGSFFLVSKLTVTKE | |
| | EWQQGFVFSCSVMHEALHNHYTQKSLSLSPGK | |
| 226 | APEAAGGPSVFLFPPKPKDTLYITREPEVTCVVVDVSHEDPEVKFNW | Clone CH3C.35.21.17.2 |
| | YVDGVEVHNAKTKPREEQYNSTYRVVSVLTVLHQDWLNGKEYKC | with hole, LALAPG, and |
| | KVSNKALGAPIEKTISKAKGQPREPQVYTLPPSRDELTKNQVSLSCA | YTE mutations |
| | VKGFYPSDIAVLWESYGTEWASYKTTPPVLDSDGSFFLVSKLTVTKE | |
| | EWQQGFVFSCSVMHEALHNHYTQKSLSLSPGK | |
| 227 | APELLGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | knob mutation |
| | KVSNKALPAPIEKTISKAKGQPREPQVYTLPPSRDELTKNQVSLWCL | |
| | VKGFYPSDIAVEWESYGTEWSNYKTTPPVLDSDGSFFLYSKLTVTKE | |
| | EWQQGFVFSCSVMHEALHNHYTQKSLSLSPGK | |
| 228 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | knob and LALA mutations |
| | KVSNKALPAPIEKTISKAKGQPREPQVYTLPPSRDELTKNQVSLWCL | |
| | VKGFYPSDIAVEWESYGTEWSNYKTTPPVLDSDGSFFLYSKLTVTKE | |
| | EWQQGFVFSCSVMHEALHNHYTQKSLSLSPGK | |
| 229 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | knob and LALAPG |
| | KVSNKALGAPIEKTISKAKGQPREPQVYTLPPSRDELTKNQVSLWCL | mutations |
| | VKGFYPSDIAVEWESYGTEWSNYKTTPPVLDSDGSFFLYSKLTVTKE | |
| | EWQQGFVFSCSVMHEALHNHYTQKSLSLSPGK | |
| 230 | APELLGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKC | knob and YTE mutations |
| | KVSNKALPAPIEKTISKAKGQPREPQVYTLPPSRDELTKNQVSLWCL | |
| | VKGFYPSDIAVEWESYGTEWSNYKTTPPVLDSDGSFFLYSKLTVTKE | |
| | EWQQGFVFSCSVMHEALHNHYTQKSLSLSPGK | |
| 231 | APEAAGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKC | knob, LALA, and YTE |
| | KVSNKALPAPIEKTISKAKGQPREPQVYTLPPSRDELTKNQVSLWCL | mutations |
| | VKGFYPSDIAVEWESYGTEWSNYKTTPPVLDSDGSFFLYSKLTVTKE | |
| | EWQQGFVFSCSVMHEALHNHYTQKSLSLSPGK | |
| 232 | APEAAGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKC | knob, LALAPG, and YTE |
| | KVSNKALGAPIEKTISKAKGQPREPQVYTLPPSRDELTKNQVSLWCL | mutations |
| | VKGFYPSDIAVEWESYGTEWSNYKTTPPVLDSDGSFFLYSKLTVTKE | |
| | EWQQGFVFSCSVMHEALHNHYTQKSLSLSPGK | |
| 233 | APELLGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | hole mutations |
| | KVSNKALPAPIEKTISKAKGQPREPQVYTLPPSRDELTKNQVSLSCA | |
| | VKGFYPSDIAVEWESYGTEWSNYKTTPPVLDSDGSFFLVSKLTVTKE | |
| | EWQQGFVFSCSVMHEALHNHYTQKSLSLSPGK | |
| 234 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | hole and LALA mutations |
| | KVSNKALPAPIEKTISKAKGQPREPQVYTLPPSRDELTKNQVSLSCA | |
| | VKGFYPSDIAVEWESYGTEWSNYKTTPPVLDSDGSFFLVSKLTVTKE | |
| | EWQQGFVFSCSVMHEALHNHYTQKSLSLSPGK | |
| 235 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | hole and LALAPG |
| | KVSNKALGAPIEKTISKAKGQPREPQVYTLPPSRDELTKNQVSLSCA | mutations |
| | VKGFYPSDIAVEWESYGTEWSNYKTTPPVLDSDGSFFLVSKLTVTKE | |
| | EWQQGFVFSCSVMHEALHNHYTQKSLSLSPGK | |
| 236 | APELLGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKC | hole and YTE mutations |
| | KVSNKALPAPIEKTISKAKGQPREPQVYTLPPSRDELTKNQVSLSCA | |
| | VKGFYPSDIAVEWESYGTEWSNYKTTPPVLDSDGSFFLVSKLTVTKE | |
| | EWQQGFVFSCSVMHEALHNHYTQKSLSLSPGK | |
| 237 | APEAAGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKC | hole, LALA, and YTE |
| | KVSNKALPAPIEKTISKAKGQPREPQVYTLPPSRDELTKNQVSLSCA | mutations |
| | VKGFYPSDIAVEWESYGTEWSNYKTTPPVLDSDGSFFLVSKLTVTKE | |
| | EWQQGFVFSCSVMHEALHNHYTQKSLSLSPGK | |
| 238 | APEAAGGPSVFLFPPKPKDTLYITREPEVTCVVVDVSHEDPEVKFNW | Clone CH3C.35.23 with |
| | YVDGVEVHNAKTKPREEQYNSTYRVVSVLTVLHQDWLNGKEYKC | hole, LALAPG, and YTE |
| | KVSNKALGAPIEKTISKAKGQPREPQVYTLPPSRDELTKNQVSLSCA | mutations |
| | VKGFYPSDIAVEWESYGTEWSNYKTTPPVLDSDGSFFLVSKLTVTKE | |
| | EWQQGFVFSCSVMHEALHNHYTQKSLSLSPGK | |
| 239 | APELLGGPSVFLFPPKPKDTLMISRTPEVTCVVVDVSHEDPEVKFNW | Clone CH3C.18.3.4-1 |
| | YVDGVEVHNAKTKPREEQYNSTYRVVSVLTVLHQDWLNGKEYKC | (CH3C.3.4-1) |
| | KVSNKALPAPIEKTISKAKGQPREPQVYTLPPSRDELTKNQVSLTCL | |
| | VKGFYPSDIAVEWESWGFVWSTYKTTPPVLDSDGSFFLYSKLTVPK | |
| | SNWQQGFVFSCSVMHEALHNHYTQKSLSLSPGK | |
| 240 | APELLGGPSVFLFPPKPKDTLMISRTPEVTCVVVDVSHEDPEVKFNW | Clone CH3C.18.3.4-19 |
| | YVDGVEVHNAKTKPREEQYNSTYRVVSVLTVLHQDWLNGKEYKC | (CH3C.3.4-19) |
| | KVSNKALPAPIEKTISKAKGQPREPQVYTLPPSRDELTKNQVSLTCL | |
| | VKGFYPSDIAVEWESWGHVWSTYKTTPPVLDSDGSFFLYSKLTVPK | |
| | SNWQQGYVFSCSVMHEALHNHYTQKSLSLSPGK | |
| 241 | APELLGGPSVFLFPPKPKDTLMISRTPEVTCVVVDVSHEDPEVKFNW | Clone CH3C.18.3.2-3 |
| | YVDGVEVHNAKTKPREEQYNSTYRVVSVLTVLHQDWLNGKEYKC | (CH3C.3.2-3) |
| | KVSNKALPAPIEKTISKAKGQPREPQVYTLPPSRDELTKNQVSLTCL | |
| | VKGFYPSDIAVEWESLGHVWVEQKTTPPVLDSDGSFFLYSKLTVPK | |
| | STWQQGWVFSCSVMHEALHNHYTQKSLSLSPGK | |
| 242 | APELLGGPSVFLFPPKPKDTLMISRTPEVTCVVVDVSHEDPEVKFNW | Clone CH3C.18.3.2-14 |
| | YVDGVEVHNAKTKPREEQYNSTYRVVSVLTVLHQDWLNGKEYKC | (CH3C.3.2-14) |
| | KVSNKALPAPIEKTISKAKGQPREPQVYTLPPSRDELTKNQVSLTCL | |
| | VKGFYPSDIAVEWESLGHVWVGVKTTPPVLDSDGSFFLYSKLTVPK | |
| | STWQQGWVFSCSVMHEALHNHYTQKSLSLSPGK | |
| 243 | APELLGGPSVFLFPPKPKDTLMISRTPEVTCVVVDVSHEDPEVKFNW | Clone CH3.18.3.2-24 |
| | YVDGVEVHNAKTKPREEQYNSTYRVVSVLTVLHQDWLNGKEYKC | (CH3C.3.2-24) |
| | KVSNKALPAPIEKTISKAKGQPREPQVYTLPPSRDELTKNQVSLTCL | |
| | VKGFYPSDIAVEWESLGHVWVHTKTTPPVLDSDGSFFLYSKLTVPK | |
| | STWQQGWVFSCSVMHEALHNHYTQKSLSLSPGK | |
| 244 | APELLGGPSVFLFPPKPKDTLMISRTPEVTCVVVDVSHEDPEVKFNW | Clone CH3C.18.3.4-26 |
| | YVDGVEVHNAKTKPREEQYNSTYRVVSVLTVLHQDWLNGKEYKC | (CH3C.4.4-26) |
| | KVSNKALPAPIEKTISKAKGQPREPQVYTLPPSRDELTKNQVSLTCL | |
| | VKGFYPSDIAVEWESWGTVWGTYKTTPPVLDSDGSFFLYSKLTVPK | |
| | SNWQQGYVFSCSVMHEALHNHYTQKSLSLSPGK | |
| 245 | APELLGGPSVFLFPPKPKDTLMISRTPEVTCVVVDVSHEDPEVKFNW | Clone CH3C.18.3.2-17 |
| | YVDGVEVHNAKTKPREEQYNSTYRVVSVLTVLHQDWLNGKEYKC | (CH3C.3.2-17) |
| | KVSNKALPAPIEKTISKAKGQPREPQVYTLPPSRDELTKNQVSLTCL | |
| | VKGFYPSDIAVEWESLGHVWVGTKTTPPVLDSDGSFFLYSKLTVPK | |
| | STWQQGWVFSCSVMHEALHNHYTQKSLSLSPGK | |
| 246 | APELLGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWSSYKTTPPVLDSDGSFFLYSKLTVSKE | |
| | EWQQGFVFSCSVMHEALHNHYTQKSLSLSPGK | |
| 247 | APELLGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWANYKTTPPVLDSDGSFFLYSKLTVSK | |
| | SEWQQGFVFSCSVMHEALHNHYTQKSLSLSPGK | |
| 248 | APELLGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWSNYKTTPPVLDSDGSFFLYSKLTVSKE | |
| | EWQQGFVFSCSVMHEALHNHYTQKSLSLSPGK | |
| 249 | APELLGGPSVFLFPPKPKDTLMISRTPEVTCVVVDVSHEDPEVKFNW | Clone CH3C.35.S413 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSSYKTTPPVLDSDGSFFLYSKLTVSKS | |
| | EWQQGFVFSCSVMHEALHNHYTQKSLSLSPGK | |
| 250 | APELLGGPSVFLFPPKPKDTLMISRTPEVTCVVVDVSHEDPEVKFNW | Clone CH3C.35.23.3.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWVNYKTTPPVLDSDGSFFLYSKLTVSK | |
| | EEWQQGFVFSCSVMHEALHNHYTQKSLSLSPGK | |
| 251 | APELLGGPSVFLFPPKPKDTLMISRTPEVTCVVVDVSHEDPEVKFNW | Clone CH3C.35.N390.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESYGTEWSNYKTTPPVLDSDGSFFLYSKLTVSKS | |
| | EWQQGFVFSCSVMHEALHNHYTQKSLSLSPGK | |
| 252 | APELLGGPSVFLFPPKPKDTLMISRTPEVTCVVVDVSHEDPEVKFNW | Clone CH3C.35.23.6.1 |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESFGTEWVNYKTTPPVLDSDGSFFLYSKLTVSKE | |
| | EWQQGFVFSCSVMHEALHNHYTQKSLSLSPGK | |
| 253 | APELLGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | knob mutation |
| | KVSNKALPAPIEKTISKAKGQPREPQVYTLPPSRDELTKNQVSLWCL | |
| | VKGFYPSDIAVWWESYGTEWSSYKTTPPVLDSDGSFFLYSKLTVTK | |
| | EEWQQGFVFSCSVMHEALHNHYTQKSLSLSPGK | |
| 254 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | knob and LALA mutations |
| | KVSNKALPAPIEKTISKAKGQPREPQVYTLPPSRDELTKNQVSLWCL | |
| | VKGFYPSDIAVWWESYGTEWSSYKTTPPVLDSDGSFFLYSKLTVTK | |
| | EEWQQGFVFSCSVMHEALHNHYTQKSLSLSPGK | |
| 255 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | knob and LALAPG |
| | KVSNKALGAPIEKTISKAKGQPREPQVYTLPPSRDELTKNQVSLWCL | mutations |
| | VKGFYPSDIAVWWESYGTEWSSYKTTPPVLDSDGSFFLYSKLTVTK | |
| | EEWQQGFVFSCSVMHEALHNHYTQKSLSLSPGK | |
| 256 | APELLGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKC | knob and YTE mutations |
| | KVSNKALPAPIEKTISKAKGQPREPQVYTLPPSRDELTKNQVSLWCL | |
| | VKGFYPSDIAVWWESYGTEWSSYKTTPPVLDSDGSFFLYSKLTVTK | |
| | EEWQQGFVFSCSVMHEALHNHYTQKSLSLSPGK | |
| 257 | APEAAGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKC | knob, LALA, and YTE |
| | KVSNKALPAPIEKTISKAKGQPREPQVYTLPPSRDELTKNQVSLWCL | mutations |
| | VKGFYPSDIAVWWESYGTEWSSYKTTPPVLDSDGSFFLYSKLTVTK | |
| | EEWQQGFVFSCSVMHEALHNHYTQKSLSLSPGK | |
| 258 | APEAAGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKC | knob, LALAPG, and YTE |
| | KVSNKALGAPIEKTISKAKGQPREPQVYTLPPSRDELTKNQVSLWCL | mutations |
| | VKGFYPSDIAVWWESYGTEWSSYKTTPPVLDSDGSFFLYSKLTVTK | |
| | EEWQQGFVFSCSVMHEALHNHYTQKSLSLSPGK | |
| 259 | APELLGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | hole mutations |
| | KVSNKALPAPIEKTISKAKGQPREPQVYTLPPSRDELTKNQVSLSCA | |
| | VKGFYPSDIAVWWESYGTEWSSYKTTPPVLDSDGSFFLVSKLTVTK | |
| | EEWQQGFVFSCSVMHEALHNHYTQKSLSLSPGK | |
| 260 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | hole and LALA mutations |
| | KVSNKALPAPIEKTISKAKGQPREPQVYTLPPSRDELTKNQVSLSCA | |
| | VKGFYPSDIAVWWESYGTEWSSYKTTPPVLDSDGSFFLVSKLTVTK | |
| | EEWQQGFVFSCSVMHEALHNHYTQKSLSLSPGK | |
| 261 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | hole and LALAPG |
| | KVSNKALGAPIEKTISKAKGQPREPQVYTLPPSRDELTKNQVSLSCA | mutations |
| | VKGFYPSDIAVWWESYGTEWSSYKTTPPVLDSDGSFFLVSKLTVTK | |
| | EEWQQGFVFSCSVMHEALHNHYTQKSLSLSPGK | |
| 262 | APELLGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKC | hole and YTE mutations |
| | KVSNKALPAPIEKTISKAKGQPREPQVYTLPPSRDELTKNQVSLSCA | |
| | VKGFYPSDIAVWWESYGTEWSSYKTTPPVLDSDGSFFLVSKLTVTK | |
| | EEWQQGFVFSCSVMHEALHNHYTQKSLSLSPGK | |
| 263 | APEAAGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKC | hole, LALA, and YTE |
| | KVSNKALPAPIEKTISKAKGQPREPQVYTLPPSRDELTKNQVSLSCA | mutations |
| | VKGFYPSDIAVWWESYGTEWSSYKTTPPVLDSDGSFFLVSKLTVTK | |
| | EEWQQGFVFSCSVMHEALHNHYTQKSLSLSPGK | |
| 264 | APEAAGGPSVFLFPPKPKDTLYITREPEVTCVVVDVSHEDPEVKFNW | Clone CH3C.35.21 with |
| | YVDGVEVHNAKTKPREEQYNSTYRVVSVLTVLHQDWLNGKEYKC | hole, LALAPG, and YTE |
| | KVSNKALGAPIEKTISKAKGQPREPQVYTLPPSRDELTKNQVSLSCA | mutations |
| | VKGFYPSDIAVWWESYGTEWSSYKTTPPVLDSDGSFFLVSKLTVTK | |
| | EEWQQGFVFSCSVMHEALHNHYTQKSLSLSPGK | |
| 265 | APELLGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with knob mutation |
| | KVSNKALPAPIEKTISKAKGQPREPQVYTLPPSRDELTKNQVSLWCL | |
| | VKGFYPSDIAVEWESFGTEWSSYKTTPPVLDSDGSFFLYSKLTVSKE | |
| | EWQQGFVFSCSVMHEALHNHYTQKSLSLSPGK | |
| 266 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with knob and LALA |
| | KVSNKALPAPIEKTISKAKGQPREPQVYTLPPSRDELTKNQVSLWCL | mutations |
| | VKGFYPSDIAVEWESFGTEWSSYKTTPPVLDSDGSFFLYSKLTVSKE | |
| | EWQQGFVFSCSVMHEALHNHYTQKSLSLSPGK | |
| 267 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with knob and LALAPG |
| | KVSNKALGAPIEKTISKAKGQPREPQVYTLPPSRDELTKNQVSLWCL | mutations |
| | VKGFYPSDIAVEWESFGTEWSSYKTTPPVLDSDGSFFLYSKLTVSKE | |
| | EWQQGFVFSCSVMHEALHNHYTQKSLSLSPGK | |
| 268 | APELLGGPSVFLFPPKPKDTLYITREPEVTCVVVDVSHEDPEVKFNW | Clone CH3C.35.20.1.1 |
| | YVDGVEVHNAKTKPREEQYNSTYRVVSVLTVLHQDWLNGKEYKC | with knob and YTE |
| | KVSNKALPAPIEKTISKAKGQPREPQVYTLPPSRDELTKNQVSLWCL | mutations |
| | VKGFYPSDIAVEWESFGTEWSSYKTTPPVLDSDGSFFLYSKLTVSKE | |
| | EWQQGFVFSCSVMHEALHNHYTQKSLSLSPGK | |
| 269 | APEAAGGPSVFLFPPKPKDTLYITREPEVTCVVVDVSHEDPEVKFNW | Clone CH3C.35.20.1.1 |
| | YVDGVEVHNAKTKPREEQYNSTYRVVSVLTVLHQDWLNGKEYKC | with knob, LALA, and |
| | KVSNKALPAPIEKTISKAKGQPREPQVYTLPPSRDELTKNQVSLWCL | YTE mutations |
| | VKGFYPSDIAVEWESFGTEWSSYKTTPPVLDSDGSFFLYSKLTVSKE | |
| | EWQQGFVFSCSVMHEALHNHYTQKSLSLSPGK | |
| 270 | APEAAGGPSVFLFPPKPKDTLYITREPEVTCVVVDVSHEDPEVKFNW | Clone CH3C.35.20.1.1 |
| | YVDGVEVHNAKTKPREEQYNSTYRVVSVLTVLHQDWLNGKEYKC | with knob, LALAPG, and |
| | KVSNKALGAPIEKTISKAKGQPREPQVYTLPPSRDELTKNQVSLWCL | YTE mutations |
| | VKGFYPSDIAVEWESFGTEWSSYKTTPPVLDSDGSFFLYSKLTVSKE | |
| | EWQQGFVFSCSVMHEALHNHYTQKSLSLSPGK | |
| 271 | APELLGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with hole mutations |
| | KVSNKALPAPIEKTISKAKGQPREPQVYTLPPSRDELTKNQVSLSCA | |
| | VKGFYPSDIAVEWESFGTEWSSYKTTPPVLDSDGSFFLVSKLTVSKE | |
| | EWQQGFVFSCSVMHEALHNHYTQKSLSLSPGK | |
| 272 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with hole and LALA |
| | KVSNKALPAPIEKTISKAKGQPREPQVYTLPPSRDELTKNQVSLSCA | mutations |
| | VKGFYPSDIAVEWESFGTEWSSYKTTPPVLDSDGSFFLVSKLTVSKE | |
| | EWQQGFVFSCSVMHEALHNHYTQKSLSLSPGK | |
| 273 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with hole and LALAPG |
| | KVSNKALGAPIEKTISKAKGQPREPQVYTLPPSRDELTKNQVSLSCA | mutations |
| | VKGFYPSDIAVEWESFGTEWSSYKTTPPVLDSDGSFFLVSKLTVSKE | |
| | EWQQGFVFSCSVMHEALHNHYTQKSLSLSPGK | |
| 274 | APELLGGPSVFLFPPKPKDTLYITREPEVTCVVVDVSHEDPEVKFNW | Clone CH3C.35.20.1.1 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVEWESFGTEWSSYKTTPPVLDSDGSFFLVSKLTVSKE | |
| | EWQQGFVFSCSVMHEALHNHYTQKSLSLSPGK | |
| 275 | APEAAGGPSVFLFPPKPKDTLYITREPEVTCVVVDVSHEDPEVKFNW | Clone CH3C.35.20.1.1 |
| | YVDGVEVHNAKTKPREEQYNSTYRVVSVLTVLHQDWLNGKEYKC | with hole, LALA, and |
| | KVSNKALPAPIEKTISKAKGQPREPQVYTLPPSRDELTKNQVSLSCA | YTE mutations |
| | VKGFYPSDIAVEWESFGTEWSSYKTTPPVLDSDGSFFLVSKLTVSKE | |
| | EWQQGFVFSCSVMHEALHNHYTQKSLSLSPGK | |
| 276 | APEAAGGPSVFLFPPKPKDTLYITREPEVTCVVVDVSHEDPEVKFNW | Clone CH3C.35.20.1.1 |
| | YVDGVEVHNAKTKPREEQYNSTYRVVSVLTVLHQDWLNGKEYKC | with hole, LALAPG, and |
| | KVSNKALGAPIEKTISKAKGQPREPQVYTLPPSRDELTKNQVSLSCA | YTE mutations |
| | VKGFYPSDIAVEWESFGTEWSSYKTTPPVLDSDGSFFLVSKLTVSKE | |
| | EWQQGFVFSCSVMHEALHNHYTQKSLSLSPGK | |
| 277 | APELLGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with knob mutation |
| | KVSNKALPAPIEKTISKAKGQPREPQVYTLPPSRDELTKNQVSLWCL | |
| | VKGFYPSDIAVEWESYGTEWANYKTTPPVLDSDGSFFLYSKLTVSK | |
| | SEWQQGFVFSCSVMHEALHNHYTQKSLSLSPGK | |
| 278 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with knob and LALA |
| | KVSNKALPAPIEKTISKAKGQPREPQVYTLPPSRDELTKNQVSLWCL | mutations |
| | VKGFYPSDIAVEWESYGTEWANYKTTPPVLDSDGSFFLYSKLTVSK | |
| | SEWQQGFVFSCSVMHEALHNHYTQKSLSLSPGK | |
| 279 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with knob and LALAPG |
| | KVSNKALGAPIEKTISKAKGQPREPQVYTLPPSRDELTKNQVSLWCL | mutations |
| | VKGFYPSDIAVEWESYGTEWANYKTTPPVLDSDGSFFLYSKLTVSK | |
| | SEWQQGFVFSCSVMHEALHNHYTQKSLSLSPGK | |
| 280 | APELLGGPSVFLFPPKPKDTLYITREPEVTCVVVDVSHEDPEVKFNW | Clone CH3C.35.23.2.1 |
| | YVDGVEVHNAKTKPREEQYNSTYRVVSVLTVLHQDWLNGKEYKC | with knob and YTE |
| | KVSNKALPAPIEKTISKAKGQPREPQVYTLPPSRDELTKNQVSLWCL | mutations |
| | VKGFYPSDIAVEWESYGTEWANYKTTPPVLDSDGSFFLYSKLTVSK | |
| | SEWQQGFVFSCSVMHEALHNHYTQKSLSLSPGK | |
| 281 | APEAAGGPSVFLFPPKPKDTLYITREPEVTCVVVDVSHEDPEVKFNW | Clone CH3C.35.23.2.1 |
| | YVDGVEVHNAKTKPREEQYNSTYRVVSVLTVLHQDWLNGKEYKC | with knob, LALA, and |
| | KVSNKALPAPIEKTISKAKGQPREPQVYTLPPSRDELTKNQVSLWCL | YTE mutations |
| | VKGFYPSDIAVEWESYGTEWANYKTTPPVLDSDGSFFLYSKLTVSK | |
| | SEWQQGFVFSCSVMHEALHNHYTQKSLSLSPGK | |
| 282 | APEAAGGPSVFLFPPKPKDTLYITREPEVTCVVVDVSHEDPEVKFNW | Clone CH3C.35.23.2.1 |
| | YVDGVEVHNAKTKPREEQYNSTYRVVSVLTVLHQDWLNGKEYKC | with knob, LALAPG, and |
| | KVSNKALGAPIEKTISKAKGQPREPQVYTLPPSRDELTKNQVSLWCL | YTE mutations |
| | VKGFYPSDIAVEWESYGTEWANYKTTPPVLDSDGSFFLYSKLTVSK | |
| | SEWQQGFVFSCSVMHEALHNHYTQKSLSLSPGK | |
| 283 | APELLGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with hole mutations |
| | KVSNKALPAPIEKTISKAKGQPREPQVYTLPPSRDELTKNQVSLSCA | |
| | VKGFYPSDIAVEWESYGTEWANYKTTPPVLDSDGSFFLVSKLTVSK | |
| | SEWQQGFVFSCSVMHEALHNHYTQKSLSLSPGK | |
| 284 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with hole and LALA |
| | KVSNKALPAPIEKTISKAKGQPREPQVYTLPPSRDELTKNQVSLSCA | mutations |
| | VKGFYPSDIAVEWESYGTEWANYKTTPPVLDSDGSFFLVSKLTVSK | |
| | SEWQQGFVFSCSVMHEALHNHYTQKSLSLSPGK | |
| 285 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with hole and LALAPG |
| | KVSNKALGAPIEKTISKAKGQPREPQVYTLPPSRDELTKNQVSLSCA | mutations |
| | VKGFYPSDIAVEWESYGTEWANYKTTPPVLDSDGSFFLVSKLTVSK | |
| | SEWQQGFVFSCSVMHEALHNHYTQKSLSLSPGK | |
| 286 | APELLGGPSVFLFPPKPKDTLYITREPEVTCVVVDVSHEDPEVKFNW | Clone CH3C.35.23.2.1 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVEWESYGTEWANYKTTPPVLDSDGSFFLVSKLTVSK | |
| | SEWQQGFVFSCSVMHEALHNHYTQKSLSLSPGK | |
| 287 | APEAAGGPSVFLFPPKPKDTLYITREPEVTCVVVDVSHEDPEVKFNW | Clone CH3C.35.23.2.1 |
| | YVDGVEVHNAKTKPREEQYNSTYRVVSVLTVLHQDWLNGKEYKC | with hole, LALA, and |
| | KVSNKALPAPIEKTISKAKGQPREPQVYTLPPSRDELTKNQVSLSCA | YTE mutations |
| | VKGFYPSDIAVEWESYGTEWANYKTTPPVLDSDGSFFLVSKLTVSK | |
| | SEWQQGFVFSCSVMHEALHNHYTQKSLSLSPGK | |
| 288 | APEAAGGPSVFLFPPKPKDTLYITREPEVTCVVVDVSHEDPEVKFNW | Clone CH3C.35.23.2.1 |
| | YVDGVEVHNAKTKPREEQYNSTYRVVSVLTVLHQDWLNGKEYKC | with hole, LALAPG, and |
| | KVSNKALGAPIEKTISKAKGQPREPQVYTLPPSRDELTKNQVSLSCA | YTE mutations |
| | VKGFYPSDIAVEWESYGTEWANYKTTPPVLDSDGSFFLVSKLTVSK | |
| | SEWQQGFVFSCSVMHEALHNHYTQKSLSLSPGK | |
| 289 | APELLGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with knob mutation |
| | KVSNKALPAPIEKTISKAKGQPREPQVYTLPPSRDELTKNQVSLWCL | |
| | VKGFYPSDIAVEWESFGTEWSNYKTTPPVLDSDGSFFLYSKLTVSKE | |
| | EWQQGFVFSCSVMHEALHNHYTQKSLSLSPGK | |
| 290 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with knob and LALA |
| | KVSNKALPAPIEKTISKAKGQPREPQVYTLPPSRDELTKNQVSLWCL | mutations |
| | VKGFYPSDIAVEWESFGTEWSNYKTTPPVLDSDGSFFLYSKLTVSKE | |
| | EWQQGFVFSCSVMHEALHNHYTQKSLSLSPGK | |
| 291 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with knob and LALAPG |
| | KVSNKALGAPIEKTISKAKGQPREPQVYTLPPSRDELTKNQVSLWCL | mutations |
| | VKGFYPSDIAVEWESFGTEWSNYKTTPPVLDSDGSFFLYSKLTVSKE | |
| | EWQQGFVFSCSVMHEALHNHYTQKSLSLSPGK | |
| 292 | APELLGGPSVFLFPPKPKDTLYITREPEVTCVVVDVSHEDPEVKFNW | Clone CH3C.35.23.1.1 |
| | YVDGVEVHNAKTKPREEQYNSTYRVVSVLTVLHQDWLNGKEYKC | with knob and YTE |
| | KVSNKALPAPIEKTISKAKGQPREPQVYTLPPSRDELTKNQVSLWCL | mutations |
| | VKGFYPSDIAVEWESFGTEWSNYKTTPPVLDSDGSFFLYSKLTVSKE | |
| | EWQQGFVFSCSVMHEALHNHYTQKSLSLSPGK | |
| 293 | APEAAGGPSVFLFPPKPKDTLYITREPEVTCVVVDVSHEDPEVKFNW | Clone CH3C.35.23.1.1 |
| | YVDGVEVHNAKTKPREEQYNSTYRVVSVLTVLHQDWLNGKEYKC | with knob, LALA, and |
| | KVSNKALPAPIEKTISKAKGQPREPQVYTLPPSRDELTKNQVSLWCL | YTE mutations |
| | VKGFYPSDIAVEWESFGTEWSNYKTTPPVLDSDGSFFLYSKLTVSKE | |
| | EWQQGFVFSCSVMHEALHNHYTQKSLSLSPGK | |
| 294 | APEAAGGPSVFLFPPKPKDTLYITREPEVTCVVVDVSHEDPEVKFNW | Clone CH3C.35.23.1.1 |
| | YVDGVEVHNAKTKPREEQYNSTYRVVSVLTVLHQDWLNGKEYKC | with knob, LALAPG, and |
| | KVSNKALGAPIEKTISKAKGQPREPQVYTLPPSRDELTKNQVSLWCL | YTE mutations |
| | VKGFYPSDIAVEWESFGTEWSNYKTTPPVLDSDGSFFLYSKLTVSKE | |
| | EWQQGFVFSCSVMHEALHNHYTQKSLSLSPGK | |
| 295 | APELLGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with hole mutations |
| | KVSNKALPAPIEKTISKAKGQPREPQVYTLPPSRDELTKNQVSLSCA | |
| | VKGFYPSDIAVEWESFGTEWSNYKTTPPVLDSDGSFFLVSKLTVSKE | |
| | EWQQGFVFSCSVMHEALHNHYTQKSLSLSPGK | |
| 296 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with hole and LALA |
| | KVSNKALPAPIEKTISKAKGQPREPQVYTLPPSRDELTKNQVSLSCA | mutations |
| | VKGFYPSDIAVEWESFGTEWSNYKTTPPVLDSDGSFFLVSKLTVSKE | |
| | EWQQGFVFSCSVMHEALHNHYTQKSLSLSPGK | |
| 297 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with hole and LALAPG |
| | KVSNKALGAPIEKTISKAKGQPREPQVYTLPPSRDELTKNQVSLSCA | mutations |
| | VKGFYPSDIAVEWESFGTEWSNYKTTPPVLDSDGSFFLVSKLTVSKE | |
| | EWQQGFVFSCSVMHEALHNHYTQKSLSLSPGK | |
| 298 | APELLGGPSVFLFPPKPKDTLYITREPEVTCVVVDVSHEDPEVKFNW | Clone CH3C.35.23.1.1 |
| | YVDGVEVHNAKTKPREEQYNSTYRVVSVLTVLHQDWLNGKEYKC | with hole and YTE |
| | KVSNKALPAPIEKTISKAKGQPREPQVYTLPPSRDELTKNQVSLSCA | mutations |
| | VKGFYPSDIAVEWESFGTEWSNYKTTPPVLDSDGSFFLVSKLTVSKE | |
| | EWQQGFVFSCSVMHEALHNHYTQKSLSLSPGK | |
| 299 | APEAAGGPSVFLFPPKPKDTLYITREPEVTCVVVDVSHEDPEVKFNW | Clone CH3C.35.23.1.1 |
| | YVDGVEVHNAKTKPREEQYNSTYRVVSVLTVLHQDWLNGKEYKC | with hole, LALA, and |
| | KVSNKALPAPIEKTISKAKGQPREPQVYTLPPSRDELTKNQVSLSCA | YTE mutations |
| | VKGFYPSDIAVEWESFGTEWSNYKTTPPVLDSDGSFFLVSKLTVSKE | |
| | EWQQGFVFSCSVMHEALHNHYTQKSLSLSPGK | |
| 300 | APEAAGGPSVFLFPPKPKDTLYITREPEVTCVVVDVSHEDPEVKFNW | Clone CH3C.35.23.1.1 |
| | YVDGVEVHNAKTKPREEQYNSTYRVVSVLTVLHQDWLNGKEYKC | with hole, LALAPG, and |
| | KVSNKALGAPIEKTISKAKGQPREPQVYTLPPSRDELTKNQVSLSCA | YTE mutations |
| | VKGFYPSDIAVEWESFGTEWSNYKTTPPVLDSDGSFFLVSKLTVSKE | |
| | EWQQGFVFSCSVMHEALHNHYTQKSLSLSPGK | |
| 301 | APELLGGPSVFLFPPKPKDTLMISRTPEVTCVVVDVSHEDPEVKFNW | Clone CH3C.35.20.1 |
| | YVDGVEVHNAKTKPREEQYNSTYRVVSVLTVLHQDWLNGKEYKC | M428L and N434S |
| | KVSNKALPAPIEKTISKAKGQPREPQVYTLPPSRDELTKNQVSLTCL | mutations |
| | VKGFYPSDIAVEWESFGTEWSSYKTTPPVLDSDGSFFLYSKLTVTKE | |
| | EWQQGFVFSCSVLHEALHSHYTQKSLSLSPGK | |
| 302 | APELLGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKC | knob and M428L and |
| | KVSNKALPAPIEKTISKAKGQPREPQVYTLPPSRDELTKNQVSLWCL | N434S mutations |
| | VKGFYPSDIAVEWESFGTEWSSYKTTPPVLDSDGSFFLYSKLTVTKE | |
| | EWQQGFVFSCSVLHEALHSHYTQKSLSLSPGK | |
| 303 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKC | knob, LALA, and M428L |
| | KVSNKALPAPIEKTISKAKGQPREPQVYTLPPSRDELTKNQVSLWCL | and N434S mutations |
| | VKGFYPSDIAVEWESFGTEWSSYKTTPPVLDSDGSFFLYSKLTVTKE | |
| | EWQQGFVFSCSVLHEALHSHYTQKSLSLSPGK | |
| 304 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKC | knob, LALAPG, and |
| | KVSNKALGAPIEKTISKAKGQPREPQVYTLPPSRDELTKNQVSLWCL | M428L and N434S |
| | VKGFYPSDIAVEWESFGTEWSSYKTTPPVLDSDGSFFLYSKLTVTKE | mutations |
| | EWQQGFVFSCSVLHEALHSHYTQKSLSLSPGK | |
| 305 | APELLGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKC | hole and M428L and |
| | KVSNKALPAPIEKTISKAKGQPREPQVYTLPPSRDELTKNQVSLSCA | N4345 mutations |
| | VKGFYPSDIAVEWESFGTEWSSYKTTPPVLDSDGSFFLVSKLTVTKE | |
| | EWQQGFVFSCSVLHEALHSHYTQKSLSLSPGK | |
| 306 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKC | hole, LALA, and M428L |
| | KVSNKALPAPIEKTISKAKGQPREPQVYTLPPSRDELTKNQVSLSCA | and N434S mutations |
| | VKGFYPSDIAVEWESFGTEWSSYKTTPPVLDSDGSFFLVSKLTVTKE | |
| | EWQQGFVFSCSVLHEALHSHYTQKSLSLSPGK | |
| 307 | APEAAGGPSVFLFPPKPKDTLMISRTPEVTCVVVDVSHEDPEVKFNW | Clone CH3C.35.20.1 with |
| | YVDGVEVHNAKTKPREEQYNSTYRVVSVLTVLHQDWLNGKEYKC | hole LALAPG, and |
| | KVSNKALGAPIEKTISKAKGQPREPQVYTLPPSRDELTKNQVSLSCA | M428L and N434S |
| | VKGFYPSDIAVEWESFGTEWSSYKTTPPVLDSDGSFFLVSKLTVTKE | mutations |
| | EWQQGFVFSCSVLHEALHSHYTQKSLSLSPGK | |
| 308 | APELLGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | M428L and N434S |
| | KVSNKALPAPIEKTISKAKGQPREPQVYTLPPSRDELTKNQVSLTCL | mutations |
| | VKGFYPSDIAVEWESYGTEWANYKTTPPVLDSDGSFFLYSKLTVTK | |
| | EEWQQGFVFSCSVLHEALHSHYTQKSLSLSPGK | |
| 309 | APELLGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | knob and M428L and |
| | KVSNKALPAPIEKTISKAKGQPREPQVYTLPPSRDELTKNQVSLWCL | N434S mutations |
| | VKGFYPSDIAVEWESYGTEWANYKTTPPVLDSDGSFFLYSKLTVTK | |
| | EEWQQGFVFSCSVLHEALHSHYTQKSLSLSPGK | |
| 310 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | knob, LALA, and M428L |
| | KVSNKALPAPIEKTISKAKGQPREPQVYTLPPSRDELTKNQVSLWCL | and N434S mutations |
| | VKGFYPSDIAVEWESYGTEWANYKTTPPVLDSDGSFFLYSKLTVTK | |
| | EEWQQGFVFSCSVLHEALHSHYTQKSLSLSPGK | |
| 311 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | knob, LALAPG, and |
| | KVSNKALGAPIEKTISKAKGQPREPQVYTLPPSRDELTKNQVSLWCL | M428L and N434S |
| | VKGFYPSDIAVEWESYGTEWANYKTTPPVLDSDGSFFLYSKLTVTK | mutations |
| | EEWQQGFVFSCSVLHEALHSHYTQKSLSLSPGK | |
| 312 | APELLGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | hole and M428L and |
| | KVSNKALPAPIEKTISKAKGQPREPQVYTLPPSRDELTKNQVSLSCA | N4345 mutations |
| | VKGFYPSDIAVEWESYGTEWANYKTTPPVLDSDGSFFLVSKLTVTK | |
| | EEWQQGFVFSCSVLHEALHSHYTQKSLSLSPGK | |
| 313 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | hole, LALA, and M428L |
| | KVSNKALPAPIEKTISKAKGQPREPQVYTLPPSRDELTKNQVSLSCA | and N4345 mutations |
| | VKGFYPSDIAVEWESYGTEWANYKTTPPVLDSDGSFFLVSKLTVTK | |
| | EEWQQGFVFSCSVLHEALHSHYTQKSLSLSPGK | |
| 314 | APEAAGGPSVFLFPPKPKDTLMISRTPEVTCVVVDVSHEDPEVKFNW | Clone CH3C.35.23.2 with |
| | YVDGVEVHNAKTKPREEQYNSTYRVVSVLTVLHQDWLNGKEYKC | hole LALAPG, and |
| | KVSNKALGAPIEKTISKAKGQPREPQVYTLPPSRDELTKNQVSLSCA | M428L and N434S |
| | VKGFYPSDIAVEWESYGTEWANYKTTPPVLDSDGSFFLVSKLTVTK | mutations |
| | EEWQQGFVFSCSVLHEALHSHYTQKSLSLSPGK | |
| 315 | APELLGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | M428L and N4345 |
| | KVSNKALPAPIEKTISKAKGQPREPQVYTLPPSRDELTKNQVSLTCL | mutations |
| | VKGFYPSDIAVEWESYGTEWVNYKTTPPVLDSDGSFFLYSKLTVTK | |
| | EEWQQGFVFSCSVLHEALHSHYTQKSLSLSPGK | |
| 316 | APELLGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | knob and M428L and |
| | KVSNKALPAPIEKTISKAKGQPREPQVYTLPPSRDELTKNQVSLWCL | N4345 mutations |
| | VKGFYPSDIAVEWESYGTEWVNYKTTPPVLDSDGSFFLYSKLTVTK | |
| | EEWQQGFVFSCSVLHEALHSHYTQKSLSLSPGK | |
| 317 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | knob, LALA, and M428L |
| | KVSNKALPAPIEKTISKAKGQPREPQVYTLPPSRDELTKNQVSLWCL | and N434S mutations |
| | VKGFYPSDIAVEWESYGTEWVNYKTTPPVLDSDGSFFLYSKLTVTK | |
| | EEWQQGFVFSCSVLHEALHSHYTQKSLSLSPGK | |
| 318 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | knob, LALAPG, and |
| | KVSNKALGAPIEKTISKAKGQPREPQVYTLPPSRDELTKNQVSLWCL | M428L and N434S |
| | VKGFYPSDIAVEWESYGTEWVNYKTTPPVLDSDGSFFLYSKLTVTK | mutations |
| | EEWQQGFVFSCSVLHEALHSHYTQKSLSLSPGK | |
| 319 | APELLGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | hole and M428L and |
| | KVSNKALPAPIEKTISKAKGQPREPQVYTLPPSRDELTKNQVSLSCA | N434S mutations |
| | VKGFYPSDIAVEWESYGTEWVNYKTTPPVLDSDGSFFLVSKLTVTK | |
| | EEWQQGFVFSCSVLHEALHSHYTQKSLSLSPGK | |
| 320 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | hole, LALA, and M428L |
| | KVSNKALPAPIEKTISKAKGQPREPQVYTLPPSRDELTKNQVSLSCA | and N434S mutations |
| | VKGFYPSDIAVEWESYGTEWVNYKTTPPVLDSDGSFFLVSKLTVTK | |
| | EEWQQGFVFSCSVLHEALHSHYTQKSLSLSPGK | |
| 321 | APEAAGGPSVFLFPPKPKDTLMISRTPEVTCVVVDVSHEDPEVKFNW | Clone CH3C.35.23.3 with |
| | YVDGVEVHNAKTKPREEQYNSTYRVVSVLTVLHQDWLNGKEYKC | hole LALAPG, and |
| | KVSNKALGAPIEKTISKAKGQPREPQVYTLPPSRDELTKNQVSLSCA | M428L and N4345 |
| | VKGFYPSDIAVEWESYGTEWVNYKTTPPVLDSDGSFFLVSKLTVTK | mutations |
| | EEWQQGFVFSCSVLHEALHSHYTQKSLSLSPGK | |
| 322 | APELLGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKC | M428L and N434S |
| | KVSNKALPAPIEKTISKAKGQPREPQVYTLPPSRDELTKNQVSLTCL | mutations |
| | VKGFYPSDIAVEWESYGTEWSNYKTTPPVLDSDGSFFLYSKLTVSKE | |
| | EWQQGFVFSCSVLHEALHSHYTQKSLSLSPGK | |
| 323 | APELLGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKC | knob and M428L and |
| | KVSNKALPAPIEKTISKAKGQPREPQVYTLPPSRDELTKNQVSLWCL | N434S mutations |
| | VKGFYPSDIAVEWESYGTEWSNYKTTPPVLDSDGSFFLYSKLTVSKE | |
| | EWQQGFVFSCSVLHEALHSHYTQKSLSLSPGK | |
| 324 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKC | knob, LALA, and M428L |
| | KVSNKALPAPIEKTISKAKGQPREPQVYTLPPSRDELTKNQVSLWCL | and N4345 mutations |
| | VKGFYPSDIAVEWESYGTEWSNYKTTPPVLDSDGSFFLYSKLTVSKE | |
| | EWQQGFVFSCSVLHEALHSHYTQKSLSLSPGK | |
| 325 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKC | knob, LALAPG, and |
| | KVSNKALGAPIEKTISKAKGQPREPQVYTLPPSRDELTKNQVSLWCL | M428L and N434S |
| | VKGFYPSDIAVEWESYGTEWSNYKTTPPVLDSDGSFFLYSKLTVSKE | mutations |
| | EWQQGFVFSCSVLHEALHSHYTQKSLSLSPGK | |
| 326 | APELLGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKC | hole and M428L and |
| | KVSNKALPAPIEKTISKAKGQPREPQVYTLPPSRDELTKNQVSLSCA | N4345 mutations |
| | VKGFYPSDIAVEWESYGTEWSNYKTTPPVLDSDGSFFLVSKLTVSKE | |
| | EWQQGFVFSCSVLHEALHSHYTQKSLSLSPGK | |
| 327 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKC | hole, LALA, and M428L |
| | KVSNKALPAPIEKTISKAKGQPREPQVYTLPPSRDELTKNQVSLSCA | and N4345 mutations |
| | VKGFYPSDIAVEWESYGTEWSNYKTTPPVLDSDGSFFLVSKLTVSKE | |
| | EWQQGFVFSCSVLHEALHSHYTQKSLSLSPGK | |
| 328 | APEAAGGPSVFLFPPKPKDTLMISRTPEVTCVVVDVSHEDPEVKFNW | Clone CH3C.35.23.4 with |
| | YVDGVEVHNAKTKPREEQYNSTYRVVSVLTVLHQDWLNGKEYKC | hole LALAPG, and |
| | KVSNKALGAPIEKTISKAKGQPREPQVYTLPPSRDELTKNQVSLSCA | M428L and N434S |
| | VKGFYPSDIAVEWESYGTEWSNYKTTPPVLDSDGSFFLVSKLTVSKE | mutations |
| | EWQQGFVFSCSVLHEALHSHYTQKSLSLSPGK | |
| 329 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with M428L and N434S |
| | KVSNKALPAPIEKTISKAKGQPREPQVYTLPPSRDELTKNQVSLTCL | mutations |
| | VKGFYPSDIAVLWESYGTEWASYKTTPPVLDSDGSFFLYSKLTVTKE | |
| | EWQQGFVFSCSVLHEALHSHYTQKSLSLSPGK | |
| 330 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with knob and M428L and |
| | KVSNKALPAPIEKTISKAKGQPREPQVYTLPPSRDELTKNQVSLWCL | N434S mutations |
| | VKGFYPSDIAVLWESYGTEWASYKTTPPVLDSDGSFFLYSKLTVTKE | |
| | EWQQGFVFSCSVLHEALHSHYTQKSLSLSPGK | |
| 331 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with knob, LALA, and |
| | KVSNKALPAPIEKTISKAKGQPREPQVYTLPPSRDELTKNQVSLWCL | M428L and N434S |
| | VKGFYPSDIAVLWESYGTEWASYKTTPPVLDSDGSFFLYSKLTVTKE | mutations |
| | EWQQGFVFSCSVLHEALHSHYTQKSLSLSPGK | |
| 332 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with knob, LALAPG, and |
| | KVSNKALGAPIEKTISKAKGQPREPQVYTLPPSRDELTKNQVSLWCL | M428L and N434S |
| | VKGFYPSDIAVLWESYGTEWASYKTTPPVLDSDGSFFLYSKLTVTKE | mutations |
| | EWQQGFVFSCSVLHEALHSHYTQKSLSLSPGK | |
| 333 | APELLGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with hole and M428L and |
| | KVSNKALPAPIEKTISKAKGQPREPQVYTLPPSRDELTKNQVSLSCA | N434S mutations |
| | VKGFYPSDIAVLWESYGTEWASYKTTPPVLDSDGSFFLVSKLTVTKE | |
| | EWQQGFVFSCSVLHEALHSHYTQKSLSLSPGK | |
| 334 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with hole, LALA, and |
| | KVSNKALPAPIEKTISKAKGQPREPQVYTLPPSRDELTKNQVSLSCA | M428L and N434S |
| | VKGFYPSDIAVLWESYGTEWASYKTTPPVLDSDGSFFLVSKLTVTKE | mutations |
| | EWQQGFVFSCSVLHEALHSHYTQKSLSLSPGK | |
| 335 | APEAAGGPSVFLFPPKPKDTLMISRTPEVTCVVVDVSHEDPEVKFNW | Clone CH3C.35.21.17.2 |
| | YVDGVEVHNAKTKPREEQYNSTYRVVSVLTVLHQDWLNGKEYKC | with hole, LALAPG, and |
| | KVSNKALGAPIEKTISKAKGQPREPQVYTLPPSRDELTKNQVSLSCA | M428L and N434S |
| | VKGFYPSDIAVLWESYGTEWASYKTTPPVLDSDGSFFLVSKLTVTKE | mutations |
| | EWQQGFVFSCSVLHEALHSHYTQKSLSLSPGK | |
| 336 | APELLGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | M428L and N434S |
| | KVSNKALPAPIEKTISKAKGQPREPQVYTLPPSRDELTKNQVSLTCL | mutations |
| | VKGFYPSDIAVEWESYGTEWSNYKTTPPVLDSDGSFFLYSKLTVTKE | |
| | EWQQGFVFSCSVLHEALHSHYTQKSLSLSPGK | |
| 337 | APELLGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | knob and M428L and |
| | KVSNKALPAPIEKTISKAKGQPREPQVYTLPPSRDELTKNQVSLWCL | N434S mutations |
| | VKGFYPSDIAVEWESYGTEWSNYKTTPPVLDSDGSFFLYSKLTVTKE | |
| | EWQQGFVFSCSVLHEALHSHYTQKSLSLSPGK | |
| 338 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | knob, LALA, and M428L |
| | KVSNKALPAPIEKTISKAKGQPREPQVYTLPPSRDELTKNQVSLWCL | and N434S mutations |
| | VKGFYPSDIAVEWESYGTEWSNYKTTPPVLDSDGSFFLYSKLTVTKE | |
| | EWQQGFVFSCSVLHEALHSHYTQKSLSLSPGK | |
| 339 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | knob, LALAPG, and |
| | KVSNKALGAPIEKTISKAKGQPREPQVYTLPPSRDELTKNQVSLWCL | M428L and N434S |
| | VKGFYPSDIAVEWESYGTEWSNYKTTPPVLDSDGSFFLYSKLTVTKE | mutations |
| | EWQQGFVFSCSVLHEALHSHYTQKSLSLSPGK | |
| 340 | APELLGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | hole and M428L and |
| | KVSNKALPAPIEKTISKAKGQPREPQVYTLPPSRDELTKNQVSLSCA | N434S mutations |
| | VKGFYPSDIAVEWESYGTEWSNYKTTPPVLDSDGSFFLVSKLTVTKE | |
| | EWQQGFVFSCSVLHEALHSHYTQKSLSLSPGK | |
| 341 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | hole, LALA, and M428L |
| | KVSNKALPAPIEKTISKAKGQPREPQVYTLPPSRDELTKNQVSLSCA | and N4345 mutations |
| | VKGFYPSDIAVEWESYGTEWSNYKTTPPVLDSDGSFFLVSKLTVTKE | |
| | EWQQGFVFSCSVLHEALHSHYTQKSLSLSPGK | |
| 342 | APEAAGGPSVFLFPPKPKDTLMISRTPEVTCVVVDVSHEDPEVKFNW | Clone CH3C.35.23 with |
| | YVDGVEVHNAKTKPREEQYNSTYRVVSVLTVLHQDWLNGKEYKC | hole LALAPG, and |
| | KVSNKALGAPIEKTISKAKGQPREPQVYTLPPSRDELTKNQVSLSCA | M428L and N434S |
| | VKGFYPSDIAVEWESYGTEWSNYKTTPPVLDSDGSFFLVSKLTVTKE | mutations |
| | EWQQGFVFSCSVLHEALHSHYTQKSLSLSPGK | |
| 343 | APELLGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | M428L and N4345 |
| | KVSNKALPAPIEKTISKAKGQPREPQVYTLPPSRDELTKNQVSLTCL | mutations |
| | VKGFYPSDIAVWWESYGTEWSSYKTTPPVLDSDGSFFLYSKLTVTK | |
| | EEWQQGFVFSCSVLHEALHSHYTQKSLSLSPGK | |
| 344 | APELLGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | knob and M428L and |
| | KVSNKALPAPIEKTISKAKGQPREPQVYTLPPSRDELTKNQVSLWCL | N4345 mutations |
| | VKGFYPSDIAVWWESYGTEWSSYKTTPPVLDSDGSFFLYSKLTVTK | |
| | EEWQQGFVFSCSVLHEALHSHYTQKSLSLSPGK | |
| 345 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | knob, LALA, and M428L |
| | KVSNKALPAPIEKTISKAKGQPREPQVYTLPPSRDELTKNQVSLWCL | and N4345 mutations |
| | VKGFYPSDIAVWWESYGTEWSSYKTTPPVLDSDGSFFLYSKLTVTK | |
| | EEWQQGFVFSCSVLHEALHSHYTQKSLSLSPGK | |
| 346 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | knob, LALAPG, and |
| | KVSNKALGAPIEKTISKAKGQPREPQVYTLPPSRDELTKNQVSLWCL | M428L and N434S |
| | VKGFYPSDIAVWWESYGTEWSSYKTTPPVLDSDGSFFLYSKLTVTK | mutations |
| | EEWQQGFVFSCSVLHEALHSHYTQKSLSLSPGK | |
| 347 | APELLGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | hole and M428L and |
| | KVSNKALPAPIEKTISKAKGQPREPQVYTLPPSRDELTKNQVSLSCA | N4345 mutations |
| | VKGFYPSDIAVWWESYGTEWSSYKTTPPVLDSDGSFFLVSKLTVTK | |
| | EEWQQGFVFSCSVLHEALHSHYTQKSLSLSPGK | |
| 348 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | hole, LALA, and M428L |
| | KVSNKALPAPIEKTISKAKGQPREPQVYTLPPSRDELTKNQVSLSCA | and N4345 mutations |
| | VKGFYPSDIAVWWESYGTEWSSYKTTPPVLDSDGSFFLVSKLTVTK | |
| | EEWQQGFVFSCSVLHEALHSHYTQKSLSLSPGK | |
| 349 | APEAAGGPSVFLFPPKPKDTLMISRTPEVTCVVVDVSHEDPEVKFNW | Clone CH3C.35.21 with |
| | YVDGVEVHNAKTKPREEQYNSTYRVVSVLTVLHQDWLNGKEYKC | hole LALAPG, and |
| | KVSNKALGAPIEKTISKAKGQPREPQVYTLPPSRDELTKNQVSLSCA | M428L and N434S |
| | VKGFYPSDIAVWWESYGTEWSSYKTTPPVLDSDGSFFLVSKLTVTK | mutations |
| | EEWQQGFVFSCSVLHEALHSHYTQKSLSLSPGK | |
| 350 | APELLGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with M428L and N434S |
| | KVSNKALPAPIEKTISKAKGQPREPQVYTLPPSRDELTKNQVSLTCL | mutations |
| | VKGFYPSDIAVEWESFGTEWSSYKTTPPVLDSDGSFFLYSKLTVSKE | |
| | EWQQGFVFSCSVLHEALHSHYTQKSLSLSPGK | |
| 351 | APELLGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with knob and M428L and |
| | KVSNKALPAPIEKTISKAKGQPREPQVYTLPPSRDELTKNQVSLWCL | N434S mutations |
| | VKGFYPSDIAVEWESFGTEWSSYKTTPPVLDSDGSFFLYSKLTVSKE | |
| | EWQQGFVFSCSVLHEALHSHYTQKSLSLSPGK | |
| 352 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with knob, LALA, and |
| | KVSNKALPAPIEKTISKAKGQPREPQVYTLPPSRDELTKNQVSLWCL | M428L and N434S |
| | VKGFYPSDIAVEWESFGTEWSSYKTTPPVLDSDGSFFLYSKLTVSKE | mutations |
| | EWQQGFVFSCSVLHEALHSHYTQKSLSLSPGK | |
| 353 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with knob, LALAPG, and |
| | KVSNKALGAPIEKTISKAKGQPREPQVYTLPPSRDELTKNQVSLWCL | M428L and N434S |
| | VKGFYPSDIAVEWESFGTEWSSYKTTPPVLDDGSFFLYSKLTVSKE | mutations |
| | EWQQGFVFSCSVLHEALHSHYTQKSLSLSPGK | |
| 354 | APELLGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with hole and M428L and |
| | KVSNKALPAPIEKTISKAKGQPREPQVYTLPPSRDELTKNQVSLSCA | N434S mutations |
| | VKGFYPSDIAVEWESFGTEWSSYKTTPPVLDSDGSFFLVSKLTVSKE | |
| | EWQQGFVFSCSVLHEALHSHYTQKSLSLSPGK | |
| 355 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with hole, LALA, and |
| | KVSNKALPAPIEKTISKAKGQPREPQVYTLPPSRDELTKNQVSLSCA | M428L and N434S |
| | VKGFYPSDIAVEWESFGTEWSSYKTTPPVLDSDGSFFLVSKLTVSKE | mutations |
| | EWQQGFVFSCSVLHEALHSHYTQKSLSLSPGK | |
| 356 | APEAAGGPSVFLFPPKPKDTLMISRTPEVTCVVVDVSHEDPEVKFNW | Clone CH3C.35.20.1.1 |
| | YVDGVEVHNAKTKPREEQYNSTYRVVSVLTVLHQDWLNGKEYKC | with hole, LALAPG, and |
| | KVSNKALGAPIEKTISKAKGQPREPQVYTLPPSRDELTKNQVSLSCA | M428L and N434S |
| | VKGFYPSDIAVEWESFGTEWSSYKTTPPVLDSDGSFFLVSKLTVSKE | mutations |
| | EWQQGFVFSCSVLHEALHSHYTQKSLSLSPGK | |
| 357 | APELLGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with M428L and N434S |
| | KVSNKALPAPIEKTISKAKGQPREPQVYTLPPSRDELTKNQVSLTCL | mutations |
| | VKGFYPSDIAVEWESYGTEWANYKTTPPVLDSDGSFFLYSKLTVSK | |
| | SEWQQGFVFSCSVLHEALHSHYTQKSLSLSPGK | |
| 358 | APELLGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with knob and M428L and |
| | KVSNKALPAPIEKTISKAKGQPREPQVYTLPPSRDELTKNQVSLWCL | N434S mutations |
| | VKGFYPSDIAVEWESYGTEWANYKTTPPVLDSDGSFFLYSKLTVSK | |
| | SEWQQGFVFSCSVLHEALHSHYTQKSLSLSPGK | |
| 359 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with knob, LALA, and |
| | KVSNKALPAPIEKTISKAKGQPREPQVYTLPPSRDELTKNQVSLWCL | M428L and N434S |
| | VKGFYPSDIAVEWESYGTEWANYKTTPPVLDSDGSFFLYSKLTVSK | mutations |
| | SEWQQGFVFSCSVLHEALHSHYTQKSLSLSPGK | |
| 360 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with knob, LALAPG, and |
| | KVSNKALGAPIEKTISKAKGQPREPQVYTLPPSRDELTKNQVSLWCL | M428L and N434S |
| | VKGFYPSDIAVEWESYGTEWANYKTTPPVLDSDGSFFLYSKLTVSK | mutations |
| | SEWQQGFVFSCSVLHEALHSHYTQKSLSLSPGK | |
| 361 | APELLGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with hole and M428L and |
| | KVSNKALPAPIEKTISKAKGQPREPQVYTLPPSRDELTKNQVSLSCA | N434S mutations |
| | VKGFYPSDIAVEWESYGTEWANYKTTPPVLDSDGSFFLVSKLTVSK | |
| | SEWQQGFVFSCSVLHEALHSHYTQKSLSLSPGK | |
| 362 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with hole, LALA, and |
| | KVSNKALPAPIEKTISKAKGQPREPQVYTLPPSRDELTKNQVSLSCA | M428L and N434S |
| | VKGFYPSDIAVEWESYGTEWANYKTTPPVLDSDGSFFLVSKLTVSK | mutations |
| | SEWQQGFVFSCSVLHEALHSHYTQKSLSLSPGK | |
| 363 | APEAAGGPSVFLFPPKPKDTLMISRTPEVTCVVVDVSHEDPEVKFNW | Clone CH3C.35.23.2.1 |
| | YVDGVEVHNAKTKPREEQYNSTYRVVSVLTVLHQDWLNGKEYKC | with hole, LALAPG, and |
| | KVSNKALGAPIEKTISKAKGQPREPQVYTLPPSRDELTKNQVSLSCA | mutations |
| | VKGFYPSDIAVEWESYGTEWANYKTTPPVLDSDGSFFLVSKLTVSK | M428L and N434S |
| | SEWQQGFVFSCSVLHEALHSHYTQKSLSLSPGK | |
| 364 | APELLGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with M428L and N434S |
| | KVSNKALPAPIEKTISKAKGQPREPQVYTLPPSRDELTKNQVSLTCL | mutations |
| | VKGFYPSDIAVEWESFGTEWSNYKTTPPVLDSDGSFFLYSKLTVSKE | |
| | EWQQGFVFSCSVLHEALHSHYTQKSLSLSPGK | |
| 365 | APELLGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with knob and M428L and |
| | KVSNKALPAPIEKTISKAKGQPREPQVYTLPPSRDELTKNQVSLWCL | N434S mutations |
| | VKGFYPSDIAVEWESFGTEWSNYKTTPPVLDSDGSFFLYSKLTVSKE | |
| | EWQQGFVFSCSVLHEALHSHYTQKSLSLSPGK | |
| 366 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with knob, LALA, and |
| | KVSNKALPAPIEKTISKAKGQPREPQVYTLPPSRDELTKNQVSLWCL | M428L and N434S |
| | VKGFYPSDIAVEWESFGTEWSNYKTTPPVLDSDGSFFLYSKLTVSKE | mutations |
| | EWQQGFVFSCSVLHEALHSHYTQKSLSLSPGK | |
| 367 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with knob, LALAPG, and |
| | KVSNKALGAPIEKTISKAKGQPREPQVYTLPPSRDELTKNQVSLWCL | M428L and N434S |
| | VKGFYPSDIAVEWESFGTEWSNYKTTPPVLDSDGSFFLYSKLTVSKE | mutations |
| | EWQQGFVFSCSVLHEALHSHYTQKSLSLSPGK | |
| 368 | APELLGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with hole and M428L and |
| | KVSNKALPAPIEKTISKAKGQPREPQVYTLPPSRDELTKNQVSLSCA | N434S mutations |
| | VKGFYPSDIAVEWESFGTEWSNYKTTPPVLDSDGSFFLVSKLTVSKE | |
| | EWQQGFVFSCSVLHEALHSHYTQKSLSLSPGK | |
| 369 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with hole, LALA, and |
| | KVSNKALPAPIEKTISKAKGQPREPQVYTLPPSRDELTKNQVSLSCA | M428L and N434S |
| | VKGFYPSDIAVEWESFGTEWSNYKTTPPVLDSDGSFFLVSKLTVSKE | mutations |
| | EWQQGFVFSCSVLHEALHSHYTQKSLSLSPGK | |
| 370 | APEAAGGPSVFLFPPKPKDTLMISRTPEVTCVVVDVSHEDPEVKFNW | Clone CH3C.35.23.1.1 |
| | YVDGVEVHNAKTKPREEQYNSTYRVVSVLTVLHQDWLNGKEYKC | with hole, LALAPG, and |
| | KVSNKALGAPIEKTISKAKGQPREPQVYTLPPSRDELTKNQVSLSCA | M428L and N434S |
| | VKGFYPSDIAVEWESFGTEWSNYKTTPPVLDSDGSFFLVSKLTVSKE | mutations |
| | EWQQGFVFSCSVLHEALHSHYTQKSLSLSPGK | |
| 371 | GGGGS | Linker sequence |
| 372 | GGGGSGGGGS | Linker sequence |
| 373 | GGGGSGGGGSGGGGS | Linker sequence |
| 374 | RTVAGGGGSGGGGSGGGGS | Linker sequence |
| 375 | ASTKGGGGSGGGGSGGGGS | Linker sequence |
| 376 | EPKSCDKTHTCPPCP | Human IgG1 hinge amino |
| | | acid sequence |
| 377 | APELLGGPSVFLFPPKPKDTLMISRTPEVTCVVVDVSHEDPEVKFNW | Fc sequence with hole |
| | YVDGVEVHNAKTKPREEQYNSTYRVVSVLTVLHQDWLNGKEYKC | mutations |
| | KVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDKS | |
| | RWQQGNVFSCSVMHEALHNHYTQKSLSLSPGK | |
| 378 | APEAAGGPSVFLFPPKPKDTLMISRTPEVTCVVVDVSHEDPEVKFNW | Fc sequence with hole and |
| | YVDGVEVHNAKTKPREEQYNSTYRVVSVLTVLHQDWLNGKEYKC | LALA mutations |
| | KVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDKS | |
| | RWQQGNVFSCSVMHEALHNHYTQKSLSLSPGK | |
| 379 | APELLGGPSVFLFPPKPKDTLYITREPEVTCVVVDVSHEDPEVKFNW | Fc sequence with hole and |
| | YVDGVEVHNAKTKPREEQYNSTYRVVSVLTVLHQDWLNGKEYKC | YTE mutations |
| | KVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDKS | |
| | RWQQGNVFSCSVMHEALHNHYTQKSLSLSPGK | |
| 380 | APEAAGGPSVFLFPPKPKDTLYITREPEVTCVVVDVSHEDPEVKFNW | Fc sequence with hole, |
| | YVDGVEVHNAKTKPREEQYNSTYRVVSVLTVLHQDWLNGKEYKC | LALA, and YTE |
| | KVSNKALPAPIEKTISKAKGQPREPQVYTLPPSRDELTKNQVSLSCA | mutations |
| | VKGFYPSDIAVEWESNGQPENNYKTTPPVLDSDGSFFLVSKLTVDKS | |
| | RWQQGNVFSCSVMHEALHNHYTQKSLSLSPGK | |
| 381 | APELLGGPSVFLFPPKPKDTLMISRTPEVTCVVVDVSHEDPEVKFNW | Fc sequence with hole and |
| | YVDGVEVHNAKTKPREEQYNSTYRVVSVLTVLHQDWLNGKEYKC | M428L and N434S |
| | KVSNKALPAPIEKTISKAKGQPREPQVYTLPPSRDELTKNQVSLSCA | mutations |
| | VKGFYPSDIAVEWESNGQPENNYKTTPPVLDSDGSFFLVSKLTVDKS | |
| | RWQQGNVFSCSVLHEALHSHYTQKSLSLSPGK | |
| 382 | APEAAGGPSVFLFPPKPKDTLMISRTPEVTCVVVDVSHEDPEVKFNW | Fc sequence with hole, |
| | YVDGVEVHNAKTKPREEQYNSTYRVVSVLTVLHQDWLNGKEYKC | LALA, and M428L and |
| | KVSNKALPAPIEKTISKAKGQPREPQVYTLPPSRDELTKNQVSLSCA | N434S mutations |
| | VKGFYPSDIAVEWESNGQPENNYKTTPPVLDSDGSFFLVSKLTVDKS | |
| | RWQQGNVFSCSVLHEALHSHYTQKSLSLSPGK | |
| 383 | APELLGGPSVFLFPPKPKDTLMISRTPEVTCVVVDVSHEDPEVKFNW | Fc sequence with knob |
| | YVDGVEVHNAKTKPREEQYNSTYRVVSVLTVLHQDWLNGKEYKC | mutation |
| | KVSNKALPAPIEKTISKAKGQPREPQVYTLPPSRDELTKNQVSLWCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 384 | APEAAGGPSVFLFPPKPKDTLMISRTPEVTCVVVDVSHEDPEVKFNW | Fc sequence with knob and |
| | YVDGVEVHNAKTKPREEQYNSTYRVVSVLTVLHQDWLNGKEYKC | LALA mutations |
| | KVSNKALPAPIEKTISKAKGQPREPQVYTLPPSRDELTKNQVSLWCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 385 | APELLGGPSVFLFPPKPKDTLYITREPEVTCVVVDVSHEDPEVKFNW | Fc sequence with knob and |
| | YVDGVEVHNAKTKPREEQYNSTYRVVSVLTVLHQDWLNGKEYKC | YTE mutations |
| | KVSNKALPAPIEKTISKAKGQPREPQVYTLPPSRDELTKNQVSLWCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 386 | APEAAGGPSVFLFPPKPKDTLYITREPEVTCVVVDVSHEDPEVKFNW | Fc sequence with knob, |
| | YVDGVEVHNAKTKPREEQYNSTYRVVSVLTVLHQDWLNGKEYKC | LALA, and YTE |
| | KVSNKALPAPIEKTISKAKGQPREPQVYTLPPSRDELTKNQVSLWCL | mutations |
| | VKGFYPSDIAVEWESNGQPENNYKTTPPVLDSDGSFFLYSKLTVDKS | |
| | RWQQGNVFSCSVMHEALHNHYTQKSLSLSPGK | |
| 387 | APELLGGPSVFLFPPKPKDTLMISRTPEVTCVVVDVSHEDPEVKFNW | Fc sequence with knob and |
| | YVDGVEVHNAKTKPREEQYNSTYRVVSVLTVLHQDWLNGKEYKC | M428L and N434S |
| | KVSNKALPAPIEKTISKAKGQPREPQVYTLPPSRDELTKNQVSLWCL | mutations |
| | VKGFYPSDIAVEWESNGQPENNYKTTPPVLDSDGSFFLYSKLTVDKS | |
| | RWQQGNVFSCSVLHEALHSHYTQKSLSLSPGK | |
| 388 | APEAAGGPSVFLFPPKPKDTLMISRTPEVTCVVVDVSHEDPEVKFNW | Fc sequence with knob, |
| | YVDGVEVHNAKTKPREEQYNSTYRVVSVLTVLHQDWLNGKEYKC | LALA, and M428L and |
| | KVSNKALPAPIEKTISKAKGQPREPQVYTLPPSRDELTKNQVSLWCL | N434S mutations |
| | VKGFYPSDIAVEWESNGQPENNYKTTPPVLDSDGSFFLYSKLTVDKS | |
| 389 | GGGGSGGGGSGGGG | Linker sequence |
| 390 | RTVAGGGGSGGGGSGGGGS | Linker sequence |
| 391 | ASTKGGGGSGGGGSGGGGS | Linker sequence |
| 392 | AQNSVIIVDKNGRLVYLVENPGGYVAYSKAATVTGKLVHANFGTK | Apical domain insert of |
| | KDFEDLYTPVNGSIVIVRAGKITFAEKVANAESLNAIGVLIYMDQTK | human tmnsferrin receptor |
| | FPIVNAELSFFGHAHLGTGDPYTPGFPSFNHTQFPPSRSSGLPNIPVQT | protein 1 (TFR1) |
| | ISRAAAEKLFGNMEGDCPSDWKTDSTCRMVTSESKNVKLTVSN | |
| 393 | AQNSVIIVDKNGGLVYLVENPGGYVAYSKAATVTGKLVHANFGTK | Apical domain of Macaca |
| | KDFEDLDSPVNGSIVIVRAGKITFAEKVANAESLNAIGVLIYMDQTK | mulatta (rhesus monkey) |
| | FPIVKADLSFFGHAHLGTGDPYTPGFPSFNHTQFPPSQSSGLPNIPVQT | TfR (NCBI Reference |
| | ISRAAAEKLFGNMEGDCPSDWKTDSTCKMVTSENKSVKLTVSN | Sequence |
| | | NP_001244232.1); it has |
| | | 95% identity to the apical |
| | | domain of the native |
| | | human TfR |
| 394 | AQNSVIIVDKNGSLVYLVENPGGYVAYSKAATVTGKLVHANFGTK | Apical domain of |
| | KDFEDLHTPVNGSIVIVRAGKITFAEKVANAESLNAIGVLIYMDQTK | chimpanzee TfR (NCBI |
| | FPIVNAELSFFGHAHLGTGDPYTPGFPSFNHTQFPPSRSSGLPNIPVQT | Reference Sequence |
| | VSRAAAEKLFGNMEGDCPSDWKTDSTCRMVTSESKNVKLTVSN | XP_003310238.1); it is |
| | | 98% identical to the apical |
| | | domain of the native |
| | | human TfR |
| 395 | AQNSVIIVDKNGGLVYLVENPGGYVAYSKAATVTGKLVHANFGTK | Apical domain of Macaca |
| | KDFEDLDSPVNGSIVIVRAGKITFAEKVANAESLNAIGVLIYMDQTK | fascicularis (cynomolgous |
| | FPIVKADLSFFGHAHLGTGDPYTPGFPSFNHTQFPPSQSSGLPNIPVQT | monkey) TfR (NCBI |
| | ISRAAAEKLFGNMEGDCPSDWKTDSTCKMVTSENKSVKLTVSN | Reference Sequence |
| | | XP_005545315); it is 96% |
| | | identical to the apical |
| | | domain of the native |
| | | human TfR |
| 396 | MMDQARSAFSNLFGGEPLSYTRFSLARQVDGDNSHVEMKLAADEEENA | Chimeric TfR polypeptide |
| | 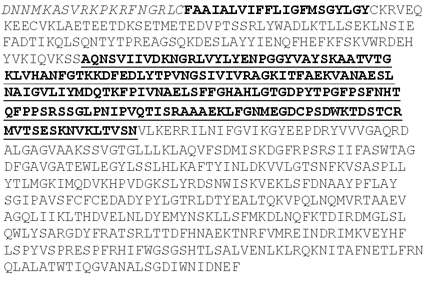 | sequence expressed in transgenic mouse (The italicized portion represents the cytoplasmic domain, the bolded portion represents the transmembrane domain, the portion in grey represents the extracellular domain, and the bold and underlined portion represents the apical domain) |
| 397 | GCTCAGAACTCCGTGATCATCGTGGATAAGAACGGCCGGCTGGT | DNA sequence of human |
| | GTACCTGGTGGAGAACCCTGGCGGATACGTGGCTTACTCTAAGG | apical domain insert |
| | CCGCTACCGTGACAGGCAAGCTGGTGCACGCCAACTTCGGAACC | |
| | AAGAAGGACTTTGAGGATCTGTACACACCAGTGAACGGCTCTAT | |
| | CGTGATCGTGCGCGCTGGAAAGATCACCTTCGCCGAGAAGGTGG | |
| | CTAACGCCGAGAGCCTGAACGCCATCGGCGTGCTGATCTACATG | |
| | GATCAGACAAAGTTTCCCATCGTGAACGCTGAGCTGTCTTTCTTT | |
| | GGACACGCTCACCTGGGCACCGGAGACCCATACACACCCGGATT | |
| | CCCTAGCTTTAACCACACCCAGTTCCCCCCTTCCAGGTCTAGCGG | |
| | ACTGCCAAACATCCCCGTGCAGACAATCAGCAGAGCCGCTGCCG | |
| | AGAAGCTGTTTGGCAACATGGAGGGAGACTGCCCCTCCGATTGG | |
| | AAGACCGACTCTACATGTAGGATGGTGACCTCCGAGTCAAAAAA | |
| 398 | MAEPRQEFEVMEDHAGTYGLGDRKDQGGYTMHQDQEGDTDAGL | full-length human Tau |
| | KESPLQTPTEDGSEEPGSETSDAKSTPTAEAEEAGIGDTPSLEDEAAG | (Tau412; 1N4R) |
| | HVTQARMVSKSKDGTGSDDKKAKGADGKTKIATPRGAAPPGQKG | |
| | QANATRIPAKTPPAPKTPPSSGEPPKSGDRSGYSSPGSPGTPGSRSRTP | |
| | SLPTPPTREPKKVAVVRTPPKSPSSAKSRLQTAPVPMPDLKNVKSKI | |
| | GSTENLKHQPGGGKVQIINKKLDLSNVQSKCGSKDNIKHVPGGGSV | |
| | QIVYKPVDLSKVTSKCGSLGNIHHKPGGGQVEVKSEKLDFKDRVQS | |
| | KIGSLDNITHVPGGGNKKIETHKLTFRENAKAKTDHGAEIVYKSPVV | |
| | SGDTSPRHLSNVSSTGSIDMVDSPQLATLADEVSASLAKQGL | |

1. A protein comprising:
(a) a first Fc polypeptide that is fused at the N-terminus to an Fd portion of a Fab that specifically binds to a first antigen;
(b) a second Fc polypeptide that is fused at the N-terminus to a single-chain variable fragment (scFv) that specifically binds to a second antigen, wherein the first and second Fc polypeptides form an Fc dimer; and
(c) a light chain polypeptide that pairs with the Fd portion to form the Fab that specifically binds to the first antigen;
wherein the first Fc polypeptide and/or the second Fc polypeptide comprises a modified CH3 domain and specifically binds to a transferrin receptor.

2. The protein of claim 1, wherein the first antigen and the second antigen are the same antigen.

3. The protein of claim 1, wherein the first antigen and the second antigen are different antigens.

4. The protein of claim 1, wherein the second Fc polypeptide is fused to the scFv via a first linker.

5. The protein of claim 4, wherein the first linker has a length from 1 to 20 amino acids.

6. The protein of claim 4, wherein the first linker comprises a GGGGS (SEQ ID NO:371; G₄S) linker, a GGGGSGGGGS (SEQ ID NO:372; (G₄S)₂) linker, a GGGGSGGGGSGGGGS (SEQ ID NO:373; (G₄S)₃) linker, or a GGGGSGGGGSGGGG (SEQ ID NO:389; (G₄S)₂-G₄) linker.

7. The protein of claim 1, wherein the scFv comprises a VL region and a VH region that are connected via a second linker, wherein the orientation of the scFv is VL region-second linker-VH region.

8. The protein of claim 1, wherein the scFv comprises a VL region and a VH region that are connected via a second linker, wherein the orientation of the scFv is VH region-second linker-VL region.

9. The protein of claim 7, wherein the second linker has a length from 10 to 25 amino acids.

10. The protein of claim 7, wherein the second linker comprises a GGGGSGGGGSGGGGS (SEQ ID NO:373; (G₄S)₃) linker, a RTVAGGGGSGGGGS (SEQ ID NO:401; RTVA(G₄S)₂) linker, a RTVAGGGGSGGGGSGGGGS (SEQ ID NO:374; RTVA(G₄S)₃) linker, a ASTKGGGGSGGGGS (SEQ ID NO:402; ASTK(G₄S)₂) linker, or a ASTKGGGGSGGGGSGGGGS (SEQ ID NO:375; ASTK(G₄S)₃) linker.

11. The protein of claim 1, wherein the scFv comprises an interchain disulfide bridge.

12. The protein of claim 1, wherein the scFv comprises a cysteine at each of positions VH44 and VL100, according to Kabat variable domain numbering.

13. The protein of claim 12, wherein the scFv comprises a disulfide bond between the cysteines at positions VH44 and VL100.

14-40. (canceled)

41. The protein of claim 1, wherein the first Fc polypeptide comprises a modified CH3 domain and specifically binds to a transferrin receptor.

42. The protein of claim 1, wherein the second Fc polypeptide comprises a modified CH3 domain and specifically binds to a transferrin receptor.

43. (canceled)

44. The protein of claim 1, wherein the first Fc polypeptide and/or the second Fc polypeptide comprises a modified CH3 domain that comprises one, two, three, four, five, six, seven, eight, nine, ten, or eleven substitutions in a set of amino acid positions comprising 380, 384, 386, 387, 388, 389, 390, 413, 415, 416, and 421, according to EU numbering.

45. The protein of claim 44, wherein the modified CH3 domain comprises Glu, Leu, Ser, Val, Trp, Tyr, or Gln at position 380; Leu, Tyr, Phe, Trp, Met, Pro, or Val at position 384; Leu, Thr, His, Pro, Asn, Val, or Phe at position 386; Val, Pro, Ile, or an acidic amino acid at position 387; Trp at position 388; an aliphatic amino acid, Gly, Ser, Thr, or Asn at position 389; Gly, His, Gln, Leu, Lys, Val, Phe, Ser, Ala, Asp, Glu, Asn, Arg, or Thr at position 390; an acidic amino acid, Ala, Ser, Leu, Thr, Pro, Ile, or His at position 413; Glu, Ser, Asp, Gly, Thr, Pro, Gln, or Arg at position 415; Thr, Arg, Asn, or an acidic amino acid at position 416; and/or an aromatic amino acid, His, or Lys at position 421, according to EU numbering.

46-53. (canceled)

54. The protein of claim 53, wherein the first Fc polypeptide and/or the second Fc polypeptide comprises 1, 2, 3, 4, 5, 6, 7, 8, 9, 10, or 11 positions selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

55. (canceled)

56. The protein of claim 54, wherein the first Fc polypeptide and/or the second Fc polypeptide has a CH3 domain with at least 85% identity, at least 90% identity, or at least 95% identity to amino acids 111-217 of any one of SEQ ID NOs:4-29, 101-164, and 239-252.

57. The protein of claim 56, wherein the first Fc polypeptide and/or the second Fc polypeptide comprises the amino acid sequence of any one of SEQ ID NOs:4-29, 101-164, and 239-252.

58. The protein of claim 56, wherein the residues for at least 5, 6, 7, 8, 9, 10, 11, 12, 13, 14, 15, or 16 of the positions corresponding to EU index positions 380, 384, 386, 387, 388, 389, 390, 391, 392, 413, 414, 415, 416, 421, 424 and 426 of any one of SEQ ID NOs:4-29, 101-164, and 239-252 are not deleted or substituted.

59. (canceled)

60. (canceled)

61. The protein of claim 1, wherein the first Fc polypeptide and the second Fc polypeptide each contain one or more modifications that promote heterodimerization.

62. (canceled)

63. The protein of claim 61, wherein the first Fc polypeptide contains the T366S, L368A, and Y407V substitutions and the second Fc polypeptide contains the T366W substitution.

64. The protein of claim 61, wherein the first Fc polypeptide contains the T366W substitution and the second Fc polypeptide contains the T366S, L368A, and Y407V substitutions.

65. The protein of claim 1, wherein the first Fc polypeptide and/or the second Fc polypeptide comprises a native FcRn binding site.

66. The protein of claim 1, wherein the first Fc polypeptide and/or the second Fc polypeptide comprises a modification that alters FcRn binding.

67. The protein of claim 1, wherein the first Fc polypeptide and/or the second Fc polypeptide comprises one or more modifications that reduce effector function.

68. The protein of claim 67, wherein the modifications that reduce effector function are substitutions of Ala at position 234 and Ala at position 235, according to EU numbering.

69. The protein of claim 67, wherein both the first Fc polypeptide and the second Fc polypeptide comprise L234A and L235A substitutions.

70. The protein of claim 1, wherein the first Fc polypeptide and/or the second Fc polypeptide comprises modifications relative to the native Fc sequence that extend serum half-life.

71. (canceled)

72. The protein of claim 70, wherein the modifications comprise substitutions of Leu at position 428 and Ser at position 434 or (ii) a substitution of Ser or Ala at position 434, according to EU numbering.

73-76. (canceled)

77. The protein of claim 1, wherein the first Fc polypeptide and/or and the second Fc polypeptide comprises an amino acid sequence of any one of SEQ ID NOs:165-238, 253-370, and 377-388.

78. A pharmaceutical composition comprising the protein of claim 1 and a pharmaceutically acceptable carrier.

79. An isolated polynucleotide comprising a nucleotide sequence encoding the protein of claim 1.

80. A vector comprising the polynucleotide of claim 79.

81. A host cell comprising the polynucleotide of claim 79.

82. A method of treating a subject, the method comprising administering to the subject the protein of claim 1.

83-93. (canceled)

94. The protein of claim 67, wherein the modifications that reduce effector function are substitutions of Ala at position 234, Ala at position 235, and Gly at position 329, according to EU numbering.
